(19) United States
(12) Patent Application Publication
(10) Pub. No.: US 2005/0107616 A1
(43) Pub. Date: May 19, 2005

(54) Alpha-ketoamide derivatives as cathepsin k inhibitors

(76) Inventors: David Gene Barrett, Hauctecht-Ulzburg (DE); David Norman Deaton, Durham, NC (US); Robert Blount Mcfadyen, Durham, NC (US); Aaron Bayne Miller, Durham, NC (US); John Albert Ray, Durham, NC (US); Francis Xavier Tavares, Durham, NC (US); Huiqiang Zhou, Durham, NC (US)

Correspondence Address: DAVID J LEVY, CORPORATE INTELLECTUAL PROPERTY;GLAXOSMITHKLINE, FIVE MOORE DR., PO BOX 13398, RESEARCH TRIANGLE PARK, NC, 27709-3398, US

(21) Appl. No.: 10/485,656

(22) PCT Filed: Jul. 23, 2002
(86) PCT No.: PCT/US02/23255

Related U.S. Application Data
(60) Provisional application No. 60/310,169, filed on Aug. 3, 2001.

(51) Int. Cl. C07D 213/55; C07D 207/46

(52) U.S. Cl. 546/335; 548/571; 558/234; 560/33

(57) ABSTRACT

Biaryl ketoamide derivatives, which are useful as cathepsin K inhibitors are described herein. The described invention also includes methods of making such biaryl ketoamide derivatives as well as methods of using the same in the treatment of disorders, including osteoporosis, associated with enhanced bone turnover which can ultimately lead to fracture.

### BACKGROUND OF THE INVENTION

The present invention relates to biaryl ketoamide derivatives, compositions and medicaments containing the same, as well as processes for the preparation and use of such compounds, compositions and medicaments. Such biaryl ketoamide derivatives are inhibitors of serine and cysteine proteases. Particularly, such biaryl ketoamide derivatives are inhibitors of cysteine proteases of the papain superfamily. More particularly, the ketoamides of the present invention are inhibitors of cathepsin family cysteine proteases such as cathepsin K. Such biaryl ketoamide derivatives are useful in the treatment of diseases associated with serine and cysteine protease activity, more particularly, in the treatment of diseases associated with cathepsin family cysteine proteases, for instance in the treatment of diseases associated with cathepsin K activity.

Osteoclasts are multinuclear cells of hematopoietic lineage, which function in the process of bone resorption. Typically, bone resorption proceeds as described following. The osteoclasts adhere to a bone surface and form a tight sealing zone. This activity is followed by extensive membrane ruffling on the surface of the osteoclasts. Such action creates an enclosed extracellular compartment on the bone surface that is acidified by proton pumps in the ruffled membrane and into which the osteoclast secretes proteolytic enzymes. The low pH of the compartment dissolves hydroxyapatite crystals at the bone surface, while the proteolytic enzymes digest the protein matrix. In this way a resorption pit is formed. At the completion of this cycle osteoblasts remodel the bone; that is, deposit a new protein matrix which is subsequently mineralized at this zone.

Normally, a balance exists between the processes of bone resorption and new bone formation during remodeling. This normal balance of bone resorption and bone formation may be disrupted resulting in a net loss of bone in each cycle of remodeling. Such net bone loss may lead to osteoporosis. Osteoporosis is characterized by reduced bone mass and disruptions in the microarchitecture of the bone. These characteristics may lead to fractures, which can result from a minimal amount of trauma. Typical sites of fractures include vertebral bodies, distal radius, and the proximal femur. However, because those suffering from osteoporosis have general skeletal weakness, fractures may occur at other sites.

Since osteoporosis is characterized by an increase in bone resorption with respect to bone remodeling, therapeutic agents that suppress bone resorption would be expected to provide a suitable treatment for osteoporosis. Administration of estrogens or calcitonin has been the bone resorption suppression treatment typically employed. However, these treatments do not always achieve the desired effect. Consequently, there is a continuing need for therapeutic agents which can attentuate bone resorption in a subject in need of such attenuation.

Cathepsin K, which has also been called cathepsin O, cathepsin O2, and cathepsin X, is a member of the cysteine cathepsin family of enzymes, which are part of the papain superfamily of cysteine proteases. Other distinct cysteine protease cathepsins, designated cathepsin B, cathepsin C, cathepsin F, cathepsin H, cathepsin L, cathepsin O, cathepsin S, cathepsin V (also called L2), cathepsin W. Et cathepsin Z (also called cathepsin X), have also been described in the literature. The Cathepsin K polypeptide and the cDNA encoding such polypeptide has been disclosed in U.S. Pat. No. 5,501,969. A crystal structure for cathepsin K has also been disclosed in PCT Patent Application WO 97/16177, published May 9, 1997. It has been reported that cathepsin K is abundantly expressed in osteoclasts under normal conditions and may be the major cysteine protease present in these cells. (See Tezuka, et al., J. Biol. Chem., 1994, 269, 1106; Inaoka, et al, Biochem. Biophys. Res. Commun., 1995, 206, 89; and Shi, et al., FEBS Lett., 1995, 357,129.) This abundant selective expression of cathepsin K in osteoclasts suggests that this enzyme is essential for bone resorption. Thus, selective inhibition of cathepsin K may provide an effective treatment for diseases of excessive bone loss, such as osteoporosis.

The selective inhibition of cathepsin K may also be useful in treating other diseases. Such disorders include autoimmune diseases such as rheumatoid arthritis, osteoarthritis, neoplastic diseases, parasitic diseases, and atherosclerosis. For instance, cathepsin K is expressed in the synovium and synovial bone destruction sites of patients with rheumatoid arthritis (see Votta, B. J. et al.; J. Bone Miner. Res. 1997, 12, 1396; Hummel, K. M. et al., J. Rheumatol. 1998, 25,1887; Nakagawa, T. Y. et al., Immunity 1999, 10, 207; Otsuka, T.et al., S. J. Antibiot. 1999, 52, 542; Li, Z.et al, Biochemistry 2000, 39, 529; Diaz, A. et al, Mol. Med. 2000, 6, 648; Moran, M. T.et al., Blood 2000, 96, 1969). Cathepsin K levels are elevated in chondroclasts of osteoarthritic synovium (See Dodds, R. A. et al., Arthritis Rheum. 1999, 42, 1588; Lang, A. et al., J. Rheumatol. 2000, 27, 1970). Neoplastic cells also have been shown to express cathepsin K (see Littlewood-Evans, A. J. et al, J. A. Cancer Res. 1997, 57, 5386; Komarova, E. A., et al., Oncogene 1998, 77, 1089; Santamaria, I., et al., Cancer Res. 1998, 58, 1624; Blagosklonny, M. V. et al., Oncogene 1999, 18, 6460; Kirschke, H.et al., Eur. J. Cancer 2000, 36, 787; Zhu, D.-M.et al., Clin. Cancer Res. 2000, 6, 2064). Cysteine protease inhibitors have been suggested as chemotherapy for parasitic diseases (see McKerrow, J. H. Int. J. Parasitol. 1999, 29, 833; Selzer, P. M.et al., Proc. Natl. Acad. Sci. U.S.A. 1999, 96, 11015; Caffrey, C. R.et al, Curr. Drug Targets 2000, 1, 155; Du, X.et al., Chem. Biol. 2000, 7, 733; Hanspal, M. Biochim. Biophys. Acta 2000, 1493, 242; Werbovetz, K. A. Curr. Med. Chem. 2000, 7, 835). Elastolytic cathepsins S and K are shown to be expressed in human atheroma (see Sukhova, G. K.et al., J. Clin. Invest. 1998, 102, 576-583; Parks, W. C. J. Clin. Invest. 1999, 104, 1167; Shi, G.-P.et al., J. Clin. Invest. 1999, 104, 1191; Cao, H.et al., J. Hum. Genet. 2000, 45, 94).

The present inventors have now discovered novel biaryl ketoamide derivative compounds, which are inhibitors of serine and cysteine protease activities, more particularly, cathepsin family cysteine protease activities, and most particularly, cathepsin K activity. Such biaryl ketoamide derivatives are useful in the treatment of disorders associated with serine and cysteine protease activity, including osteoporosis, Paget's disease, hypercalcemia of malignancy, metabolic bone disease, osteoarthritis, rheumatoid arthritis, periodontitis, gingivitis, atherosclerosis, and neoplastic diseases associated with cathepsin K activity.

### BRIEF SUMMARY OF THE INVENTION

In a first aspect of the present invention, there is provided a compound of Formula (I): 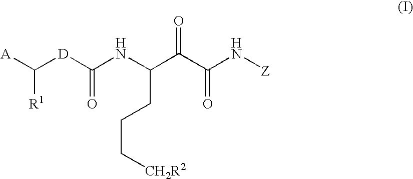
or a salt, solvate, or physiologically functional derivative thereof:
wherein
A is the group defined by (Q³)ₚ-(Q²)ₙ-(Q¹)-(Q)ₘ-, wherein
   Q is CH₂ and m is 0, 1, or 2, or
   Q is OCH₂ and m is 1, or
   Q is N(R′)CH₂ and m is 1, where R′ is hydrogen or C₁-C₆ alkyl;
   Q₁ is aryl or heteroaryl;
   Q² is CH₂ and n is 0, or 1, or
   Q² is CH₂O and n is 1, or
   Q² is N(R′) and n is 1, where R′ is hydrogen or C₁-C₆ alkyl;
   Q³ is aryl or heteroaryl and p is 0 or 1;
R¹ is C₁-C₆ alkyl, C₃-C₆ cycloalkyl or C₃-C₆ cycloalkyl substituted with C₁-C₆ alkyl;
D is O or S;
R² is hydrogen or —NR³R⁴;
R³, R⁶, and R⁷ are independently selected from hydrogen or C₁-C₆ alkyl;
R⁴ is hydrogen, C₁-C₆ alkyl, —C(O)R⁵, —C(O)OR⁵, —S(O)₂R⁵;
R⁵ is hydrogen, C₁-C₆ alkyl, or —NR⁶R⁷;
Z is the group defined by —(X)ₘ—(X¹), wherein
   X is C(R″)(R′″), wherein R″ is hydrogen or C₁-C₆ alkyl, R′″ is hydrogen and C₁-C₆ alkyl, and m is 0, 1, or 2; and
   X¹ is aryl, heteroaryl, or heterocyclyl.

In a second aspect of the present invention, there is provided a compound of Formula (II): 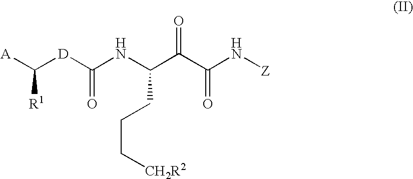
or a salt, solvate, or physiologically functional derivative thereof:
wherein
A is the group defined by (Q³)ₚ-(Q²)ₙ-(Q¹)-(Q)ₘ-, wherein
   Q is CH₂ and m is 0, 1, or 2, or
   Q is OCH₂ and m is 1, or
   Q is N(R′)CH₂ and m is 1, where R′ is hydrogen or C₁-C₆ alkyl;
   Q₁ is aryl or heteroaryl;
   Q² is CH₂ and n is 0, or 1, or
   Q² is CH₂O and n is 1, or
   Q² is N(R′) and n is 1, where R′ is hydrogen or C₁-C₆ alkyl;
   Q³ is aryl or heteroaryl and p is 0 or 1;
R¹ is C₁-C₆ alkyl, C₃-C₆ cycloalkyl or C₃-C₆ cycloalkyl substituted with C₁-C₆ alkyl;
D is O or S;
R² is hydrogen or —NR³R⁴;
R³, R⁶, and R⁷ are independently selected from hydrogen or C₁-C₆ alkyl;
R⁴ is hydrogen, C₁-C₆ alkyl, —C(O)R⁵, —C(O)OR⁵, —S(O)₂R⁵;
R⁵ is hydrogen, C₁-C₆ alkyl, or —NR⁶R⁷;
Z is the group defined by —(X)ₘ—(X¹), wherein
   X is C(R″)(R′″), wherein R″ is hydrogen or C₁-C₆ alkyl, R′″ is hydrogen and C₁-C₆ alkyl, and m is 0, 1, or 2; and
   X¹ is aryl, heteroaryl, or heterocyclyl.

In a third aspect of the present invention, there is provided a pharmaceutical composition, comprising: a therapeutically effective amount of a compound of formula (I), or a salt, solvate, or a physiologically functional derivative thereof and one or more of pharmaceutically acceptable carriers, diluents and excipients.

In a fourth aspect of the present invention, there is provided a method of treating a disorder in a mammal, said disorder being characterized by bone loss, comprising: administering to said mammal a therapeutically effective amount of a compound of formula (I) or a salt, solvate or a physiologically functional derivative thereof.

In a fifth aspect of the present invention, there is provided a compound of formula (I), or a salt, solvate, or a physiologically functional derivative thereof for use in therapy.

In a sixth aspect of the present invention, there is provided the use of a compound of formula (I), or a salt, solvate, or a physiologically functional derivative thereof in the preparation of a medicament for use in the treatment of a disorder characterized by bone loss.

In a seventh aspect of the present invention, there is provided a method of treating osteoporosis, comprising: administering to said mammal a therapeutically effective amount of a compound of formula (I), or a salt, solvate or physiologically functional derivative thereof.

In an eighth aspect of the present invention, there is provided a method of treating osteoporosis, comprising: administering to said mammal therapeutically effective amounts of (i) a compound of formula (I), or a salt, solvate or physiologically functional derivative thereof and (ii) at least one bone building agent such as parathyroid hormone (PTH).

### DETAILED DESCRIPTION OF THE INVENTION

As used herein, the term “effective amount” means that amount of a drug or pharmaceutical agent that will elicit the biological or medical response of a tissue, system, animal or human that is being sought, for instance, by a researcher or clinician. Furthermore, the term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

As used herein, the term “lower” refers to a group having between one and six carbons.

As used herein, the term “alkyl” refers to a straight or branched-chain hydrocarbon having from one to twelve carbon atoms, optionally substituted with substituents selected from the group consisting of lower alkyl, lower alkoxy, lower alkylsulfanyl, lower alkylsulfenyl, lower alkylsulfonyl, oxo, mercapto, amino optionally substituted by alkyl, carboxy, carbamoyl optionally substituted by alkyl, aminosulfonyl optionally substituted by alkyl, nitro, or lower perfluoroalkyl, multiple degrees of substitution being allowed. Examples of “alkyl” as used herein include, but are not limited to, methyl, ethyl, n-propyl, n-butyl, n-pentyl, isobutyl, isopropyl, and the like.

As used herein, the terms “C₁-C₂ alkyl” and “C₁-C₆ alkyl” refer to an alkyl group, as defined above, which contains at least 1, and at most 2 or 6, carbon atoms. Examples of “C₁-C₂ alkyl” and “C₁-C₆ alkyl” groups useful in the present invention include, but are not limited to, methyl, ethyl, propyl, isopropyl, isobutyl, n-butyl, tert-butyl, and isopentyl.

As used herein, the term “alkylene” refers to a straight or branched-chain divalent hydrocarbon radical having from one to ten carbon atoms, and being optionally substituted with substituents selected from the group which includes lower alkyl, lower alkoxy, lower alkylsulfanyl, lower alkylsulfenyl, lower alkylsulfonyl, oxo, hydroxy, mercapto, amino optionally substituted by alkyl, carboxy, carbamoyl optionally substituted by alkyl, aminosulfonyl optionally substituted by alkyl, nitro, cyano, halogen and lower perfluoroalkyl, multiple degrees of substitution being allowed. Examples of “alkylene” as used herein include, but are not limited to, methylene, ethylene, n-propylene, n-butylene, and the like.

As used herein, the terms “C₁-C₃ alkylene” and “C₁-C₄ alkylene” refer to an alkylene group, as defined above, which contains at least 1, and at most 3 or 4, carbon atoms respectively. Examples of “C₁-C₃ alkylene” groups useful in the present invention include, but are not limited to, methylene, ethylene, and n-propylene.

As used herein, the term “halogen” refers to fluorine, chlorine, bromine, or iodine and the term “halo” refers to fluoro (—F), chloro (—Cl), bromo (—Br), and iodo (—I).

As used herein, the term “haloalkyl” refers to an alkyl group, as defined herein, substituted with at least one halogen, halogen being as defined herein. Examples of branched or straight chained “haloalkyl” groups useful in the present invention include, but are not limited to, methyl, ethyl, propyl, isopropyl, n-butyl, and t-butyl substituted independently with one or more halo groups, e.g., fluoro, chloro, bromo and iodo.

As used herein, the terms “C₁-C₂ haloalkyl” and “C₁-C₃ haloalkyl” refer to haloalkyl as defined above containing at least 1, and at most 2 or 3 carbon atoms substituted with at least one halogen, halogen being as defined herein. Examples of branched or straight chained “C₁-C₂ haloalkyl” and “C₁-C₃ haloalkyl” groups useful in the present invention include, but are not limited to methyl, ethyl, propyl, and isopropyl, substituted independently with one or more halo groups, e.g., fluoro, chloro, bromo, and iodo.

As used herein, the term “C₃-C₆ cycloalkyl” refers to a non-aromatic cyclic hydrocarbon ring having from three to six carbon atoms, which optionally includes a C₁-C₄ alkylene linker through which it may be attached. Exemplary “C₃-C₆ cycloalkyl” groups include, but are not limited to cyclopropyl, cyclobutyl, cyclopentyl, and cyclohexyl.

As used herein, the term “aryl” refers to an optionally substituted benzene ring or to an optionally substituted benzene ring system fused to one or more optionally substituted benzene rings to form, for example, anthracene, phenanthrene, or napthalene ring systems. Exemplary optional substituents include lower alkyl, C₃-C₇ cycloalkyl, lower haloakyl, lower alkoxy, lower alkylsulfanyl, lower alkylsulfenyl, lower alkylsulfonyl, oxo, hydroxy, mercapto, amino optionally substituted by alkyl, carboxy, tetrazolyl, carbamoyl optionally substituted by alkyl, aminosulfonyl optionally substituted by alkyl, acyl, aroyl, heteroaroyl, acyloxy, aroyloxy, heteroaroyloxy, alkoxycarbonyl, nitro, cyano, halogen, lower perfluoroalkyl, heteroaryl, or aryl, multiple degrees of substitution being allowed. Examples of “aryl” groups include, but are not limited to, phenyl, 2-naphthyl, 1-naphthyl, and biphenyl, as well as substituted derivatives thereof.

As used herein, the term “aralkyl” refers to an aryl or heteroaryl group, as defined herein, attached through a lower alkylene linker, wherein the lower alkylene is as defined herein. Examples of “aralkyl” include, but are not limited to benzyl, phenylpropyl, 2-pyridylmethyl, 3-isoxazolylmethyl, 5-methyl-3-isoxazolylmethyl, and 2-imidazoyly ethyl.

As used herein, the term “arylamino” refers to an aryl or heteroaryl group, as defined herein, attached through an amino group —NR′—, wherein R′ is as defined herein.

As used herein, the term “heteroaryl” refers to a monocyclic five to seven membered aromatic ring, or to a fused bicyclic aromatic ring system comprising two of such monocyclic five to seven membered aromatic rings. These heteroaryl rings contain one or more nitrogen, sulfur, and/or oxygen atoms, where N-oxides and sulfur oxides and dioxides are permissible heteroatom substitutions and may be optionally substituted with up to three members selected from a group consisting of lower alkyl, lower haloalkyl, C₃-C₇ cycloalkyl, lower alkoxy, lower alkylsulfanyl, lower alkylsulfenyl, lower alkylsulfonyl, oxo, hydroxy, mercapto, amino optionally substituted by alkyl, carboxy, tetrazolyl, carbamoyl optionally substituted by alkyl, aminosulfonyl optionally substituted by alkyl, acyl, aroyl, heteroaroyl, acyloxy, aroyloxy, heteroaroyloxy, alkoxycarbonyl, nitro, cyano, halogen, lower perfluoroalkyl, heteroaryl, or aryl, multiple degrees of substitution being allowed. Examples of “heteroaryl” groups used herein include furan, thiophene, pyrrole, imidazole, pyrazole, triazole, tetrazole, thiazole, oxazole, isoxazole, oxadiazole, thiadiazole, isothiazole, pyridine, pyridazine, pyrazine, pyrimidine, quinoline, isoquinoline, benzofuran, benzothiophene, indole, indazole, and substituted versions thereof.

As used herein, the term “heterocyclic” or the term “heterocyclyl” refers to a three to twelve-membered non-aromatic heterocyclic ring being saturated or having one or more degrees of unsaturation containing one or more heteroatomic substitutions selected from S, S(O), S(O)₂, O, or N, optionally substituted with substituents selected from the group consisting of lower alkyl, lower haloalkyl, lower alkoxy, lower alkylsulfanyl, lower alkylsulfenyl, lower alkylsulfonyl, oxo, hydroxy, mercapto, amino optionally substituted by alkyl, carboxy, carbamoyl optionally substituted by alkyl, aminosulfonyl optionally substituted by alkyl, nitro, cyano, halogen, or lower perfluoroalkyl, multiple degrees of substitution being allowed. Such a ring may be optionally fused to one or more of another “heterocyclic” ring(s) or cycloalkyl ring(s). Examples of “heterocyclic” include, but are not limited to, tetrahydrofuran, pyran, 1,4-dioxane, 1,3-dioxane, piperidine, pyrrolidine, morpholine, tetrahydrothiopyran, tetrahydrothiophene, and the like.

As used herein the term “heteroarylalkyl” refers to a heteroaryl group as described above substituted with an alkyl group containing the specified number of carbon atoms. The “heteroarylalkyl” group may be optionally substituted with up to three members selected from a group consisting of lower alkyl, lower alkoxy, lower alkylsulfanyl, lower alkylsulfenyl, lower alkylsulfonyl, oxo, hydroxy, mercapto, amino optionally substituted by alkyl, carboxy, tetrazolyl, carbamoyl optionally substituted by alkyl, aminosulfonyl optionally substituted by alkyl, acyl, aroyl, heteroaroyl, acyloxy, aroyloxy, heteroaroyloxy, alkoxycarbonyl, nitro, cyano, halogen, lower perfluoroalkyl, heteroaryl, or aryl, multiple degrees of substitution being allowed. An example of a “heteroarylalkyl” as used herein includes, but is not limited to, 4-pyridinylmethyl.

As used herein, the term “alkoxy” refers to the group RₐO—, where Rₐ is alkyl as defined above and the term “C₁-C₂ alkoxy” refers to the group RₐO—, where Rₐ is C₁-C₂ alkyl.

As used herein, the term “haloalkoxy” refers to the group RₐO—, where Rₐ is haloalkyl as defined above and the term “C₁-C₂ haloalkoxy” refers to the group RₐO—, where Rₐ is C₁-C₂ haloalkyl as defined above.

As used herein the term “aralkoxy” refers to the group R_{b}RₐO—, where Rₐ is alkylene and R_{b} is aryl, both as defined above.

As used herein, the term “alkylsulfanyl” refers to the group RₐS', where Rₐ is alkyl as defined above.

As used herein, the term “alkylsulfenyl” refers to the group RₐS(O)—, where Rₐ is alkyl as defined above.

As used herein, the term “alkylsulfonyl” refers to the group RₐS(O)₂—, where Rₐ is alkyl as defined above.

As used herein, the term “oxo” refers to the group ═O

As used herein, the term “mercapto” refers to the group —SH.

As used herein, the term “carboxy” refers to the group —COOH.

As used herein, the term “cyano” refers to the group —CN.

As used herein the term “cyanoalkyl” refers to the group —RₐCN wherein Rₐ is C₁-C₃ alkylene as defined above. Exemplary “cyanoalkyl” groups useful in the present invention include, but are not limited to cyanomethyl, cyanoethyl, and cyanopropyl.

As used herein, the term “aminosulfonyl” refers to the group —S(O)₂NH₂.

As used herein, the term “carbamoyl” refers to the group —C(O)NH₂.

As used herein, the term “sulfanyl” shall refer to the group —S—.

As used herein, the term “sulfenyl” shall refer to the group —S(O)—.

As used herein, the term “sulfonyl” shall refer to the group —S(O)₂—.

As used herein, the term “acyl” refers to the group RₐC(O)—, where Rₐ is alkyl, cycloalkyl, or heterocyclyl as defined herein.

As used herein, the term “aroyl” refers to the group RₐC(O)—, where Rₐ is aryl as defined herein.

As used herein, the term “heteroaroyl” refers to the group RₐC(O)—, where Rₐ is heteroaryl as defined herein.

As used herein, the term “alkoxycarbonyl” refers to the group RₐOC(O)—, where Rₐ is alkyl as defined herein.

As used herein, the term “acyloxy” refers to the group RₐC(O)O—, where Rₐ is alkyl, cycloalkyl, or heterocyclyl as defined herein.

As used herein, the term “aroyloxy” refers to the group RₐC(O)O—, where Rₐ is aryl as defined herein.

As used herein, the term “heteroaroyloxy” refers to the group RₐC(O)O—, where Rₐ is heteroaryl as defined herein.

As used herein, the term “optionally” means that the subsequently described event(s) may or may not occur, and includes both event(s) that occur, and events that do not occur.

As used herein, the term “physiologically functional derivative” refers to any pharmaceutically acceptable derivative of a compound of the present invention; for example, an ester or an amide, which upon administration to a mammal is capable of providing (directly or indirectly) a compound of the present invention or an active metabolite thereof. Such derivatives are clear to those skilled in the art, without undue experimentation, and with reference to the teaching of Burger's Medicinal Chemistry And Drug Discovery, 5^{th} Edition, Vol 1: Principles and Practice, which is incorporated herein by reference to the extent that it teaches physiologically functional derivatives.

As used herein, the term “solvate” refers to a complex of variable stoichiometry formed by a solute (in this invention, a compound of formula (I)), or a salt or physiologically functional derivative thereof) and a solvent. Such solvents for the purpose of the invention may not interfere with the biological activity of the solute. Examples of suitable solvents include, but are not limited to water, methanol, ethanol, and acetic acid. Preferably the solvent used is a pharmaceutically acceptable solvent. Examples of suitable pharmaceutically acceptable solvents include water, ethanol, and acetic acid. Most preferably the solvent used is water.

The compounds of formula (I) have the ability to crystallize in more than one form, a characteristic known as polymorphism, and it is understood that such polymorphic forms (“polymorphs”) are within the scope of formula (I). Polymorphism generally can occur as a response to changes in temperature or pressure or both and can also result from variations in the crystallization process. Polymorphs can be distinguished by various physical characteristics known in the art such as x-ray diffraction patterns, solubility, and melting point

As used herein, the term “substituted” refers to substitution with the named substituent or substituents, multiple degrees of substitution being allowed unless otherwise stated.

Certain of the compounds described herein contain one or more chiral centers, or may otherwise be capable of existing as multiple stereoisomers. The compounds of this invention include mixtures of stereoisomers as well as purified enantiomers or enantiomerically or diastereomerically enriched mixtures. Also included within the scope of the invention are the individual isomers of the compounds represented by formula (I) above as well as any wholly or partially equilibrated mixtures thereof. The present invention also covers the individual isomers of the compounds represented by the formulas above as mixtures with isomers thereof in which one or more chiral centers are inverted.

It is to be understood that the following embodiments refer to compounds within the scope of both formula (I) and formula (II) as defined above unless specifically limited by the definition of each formula or specifically limited otherwise. It is also understood that the embodiments of the present invention described herein, including uses and compositions, are applicable to both formula (I) and formula (II).

As recited above A is the group defined by (Q³)ₚ-(Q²)ₙ-(Q¹)-(Q)ₘ-. In one embodiment, n is 0 and A is (Q³)ₚ-(Q¹)-(Q)ₘ-. In another embodiment, m is 0 and A is (Q³)ₚ-(Q²)ₙ-(Q¹)-. In a further embodiment, m and n are both 0 and A is (Q³)ₚ-(Q¹)-. In an alternative embodiment, p and n are both 0 and A is (Q¹)-(Q)ₘ-.

In one embodiment, Q is CH₂ and m is 0, 1, or 2, preferably m is 0 or 1, more preferably m is 1. In another embodiment, Q is OCH₂ and m is 1. In a further embodiment, Q is N(R′)CH₂ and m is 1, where R′ is hydrogen or C₁-C₆ alkyl.

In one embodiment Q¹ is aryl. In a preferred embodiment Q¹ is selected from the group

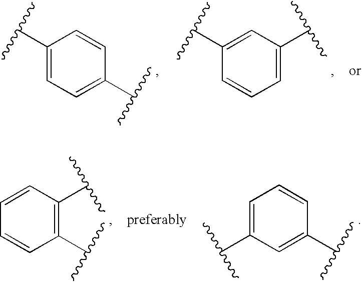

In another embodiment, Q¹ is selected from the group 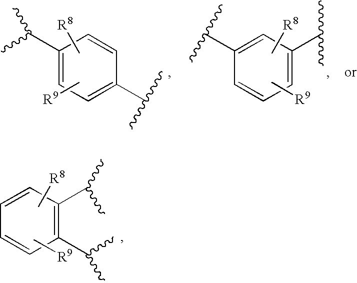
wherein R⁸ and R⁹ are independently selected from hydrogen, halogen, preferably flourine or chlorine, or C₁-C₃ haloalkyl, preferably trifluoromethyl. More preferably, R⁸ and R⁹ is hydrogen and the other is flourine or trifluoromethyl.

In another embodiment, Q¹ is heteroaryl. In a preferred embodiment Q¹ is selected from

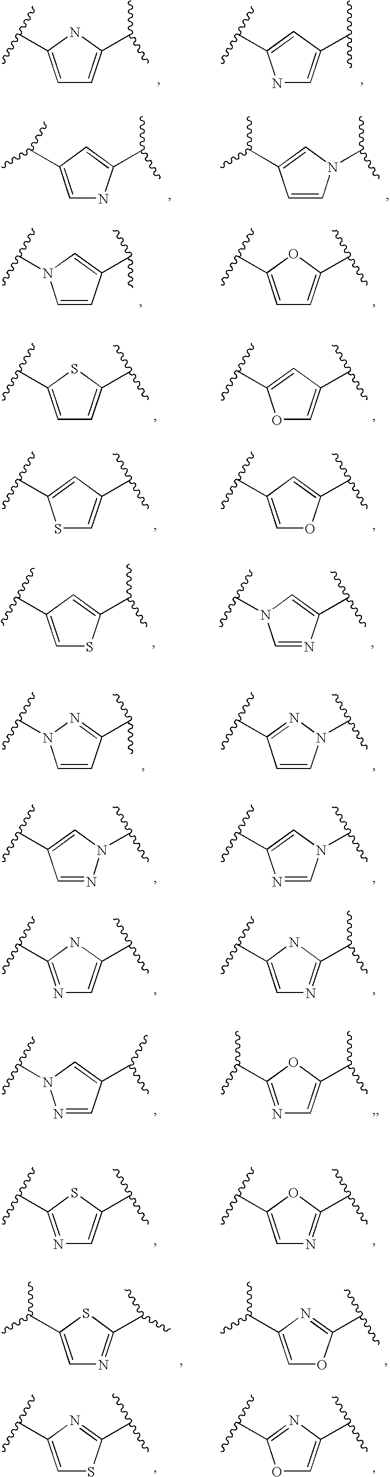

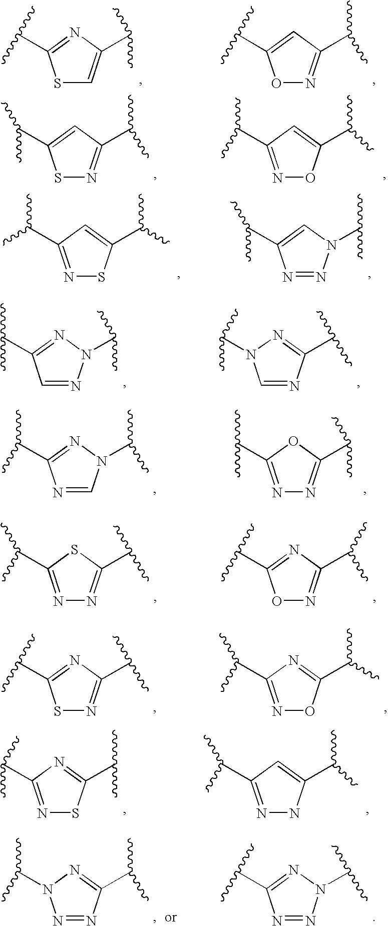

In a more preferred embodiment, Q¹ is

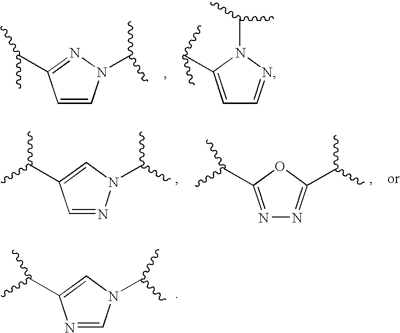

In alternative embodiment, Q¹ is 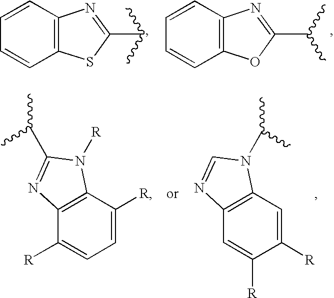
wherein each R is independently hydrogen, halogen, preferably —F or —Cl, C₁-C₆ alkyl, preferably methyl, C₁-C₆ haloalkyl, preferably —CF₃, or C₁-C₆ alkoxy, preferably ethoxy.

In one embodiment, Q² is CH₂ and m is 0 or 1, preferably m is 0. In another embodiment, Q² is CH₂O and m is 1. In a further embodiment, Q² is N(R′) and m is 1, where R′ is hydrogen or C₁-C₆ alkyl.

In one embodiment Q³ is aryl. In a preferred embodiment Q³ is selected from the group 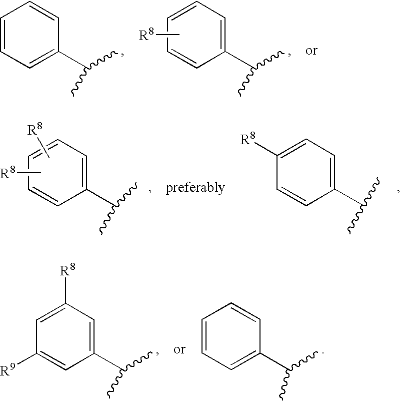
wherein R⁸ and R⁹ are independently selected from halogen or C₁-C₃ haloalkyl, preferably R⁸ and R⁹ are independently selected from flourine, chlorine, or trifluoromethyl.

In one embodiment Q³ is heteroaryl. In a preferred embodiment Q³ is selected from the group

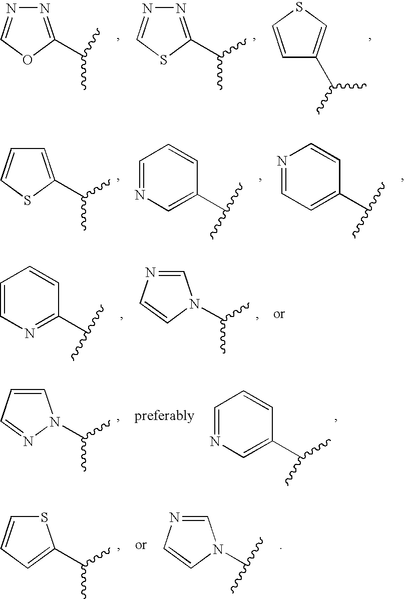

It is understood that Q¹ and Q³ as well as X¹ below are attached to the indicated linking group of Formula (I) or (II) through the bond or bonds of Q¹, Q³, and X¹ having an unfilled valence and being indicated by “ 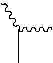
”. The appropriate attachments are further illustrated in the working examples recited below.

In one embodiment, R¹ is C₁-C₆ alkyl. In a preferred embodiment, R¹ is isopropyl, tert-butyl, 1,1-dimethylpropyl, 1-methyl-1-ethylpropyl, or 1,1-diethylpropyl. In a more preferred embodiment, R¹ is tert-butyl.

In one embodiment, R¹ is C₃-C₆ cycloalkyl or C₃-C₆ cycloalkyl substituted with C₁-C₆ alkyl. In a preferred embodiment, R¹ is cyclopropyl, cyclobutyl, cyclopentyl, methyl substituted cyclobutyl, or methyl substituted cyclopentyl. In a more preferred embodiment, R¹ is cyclobutyl.

As recited above D is 0 or S. In one embodiment D is S. In a preferred embodiment D is 0.

In one embodiment, R² is hydrogen. In another embodiment, R² is —NR³R⁴, wherein R³ is hydrogen or C₁-C₆ alkyl and R⁴ is hydrogen, C₁-C₆ alkyl, —C(O)R⁵, —C(O)OR⁵, or —S(O)₂R⁵.

As recited above Z is the group defined by —(X)ₘ—(X¹). In one embodiment, m is 0 and Z is —(X¹). In another embodiment, m is 1 and Z is the group defined by —(X)—(X¹).

In one embodiment, X is C(R′)(R′″), wherein R″ is hydrogen or C₁-C₆ alkyl, R′″ is hydrogen and C₁-C₆ alkyl, and m is 0, 1, or 2. In another embodiment, X is C(H)(R″) where R″ is hydrogen and m is 0, 1, or 2, preferably m is 0 or 1, more preferably m is 0. In another embodiment, X is C(H)(R″) where R″ is —CH₃ and m is 1.

In one embodiment X¹ is aryl. In a preferred embodiment X¹ is

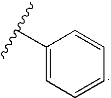

In one embodiment X¹ is heteroaryl or heterocyclyl. In a preferred embodiment X¹ is selected from the group

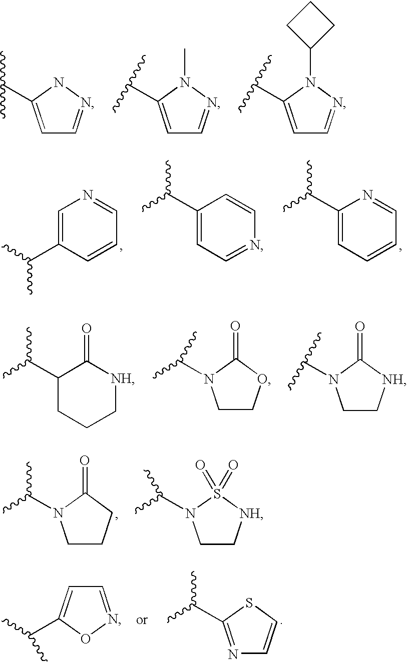

Specific examples of compounds of the present invention include the following:
(1S)-2,2-dimethyl-1-({3-[4-(trifluoromethyl)phenyl]-1H-pyrazol-1-yl}methyl)propyl(1S)-1-{oxo[(1H-pyrazol-5-ylmethyl)amino]acetyl}pentylcarbamate;
(1R)-2,2-dimethyl-1-({3-[4-(trifluoromethyl)phenyl]-1H-pyrazol-1-yl}methyl)propyl(1S)-1-[oxo(1H-pyrazol-3-ylamino)acetyl]pentylcarbamate;
(1R)-2,2-dimethyl-1-({5-[4-(trifluoromethyl)phenyl]-1,3,4-oxadiazol-2-yl}methyl)propyl(1S)-1-[oxo(1H-pyrazol-3-ylamino)acetyl]pentylcarbamate;
(1R)-1-{[5-(4-fluorophenyl)-1,3,4-oxadiazol-2-yl]methyl}-2,2-dimethylpropyl(1S)-1-{oxo[(3-pyridinylmethyl)amino]acetyl}pentylcarbamate;
(1S)-2,2-dimethyl-1-({3-[4-(trifluoromethyl)phenyl]-1H-pyrazol-1-yl}methyl)propyl(1S)-1-[oxo(2-pyridinylamino)acetyl]pentylcarbamate;
(1S)-1-{[4-(4-fluorophenyl)-1H-imidazol-1-yl]methyl}-2,2-dimethylpropyl(1S)-1-(oxo{[(1R)-1-phenylethyl]amino}acetyl)pentylcarbamate;
(1S)-2,2-dimethyl-1-({4-[4-(trifluoromethyl)phenyl]-1H-imidazol-1-yl}methyl)propyl(1S)-1-[oxo(1H-pyrazol-5-ylamino)acetyl]pentylcarbamate;
(1R)-2,2-dimethyl-1-[(5-phenyl-1,3,4-oxadiazol-2-yl)methyl]butyl(1S)-1-(oxo{[(1R)-1-phenylethyl]amino}acetyl)pentylcarbamate;
(1R)-2,2-dimethyl-1-[(5-phenyl-1,3,4-oxadiazol-2-yl)methyl]butyl(1S)-1-(oxo{[(1R)-1-phenylethyl]amino}acetyl)pentylcarbamate;
(1R)-2-methyl-1-[(5-phenyl-1,3,4-oxadiazol-2-yl)methyl]propyl(1S)-1-(oxo{[(1R)-1-phenylethyl]amino}acetyl)pentylcarbamate;
(1S)-2-methyl-1-[(5-phenyl-1,3,4-oxadiazol-2-yl)methyl]propyl(1S)-1-(oxo{[(1R)-1-phenylethyl]amino}acetyl)pentylcarbamate;
(1S)-2,2-dimethyl-1-[(4-phenyl-1H-imidazol-1-yl)methyl]propyl(1S)-1-(oxo{[(1R)-1-phenylethyl]amino}acetyl)pentylcarbamate;
(1R)-2,2-dimethyl-1-[(4-phenyl-1H-imidazol-1-yl)methyl]propyl(1S)-1-(oxo{[(1R)-1-phenylethyl]amino}acetyl)pentylcarbamate;
(1S)-2,2-dimethyl-1-({4-[4-(trifluoromethyl)phenyl]-1H-pyrazol-1-yl}methyl)propyl(1S)-1-[oxo(1H-pyrazol-5-ylamino)acetyl]pentylcarbamate;
(1R)-2,2-dimethyl-1-[(5-phenyl-1,3,4-oxadiazol-2-yl)methyl]propyl(1S)-1-(oxo{[(1R)-1-phenylethyl]amino}acetyl)pentylcarbamate;
(1S)-2,2-dimethyl-1-[(5-phenyl-1,3,4-oxadiazol-2-yl)methyl]propyl(1S)-1-(oxo{[(1R)-1-phenylethyl]amino}acetyl)pentylcarbamate;
(1R)-2,2-dimethyl-1-({5-[4-(trifluoromethyl)phenyl]-1,3,4-oxadiazol-2-yl}methyl)propyl(1S)-1-(oxo{[(1R)-1-phenylethyl]amino}acetyl)pentylcarbamate;
(1S)-2,2-dimethyl-1-({5-[4-(trifluoromethyl)phenyl]-1,3,4-oxadiazol-2-yl}methyl)propyl(1S)-1-(oxo{[(1R)-1-phenylethyl]amino}acetyl)pentylcarbamate;
(1R)-1-{[5-(4-fluorophenyl)-1,3,4-oxadiazol-2-yl]methyl}-2,2-dimethylpropyl(1S)-1-(oxo{[(1R)-1-phenylethyl]amino}acetyl)pentylcarbamate;
(1S)-1-{[5-(4-fluorophenyl)-1,3,4-oxadiazol-2-yl]methyl}-2,2-dimethylpropyl(1S)-1-(oxo{[(1R)-1-phenylethyl]amino}acetyl)pentylcarbamate;
(1R)-1-{[5-(4-fluorophenyl)-1,3,4-oxadiazol-2-yl]methyl}-2,2-dimethylpropyl(1S)-1-[oxo(2-pyridinylamino)acetyl]pentylcarbamate;
(1R)-1-{[5-(4-fluorophenyl)-1,3,4-oxadiazol-2-yl]methyl}-2,2-dimethylpropyl(1S)-1-[[(1-methyl-1H-pyrazol-5-yl)amino](oxo)acetyl]pentylcarbamate;
(1R)-1-{[5-(4-fluorophenyl)-1,3,4-oxadiazol-2-yl]methyl}-2,2-dimethylpropyl(1S)-1-[oxo(1H-pyrazol-5-ylamino)acetyl]pentylcarbamate;
(1R)-1-{[5-(4-fluorophenyl)-1,3,4-oxadiazol-2-yl]methyl}-2,2-dimethylpropyl(1S)-1-{oxo[(4-pyridinylmethyl)amino]acetyl}pentylcarbamate;
(1R)-1-{[5-(4-fluorophenyl)-1,3,4-oxadiazol-2-yl]methyl}-2,2-dimethylpropyl(1S)-1-(oxo{[(3S)-2-oxopiperidinyl]amino}acetyl)pentylcarbamate;
(1R)-2,2-dimethyl-1-(2-{5-[4-(trifluoromethyl)phenyl]-1,3,4-oxadiazol-2-yl}ethyl)propyl(1S)-1-[oxo(1H-pyrazol-5-ylamino)acetyl]pentylcarbamate;
(1S)-1-(1H-benzimidazol-1-ylmethyl)-2,2-dimethylpropyl(1S)-1-[oxo(1H-pyrazol-5-ylamino)acetyl]pentylcarbamate;
(1R)-2,2-dimethyl-1-({5-[4-(trifluoromethyl)phenyl]-1,3,4-oxadiazol-2-yl}methyl)propyl(1S)-1-{oxo[(2-oxo-1,3-oxazolidin-3-yl)amino]acetyl}pentylcarbamate;
(1S)-2,2-dimethyl-1-{[3-(trifluoromethyl)-1H-pyrazol-1-yl]methyl}propyl(1S)-1-[oxo(1H-pyrazol-5-ylamino)acetyl]pentylcarbamate;
(1S)-2,2-dimethyl-1-({5-[4-(trifluoromethyl)phenyl]-1H-pyrazol-1-yl}methyl)propyl(1S)-1-[oxo(1H-pyrazol-5-ylamino)acetyl]pentylcarbamate;
(1S*)-1(1,3-benzothiazol-2-yl)-2,2-dimethylpropyl(1S)-1-[oxo(1H-pyrazol-3-ylamino)acetyl]pentylcarbamate
(1R)-1-(1,3-benzothiazol-2-yl)-2,2-dimethylpropyl(1S)-1-[oxo(1H-pyrazol-5-ylamino)acetyl]pentylcarbamate;
(1S)-2,2-dimethyl-1-{[3-(3-pyridinyl)-1H-pyrazol-1-yl]methyl}propyl(1S)-1-[oxo(1,3-thiazol-2-ylamino)acetyl]pentylcarbamate;
(1S)-1-[(4-benzyl-1H-imidazol-1-yl)methyl]-2,2-dimethylpropyl(1R)-1-[oxo(1H-pyrazol-5-ylamino)acetyl]pentylcarbamate;
(1S)-1-[(4-benzyl-1H-imidazol-1-yl)methyl]-2,2-dimethylpropyl(1R)-1-[oxo(1H-pyrazol-5-ylamino)acetyl]pentylcarbamate;
(1S)-2,2-dimethyl-1-({3-[4-(trifluoromethyl)phenyl]-1H-pyrazol-1-yl}methyl)propyl(1S)-1-[[(5-isoxazolylmethyl)amino)(oxo)acetyl]pentylcarbamate
(1S)-1-[(5,6-dichloro-1H-benzimidazol-1-yl)methyl]-2,2-dimethylpropyl(1S)-1-[oxo(1H-pyrazol-5-ylamino)acetyl]pentylcarbamate;
(1S)-2,2-dimethyl-1-{5-[4-(trifluoromethyl)phenyl]-1,3,4-oxadiazol-2-yl}propyl(1S)-1-[oxo(1H-pyrazol-5-ylamino)acetyl]pentylcarbamate;
(1S)-2,2-dimethyl-1-{5-[4-(trifluoromethyl)phenyl]-1,3,4-oxadiazol-2-yl}propyl(1S)-1-{oxo[(2-oxo-1,3-oxazolidin-3-yl)amino]acetyl}pentylcarbamate;
(1R)-2,2-dimethyl-1-{5-[4-(trifluoromethyl)phenyl]-1,3,4-oxadiazol-2-yl}propyl(1S)-1-[oxo(1H-pyrazol-5-ylamino)acetyl]pentylcarbamate;
(1R)-1-[1,1′-biphenyl]-3-yl-2,2-dimethylpropyl(1S)-1-[oxo(1H-pyrazol-5-ylamino)acetyl]pentylcarbamate;
(1S)-1-[1,1′-biphenyl]-3-yl-2,2-dimethylpropyl(1S)-1-[oxo(1H-pyrazol-5-ylamino)acetyl]pentylcarbamate;
1-(4,7-diethoxy-1-methyl-1H-benzimidazol-2-yl)-2,2-dimethylpropyl(1S)-1-[(1H-pyrazol-5-ylamino)carbonyl]pentylcarbamate;
(1S)-2,2-dimethyl-1-{[3-(3-pyridinyl)-1H-pyrazol-1-yl]methyl}propyl(1S)-1-[oxo(1H)-pyrazol-5-ylamino)acetyl]pentylcarbamate;
(1S)-2,2-dimethyl-1-{[3-(4-pyridinyl)-1H-pyrazol-1-yl]methyl}propyl(1S)-1-[oxo(1H)-pyrazol-5-ylamino)acetyl]pentylcarbamate;
(1S)-2,2-dimethyl-1-({3-[4-(trifluoromethyl)phenyl]-1H-pyrazol-1-yl}methyl)propyl(1S)-1-[oxo(1,3-thiazol-2-ylamino)acetyl]pentylcarbamate;
(1S)-1-{[4-(benzyloxy)phenoxy]methyl}-2,2-dimethylpropyl(1S)-1-[oxo(1H-pyrazol-5-ylamino)acetyl]pentylcarbamate;
(1S)-1-{[4-(aminocarbonyl)phenoxy]methyl}-2,2-dimethylpropyl(1S)-1-[oxo(1H-pyrazol-5-ylamino)acetyl]pentylcarbamate;
(1S)-1-{[4-(1H-imidazol-1-yl)phenoxy]methyl}-2,2-dimethylpropyl(1S)-1-[oxo(1H-pyrazol-5-ylamino)acetyl]pentylcarbamate;
(1S)-1-({4-[3,5-bis(trifluoromethyl)phenyl]-1H-imidazol-1-yl}methyl)-2,2-dimethylpropyl(1S)-1-[oxo(1H-pyrazol-5-ylamino)acetyl]butylcarbamate;
(1S)-2,2-dimethyl-1-({4-[4-(trifluoromethyl)phenyl]-1H-imidazol-1-yl}methyl)propyl(1S)-1-[oxo(1,3-thiazol-2-ylamino)acetyl]pentylcarbamate;
(1S)-2,2-dimethyl-1-[5-(3-pyridinyl)-1,3,4-oxadiazol-2-yl]propyl(1S)-1-[oxo(1H)-pyrazol-5-ylamino)acetyl]pentylcarbamate;
(1R)-2,2-dimethyl-1-[5-(3-pyridinyl)-1,3,4-oxadiazol-2-yl]propyl(1S)-1-[oxo(1H-pyrazol-5-ylamino)acetyl]pentylcarbamate;
(1S)-2,2-dimethyl-1-[5-(4-pyridinyl)-1,3,4-oxadiazol-2-yl]propyl(1S)-1-[oxo(1H-pyrazol-5-ylamino)acetyl]pentylcarbamate; and
(1R)-2,2-dimethyl-1-[5-(4-pyridinyl)-1,3,4-oxadiazol-2-yl]propyl(1S)-1-[oxo(1H-pyrazol-5-ylamino)acetyl]pentylcarbamate;
or a salt, solvate, or physiologically functional derivative thereof.

Further specific examples of compounds of the present invention include the following:
(1S)-2,2-dimethyl-1-(3-thien-2-ylphenyl)propyl(1S)-1-[oxo(1H-pyrazol-5-ylamino)acetyl]pentylcarbamate;
(1R)-2,2-dimethyl-1-(3-thien-2-ylphenyl)propyl(1S)-1-[oxo(1H-pyrazol-5-ylamino)acetyl]pentylcarbamate;
(1S)-1-[(5,6-dichloro-1H-benzimidazol-1-yl)methyl]-2,2-dimethylpropyl(1S)-1-[oxo(pyridin-2-ylamino)acetyl]pentylcarbamate;
(1S)-1-[5-(2,6-dichloropyridin-4-yl)-1,3,4-oxadiazol-2-yl]-2,2-dimethylpropyl(1S)-1-[oxo(1H-pyrazol-5-ylamino)acetyl]pentylcarbamate;
(1R)-1-[5-(2,6-dichloropyridin-4-yl)-1,3,4-oxadiazol-2-yl]-2,2-dimethylpropyl(1S)-1-[oxo(1H-pyrazol-5-ylamino)acetyl]pentylcarbamate;
(1S)-1-(4,7-diethoxy-1-methyl-1H-benzimidazol-2-yl)-2,2-dimethylpropyl(1S)-1-[oxo(1H-pyrazol-5-ylamino)acetyl]pentylcarbamate;
(1R)-1-{5-[3,5-bis(trifluoromethyl)phenyl]-1,3,4-oxadiazol-2-yl}-2,2-dimethylpropyl(1S)-1-[oxo(1H-pyrazol-5-ylamino)acetyl]pentylcarbamate;
(1S)-1-{5-[3,5-bis(trifluoromethyl)phenyl]-1,3,4-oxadiazol-2-yl}-2,2-dimethylpropyl(1S)-1-[oxo(1H-pyrazol-5-ylamino)acetyl]pentylcarbamate;
(1S)-2,2-dimethyl-1-{5-[4-(trifluoromethyl)phenyl]-1,3,4-oxadiazol-2-yl}butyl(1S)-1-[oxo(1H-pyrazol-5-ylamino)acetyl]pentylcarbamate; and
(1R)-2,2-dimethyl-1-{5-[4-(trifluoromethyl)phenyl]-1,3,4-oxadiazol-2-yl}butyl(1S)-1-[oxo(1H-pyrazol-5-ylamino)acetyl]pentylcarbamate;
or a salt, solvate, or physiologically functional derivative thereof.

Typically, the salts of the present invention are pharmaceutically acceptable salts. Salts encompassed within the term “pharmaceutically acceptable salts” refer to non-toxic salts of the compounds of this invention. Salts of the compounds of the present invention may comprise acid addition salts derived from a nitrogen on a substituent in the compound of formula (I) or formula (II). Representative salts include the following salts: acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionate, lactate, lactobionate, laurate, malate, maleate, mandelate, mesylate, methylbromide, methylnitrate, methylsulfate, monopotassium maleate, mucate, napsylate, nitrate, N-methylglucamine, oxalate, pamoate (embonate), palmitate, pantothenate, phosphate/diphosphate, polygalacturonate, potassium, salicylate, sodium, stearate, subacetate, succinate, tannate, tartrate, teoclate, tosylate, triethiodide, trimethylammonium, and valerate. Other salts, which are not pharmaceutically acceptable, may be useful in the preparation of compounds of this invention and these form a further aspect of the invention.

While it is possible that, for use in therapy, therapeutically effective amounts of a compound of formula (I) or formula (II), as well as salts, solvates and physiological functional derivatives thereof, may be administered as the raw chemical, it is possible to present the active ingredient as a pharmaceutical composition. Accordingly, the invention further provides pharmaceutical compositions which include therapeutically effective amounts of compounds of the formula (I) or (II) and salts, solvates and physiological functional derivatives thereof, and one or more pharmaceutically acceptable carriers, diluents, or excipients. The compounds of the formula (I) or (II) and salts, solvates and physiologically functional derivatives thereof, are as described above. The carrier(s), diluent(s) or excipient(s) must be acceptable in the sense of being compatible with the other ingredients of the formulation and not deleterious to the recipient thereof. In accordance with another aspect of the invention there is also provided a process for the preparation of a pharmaceutical formulation including admixing a compound of the formula (I) or (II), or salts, solvates and physiological functional derivatives thereof, with one or more pharmaceutically acceptable carriers, diluents or excipients.

Pharmaceutical formulations may be presented in unit dose forms containing a predetermined amount of active ingredient per unit dose. Such a unit may contain, for example, 0.5 mg to 1 g, preferably 1 mg to 700 mg, of a compound of the formula (I) or (II) depending on the condition being treated, the route of administration and the age, weight and condition of the patient. Preferred unit dosage formulations are those containing a daily dose or sub-dose, as herein above recited, or an appropriate fraction thereof, of an active ingredient. Furthermore, such pharmaceutical formulations may be prepared by any of the methods well known in the pharmacy art.

Pharmaceutical formulations may be adapted for administration by any appropriate route, for example by the oral (including buccal or sublingual), rectal, nasal, topical (including buccal, sublingual or transdermal), vaginal or parenteral (including subcutaneous, intramuscular, intravenous or intradermal) route. Such formulations may be prepared by any method known in the art of pharmacy, for example by bringing into association the active ingredient with the carrier(s) or excipient(s).

Pharmaceutical formulations adapted for oral administration may be presented as discrete units such as capsules or tablets; powders or granules; solutions or suspensions in aqueous or non-aqueous liquids; edible foams or whips; or oil-in-water liquid emulsions or water-in-oil liquid emulsions.

For instance, for oral administration in the form of a tablet or capsule, the active drug component can be combined with an oral, non-toxic pharmaceutically acceptable inert carrier such as ethanol, glycerol, water, and the like. Powders are prepared by comminuting the compound to a suitable fine size and mixing with a similarly comminuted pharmaceutical carrier such as an edible carbohydrate, as, for example, starch or mannitol. Flavoring, preservative, dispersing and coloring agent can also be present.

Capsules are made by preparing a powder mixture as described above, and filling formed gelatin sheaths. Glidants and lubricants such as colloidal silica, talc, magnesium stearate, calcium stearate or solid polyethylene glycol can be added to the powder mixture before the filling operation. A disintegrating or solubilizing agent such as agar-agar, calcium carbonate or sodium carbonate can also be added to improve the availability of the medicament when the capsule is ingested.

Moreover, when desired or necessary, suitable binders, lubricants, disintegrating agents, and coloring agents can also be incorporated into the mixture. Suitable binders include starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Lubricants used in these dosage forms include sodium oleate, sodium stearate, magnesium stearate, sodium benzoate, sodium acetate, sodium chloride, and the like. Disintegrators include, without limitation, starch, methyl cellulose, agar, bentonite, xanthan gum and the like. Tablets are formulated, for example, by preparing a powder mixture, granulating or slugging, adding a lubricant and disintegrant, and pressing into tablets. A powder mixture is prepared by mixing the compound, suitably comminuted, with a diluent or base as described above, and optionally, with a binder such as carboxymethylcellulose, an aliginate, gelatin, or polyvinyl pyrrolidone, a solution retardant such as paraffin, a resorption accelerator such as a quaternary salt and/or an absorption agent such as bentonite, kaolin or dicalcium phosphate. The powder mixture can be granulated by wetting with a binder such as syrup, starch paste, acadia mucilage or solutions of cellulosic or polymeric materials, and forcing through a screen. As an alternative to granulating, the powder mixture can be run through the tablet machine and the result is imperfectly formed slugs broken into granules. The granules can be lubricated to prevent sticking to the tablet forming dies by means of the addition of stearic acid, a stearate salt, talc or mineral oil. The lubricated mixture is then compressed into tablets. The compounds of the present invention can also be combined with a free flowing inert carrier and compressed into tablets directly without going through the granulating or slugging steps. A clear or opaque protective coating consisting of a sealing coat of shellac, a coating of sugar or polymeric material and a polish coating of wax can be provided. Dyestuffs can be added to these coatings to distinguish different unit dosages.

Oral fluids such as solution, syrups and elixirs can be prepared in dosage unit form so that a given quantity contains a predetermined amount of the compound. Syrups can be prepared by dissolving the compound in a suitably flavored aqueous solution, while elixirs are prepared through the use of a non-toxic alcoholic vehicle. Suspensions can be formulated by dispersing the compound in a non-toxic vehicle. Solubilizers and emulsifiers such as ethoxylated isostearyl alcohols and polyoxy ethylene sorbitol ethers, preservatives; flavor additives such as peppermint oil, or natural sweeteners, saccharin, or other artificial sweeteners; and the like can also be added.

Where appropriate, dosage unit formulations for oral administration can be microencapsulated. The formulation can also be prepared to prolong or sustain the release as for example by coating or embedding particulate material in polymers, wax or the like.

The compounds of formula (I) or (II) and salts, solvates and physiological functional derivatives thereof, can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

The compounds of formula (I) or (II) and salts, solvates and physiologically functional derivatives thereof may also be delivered by the use of monoclonal antibodies as individual carriers to which the compound molecules are coupled. The compounds may also be coupled with soluble polymers as targetable drug carriers. Such polymers can include polyvinylpyrrolidone, pyran copolymer, polyhydroxypropylmethacrylamide-phenol, polyhydroxyethylaspartamidephenol, or polyethyleneoxidepolylysine substituted with palmitoyl residues. Furthermore, the compounds may be coupled to a class of biodegradable polymers useful in achieving controlled release of a drug; for example, polylactic acid, polepsilon caprolactone, polyhydroxy butyric acid, polyorthoesters, polyacetals, polydihydropyrans, polycyanoacrylates, and cross-linked or amphipathic block copolymers of hydrogels.

Pharmaceutical formulations adapted for transdermal administration may be presented as discrete patches intended to remain in intimate contact with the epidermis of the recipient for a prolonged period of time. For example, the active ingredient may be delivered from the patch by iontophoresis as generally described in Pharmaceutical Research, 3(6), 318 (1986).

Pharmaceutical formulations adapted for topical administration may be formulated as ointments, creams, suspensions, lotions, powders, solutions, pastes, gels, sprays, aerosols, or oils.

For treatments of the eye or other external tissues, for example mouth and skin, the formulations are preferably applied as a topical ointment or cream. When formulated in an ointment, the active ingredient may be employed with either a paraffinic or a water-miscible ointment base. Alternatively, the active ingredient may be formulated in a cream with an oil-in-water cream base or a water-in-oil base.

Pharmaceutical formulations adapted for topical administrations to the eye include eye drops wherein the active ingredient is dissolved or suspended in a suitable carrier, especially an aqueous solvent.

Pharmaceutical formulations adapted for topical administration in the mouth include lozenges, pastilles, and mouth washes.

Pharmaceutical formulations adapted for rectal administration may be presented as suppositories or as enemas.

Pharmaceutical formulations adapted for nasal administration wherein the carrier is a solid include a coarse powder having a particle size for example in the range 20 to 500 microns, which is administered in the manner in which snuff is taken, i.e., by rapid inhalation through the nasal passage from a container of the powder held close up to the nose. Suitable formulations wherein the carrier is a liquid, for administration as a nasal spray or as nasal drops, include aqueous or oil solutions of the active ingredient.

Pharmaceutical formulations adapted for administration by inhalation include fine particle dusts or mists, which may be generated by means of various types of metered, dose pressurised aerosols, nebulizers, or insufflators.

Pharmaceutical formulations adapted for vaginal administration may be presented as pessaries, tampons, creams, gels, pastes, foams, or spray formulations.

Pharmaceutical formulations adapted for parenteral administration include aqueous and non-aqueous sterile injection solutions which may contain anti-oxidants, buffers, bacteriostats, and solutes that render the formulation isotonic with the blood of the intended recipient; and aqueous and non-aqueous sterile suspensions which may include suspending agents and thickening agents. The formulations may be presented in unit-dose or multi-dose containers, for example sealed ampules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules, and tablets.

It should be understood that in addition to the ingredients particularly mentioned above, the formulations may include other agents conventional in the art having regard to the type of formulation in question, for example those suitable for oral administration may include flavouring agents.

A therapeutically effective amount of a compound of the present invention will depend upon a number of factors including, for example, the age and weight of the animal, the precise condition requiring treatment and its severity, the nature of the formulation, and the route of administration, and will ultimately be at the discretion of the attendant physician or veterinarian. However, an effective amount of a compound of formula (I) or (II) for the treatment of osteoporosis will generally be in the range of 0.1 to 100 mg/kg body weight of recipient (mammal) per day and more usually in the range of 1 to 10 mg/kg body weight per day. Thus, for a 70 kg adult mammal, the actual amount per day would usually be from 70 to 700 mg and this amount may be given in a single dose per day or more usually in a number (such as two, three, four, five or six) of sub-doses per day such that the total daily dose is the same. An effective amount of a salt or solvate, or physiologically functional derivative thereof, may be determined as a proportion of the effective amount of the compound of formula (I) or (II) per se. It is envisaged that similar dosages would be appropriate for treatment of the other conditions referred to above.

The compounds of the present invention and their salts and solvates, and physiologically functional derivatives thereof, may be employed alone or in combination with other therapeutic agents for the treatment of the above-mentioned conditions. In particular, in osteoporosis therapy, combination with other osteoporosis therapeutic agents is envisaged. Combination therapies according to the present invention thus comprise the administration of at least one compound of formula (I) or (II) or a pharmaceutically acceptable salt or solvate thereof, or a physiologically functional derivative thereof, and the use of at least one other osteoporosis treatment method. Preferably, combination therapies according to the present invention comprise the administration of at least one compound of formula (I) or (II) or a pharmaceutically acceptable salt or solvate thereof, or a physiologically functional derivative thereof, and at least one other osteoporosis treatment agent, preferably a bone building agent. The compound(s) of formula (I) or (II) and the other pharmaceutically active agent(s) may be administered together or separately and, when administered separately, this may occur simultaneously or sequentially in any order. The amounts of the compound(s) of formula (I) or (II) and the other pharmaceutically active agent(s) and the relative timings of administration will be selected in order to achieve the desired combined therapeutic effect. The administration in combination of a compound of formula (I) or (II) or salts, solvates, or physiologically functional derivatives thereof with other osteoporosis treatment agents may be in combination in accordance with the invention by administration concomitantly in (1) a unitary pharmaceutical composition including both compounds or (2) separate pharmaceutical compositions each including one of the compounds. Alternatively, the combination may be administered separately in a sequential manner wherein one osteoporosis treatment agent is administered first and the other second or vice versa. Such sequential administration may be close in time or remote in time.

A preferred additional osteoporosis treatment agent is a bone building (anabolic) agent. Bone building agents can lead to increases in parameters such as bone mineral density greater than those than can be achieved with anti-resorptive agents. In some cases, such anabolic agents can increase trabecular connectivity leading to greater structural integrity of the bone. A combination therapy composed of a bone forming agent with an anti-resorptive drug such as a cathepsin K inhibitor could provide even greater efficacy than treatment with either agent alone.

The present invention is directed to methods of regulating, modulating, or inhibiting cathepsin K for the prevention and/or treatment of disorders related enhanced bone turnover, which can ultimately lead to fracture. In particular, the compounds of the present invention can also be used in the treatment of osteoporosis. Furthermore, the compounds of the present invention can be used to provide additive or synergistic effects with existing osteoporosis therapies.

The present invention thus also provides compounds of formula (I) or (II) and pharmaceutically acceptable salts or solvates thereof, or physiologically functional derivatives thereof, for use in medical therapy, and particularly in the treatment of disorders mediated by enhanced bone turnover which can ultimately leading to fracture.

The present invention also provides compounds of formula (I) or (II) and pharmaceutically acceptable salts or solvates thereof, or physiologically functional derivatives thereof, for use in medical therapy, and particularly in the treatment of disorders characterized by bone loss or characterized by excessive cartilage or matrix degradation.

The compounds of the present invention are also useful in the treatment of one or more diseases afflicting mammals that are characterized by potential involvement of cathepsin K in autoimmune diseases such as rheumatoid arthritis, osteoathritis, neoplastic disdeases, parasitic diseases, and atherosclerosisis.

A further aspect of the invention provides a method of treatment of a mammal suffering from a disorder mediated by enhanced bone turnover that can ultimately lead to fracture, which includes administering to said subject an effective amount of a compound of formula (I) or (II) or a pharmaceutically acceptable salt, solvate, or a physiologically functional derivative thereof.

A further aspect of the invention provides a method of treatment of a mammal suffering from a disorder characterized by bone loss, which includes administering to said subject an effective amount of a compound of formula (I) or (II) or a pharmaceutically acceptable salt, solvate, or a physiologically functional derivative thereof. In a preferred embodiment, the disorder is osteoporosis.

A further aspect of the invention provides a method of treatment of a mammal suffering from osteoporosis, which includes administering to said subject an effective amount of a compound of formula (I) or (II) or a pharmaceutically acceptable salt or solvate thereof, or a physiologically functional derivative thereof.

A further aspect of the present invention provides the use of a compound of formula (I) or (II), or a pharmaceutically acceptable salt or solvate thereof, or a physiologically functional derivative thereof, in the preparation of a medicament for the treatment of a disorder characterized by enhanced bone turnover that can ultimately lead to fracture. In a preferred embodiment, the disorder is osteoporosis.

A further aspect of the present invention provides the use of a compound of formula (I) or (II), or a pharmaceutically acceptable salt or solvate thereof, or a physiologically functional derivative thereof, in the preparation of a medicament for the treatment of a disorder characterized by bone loss. In a preferred embodiment, the disorder is osteoporosis.

A further aspect of the present invention provides the use of a compound of formula (I) or (II), or a pharmaceutically acceptable salt or solvate thereof, or a physiologically functional derivative thereof, in the preparation of a medicament for the treatment of osteoporosis.

The mammal requiring treatment with a compound of the present invention is typically a human being.

In another embodiment, therapeutically effective amounts of the compounds of formula (I) or (II) or salts, solvates or physiologically derived derivatives thereof and at least one bone building agent may be administered in combination to a mammal for treatment of osteoporosis.

The compounds of this invention may be made by a variety of methods, including standard synthetic methods. Any previously defined variable will continue to have the previously defined meaning unless otherwise indicated. Illustrative general synthetic methods are set out below and then specific compounds of the invention are prepared in the working Examples.

Compounds of general formula (I) or formula (II) may be prepared by methods known in the art of organic synthesis as set forth in part by the following synthetic schemes. Generally, the following schemes are illustrated using compounds of formula (II), but it is recognized that such schemes are easily adaptable by the skilled artisan to prepare other compounds of formula (I). It is also recognized that in all of the schemes described below, it is well understood that protecting groups for sensitive or reactive groups are employed where necessary in accordance with general principles of synthetic chemistry. Protecting groups are manipulated according to standard methods of organic synthesis (T. W. Green and P. G. M. Wuts (1991) Protecting Groups in Organic Synthesis, John Wiley a Sons). These groups are removed at a convenient stage of the compound synthesis using methods that are readily apparent to those skilled in the art. The selection of processes as well as the reaction conditions and order of their execution shall be consistent with the preparation of compounds of formula (I) or (II). Those skilled in the art will recognize if a stereocenter exists in compounds of formula (I) or (II). Accordingly, the present invention includes all possible stereoisomers and includes not only racemic compounds but the individual enantiomers as well. When a compound is desired as a single enantiomer, it may be obtained by stereospecific synthesis or by resolution of the final product or any convenient intermediate. Resolution of the final product, an intermediate, or a starting material may be effected by any suitable method known in the art. See, for example, Stereochemistry of Organic Compounds by E. L. Eliel, S. H. Wilen, and L. N. Mander (Wiley-Interscience, 1994).

Compounds of formula (I) and (II), can be prepared according to the synthetic sequences shown in Schemes I, II, and III, which are further detailed in the Examples section following. 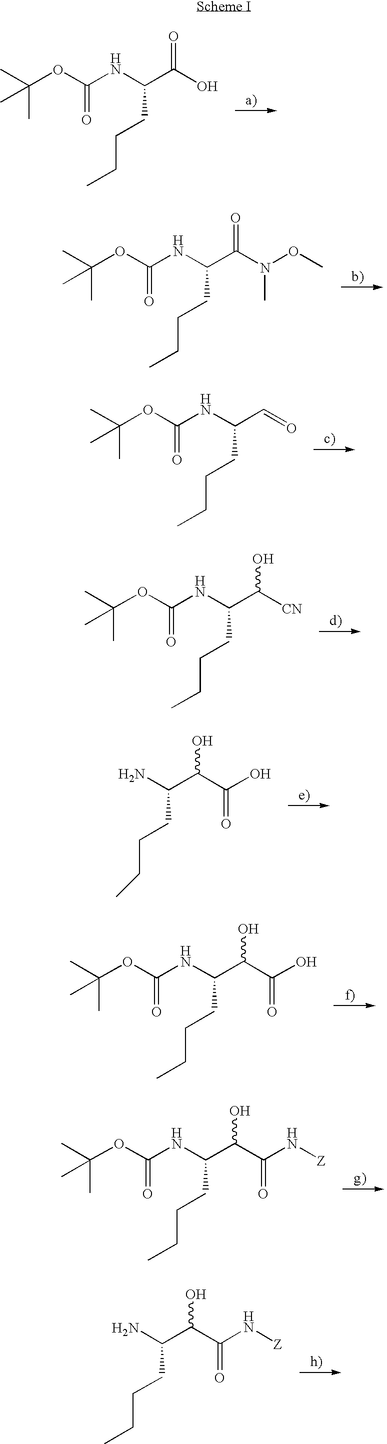 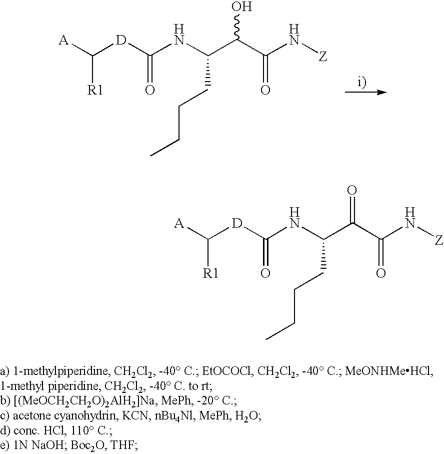
^{f)} 1-methylpiperidine, CH₂Cl₂, −40° C.; EtOCOCl, CH₂Cl₂, −40° C.; N,N′-carbonyldiimidazole; amine; K₂CO₃, MeOH, H₂O;

^{g)} 4N HCl in dioxane, dioxane;

^{h)} carbonate or chloroformate, DMF; iPr₂NEt;

ⁱ⁾ Dess-Martin Periodinane, CH₂Cl₂; or pyridine.SO₃, DMSO, CH₂Cl₂, NEt₃; or COCl₂, DMSO, CH₂Cl₂, NEt₃.

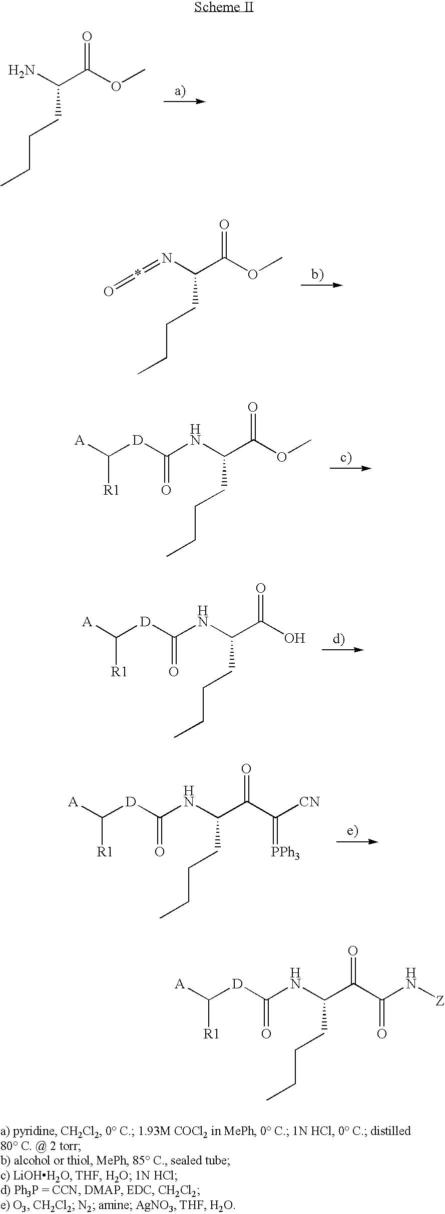

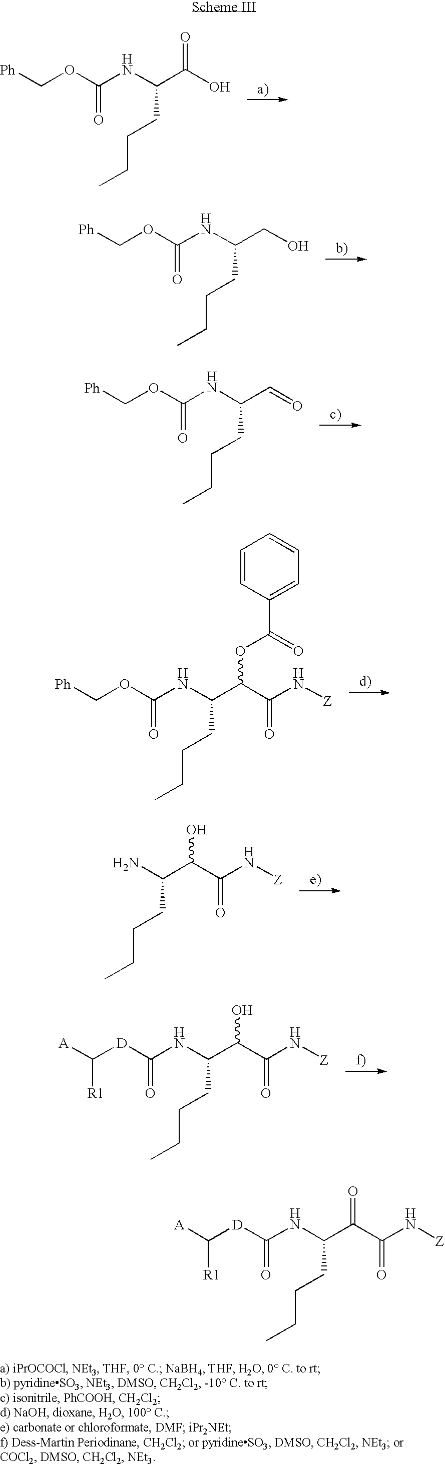

Certain embodiments of the present invention will now be illustrated by way of example only. The physical data given for the compounds exemplified is consistent with the assigned structure of those compounds.

### EXAMPLES

As used herein the symbols and conventions used in these processes, schemes and examples are consistent with those used in the contemporary scientific literature, for example, the Journal of the American Chemical Society or the Journal of Biological Chemistry. Standard single-letter or three-letter abbreviations are generally used to designate amino acid residues, which are assumed to be in the L-configuration unless otherwise noted. Unless otherwise noted, all starting materials were obtained from commercial suppliers and used without further purification. Specifically, the following abbreviations may be used in the examples and throughout the specification:

| | |
|---|---|
| g (grams); | mg (milligrams); |
| L (liters); | mL (milliliters); |
| μL (microliters); | psi (pounds per square inch); |
| M (molar); | mM (millimolar); |
| h (hour(s)); | Hz (Hertz); |
| MHz (megahertz); | mol (moles); |
| mmol (millimoles); | RT (room temperature); |
| min (minutes); | h (hours); |
| mp (melting point); | TLC (thin layer chromatography); |
| Tᵣ (retention time); | RP (reverse phase); |
| MeOH (methanol); | I-PrOH (isopropanol); |
| TEA (triethylamine); | TFA (trifluoroacetic acid); |
| TFAA (trifluoroacetic anhydride); | THF (tetrahydrofuran); |
| DMSO (dimethylsulfoxide); | EtOAc (ethyl acetate); |
| DME (1,2-dimethoxyethane); | DCM (dichloromethane); |
| DCE (dichloroethane); | DMF (N,N-dimethylformamide); |
| DMPU (N,N′-dimethylpropyleneurea); | CDI (1,1-carbonyldiimidazole); |
| IBCF (isobutyl chloroformate); | HOAc (acetic acid); |
| HOSu (N-hydroxysuccinimide); | HOBT (1-hydroxybenzotriazole); |
| mCPBA (meta-chloroperbenzoic acid; | EDC (ethylcarbodiimide hydrochloride); |
| BOC (tert-butyloxycarbonyl); | FMOC (9-fluorenylmethoxycarbonyl); |
| DCC (dicyclohexylcarbodiimide); | CBZ (benzyloxycarbonyl); |
| Ac (acetyl); | atm (atmosphere); |
| TMSE (2-(trimethylsilyl)ethyl); | TMS (trimethylsilyl); |
| TIPS (triisopropylsilyl); | TBS (t-butyldimethylsilyl); |
| DMAP (4-dimethylaminopyridine); | Me (methyl); |
| HPLC (high pressure liquid chromatography); | tBu (tert-butyl). |
| BOP (bis(2-oxo-3-oxazolidinyl)phosphinic chloride); | |
| TBAF (tetra-n-butylammonium fluoride); | |
| Et (ethyl); | |

All references to ether are to diethyl ether; brine refers to a saturated aqueous solution of NaCl. Unless otherwise indicated, all temperatures are expressed in ° C. (degrees Centigrade). All reactions were conducted under an inert atmosphere at room temperature unless otherwise noted.

¹H NMR spectra were recorded on a Varian VXR-300, a Varian Unity-300, a Varian Unity-400 instrument, or a General Electric QE-300. Chemical shifts are expressed in parts per million (ppm, δ units). Coupling constants are in units of hertz (Hz). Splitting patterns describe apparent multiplicities and are designated as s (singlet), d (doublet), t (triplet), q (quartet), m (multiplet), br (broad).

Low-resolution mass spectra (MS) were recorded on a JOEL JMS-AX505HA, JOEL SX-102, or a SCIEX-APliii spectrometer; high resolution MS were obtained using a JOEL SX-102A spectrometer. All mass spectra were taken under electrospray ionization (ESI), chemical ionization (Cl), electron impact (El) or by fast atom bombardment (FAB) methods. Infrared (IR) spectra were obtained on a Nicolet 510 FT-IR spectrometer using a 1-mm NaCl cell. All reactions were monitored by thin-layer chromatography on 0.25 mm E. Merck silica gel plates (60F-254), visualized with UV light, 5% ethanolic phosphomolybdic acid, iodine, iodoplatinate(potassium), permanganate(potassium), or p-anisaldehyde solution. Flash column chromatography was performed on silica gel (230-400 mesh, Merck). Optical rotations were obtained using a Perkin Elmer Model 241 Polarimeter. Melting points were determined using a Mel-Temp II apparatus and are uncorrected.

The following examples describe the syntheses of compounds of Formula (I) and (II) as well as intermediates particularly useful in the synthesis of compounds of Formula (I) and (II):

### Example 1

### (1S)-2,2-dimethyl-1-({3-[4-(trifluoromethyl)phenyl]-1H-pyrazol-1-yl}methyl)propyl(1S)-1-{oxo[(1H-pyrazol-5-ylmethyl)amino]acetyl}pentylcarbamate

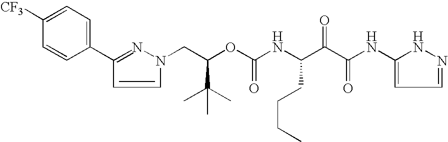

### Example 1a

### Preparation of (S)-2-hydroxy-3,3-dimethylbutanoic acid

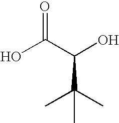

To a solution of 30 g (0.229 mol) of L-tert-leucine in 345 mL of 1 N sulfuric acid, cooled to 0° C., was added over 2 h a solution of 23.7 g (0.34 mol) of sodium nitrite in 83 mL of water. The temperature was maintained below 5° C. during the addition, and the mixture was then refrigerated for 24 h. The solution was then extracted with 150 mL of ether (3×) and the extract was washed with 100 mL of saturated aqueous sodium chloride. The extract was dried over anhydrous magnesium sulfate, filtered, and concentrated to afford 19.5 g (65%) of a pale yellow oil. The crude product was taken to the next step. ¹H NMR (300 MHz, CDCl₃) δ 3.91 (s, 1H), 1.01 (s, 9H).

### Example 1b

### Preparation of (2S)-3,3-dimethyl-1,2-butanediol

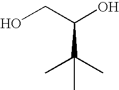

To a solution of 18.0 g (0.136 mol) of (S)-2-hydroxy-3,3-dimethylbutanoic acid in 150 mL of ether, cooled to 0° C., was added 272 mL (272 mmol) of a 1.0 M solution of lithium aluminum hydride in tetrahydrofuran over a period of 30 min. The reaction mixture was then warmed to room temperature and stirred for 16 h. To the reaction mixture was added 100 mL of 50% concentrated hydrochloric acid. The layers were separated, the aqueous layer was extracted with 200 mL of ether (3×), and the extract was dried over anhydrous magnesium sulfate. After filtration and concentration, the crude product was purified by column chromatography on silica gel with hexane:ethyl acetate (1:9) as the eluent to afford 10.2 g (63%) of (2S)-3,3-dimethyl-1,2-butanediol as a colorless solid. ¹H NMR (300 MHz, CDCl₃) δ 3.74 (dd, J=10 Hz, J=3 Hz, 1H), 3.48 (t, J=10 Hz, 1H), 3.36 (dd, J=10 Hz, J=3 Hz, 1H), 2.70 (br s, 2H), 0.91 (s, 9H).

### Example 1c

### Preparation of (2S)-2-hydroxy-3,3-dimethylbutyl 4-methylbenzenesulfonate

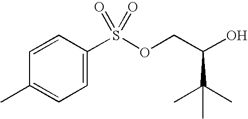

To 6.65 g (56 mmol) of (2S)-3,3-dimethyl-1,2-butanediol in 13 mL of pyridine at 0° C. was added dropwise a solution of p-toluenesulfonyl chloride in 20 mL of pyridine. The solution was maintained at 0° C. for 5 h and then let warm to ambient temperature. After stirring overnight, the mixture was concentrated, and the residue was taken up in 200 mL of diethyl ether. The ether solution was washed with 50 mL of 1N hydrochloric acid, 50 mL of saturated aqueous sodium bicarbonate, and 50 mL of water, dried over anhydrous magnesium sulfate, and concentrated. The residue was purified by silica gel chromatography eluting with ethyl acetate:hexanes (3.5:6.5) to give 13 g (85%) of (2S)-2-hydroxy-3,3-dimethylbutyl 4-methylbenzenesulfonate. ¹H NMR (300 MHz, DMSO-d₆) δ 7.79 (d, J=8 Hz, 2H), 7.47 (d, J=8 Hz, 2H), 5.11 (br s, 1H), 4.08 (dd, J=10 Hz, J=3 Hz, 1H), 3.76 (dd, J=10 Hz, J=8 Hz, 1H), 3.23 (d, J=8 Hz, 1H), 2.41 (s, 3H), 0.76 (s, 9H). ES-LCMS m/z 273 (M+H), 295 (M+Na).

### Example 1d

### Preparation of (S)-3,3-dimethyl-1,2-epoxybutane

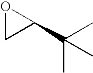

To 20.05 g (73.7 mmol) of (2S)-2-hydroxy-3,3-dimethylbutyl 4-methylbenzenesulfonate in 300 mL of methanol at 0° C. was added dropwise 75.2 mL (75.2 mmol) of 1M sodium hydroxide, and the mixture was stirred for 30 min. It was then diluted with 10 mL of saturated potassium dihydrogen phosphate, and poured into 1400 mL of water. The mixture was extracted three times with 50 mL of pentane. The extracts were combined and dried over anhydrous magnesium sulfate, and the pentane was distilled off to afford 7.94 g (830%) of (S)-3,3-dimethyl-1,2-epoxybutane with 0.4 mole equivalents of residual pentane. ¹H NMR (300 MHz, DMSO-d₆) δ 2.69 (dd, J=4 Hz, J=3 Hz, 1H), 2.52-2.57 (m, 2H), 0.85 (s, 9H).

### Example 1e

### Preparation of 3-[4-(Trifluoromethyl)phenyl]-1H-pyrazole

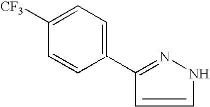

First, 3.18 g (79.4 mmol) of a 60% sodium hydride suspension in mineral oil was added in portions to a solution of 9.96 g (52.9 mmol) of 4-trifluoromethylacetophenone and 12.6 mL (158.76 mmol) of ethyl formate in 75 mL of anhydrous tetrahydrofuran at 0° C. The mixture was allowed to reach ambient temperature, at which an exothermic reaction occurred, which subsided in 5 min. After 1 h, the mixture was concentrated and the residue was triturated with diethyl ether to provide a tan solid in two crops. The solid was suspended in 1N hydrochloric acid and the resulting bright yellow solid was filtered and washed with water. The solid was dissolved in 150 mL of methanol and stirred with 4.7 mL (96.9 mmol) of hydrazine hydrate for 3 h at ambient temperature. Solvent was evaporated and the resulting solid was suspended in water, stirring for 18 h. The solid was filtered, washed with water, and dried under vacuum to provide 8.9 g (80%) of 3-[4-(trifluoromethyl)phenyl]-1H-pyrazole as a yellow solid. ¹H NMR (DMSO-d₆): δ 13.05 (br s, 1H), 7.99 (d, J=8 Hz, 2H), 7.8 (br s, 1H), 7.71 (d, J=8 Hz, 2H), 6.81 (s, 1H); ES-LCMS m/z 213 (M+H).

### Example 1f

### Preparation of (2S)-3,3-Dimethyl-1-{3-[4-(trifluoromethyl)phenyl]-1H-pyrazol-1-yl}-2-butanol

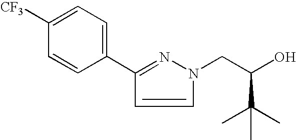

A mixture of 4.11 g (19.4 mmol) of 3-[4-(trifluoromethyl)phenyl]-1H-pyrazole, 2.0 g (19.9 mmol) of (S)-3,3-dimethyl-1,2-epoxybutane, 3.1 mL (22.3 mmol) of triethylamine, and 10 mL of isopropyl alcohol was placed in a sealed tube and heated at 85° C. for 48 h. Solvent was evaporated and the residue was purified by silica gel chromatography eluting with ethyl acetate:hexanes (1:7) to give 2.92 g (49%) of (25S)-3,3-Dimethyl-1-{3-[4-(trifluoromethyl)phenyl]-1H-pyrazol-1-yl}-2-butanol as a pale yellow solid and 0.4 g (7%) of its isomer (2S)-3,3-dimethyl-1-{5-[4-(trifluoromethyl)phenyl]-1H-pyrazol-1-yl}-2-butanol. Data for (2S)-3,3-dimethyl-1-{3-[4-(trifluoromethyl)phenyl]-1H-pyrazol-1-yl}-2-butanol: ¹H NMR (DMSO-d₆): δ 7.96 (d, J=8 Hz, 2H), 7.75 (s, 1H), 7.69 (d, J=8 Hz, 2H), 6.76 (d, J=2 Hz, 1H), 4.86 (d, J=6 Hz, 1H), 4.26 (dd, J=14 Hz, J=2 Hz, 1H), 3.88 (dd, J=14 Hz, J=10 Hz, 1H), 3.45-3.50 (m, 1H), 0.88 (s, 9H); ES-LCMS m/z 313 (M+H). Data for (2S)-3,3-dimethyl-1-{5-[4-(trifluoromethyl)phenyl]-1H-pyrazol-1-yl}-2-butanol: ¹H NMR (DMSO-d₆): δ 7.87 (d, J=8 Hz, 2H), 7.80 (d, J=8 Hz, 2H), 7.52 (d, J=2 Hz, 1H), 6.42 (d, J=2 Hz, 1H), 5.01 (d, J=6 Hz, 1H), 4.10 (dd, J=14 Hz, J=2 Hz, 1H), 3.92 (dd, J=14 Hz, J=10 Hz), 3.62-3.67 (m, 1H), 0.82 (s, 9H); ES-LCMS m/z 313 (M+H).

### Example 1g

### Preparation of (1S)-2,2-Dimethyl-1-({3-[4-(trifluoromethyl)phenyl]-1H-pyrazol-1-yl}methyl)propyl 4-nitrophenyl carbonate

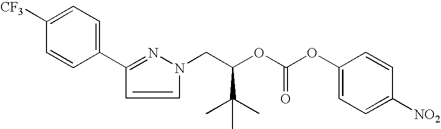

To a solution of 2.56 g (8.19 mmol) of (2S)-3,3-dimethyl-1-{3-[4-(trifluoromethyl)phenyl]-1H-pyrazol-1-yl}-2-butanol and 1.54 g (19.6 mmol) of pyridine in 29 mL of anhydrous dichloromethane was added 1.98 g (9.83 mmol) of p-nitrophenyl chloroformate. The mixture was stirred at ambient temperature for 18 h. It was washed with 5% citric acid and then stirred with ammonium hydroxide:water (1:4) for 15 min. The organic phase was washed with sodium bicarbonate:water, dried with sodium sulfate, and concentrated to provide 3.7 g (94%) of (1S)-2,2-Dimethyl-1-({3-[4-(trifluoromethyl)phenyl]-1H-pyrazol-1-yl}methyl)propyl 4-nitrophenyl carbonate as a pale yellow solid. ¹H NMR (DMSO-d₆): δ 8.10 (d, J=9 Hz, 2H), 7.97 (d, J=8 Hz, 2H), 7.91, (d, J=2 Hz, 1H), 7.70 (d, J=8 Hz, 2H), 7.21 (d, J=9 Hz, 2H), 6.85 (d, J=2 Hz, 1H), 4.87 (dd, J=10 Hz, J=2 Hz, 1H), 4.61 (dd, J=14 Hz, J=2 Hz, 1H), 4.33 (dd, J=14 Hz, J=10 Hz, 1H), 1.02 (s, 9H); ES-LCMS m/z 478 (M+H).

### Example 1h

### Preparation of tert-butyl(1S)-1-{[methoxy(methyl)amino]carbonyl}pentylcarbamate

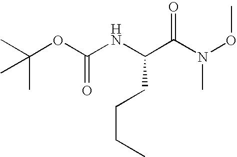

To a stirred solution of 27.8 g (120.0 mmol) of N-Boc-L-Norleucine in 150 mL of dichloromethane at −40° C. was added a solution of 18.4 mL (151.5 mmol) of 1-methylpiperidine in 40 mL of dichloromethane over 20 min. Then, 13.9 mL (145.4 mmol) of ethyl chloroformate in 40 mL of dichloromethane was then added over 30 min and the reaction mixture was stirred at −40° C. for 2.5 h. A solution of 14.2 g (145.4 mmol) of N,O-dimethylhydroxylamine hydrochloride and 18.4 mL (151.5 mmol) of 1-methylpiperidine in 90 mL of dichloromethane was added over 45 min, and the reaction mixture was allowed to slowly warm to ambient temperature and stir for 18 h. It was then washed with 100 mL of water, 100 mL of 1% hydrochloric acid (2×), 100 mL of saturated aqueous sodium bicarbonate, and dried over anhydrous magnesium sulfate. Concentration in vacuo afforded 35.0 g (quantitative yield) of crude tert-butyl (1S)-1-{[methoxy(methyl)amino]carbonyl}pentylcarbamate as a thick oil. ¹H NMR (400 MHz, DMSO-d₆): δ 6.94 (d, J=8 Hz, 1H), 4.35-4.25 (m, 1H), 3.68 (s, 3H), 3.05 (s, 3H), 1.52-1.36 (m, 2H), 1.32 (s, 9H), 1.30-1.14 (m, 4H), 0.80 (t, J=6 Hz, 3H).

### Example 1i

### Preparation of tert-butyl(1S)-1-formylpentylcarbamate

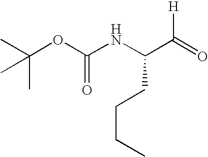

To a stirred solution of 54.0 mL (180.0 mmol) of 65 wt % bis(2-methoxyethoxy) aluminum hydride in toluene in 100 mL of toluene at −20° C. was added a solution of 35.0 g (120.0 mmol) of tert-butyl(1S)-1-{[methoxy(methyl)amino]carbonyl}pentylcarbamate in 100 mL of toluene over 30 min. After stirring at −20° C. for 2 h, 300 mL of 3M sodium chloride was added dropwise and the layers were separated. The toluene portion was washed with 100 mL of 1 N hydrochloric acid (2×), 50 mL of 0.1 N sodium hydroxide (2×), 50 mL of brine, and dried over anhydrous magnesium sulfate, then concentrated to ˜200 mL. The aldehyde was used immediately in a solution. An aliquot of the solution was removed and the concentrated aldehyde was analyzed immediately. ¹H NMR (400 MHz, DMSO-d₆): δ 9.39 (s, 1H), 7.23 (d, J=7 Hz, 1H), 3.75 (m, 1H), 1.70-1.08 (m, 6H), 1.36 (s, 9H), 0.81 (t, J=6 Hz, 3H).

### Example 1j

### Preparation of tert-butyl(1S)-1-[(R)-cyano(hydroxy)methyl]pentylcarbamate Et tert-butyl(1S)-1-[(S)-cyano(hydroxy)methyl]pentylcarbamate

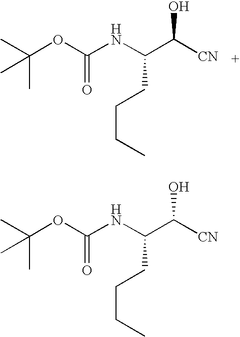

To a stirred solution of tert-butyl(1S)-1-formylpentylcarbamate in toluene was added 50 mL of water, 16.4 mL (180.0 mmol) of acetone cyanohydrin, 250 mg (2.9 mmol) of potassium cyanide and 300 mg (0.8 mmol) of tetrabutylammonium iodide. The mixture was stirred at ambient temperature for 20 h, and then the layers were separated. The extract was washed with 60 mL of water (5×), dried over anhydrous magnesium sulfate and concentrated in vacuo to afford 26.7 g (92%) of tert-butyl (1S)-1-[(R)-cyano(hydroxy)methyl]pentylcarbamate Et tert-butyl(1S)-1-[(S)-cyano(hydroxy)methyl]pentylcarbamate as a thick oil. The ¹H NMR spectrum showed an approximately equal mixture of diastereomers. ¹H NMR (400 MHz, DMSO-d₆): δ 6.91, 6.83 (2d, J=8 Hz, J=9 Hz, 1H), 6.54, 6.47 (2d, J=7 Hz, J=6 Hz, 1H), 4.44, 4.17 (2t, J=5 Hz, J=8 Hz, 1H), 3.60-3.50 (m, 1H), 1.65-1.10 (m, 6H), 1.34 (s, 9H), 0.80 (t, J=6 Hz, 3H).

### Example 1k

### Preparation of (2R,3S)-3-amino-2-hydroxyheptanoic acid hydrochloride Et(2S,3S)-3-amino-2-hydroxyheptanoic acid hydrochloride

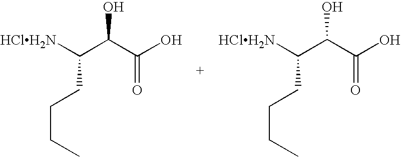

A mixture of 26.7 g (110 mmol) of tert-butyl(1S)-1-[(R)-cyano(hydroxy)methyl]pentylcarbamate Et tert-butyl(1S)-1-[(S)-cyano(hydroxy)methyl]pentylcarbamate and 200 mL of concentrated hydrochloric acid was stirred at 110° C. for 6 h and then allowed to stand at ambient temperature for 18 h. The reaction mixture was concentrated in vacuo, 200 mL of toluene was added and concentrated again to afford 25.6 g (quantitative yield) of crude (2R,3S)-3-amino-2-hydroxyheptanoic acid hydrochloride Et(2S,3S)-3-amino-2-hydroxyheptanoic acid hydrochloride as a white paste. The ¹H NMR spectrum showed the desired product to be a mixture of diastereomers and ammonium chloride. The material was used without further purification. ¹H NMR (400 MHz, DMSO-d₆): δ 8.25, 8.02 (2br s, 3H), 4.38, 4.05 (2d, J=5 Hz, J=7 Hz, 1H), 3.35-3.10 (m, 1H), 1.70-1.10 (m, 6H), 0.80 (2t, J=6 Hz, 3H).

### Example 1l

### Preparation of (2R,3S)-3-[(tert-butoxycarbonyl)amino]-2-hydroxyheptanoic acid Et(2S,3S)-3-[(tert-butoxycarbonyl)amino]-2-hydroxyheptanoic acid

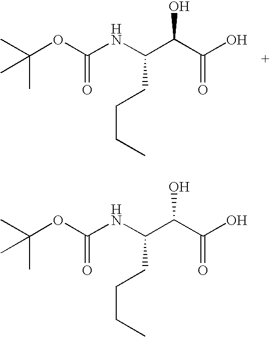

To a stirred solution of 25.6 g (110.0 mmol) of (2R,3S)-3-amino-2-hydroxyheptanoic acid hydrochloride Et(2S,3S)-3-amino-2-hydroxyheptanoic acid hydrochloride in 300 mL of 1N sodium hydroxide was added a solution of 26.4 g (121.2 mmol) of di-tert-butyldicarbonate in 75 mL of tetrahydrofuran over 30 min. After stirring at ambient temperature for 20 h, the reaction mixture was diluted with 100 mL of ether and the layers were separated. The aqueous layer was cooled in an ice bath, acidified to pH 2 with concentrated hydrochloric acid, and extracted with 150 mL of dichloromethane (2×). The dichloromethane layer was washed with 100 mL of brine, dried over anhydrous magnesium sulfate, and concentrated in vacuo to afford 21.7 g (75% over 2 steps) of (2R,3S)-3-[(tert-butoxycarbonyl)amino]-2-hydroxyheptanoic acid Et(2S,3S)-3-[(tert-butoxycarbonyl)amino]-2-hydroxyheptanoic acid as a thick paste. ¹H NMR (400 MHz, DMSO-d₆): δ 12.40 (br s, 1H), 6.45, 6.15 (2d, J=9 Hz, J=10 Hz, 1H), 5.20, 4.98 (2br, 1H), 3.89 (2d, J=12 Hz, J=10 Hz, 1H), 3.73-3.60 (m, 1H), 1.46-1.08 (m, 6H), 1.32, 1.31 (2s, 9H), 0.80 (t, J=6 Hz, 3H).

### Example 1m

### Preparation of tert-butyl(1S)-1-[(1R)-1-hydroxy-2-oxo-2-(1H-pyrazol-5-ylamino)ethyl]pentylcarbamate Et tert-butyl(1S)-1-[(1S)-1-hydroxy-2-oxo-2-(1H-pyrazol-5-ylamino)ethyl]pentylcarbamate

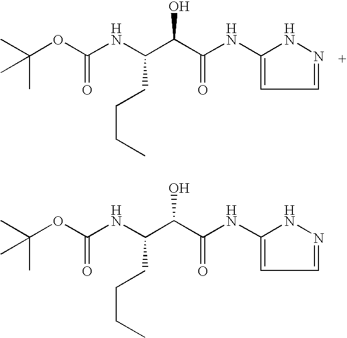

To a stirred solution of 5.0 g (19.1 mmol) of (2R,3S)-3-[(tert-butoxycarbonyl)amino]-2-hydroxyheptanoic acid Et(2S,3S)-3-[(tert-butoxycarbonyl)amino]-2-hydroxyheptanoic acid and 3.5 mL (28.7 mmol) of 1-methylpiperidine in 40 mL of dichloromethane at −40° C. was added a solution of 2.0 mL (21.0 mmol) of ethyl chloroformate in 20 mL of dichloromethane over 20 min. The reaction mixture was stirred at −40° C. for 10 min and then allowed to warm to 5° C. over 30 min. Then, 3.4 g (21.0 mmol) of N,N¹-Carbonyidiimidazole was added. After 1 h, 4.5 g (54.2 mmol) of 3-aminopyrazole was added and the reaction mixture was allowed to warm to ambient temperature. It was diluted with 60 mL of toluene and the flask was equipped with a short path still to remove the dichloromethane as the temperature was slowly increased to 110° C. The mixture was stirred for 20 h at that temperature. The toluene was removed in vacuo and the residue was taken up in 150 mL of ether. The solution was washed with 50 mL of water (3×), and then concentrated in vacuo. The resulting foam was dissolved in 75 mL of methanol. Then, 15 mL of 10% aqueous potassium carbonate was added and the solution stirred at ambient temperature for 48 h. The methanol was removed in vacuo and 150 mL of ether was added. The resulting solution was washed with 50 mL of water (3×), dried over anhydrous magnesium sulfate, and concentrated in vacuo to afford 4.8 g (77%) of tert-butyl(1S)-1-[(1R)-1-hydroxy-2-oxo-2-(1H-pyrazol-5-ylamino)ethyl]pentylcarbamate Et tert-butyl(1S)-1-[(1S)-1-hydroxy-2-oxo-2-(1H-pyrazol-5-ylamino)ethyl]pentylcarbamate as an off-white solid. ¹H NMR (400 MHz, DMSO-d₆): δ 12.29 (br s, 1H), 9.73, 9.50 (2br s, 1H), 7.54 (s, 1H), 6.45 (s, 1H), 6.42, 6.15 (2d, J=7 Hz, J=9 Hz, 1H), 5.87, 5.57 (2br s, 1H), 3.98 (m, 1H), 3.77-3.72 (m, 1H), 1.47-1.09 (m, 6H), 1.31, 1.25 (2s, 9H), 0.81, 0.76 (2t, J=6 Hz, 3H); ES-LCMS m/z 327 (M+H).

### Example 1n

### Preparation of (1S)-2,2-dimethyl-1-({3-[4-(trifluoromethyl)phenyl]-1H-pyrazol-1-yl}methyl)propyl(1S)-1-[(1R)-1-hydroxy-2-oxo-2-(1H-pyrazol-5-ylamino)ethyl]pentylcarbamate Et(1S)-2,2-dimethyl-1-({3-[4-(trifluoromethyl)phenyl]-1H-pyrazol-1-yl}methyl)propyl(1S)-1-[(1S)-1-hydroxy-2-oxo-2-(1H-pyrazol-5-ylamino)ethyl]pentylcarbamate

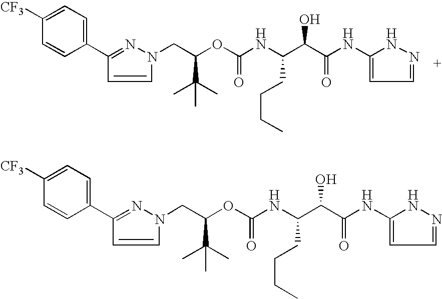

First, 0.10 g (0.31 mmol) of tert-butyl(1S)-1-[(1R)-1-hydroxy-2-oxo-2-(1H-pyrazol-5-ylamino)ethyl]pentylcarbamate Et tert-butyl(1S)-1-[(1S)-1-hydroxy-2-oxo-2-(1H-pyrazol-5-ylamino)ethyl]pentylcarbamate were dissolved in 2 mL of 4N hydrogen chloride in 1,4-dioxane, and the mixture was stirred at ambient temperature for 30 min. Solvent was evaporated and ethyl acetate was added to the residue, then distilled off. The residue was dissolved in 3 mL of N,N-dimethylformamide. Then, 0.15 g (0.31 mmol) of (1S)-2,2-dimethyl-1-({3-[4-(trifluoromethyl)phenyl]-1H-pyrazol-1-yl}methyl)propyl 4-nitrophenyl carbonate was added, followed by 0.10 g (0.93 mmol) of triethylamine, and the mixture was stirred at ambient temperature under nitrogen atmosphere for 72 h. The mixture was poured into 25 mL of water, and the solid was isolated by filtration, and dissolved in dichloromethane. The solution was washed with saturated aqueous sodium bicarbonate, dried with sodium sulfate, and concentrated. The residue was then purified by silica gel chromatography eluting with hexanes:ethyl acetate (4:1) to provide 0.1 g (57%) of (1S)-2,2-dimethyl-1-({3-[4-(trifluoromethyl)phenyl]-1H-pyrazol-1-yl}methyl)propyl(1S)-1-[(1R)-1-hydroxy-2-oxo-2-(1H-pyrazol-5-ylamino)ethyl]pentylcarbamate Et(1S)-2,2-dimethyl-1-({3-[4-(trifluoromethyl)phenyl]-1H-pyrazol-1-yl}methyl)propyl(1S)-1-[(1S)-1-hydroxy-2-oxo-2-(1H-pyrazol-5-ylamino)ethyl]pentylcarbamate as a ˜1:1 mixture of diastereomers. ¹H NMR (DMSO-d₆): δ 12.35, 12.25 (2 br s, 1H), 9.65, 9.55 (2 br s, 1H), 7.93 (d, J=8 Hz, 2H), 7.71, 7.70 (2 d, J=2 Hz, 1H), 7.67 (d, J=8 Hz, 2H), 7.55-7.60 (m, 1H), 6.7, 6.6 (2 s, 1H), 6.3-6.4 (m, 2H), 5.5, 5.9 (2 br s, 1H), 4.63, 4.82 (2 d, J=8 Hz, 1H), 4.41, 4.39 (2d, J=13 Hz, 1H), 4.1 (2 dd, J=13 Hz, J=8 Hz, 1H), 3.96-3.92 (2 m, 1H), 3.7-3.6 (2 m, 1H), 1.5-1.2 (m, 4H), 1.00-0.75 (m, 11H), 0.68 (2 t, 3H); ES-LCMS m/z 565 (M+H).

### Example 1o

### (1S)-2,2-Dimethyl-1-({3-[4-(trifluoromethyl)phenyl]-1H-pyrazol-1-yl}methyl)propyl(1S)-1-[oxo(1H-pyrazol-3-ylamino)acetyl]pentylcarbamate

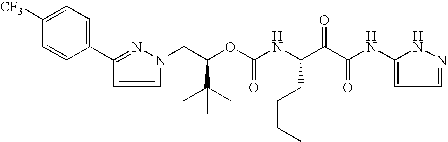

To a solution of 0.10 g (0.17 mmol) of (1S)-2,2-dimethyl-1-({3-[4-(trifluoromethyl)phenyl]-1H-pyrazol-1-yl}methyl)propyl(1S)-1-[(1R)-1-hydroxy-2-oxo-2-(1H-pyrazol-5-ylamino)ethyl]pentylcarbamate Et(1S)-2,2-dimethyl-1-({3-[4-(trifluoromethyl)phenyl]-1H-pyrazol-1-yl}methyl)propyl(1S)-1-[(1S)-1-hydroxy-2-oxo-2-(1H-pyrazol-5-ylamino)ethyl]pentylcarbamate in 2.5 mL of anhydrous dichloromethane was added 0.076 g (0.18 mmol) of Dess-Martin periodinane, and the mixture was stirred at ambient temperature for 1 h. The mixture was diluted with dichloromethane and washed with saturated aqueous sodium bicarbonate containing 5% sodium thiosulfate. The dichloromethane layer was dried with sodium sulfate and concentrated, and the residue was purified by silica gel chromatography eluting with hexanes:ethyl acetate (5:1) to provide 0.076 (76%) of (1S)-2,2-Dimethyl-1-({3-[4-(trifluoromethyl)phenyl]-1H-pyrazol-1-yl}methyl)propyl(1S)-1-[oxo(1H-pyrazol-3-ylamino)acetyl]pentylcarbamate as a solid foam. ¹H NMR (DMSO-d_{d}): δ 12.5 (br s, 1H), 10.79 (br s, 1H), 7.94 (d, J=8 Hz, 2H), 7.69 (s, 1H), 7.68 (d, J=8 Hz, 2H), 7.60 (s, 1H), 7.42 (d, J=8 Hz, 1H), 6.72 (d, J=1.5 Hz, 1H), 6.43 (br s, 1H), 4.77 (d, J=8.5 Hz, 1H), 4.55-4.62 (m, 1H), 4.42 (d, J=13 Hz, 1H), 4.14 (dd, J=13 Hz, J=10 Hz, 1H), 1.5-1.0 (m, 6H), 0.92 (s, 9H), 0.75 (t, 3H); ES-LCMS m/z 563 (M+H); Anal. calcd. for C₂₇H₃₃F₃N₆O₄.0.25 H₂O: C, 57.18; H, 5.95; N, 14.82. Found: C, 57.31; H, 5.94; N, 14.66.

### Example 2

### Preparation of (1R)-2,2-dimethyl-1-({3-[4-(trifluoromethyl)phenyl]-1H-pyrazol-1-yl}methyl)propyl(1S)-1-[oxo(1H-pyrazol-3-ylamino)acetyl]pentylcarbamate

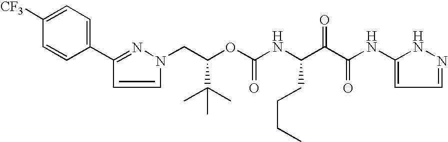

### Example 2a

### (2R)-3,3-Dimethyl-1-{3-[4-(trifluoromethyl)phenyl]-1H-pyrazol-1-yl}-2-butanol

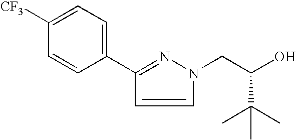

A mixture of 1.36 g (6.41 mmol) of 3-[4-(trifluoromethyl)phenyl]-1H-pyrazole, 5.14 g (51.2 mmol) of 3,3-dimethyl-1,2-epoxybutane, 1.1 mL (7.9 mmol) of triethylamine, and 40 mL of isopropyl alcohol was heated at 85° C. for 48 h. Solvent was evaporated and the residue was purified by silica gel chromatography eluting with ethyl acetate:hexanes (1:7) to give 1.17 g (59%) of 3,3-dimethyl-1-{3-[4-(trifluoromethyl)phenyl]-1H-pyrazol-1-yl}-2-butanol. Chiral Supercritical Fluid Chromatography (Chiralpak AD, 10 micron, 2.0×25 cm, 5% methanol:95% carbon dioxide) provided both the title compound (0.4 g) and its enantiomer (2S)-3,3-dimethyl-1-{3-[4(trifluoromethyl)phenyl]-1H-pyrazol-1-yl}-2-butanol (0.4 g) as pale yellow solids.

¹H NMR and ES-LCMS data identical to the title compound in Example 1f.

### Example 2b

### (1R)-2,2-Dimethyl-1-({3-[4-(trifluoromethyl)phenyl]-1H-pyrazol-1-yl}methyl)propyl 4-nitrophenyl carbonate

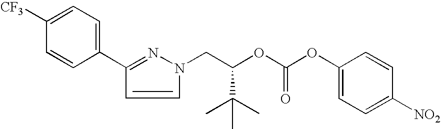

(2R)-3,3-dimethyl-1-{3-[4-(trifluoromethyl)phenyl]-1H-pyrazol-1-yl}-2-butanol was subjected to the procedure of Example 1g to provide the title compound with ¹H NMR and ES-LCMS data identical to that of the product of Example 1g.

### Example 2c

### (1R)-2,2-Dimethyl-1-({3-[4-(trifluoromethyl)phenyl]-1H-pyrazol-1-yl}methyl)propyl(1S)-1-[oxo(1H-pyrazol-3-ylamino)acetyl]pentylcarbamate

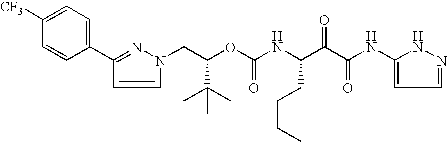

(1R)-2,2-Dimethyl-1-({3-[4-(trifluoromethyl)phenyl]-1H-pyrazol-1-yl}methyl)propyl 4-nitrophenyl carbonate was subjected sequentially to the procedures of Examples 1n and 1o to provide the title compound as a solid foam. ¹H NMR (DMSO-d₆): δ 12.5 (br s, 1H), 10.79 (br s, 1H), 8.0-7.5 (m, 2H), 7.75-7.60 (m, 4H), 7.0-6.4 (m, 3H), 5.0-4.6 (m, 2H), 4.49-4.38 (m, 1H), 4.2-4.1 (m, 1H), 1.5-1.0 (m, 6H), 0.92 (2s, 9H), 0.75 (2t, 3H); ES-LCMS m/z 563 (M+H); Anal. calcd. for C₂₇H₃₃F₃N₆O₄.0.26 H₂O: C, 57.17; H, 5.96; N, 14.81. Found: C, 57.16; H, 5.93; N, 14.54.

### Example 3

### Preparation of (1R)-2,2-dimethyl-1-({5-[4-(trifluoromethyl)phenyl]-1,3,4-oxadiazol-2-yl}methyl)propyl(1S)-1-[oxo(1H-pyrazol-3-ylamino)acetyl]pentylcarbamate

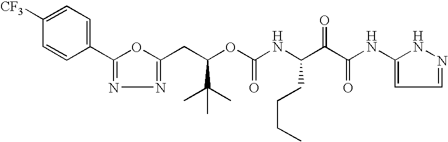

### Example 3a

### Preparation of 2-methyl-5-[4-(trifluoromethyl)phenyl-1,3,4-oxadiazole

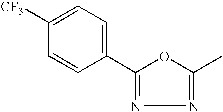

A solution of 24.69 g (121 mmol) of methyl 4-(trifluoromethyl)benzoate and 4.17 mL (133 mmol) of anhydrous hydrazine in 100 mL of absolute ethanol was heated at reflux for 18 h under nitrogen. The light yellow solution was then cooled, and volatiles were removed under vacuum to afford an off-white crystalline solid, which was further dried under vacuum to afford 42.41 g of crude 4-(trifluoromethyl)benzhydrazide. This material was then dissolved in 400 mL of hot xylenes. Then, 33 mL (180 mmol) of triethylorthoacetate was added, and the solution was heated to reflux in a flask outfitted with a Dean-Stark trap. Initially, ca 50 mL of pink solution distilled over and was drained off. After 18 h, volatiles were removed under vacuum, leaving a slightly pink solid, which was recrystallized from ethyl acetate to afford two crops of colorless crystals giving 19.357 g (70%) of 2-methyl-5-[4-(trifluoromethyl)phenyl]-1,3,4-oxadiazole. ¹H NMR (400 MHz, DMSO-d₆) δ 8.06 (AA′BB′quartet, J=8 Hz, Δv=86 Hz, 4H), 2.60 (s, 3H); ES-LCMS m/z 229 (M+H).

### Example 3b

### Preparation of (2R)-3,3-dimethyl-1-{5-[4-(trifluoromethyl)phenyl]-1,3,4-oxadiazol-2-yl}-2-butanol Et(2S)-3,3-dimethyl-1-{5-[4-(trifluoromethyl)phenyl]-1,3,4-oxadiazol-2-yl}-2-butanol

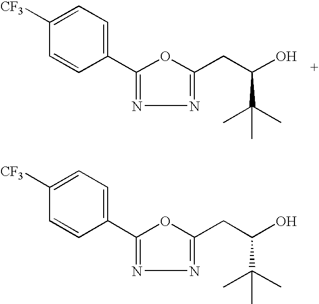

A solution of 45.0 mL (72.0 mmol) of a 1.6 M solution of n-butyllithium in hexanes was added to 12.15 mL (72.0 mmol) of neat 2,2,6,6-tetramethylpiperidine at 0° C. under nitrogen. After 1 h, the resulting yellow slurry was dissolved in 100 mL of anhydrous tetrahydrofuran, and added over 20 min to a solution of 8.21 g (36 mmol) of 2-methyl-5-[4-(trifluoromethyl)phenyl]-1,3,4-oxadiazole and 23.46 g (216 mmol) of trimethylacetaldehyde in 150 mL of anhydrous tetrahydrofuran in a dry ice-acetone bath. When solids began to precipitate, the cold bath was removed briefly. After the addition was complete, the resulting light orange solution was allowed to warm to room temperature. After 1.5 h, the now yellow solution was poured onto a mixture of 200 mL of 1 N hydrochloric acid/ice and 200 mL of ethyl acetate. The layers were separated, and the lower layer was extracted with three 150 mL portions of ethyl acetate. The organic phases were then combined, washed with two 50 mL portions of 1 N hydrochloric acid, followed by 50 mL of saturated aqueous sodium chloride. After drying over anhydrous magnesium sulfate, the organic phase was concentrated under vacuum. The resulting oil was further purified by silica gel column chromatography eluting with hexane:ethyl acetate (5:1), followed by recrystallization from hexanes:ethyl acetate to afford 7.26 g (64%) in two crops of colorless crystals of 3,3-dimethyl-1-{5-[4-(trifluoromethyl)phenyl]-1,3,4-oxadiazol-2-yl}-2-butanol. ¹H NMR (300 MHz, DMSO-d₆) δ 8.08 (AA′BB′, J=9 Hz, Δv=70 Hz, 4H), 4.98 (d, J=6 Hz, 1H), 3.69-3.62 (m, 1H), 3.09 (app dd, J=15 Hz, J=3 Hz, 1H), 2.83 (app dd, J=15 Hz, J=11 Hz, 1H), 0.91 (s, 9H); ES-LCMS m/z 315 (M+H).

The individual enantiomers were obtained via preparative supercritical fluid chromatography on a Chiralpak AD column (20×250 mm) using a Super C-20 supercritical fluid chromatograph equipped with a carbon dioxide pump, a modifier pump, an automated injector, a column oven, and a UV detector (Novasep, France). 7.26 g of 3,3-dimethyl-1-{5-[4-(trifluoromethyl)phenyl]-1,3,4-oxadiazol-2-yl}-2-butanol was dissolved in 32 mL of isopropyl alcohol. Aliquots of this solution (0.7 mL) were injected onto the Chiralpak AD column, which was eluted with carbon dioxide (41 g/min) and isopropanol (4.4 mL/min) at a pressure of 140 bar. The column was maintained at 27° C., and compounds were detected at 290 nm. In this manner, 3.236 g of isomer 1 was obtained as a colorless crystalline solid (>99% ee). Isomer 2 was also obtained as a colorless crystalline solid (3.268 g, >99% ee).

### Example 3c

### Preparation of (1R)-2,2-dimethyl-1-({5-[4-(trifluoromethyl)phenyl]-1,3,4-oxadiazol-2-yl}methyl)propyl 4-nitrophenyl carbonate

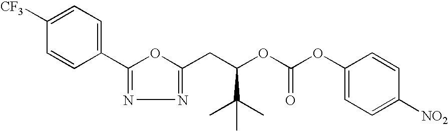

To 3.34 g (10.6 mmol) of (2R)-3,3-dimethyl-1-{5-[4-(trifluoromethyl)phenyl]-1,3,4-oxadiazol-2-yl}-2-butanol in 100 mL of anhydrous tetrahydrofuran at 0° C. was added dropwise 6.98 mL (11.2 mmol) of 1.6 M n-butyllithium in hexanes, and the resulting solution was stirred for 10 min. A solution of 3.20 g (16.0 mmol) of 4-nitrophenyl chloroformate in 10 mL of tetrahydrofuran was added in one portion. The solution was allowed to warm to room temperature and stirred overnight. The reaction mixture was diluted with 200 mL of ethyl acetate and washed with 50 mL of 1 M NaOH. The extract was dried over anhydrous magnesium sulfate, filtered, and concentrated. The residue was purified by silica gel column chromatography eluting with an ethyl acetate:dichloromethane solution (0.25:9.75) to give 2.34 g (46% yield) of (1R)-2,2-dimethyl-1-({5-[4-(trifluoromethyl)phenyl]-1,3,4-oxadiazol-2-yl}methyl)propyl 4-nitrophenyl carbonate. ¹H NMR (300 MHz, DMSO-d₆) δ 8.24 (d, J=9 Hz 2H), 8.25 (d, J=8 Hz, 2H), 7.95(d, J=8 Hz, 2H), 7.39(d, J=9 Hz, 2H), 5.00 (dd, J=11 Hz, J=2 Hz, 1H), 3.50 (dd, J=16 Hz, J=2 Hz,1H), 3.34-3.25 (m overlapping H₂O, 1H), 1.04 (s, 9H) ; Cl-LCMS m/z 480 (M+H).

### Example 3d

### Preparation of (1R)-2,2-dimethyl-1-({5-[4-(trifluoromethyl)phenyl]-1,3,4-oxadiazol-2-yl}methyl)propyl(1S)-1-[(1R)-1-hydroxy-2-oxo-2-(1H-pyrazol-5-ylamino)ethyl]pentylcarbamate Et(1R)-2,2-dimethyl-1-({5-[4-(trifluoromethyl)phenyl]-1,3,4-oxadiazol-2-yl}methyl)propyl(1S)-1-[(1S)-1-hydroxy-2-oxo-2-(1H-pyrazol-5-ylamino)ethyl]pentylcarbamate

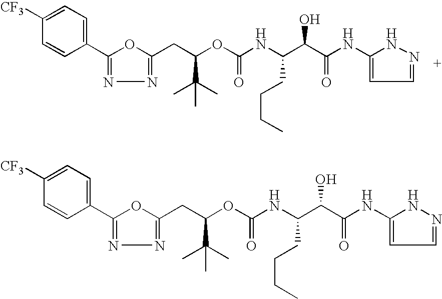

To 1.77 g (5.40 mmol) of tert-butyl(1S)-1-[(1R)-1-hydroxy-2-oxo-2-(1H-pyrazol-5-ylamino)ethyl]pentylcarbamate Et tert-butyl(1S)-1-[(1S)-1-hydroxy-2-oxo-2-(1H-pyrazol-5-ylamino)ethyl]pentylcarbamate in 10 mL of 1,4-dioxane was added 60 mL of 4 M hydrochloric acid in 1,4-dioxane. The reaction mixture was stirred for 30 min, concentrated, and dried under vacuum before being dissolved in 20 mL of N,N-dimethylformamide. To the resulting solution was added 2.5 mL (14.3 mmol) of diisopropylamine. This solution was added to 2.29 g (4.80 mmol) of (1R)-2,2-dimethyl-1-({5-[4-(trifluoromethyl)phenyl]-1,3,4-oxadiazol-2-yl}methyl)propyl 4-nitrophenyl carbonate, and the resulting solution was stirred overnight The reaction mixture was diluted with 100 mL of ethyl acetate and washed successively with 30 mL of saturated aqueous sodium bicarbonate, 20 mL of 1 M sodium hydroxide, and 30 mL of saturated aqueous sodium chloride. The organic phase was dried over anhydrous magnesium sulfate and concentrated. The residue was purified by silica gel column chromatography eluting with methanol:dichloromethane (1:9) to give 1.77 g (65%) of (1R)-2,2-dimethyl-1-({5-[4-(trifluoromethyl)phenyl]-1,3,4-oxadiazol-2-yl}methyl)propyl(1S)-1-[(1R)-1-hydroxy-2-oxo-2-(1H-pyrazol-5-ylamino)ethyl]pentylcarbamate Et(1R)-2,2-dimethyl-1-({5-[4-(trifluoromethyl)phenyl]-1,3,4-oxadiazol-2-yl}methyl)propyl(1S)-1-[(1S)-1-hydroxy-2-oxo-2-(1H-pyrazol-5-ylamino)ethyl]pentylcarbamate. Analytical samples of each diastereomer were isolated separately and qualitatively analyzed. Isomer 1: R_{f}=0.30 (1:9 methanol:dichloromethane); ¹H NMR (300 MHz, DMSO-d₆) δ 12.33 (s, 1H), 9.48 (s, 1H), 8.16 (d, J=8 Hz, 2H), 7.96 (d, J=8 Hz, 2H), 7.57 (s, 1H), 6.74 (d, J=10 Hz, 1H), 6.43 (s, 1H), 5.80 (s, 1H), 4.91 (d, J=11 Hz, 1H), 3.89 (t, J=5 Hz, 1H), 3.60-3.53 (m, 1H), 3.08 (t, J=15 Hz, 1H), 1.27-1.08 (m, 3H), 0.98-0.70 (m, 12 H), 0.50 (t, J=7 Hz, 3H); Cl-LCMS m/z 567 (M+H). Isomer 2: R_{f}=0.22 (1:9 methanol:chloroform); ¹H NMR (300 MHz, DMSO-d₆) δ 12.26 (s, 1H), 9.69 (s, 1H), 8.16 (d, J=8 Hz, 2H), 7.96 (d, J=9 Hz, 2H), 7.54 (s, 1H), 6.48 (d, J=9 Hz, 1H), 6.38 (s, 1H), 5.45 (s, 1H), 4.80 (d, J=11 Hz, 1H), 3.93 (d, J=7 Hz, 1H), 3.63 (m, 1H), 3.25 (d, J=16 Hz, 1H) 3.03 (t, J=15 Hz, 1H), 1.25 (m, 1H), 1.11 (m, 1H), 0.93-0.79 (m, 13H), 0.54 (t, J=7 Hz, 3H); Cl-LCMS m/z 567 (M+H).

### Example 3e

### Preparation of (1R)-2,2-dimethyl-1-({5-[4-(trifluoromethyl)phenyl]-1,3,4-oxadiazol-2-yl}methyl)propyl(1S)-1-[oxo(1H-pyrazol-3-ylamino)acetyl]pentylcarbamate

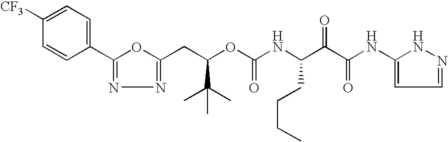

To 1.66 g (2.93 mmol) of (1R)-2,2-dimethyl-1-({5-[4-(trifluoromethyl)phenyl]-1,3,4-oxadiazol-2-yl}methyl)propyl(1S)-1-[(1R)-1-hydroxy-2-oxo-2-(1H-pyrazol-5-ylamino)ethyl]pentylcarbamate Et(1R)-2,2-dimethyl-1-({5-[4-(trifluoromethyl)phenyl]-1,3,4-oxadiazol-2-yl}methyl)propyl(1S)-1-[(1S)-1-hydroxy-2-oxo-2-(1H-pyrazol-5-ylamino)ethyl]pentylcarbamate in 20 mL of dichloromethane at room temperature was added 1.55 g (3.67 mmol) of Dess-Martin periodinane. The reaction mixture was stirred for 20 min and filtered through a celite plug. The filtrate was concentrated and the residue was partially purified by silica gel chromatography eluting with acetone:dichloromethane (1:9, then 2:9). The impure product was taken up in dichloromethane and the resulting solution was washed with brine, dried over anhydrous magnesium sulfate, and concentrated to give 1.3 g (79%) of (1R)-2,2-dimethyl-1-({5-[4-(trifluoromethyl)phenyl]-1,3,4-oxadiazol-2-yl}methyl)propyl(1S)-1-[oxo(1H-pyrazol-3-ylamino)acetyl]pentylcarbamate after drying under vacuum. ¹H NMR (300 MHz, DMSO-d₆, Temp=100° C.) δ 10.33 (s, 1H), 8.17 (d, J=8 Hz, 2H), 7.91 (d, 8 Hz, 2H), 7.56 (s, 1H), 6.44 (s, 1H), 4.90 (d, J=10 Hz, 1H), 4.69 (m, 1H), 3.29 (d, J=15 Hz, 1H), 3.02-3.12 (m overlapping H₂O, 1H), 1.66 (m, 1H), 1.43 (m, 1H), 1.28-1.06 (m, 4H), 0.97 (s, 9H), 0.73 (br s, 3H); Cl-LCMS m/z 565 (M+H); HRMS C₂₆H₃₁F₃N₆O₅ m/z 565.2386 (M+H)_{Cal}; 565.2385(M+H)_{Obs}.

### Example 4

### Preparation of (1R)-1-{[5-(4-fluorophenyl)-1,3,4-oxadiazol-2-yl]methyl}-2,2-dimethylpropyl(1S)-1-{oxo[(3-pyridinylmethyl)amino]acetyl}pentylcarbamate

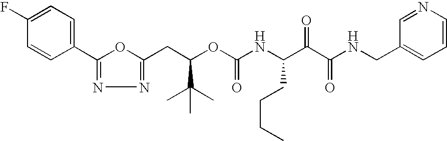

### Example 4a

### Preparation of 2-(4-fluorophenyl)-5-methyl-1,3,4-oxadiazole

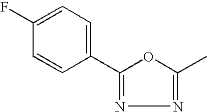

First, 7.7 g (50 mmol) of 4-fluorobenzhydrazide was dissolved in 150 mL of hot xylenes. Then, 13.73 mL (75 mmol) of triethylorthoacetate was added, and the solution was heated at reflux for 17 h in a flask outfitted with a Dean-Stark trap. Volatiles were removed under vacuum, leaving a slightly tan solid, which was triturated with 50 mL of hexanes, and filtered to afford 7.327 g of 2-(4-fluorophenyl)-5-methyl-1,3,4-oxadiazole as an off-white crystalline solid. The filtrate was partially concentrated, and the resulting solid was isolated by filtration to afford 0.920 g more of the off-white solid (92% total yield). ¹H NMR (300 MHz, DMSO-d₆) δ 8.03 (dd, J=9 Hz, J=5 Hz, 2H), 7.43 (t, J=9 Hz, 2H), 2.57 (s, 3H); ES-LCMS m/z 179 (M+H).

### Example 4b

### Preparation of (2R)-1-[5-(4-fluorophenyl)-1,3,4-oxadiazol-2-yl]-3,3-dimethyl-2-butanol Et(2S)-1-[5-(4-fluorophenyl)-1,3,4-oxadiazol-2-yl]-3,3-dimethyl-2-butanol

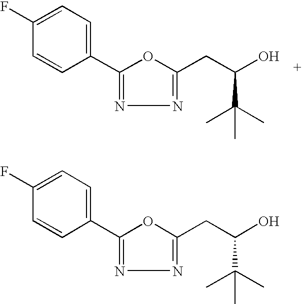

A solution of 57.75 mL (92.4 mmol) of a 1.6 M solution of n-butyllithium in hexanes was added to 15.6 mL (92.4 mmol) of neat 2,2,6,6-tetramethylpiperidine at 0° C. under nitrogen. After 1 h, the resulting yellow slurry was dissolved in 100 mL of anhydrous tetrahydrofuran, and added over 15 min to a solution of 8.24 g (46.2 mmol) of 2-(4-fluorophenyl)-5-methyl-1,3,4-oxadiazole and 15.6 mL (92.4 mmol) of trimethylacetaldehyde in 150 mL of anhydrous tetrahydrofuran in a dry ice-acetone bath. When solids began to precipitate, the cold bath was removed briefly. After the addition was complete, the resulting light orange solution was allowed to warm to room temperature. After 2.5 h, the orange solution was poured onto a mixture of 300 mL of 1 N hydrochloric acid/ice. The resulting mixture was then extracted with four 300 mL portions of ethyl acetate. The organic phases were combined, and washed with two 100 mL portions of 1 N hydrochloric acid, followed by 100 mL of saturated aqueous sodium chloride. After drying over anhydrous magnesium sulfate, the organic phase was concentrated under vacuum to afford a tan oil, which was recrystallized from hexanes to afford 8.72 g (72%) of (2R)-1-[5-(4-fluorophenyl)-1,3,4-oxadiazol-2-yl]-3,3-dimethyl-2-butanol Et(2S)-1-[5-(4-fluorophenyl)-1,3,4-oxadiazol-2-yl]-3,3-dimethyl-2-butanol as an off-white crystalline solid. ¹H NMR (300 MHz, DMSO-d₆) δ 8.04 (app dd, J=9 Hz, J=5 Hz, 2H), 7.43 (app t, J=9 Hz, 2H), 3.68-3.59 (m, 1H), 3.06 (app dd, J=15 Hz, J=3 Hz, 1H), 2.80 (app dd, J=15 Hz, J=11 Hz, 1H), 0.90 (s, 9H); ES-LCMS m/z 265 (M+H).

The individual enantiomers were obtained via preparative supercritical fluid chromatography on a Chiralpak AD column (20×250 mm) using a Super C-20 supercritical fluid chromatograph equipped with a carbon dioxide pump, a modifier pump, an automated injector, a column oven, and a UV detector (Novasep, France). 8.72 g of 1-[5-(4-Fluorophenyl)-1,3,4-oxadiazol-2-yl]-3,3-dimethyl-2-butanol was dissolved in 50 mL of isopropyl alcohol. Aliquots of this solution (0.25 mL) were injected onto the Chiralpak AD column, which was eluted with carbon dioxide (42 g/min) and methanol (2.2 mL/min) at a pressure of 140 bar. The column was maintained at 27° C., and compounds were detected at 290 nm. In this manner, 3.52 g of isomer 1 was obtained as a colorless crystalline solid in >99% ee and 3.78 g of isomer 2 was also obtained as a colorless crystalline solid in 82% ee.

### Example 4c

### Preparation of (1R)-1-{[5-(4-fluorophenyl)-1,3,4-oxadiazol-2-yl]methyl}-2,2-dimethylpropyl 4-nitrophenyl carbonate

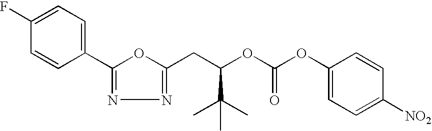

A solution of 1.50 mL (2.38 mmol) of a 1.6 M solution of n-butyllithium in hexanes was added dropwise to a solution of 571 mg (2.16 mmol) of (2R)-1-[5-(4-fluorophenyl)-1,3,4-oxadiazol-2-yl]-3,3-dimethyl-2-butanol in 20 mL of anhydrous tetrahydrofuran at 0° C. under nitrogen. The resulting light orange solution was stirred for 45 min at 0° C., before 868 mg (4.32 mmol) of 4-nitrophenyl chloroformate was added as a solid. The resulting solution was left to stir and warm to room temperature. After 3 h, the solution was diluted with 150 mL of ethyl acetate, and washed with three 70 mL aliquots of saturated aqueous sodium bicarbonate, followed by 70 mL of saturated aqueous sodium chloride. After drying over magnesium sulfate, volatiles were removed under vacuum to afford a yellow oil, which was further purified by column chromatography on silica gel. Elution with 4% ethyl acetate in dichloromethane provided a yellow oil, which was dissolved in 40 mL of ethyl acetate and washed with three 25-mL aliqouts of 1 N sodium hydroxide, followed by 25 mL of saturated aqueous sodium chloride. After drying over magnesium sulfate, and concentration under vacuum, 494 mg (53%) of (1R)-1-{[5-(4-fluorophenyl)-1,3,4-oxadiazol-2-yl]methyl}-2,2-dimethylpropyl 4-nitrophenyl carbonate was obtained as a pale yellow oil that slowly solidified upon standing. ¹H NMR (300 MHz, DMSO-d₆) δ 8.26 (d, J=9 Hz, 2H), 8.02 (dd, J=9 Hz, J=5 Hz, 2H), 7.46-7.37 (m, 2H), 4.98 (app dd, J=10 Hz, J=2 Hz, 1H), 3.46 (app dd, J=16 Hz, J=3 Hz, 1H), 3.27 (app dd, J=16 Hz, J=10 Hz, 1H), 0.90 (s, 9H); ES-LCMS m/z 430 (M+H).

### Example 4d

### Preparation of tert-butyl(1S)-1-{(1R)-1-hydroxy-2-oxo-2-[(3-pyridinylmethyl)amino]ethyl}pentylcarbamate Et tert-butyl(1S)-1-{(1S)-1-hydroxy-2-oxo-2-[(3-pyridinylmethyl)amino]ethyl}pentylcarbamate

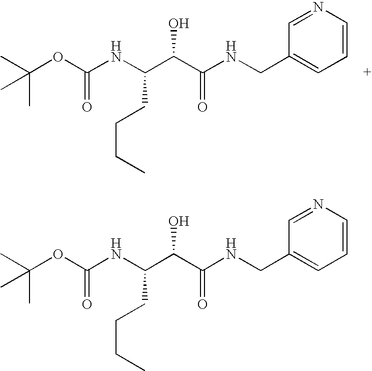

To a stirred solution of 4.1 g (15.7 mmol) of (2R,3S)-3-[(tert-butoxycarbonyl)amino]-2-hydroxyheptanoic acid Et(2S,3S)-3-[(tert-butoxycarbonyl)amino]-2-hydroxyheptanoic acid in 30 mL of dichloromethane at 5° C. was added a solution of 4.2 mL (34.5 mmol) of 1-methylpiperidine in 10 mL of dichloromethane over 10 min. Then, 3.0 mL (34.5 mmol) of ethyl chloroformate in 20 mL of dichloromethane was added over 30 min, and the reaction mixture was stirred at 5° C. for an additional 1 h. A solution of 2.6 g (23.5 mmol) of 3-(aminomethyl)pyridine in 20 mL of dichloromethane was added over 15 min and the reaction mixture was allowed to warm to ambient temperature. After stirring for 20 h, the reaction mixture was concentrated and the residue partitioned in 150 mL of ether and 50 mL of water. The layers were separated and the ether was washed with 50 mL of water, dried over anhydrous magnesium sulfate and concentrated in vacuo to afford 3.8 g of a yellow gum, which was dissolved in 50 mL of anhydrous methanol. The solution was cooled to 5° C., and 1.1 g (8.0 mmol) of potassium carbonate was added. The reaction mixture was stirred at 5° C. for 3 h then for 1.5 h at ambient temperature. The mixture was filtered, and the filtrate was concentrated. The residue was taken up in 150 mL of ether, and the solution was washed with 50 mL of water (2×), dried over anhydrous magnesium sulfate, and concentrated in vacuo to afford 2.2 g (40%) of tert-butyl(1S)-1-{(1R)-1-hydroxy-2-oxo-2-[(3-pyridinylmethyl)amino]ethyl}pentylcarbamate Et tert-butyl(1S)-1-{(1S)-1-hydroxy-2-oxo-2-[(3-pyridinylmethyl)amino]ethyl}pentylcarbamate as an off-white solid. ¹H NMR (400 MHz, DMSO-d₆): δ 8.43-8.38 (m, 3H), 7.60 (t, J=8 Hz, 1H), 7.28-7.23 (m, 1H), 6.38, 5.99 (2d, J=9 Hz, J=10 Hz, 1H), 5.65, 5.61 (2d, J=6 Hz, J=7 Hz, 1H), 4.33-4.14 (m, 2H), 3.92-3.85 (m, 1H), 3.71-3.66 (m, 1H), 1.42-1.05 (m, 6H), 1.33, 1.31 (2s, 9H), 0.79, 0.72 (2t, J=6 Hz, J=7 Hz, 3H); ES-LCMS m/z 352 (M+H).

### Example 4e

### Preparation of (1R)-1-{[5-(4-fluorophenyl)-1,3,4-oxadiazol-2-yl]methyl}-2,2-dimethylpropyl(1S)-1-{(1R)-1-hydroxy-2-oxo-2-[(3-pyridinylmethyl)amino]ethyl}pentylcarbamate Et(1R)-1-{[5-(4-fluorophenyl)-1,3,4-oxadiazol-2-yl]methyl}-2,2-dimethylpropyl(1S)-1-{(1S)-1-hydroxy-2-oxo-2-[(3-pyridinylmethyl)amino]ethyl}pentylcarbamate

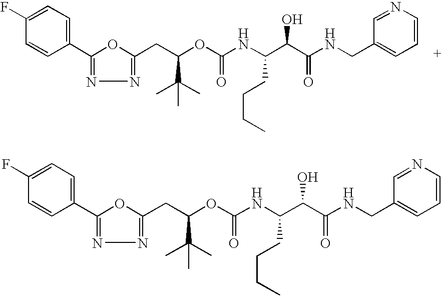

First, 300 mg (0.86 mmol) of tert-butyl(1S)-1-{(1R)-1-hydroxy-2-oxo-2-[(3-pyridinylmethyl)amino]ethyl}pentylcarbamate Et tert-butyl(1S)-1-{(1S)-1-hydroxy-2-oxo-2-[(3-pyridinylmethyl)amino]ethyl}pentylcarbamate was dissolved in 2 mL of anhydrous dioxane. Then 2 mL of a 4 N solution of hydrogen chloride in dioxane was added, and the resulting solution was stirred for 25 min, during which a white precipitate formed. The mixture was diluted with 5 mL of methanol, and then concentrated under vacuum. The resulting (3S)-3-amino-2-hydroxy-N-(3-pyridinylmethyl)heptanamide dihydrochloride, a white foam, was dried under vacuum, and then slurried in 2 mL of anhydrous N,N-dimethylformamide. Addition of 460 μL (2.6 mmol) of diisopropylethylamine resulted in a light yellow solution, to which a solution of 368 mg (0.86 mmol) of (1R)-1-{[5-(4-fluorophenyl)-1,3,4-oxadiazol-2-yl]methyl}-2,2-dimethylpropyl 4-nitrophenyl carbonate in 3 mL of N,N-dimethylformamide was added. The resulting solution was stirred for 61 h under nitrogen. It was then diluted with 40 mL of saturated aqueous sodium bicarbonate, and extracted with three 80 mL portions of ethyl acetate. The extracts were combined, and washed three times with 30 mL of saturated aqueous sodium bicarbonate and once with saturated aqueous sodium chloride. After drying over magnesium sulfate, volatiles were removed under vacuum to afford a yellow oil, which was further purified by column chromatography on silica gel. Elution with 5% methanol in chloroform afforded 352 mg (76%) of (1R)-1-{[5-(4-fluorophenyl)-1,3,4-oxadiazol-2-yl]methyl}-2,2-dimethylpropyl(1S)-1-{(1R)-1-hydroxy-2-oxo-2-[(3-pyridinylmethyl)amino]ethyl}pentylcarbamate Et(1R)-1-{[5-(4-fluorophenyl)-1,3,4-oxadiazol-2-yl]methyl}-2,2-dimethylpropyl(1S)-1-{(1S)-1-hydroxy-2-oxo-2-[(3-pyridinylmethyl)amino]ethyl}pentylcarbamate as a yellow foam. ¹H NMR (300 MHz, DMSO-d₆) δ 8.42-8.35 (m, 3H), 8.01-7.97 (m, 2H), 7.59 (m, 1H), 7.41 (t, J=9 Hz, 2H), 7.29-7.25 (m, 1H), 6.70 (d, J=9 Hz) and 6.26 (d, J=9 Hz) total 1H, 5.63 (d, J=5 Hz) and 5.54 (d, 6 Hz) total 1H, 4.93-4.84 (m, 1H), 4.28-4.15 (m, 2H), 3.81 (m, 1H), 3.53 (br m, 1H), 3.26-3.21 (m, 1H), 3.07-2.96 (m, 1H), 0.94 (s) and 0.91 (s) total 9H, 1.21-0.67 (m, 6H); ES-LCMS m/z 542 (M+H).

### Example 4f

### Preparation of (1R)-1-{[5-(4-fluorophenyl)-1,3,4-oxadiazol-2-yl]methyl}-2,2-dimethylpropyl(1S)-1-{oxo[(3-pyridinylmethyl)amino]acetyl}pentylcarbamate

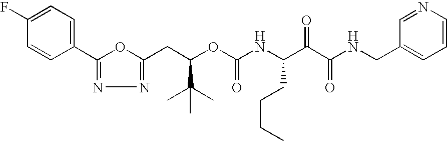

First, 115 mg (0.30 mmol) of Dess Martin periodinane was added to a stirred solution of 130 mg (0.24 mmol) of (1R)-1-{[5-(4-fluorophenyl)-1,3,4-oxadiazol-2-yl]methyl}-2,2-dimethylpropyl(1S)-1-{(1R)-1-hydroxy-2-oxo-2-[(3-pyridinylmethyl)amino]ethyl}pentylcarbamate Et(1R)-1-{[5-(4-fluorophenyl)-1,3,4-oxadiazol-2-yl]methyl}-2,2-dimethylpropyl(1S)-1-{(1S)-1-hydroxy-2-oxo-2-[(3-pyridinylmethyl)amino]ethyl}pentylcarbamate in 1.25 mL of dichloromethane. After 20 min, the red mixture was diluted with 20 mL of dichloromethane, and washed with 10 mL of saturated aqueous sodium thiosulfate. The aqueous layer was then extracted twice with 20 mL of dichloromethane. The extracts were combined, washed with saturated aqueous sodium chloride, dried over anhydrous magnesium sulfate, and concentrated to an oil, which was immediately purified by column chromatography on silica gel. Elution with 5% methanol in chloroform afforded a viscous gum, from which three 10 mL portions of chloroform were distilled under vacuum. 92 mg (71%) of (1R)-1-{[5-(4-fluorophenyl)-1,3,4-oxadiazol-2-yl]methyl}-2,2-dimethylpropyl(1S)-1-{oxo[(3-pyridinylmethyl)amino]acetyl}pentylcarbamate was isolated as a viscous gum after drying for 30 min under vacuum. ¹H NMR (300 MHz, DMSO-d₆) δ 8.92-8.84 (br m, 1 H), 8.46 (d, J=2 Hz, 1H), 8.43 (dd, J=5 Hz, J=2 Hz, 1H), 8.03-7.98 (m, 2H), 7.64-7.61 (m, 1H), 7.37 (app t, J=9 Hz, 2H), 7.28 (app dd, J=8 Hz, J=5 Hz, 1H), 7.06-6.98 (br m, 1H), 4.87 (app dd, J=10 Hz, J=3 Hz, 1H), 4.71-4.61 (br m, 1H), 4.33 (d, J=6 Hz, 2H), 3.24 (app dd, J=15 Hz, J=3 Hz, 1H), 3.0 (m, 1H, overlapping H₂O signal), 1.72-1.57 (m, 1H), 1.52-1.38 (m, 1H), 1.28-1.10 (m, 4H), 0.96 (s, 9H), 0.75 (m, 3H); ES-LCMS m/z 539 (M+H); HRMS C₂₁H₂₀N₃O₆F m/z 540.2622 (M+H)_{Cal,} 540.2612 (M+H)_{Obs}.

### Example 5

### Preparation of (1S)-2,2-dimethyl-1-({3-[4-(trifluoromethyl)phenyl]-1H-pyrazol-1-yl}methyl)propyl(1S)-1-[oxo(2-pyridinylamino)acetyl]pentylcarbamate

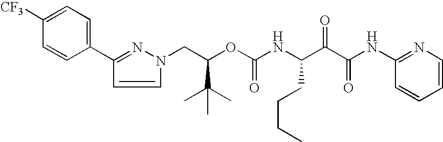

### Example 5a

### Preparation of tert-butyl(1S)-1-[(1R)-1-hydroxy-2-oxo-2-(2pyridinylamino)ethyl]pentylcarbamate Et tert-butyl(1S)-1-[(1S)-1-hydroxy-2-oxo-2-(2-pyridinylamino)ethyl]pentylcarbamate

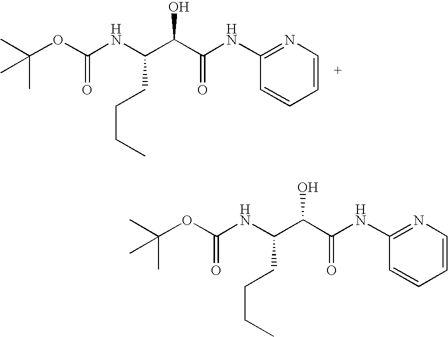

tert-butyl(1S)-1-[(1R)-1-hydroxy-2-oxo-2-(2pyridinylamino)ethyl]pentylcarbamate Et tert-butyl(1S)-1-[(1S)-1-hydroxy-2-oxo-2-(2-pyridinyl amino) ethyl]pentylcarbamate were obtained as a white solid in 39% yield following the procedure described in example 4d. ¹H NMR (400 MHz, DMSO-d₆): δ 9.61, 9.42 (2s, 1H), 8.26 (t, J=5 Hz, 1H), 8.05-7.99 (m, 1H), 7.78-7.71 (m, 1H), 7.11-7.04 (m, 1H), 6.52, 6.26 (2d, J=9 Hz, J=10 Hz, 1H), 6.10, 5.76 (2d, J=6 Hz, J=7 Hz, 1H), 4.04-4.01 (m, 1H), 3.83-3.74 (m, 1H), 1.46-1.09 (m, 6H), 1.29, 1.18 (2s, 9H), 0.82, 0.76 (2t, J=6 Hz, J=7 Hz, 3H); ES-LCMS m/z 338 (M+H).

### Example 5b

### Preparation of (1S)-2,2-dimethyl-1-({3-[4-(trifluoromethyl)phenyl]-1H-pyrazol-1-yl}methyl)propyl(1S)-1-[(1R)-1-hydroxy-2-oxo-2-(2-pyridinylamino)ethyl]pentylcarbamate Et(1S)-2,2-dimethyl-1-({3-[4-(trifluoromethyl)phenyl]-1H-pyrazol-1-yl}methyl)propyl(1S)-1-[(1S)-1-hydroxy-2-oxo-2-(2-pyridinylamino)ethyl]pentylcarbamate

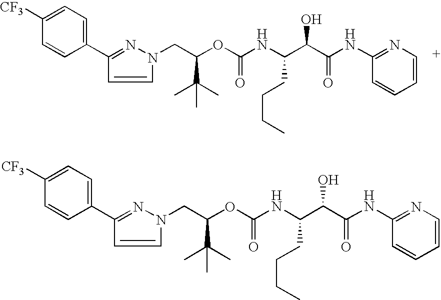

First, 0.37 g (1.1 mmol) of tert-butyl(1S )-1-[(1R)-1-hydroxy-2-oxo-2-(2-pyridinylamino)ethyl]pentylcarbamate Et tert-butyl(1S)-1-[(1S)-1-hydroxy-2-oxo-2-(2-pyridinylamino)ethyl]pentylcarbamate were dissolved in 2.0 mL of 4N hydrogen chloride in 1,4-dioxane and the mixture was stirred at ambient temperature for 30 min. Solvent was evaporated and ethyl acetate was distilled from the residue, which was then dissolved in 9 mL of N,N-dimethylformamide. Then, 0.52 g (1.1 mmol) of (1S)-2,2-Dimethyl-1-({3-[4-(trifluoromethyl)phenyl]-1H-pyrazol-1-yl}methyl)propyl 4-nitrophenyl carbonate was added followed by 0.33 g (3.3 mmol) of triethylamine, and the mixture was stirred at ambient temperature under nitrogen atmosphere for 72 h. The mixture was poured into 25 mL of water, and the resulting solid was isolated by filtration, and purified by silica gel chromatography eluting with dichloromethane:methanol (4:1) to provide 0.46 g (73%) of the title ccomoundna as a ˜1:1 mixture of diastereomers. ¹H NMR (DMSO-d₆): δ 9.54, 9.45 (2s, 1H), 8.24 (2d, J=4 Hz, 1), 8.0-7.9 (m, 3H), 7.8-7.6 (m, 4H), 7.15-7.00 (m, 1H), 6.65-6.55 (m, 1H), 6.47 (d, J=10 Hz, 1H), 6.0, 5.7 (2d, J=4 Hz, 1H), 4.7, 4.6 (2dd, J=10 Hz, J=2 Hz, 1H), 4.36 (2dd, J=12 Hz, J=2 Hz, 1H), 4.15-3.85 (m, 2H), 3.7-3.5 (2m, 1H), 1.2-0.9 (m, 6H), 0.72, 0.65 (2s, 9H), 0.62, 0.58 (2t, 3H); ES-LCMS m/z 576 (M+H).

### Example 5c

### Preparation of (1S)-2,2-Dimethyl-1-({3-[4-(trifluoromethyl)phenyl]-1H-pyrazol-1-yl}methyl)propyl(1S)-1-[oxo(2-pyridinylamino)acetyl]pentylcarbamate

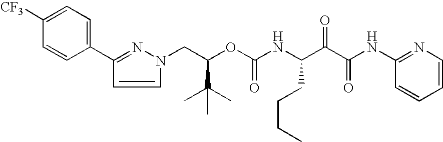

(1S)-2,2-dimethyl-1-({3-[4-(trifluoromethyl)phenyl]-1H-pyrazol-1-yl}methyl)propyl (1S)-1-[(1R)-1-hydroxy-2-oxo-2-(2-pyridinylamino)ethyl]pentyl carbamate Et (1S)-2,2-dimethyl-1-({3-[4-(trifluoromethyl)phenyl]-1H-pyrazol-1-yl}methyl)propyl (1S)-1-[(1S)-1-hydroxy-2-oxo-2-(2-pyridinylamino)ethyl]pentylcarbamate were subjected to the procedure outlined in example 1o to provide the title compound as a solid foam. ¹H NMR (DMSO-d₆, Temp=100° C.): δ 10.0 (br s, 1H), 8.3 (br s, 1H), 7.92 (d, J=8 Hz, 2H), 7.8-7.7 (m, 3H), 7.65 (d, J=8 Hz, 2H), 7.15 (br s, 1H), 6.99 (br s, IH), 6.6 (s, 1H), 4.81 (br s, 1H), 4.7-4.6 (m, 1H), 4.4 (d, J=13 Hz, 1H), 4.15 (dd, J=13 Hz, J=10 Hz, 1H), 1.7 (br s, 1H), 1.5 (br s, 1H), 1.15-1.05 (m, 4H), 0.91 (s, 9H), 0.73 (br s, 3H); ES-LCMS m/z 574 (M+H); Anal. calcd. for C₂₉H₃₄F₃N₅O₄·0.5 H₂O: C, 60.25; H, 6.02; N, 12.11. Found: C, 60.21; H. 6.02; N. 12.07.

### Example 6

### Preparation of (1S)-1-{[4-(4-fluorophenyl)-1H-imidazol-1-yl]methyl}-2,2-dimethylpropyl (1S)-1-(oxo{[(1R)-1-phenylethyl]amino}acetyl)pentylcarbamate

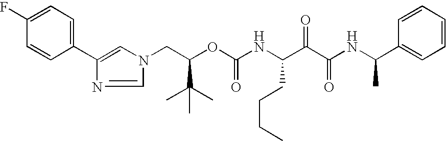

### Example 6a

### Preparation of benzyl(1S)-1-(hydroxymethyl)pentylcarbamate

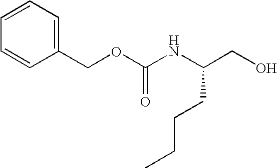

A solution of 95.0 mL (95.0 mmol) of 1 M isopropylchloroformate in toluene was added dropwise to a solution of 13.2 mL (95.0 mmol) of triethylamine and 25.16 g (95.0 mmol) of (2S)-2-{[(benzyloxy)carbonyl]amino}hexanoic acid in 200 mL of anhydrous tetrahydrofuran at 0° C. under nitrogen. After 2 h, the resulting mixture was filtered directly into a solution of 7.2 g (190 mmol) of sodium borohydride in 200 mL water. The resulting mixture was stirred for 18 h, and then was extracted with ethyl acetate. The extract was washed with saturated aqueous sodium bicarbonate, dried over anhydrous magnesium sulfate, and concentrated under vacuum. The oily solid was further purified by column chromatography on silica gel, eluting with 4:6 ethyl acetate:hexane to afford 9.63 g (40%) of (1S)-1-(hydroxymethyl)pentylcarbamate. ¹H NMR (300 MHz, DMSO-d₆) δ 7.37 (m, 5H), 6.97 (d, J=9 Hz, 1H), 5.03 (s, 2H), 4.62 (br s, 1H), 3.46-3.23 (m overlapping water peak, 3H), 1.53 (m, 1H), 1.26 (m, 5H), 0.87 (t, J=6 Hz, 3H); ES-LCMS m/z 274 (M+Na).

### Example 6b

### Preparation of benzyl(1S)-1-formylpentylcarbamate

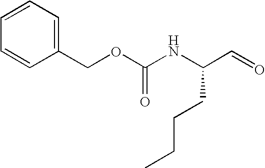

A solution of 16.38 g (103 mmol) of sulfur trioxide pyridine complex in 130 mL of dimethylsulfoxide was added to a solution of 8.64 g (34.4 mmol) of (1S)-1-(hydroxymethyl)pentylcarbamate and 14.4 mL (103 mmol) of triethylamine in 130 mL of dichloromethane at −10° C. After 1 h, the cold bath was removed, and the reaction mixture was stirred for 18 h. It was then poured slowly into a mixture of ice and saturated aqueous sodium chloride. The resulting mixture was extracted with ether. The ether extracts were then washed with 5% aqueous citric acid, and saturated aqueous sodium chloride. After drying over magnesium sulfate, volatiles were removed under vacuum to afford 7.27 g (85%) of benzyl(1S)-1-formylpentylcarbamate. ¹H NMR (400 MHz, DMSO-d₆) δ 9.41 (s, 1H), 7.66 (d, J=8 Hz, 1H), 7.33-7.28 (m, 5H), 5.00 (s, 2H), 3.85 (m, 1H), 1.65 (m, 1H), 1.40 (m, 1H), 1.27-1.18 (m 4H), 0.79 (m, 3H); ES-LCMS m/z 248 (M−H).

### Example 6c

### Preparation of (1R)-α-methylbenzylisonitrile

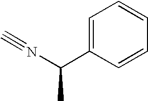

50 mL of 50% (w/w) aqueous sodium hydroxide was added to a solution of 17.5 mL (136 mmol) of (1R)-1-phenylethanamine, 10.8 mL (136 mmol) of chloroform, and 0.5 g (2.2 mmol) of benzyltriethylammonium chloride in 50 mL of dichloromethane. The resulting mixture was stirred for 3 h, and was then diluted with 100 mL of water and extracted with three 150 mL portions of dichloromethane. The combined extracts were washed with 50 mL portions of water and saturated aqueous sodium chloride, dried over anhydrous magnesium sulfate, and concentrated under vacuum to afford a dark liquid, which was further purified by column chromatography on silica gel. Elution with dichloromethane afforded 9.86 g (55%) of (1R)-α-methylbenzylisonitrile. ¹H NMR (400 MHz, DMSO-d₆) δ 7.38-7.36 (m, 4H), 7.32 (m, 1H), 5.08 (m, 1H), 1.53 (m, 3H).

### Example 6d

### Preparation of (2S)-2-{[(benzyloxy)carbonyl]amino}-1-({[(1R)-1-phenylethyl]amino}carbonyl)hexyl benzoate

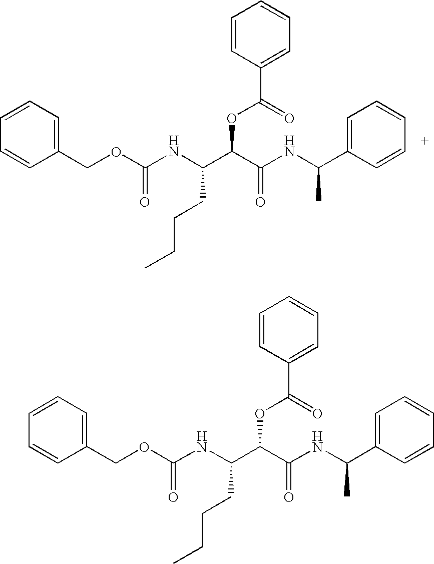

To a solution of 7.27 g (29 mmol) of benzyl(1S)-1-formylpentylcarbamate in 300 mL of dichloromethane was added 3.8 g (29 mmol) of (1R)-α-methylbenzylisonitrile and 3.54 g (29 mmol) of benzoic acid. The reaction mixture was stirred at room temperature for 48 h, and was then diluted with a copious amount of hexanes. The precipitate was collected by filtration, and the filtrate was passed through a silica plug eluting with 1:9 diethyl ether:dichloromethane. The resulting filtrate was concentrated, and the residue was combined with the collected precipitate to afford 8.7 g (60%) of (2S)-2-{[(benzyloxy)carbonyl]amino}-1-({[(1R)-1-phenylethyl]amino}carbonyl)hexyl benzoate. ¹H NMR (400 MHz, DMSO-d₆) δ 8.60 (d, J=8 Hz, 1H), 8.05 (d, J=7 Hz, 2H), 7.62 (t, J=8 Hz, 1H), 7.47 (t, J=8 Hz, 2H), 7.31-7.07 (m, 11H), 4.96 (m, 3H), 4.85 (qnt, J=7 Hz, 1H), 4.06 (m, 1H), 1.45-1.05 (m, 9H), 0.73 (t, J=7 Hz, 3H); ES-LCMS m/z 525 (M+Na).

### Example 6e

### Preparation of (3S)-3-amino-2-hydroxy-N-[(1R)-1-phenylethyl]heptanamide

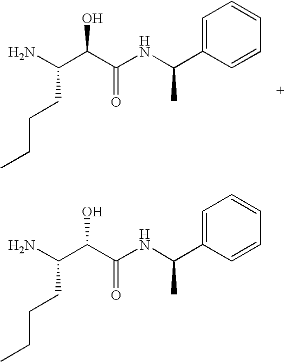

A mixture of 8.75 g (17.4 mmol) of (2S)-2-{[(benzyloxy)carbonyl]amino}-1-({[(1R)-1-phenylethyl]amino}carbonyl)hexyl benzoate and 6.97 g (174 mmol) of sodium hydroxide in 175 mL of dioxane and 75 mL of water was heated to reflux for 3 h and then let cool to room temperature. The reaction mixture was diluted with 100 mL of water and extracted with ethyl acetate. The combined ethyl acetate layers were dried over potassium carbonate and concentrated to afford 4.38 g (95%) of (3S)-3-amino-2-hydroxy-N-[(1R)-1-phenylethyl]heptanamide as a white solid. ¹H NMR (300 MHz, DMSO-d₆) δ 8.08 (d, J=8 Hz, 1H), 7.39-7.20 (m, 5H), 4.99 (m, 1H), 4.51 (br s, 1H), 3.71 (d, J=3 Hz, 1H), 2.81 (m, 1H), 1.50-1.05 (m, 9H), 0.87 (t, 3H); ES-LCMS m/z 265 (M+H).

### Example 6f

### Preparation of 4-(4-fluorophenyl)-1H-imidazole

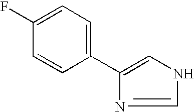

A mixture of 10.00 g (46 mmol) of 4-fluorophenacylbromide and 48 mL of formamide was heated at 175° C. for 3.5 h. 75 mL of 1N hydrochloric acid was added to the resulting solution, which was then heated to reflux. The mixture was filtered and allowed to cool to room temperature before neutralization with concentrated ammonium hydroxide. The resulting mixture was partitioned between ethyl acetate and brine. The extract was dried over anhydrous magnesium sulfate, filtered, and evaporated. The residual oil was purified by silica gel column chromatography, eluting with an ethyl acetate:methanol solution (9:1). Further purification by silica gel column chromatography eluting with a hexane:acetone solution (1:1) yielded a solid which was slurried in hexane and filtered to yield 1.22 g (16%) of 4-(4-fluorophenyl)-1H-imidazole. ¹H NMR (DMSO-d₆): δ 12.55 (br s, 1H), 7.80 (m, 2H), 7.72 (s, 1H), 7.58 (s, 1H), 7.20 (m, 2H).

### Example 6g

### Preparation of (2S)-1-[4-(4-fluorophenyl)-1H-imidazol-1-yl]-3,3-dimethyl-2-butanol

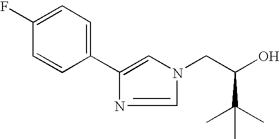

A solution of 266 mg (sample contains •0.3 equivalents of pentane by ¹H NMR for an effective weight of 164 mg, 1.64 mmol) of (S)-3,3-dimethyl-1,2-epoxybutane and 200 mg (1.64 mmol) of 4-(4-fluorophenyl)-1H-imidazole in 0.71 mL of isopropanol was placed in a sealed tube and heated at 85° C. for 18.75 h. The mixture was evaporated, and the residue was purified by silica gel column chromatography eluting with an ethyl acetate:hexanes solution (1:9) to yield 0.24 g (56%) of (2S)-1-[4(4-fluorophenyl)-1H-imidazol-1-yl]-3,3-dimethyl-2-butanol. ¹H NMR (DMSO-d₆): δ 7.76 (m, 2H), 7.67 (s, 1H), 7.66 (s, 1H), 7.18 (t, J=9 Hz, 2H), 5.02 (d, J=6 Hz, 1H), 4.15 (d, J=12 Hz, 1H), 3.74 (dd, J=10 Hz, J=4 Hz, 1H), 3.35 (m, overlapping H₂O), 0.94 (s, 9H).

### Example 6h

### Preparation of (1S)-1-{[4-(4-fluorophenyl)-1H-imidazol-1-yl]methyl}-2,2-dimethylpropyl(1S)-1-((1R)-1-hydroxy-2-oxo-2-{[(1R)-1-phenylethyl]amino}ethyl)pentylcarbamate Et(1S)-1-{[4-(4-fluorophenyl)-1H-imidazol-1-yl]methyl}-2,2-dimethylpropyl(1S)-1-((1S)-1-hydroxy-2-oxo-2-{[(1-phenylethyl]amino}ethyl)pentylcarbamate

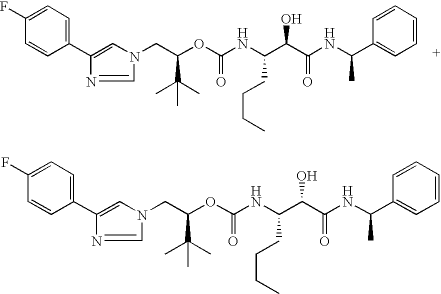

A solution of 57 mg (0.22 mmol) of (2S)-1-[4-(4-fluorophenyl)-1H-imidazol-1-yl]-3,3-dimethyl-2-butanol in 1.1 mL of tetrahydrofuran was stirred as 0.14 mL (0.26 mmol) of a 1.92 M solution of phosgene in toluene was added. The resulting solution was stirred for 17 h. The dark solution was then evaporated, and the residue was dissolved in 1 mL of methanol and added to a solution of 58 mg (0.22 mmol) of (2S,3S)-3-amino-2-hydroxy-N-[(1R)-1-phenylethyl]heptanamide Et(2R,3S)-3-amino-2-hydroxy-N-[(1R)-1-phenylethyl]heptanamide and 38 μL (0.22 mmol) of N,N-diisopropylethylamine in 1.2 mL of methanol. The resulting solution was stirred for 17 h. The mixture was evaporated and the residue was purified by silica gel column chromatography eluting with an ethyl acetate:hexanes solution (1:9) to yield 60 mg (50%) of (1S)-1-{[4-(4-fluorophenyl)-1H-imidazol-1-yl]methyl}-2,2-dimethylpropyl (1S)-1-((1R)-1-hydroxy-2-oxo-2-{[(1R)-1-phenylethyl]amino}ethyl)pentylcarbamate Et(1S)-1-{[4-(4-fluorophenyl)-1H-imidazol-1-yl]methyl}-2,2-dimethylpropyl(1S)-1-((1S)-1-hydroxy-2-oxo-2-{[(1R)-1-phenylethyl]amino}ethyl)pentylcarbamate. ES-MS m/z 553 (M+H)⁺.

### Example 6i

### Preparation of (1S)-1-{[4-(4-fluorophenyl)-1H-imidazol-1-yl]methyl}-2,2-dimethylpropyl(1S)-1-(oxo{[(1R)-1-phenylethyl]amino}acetyl)pentylcarbamate

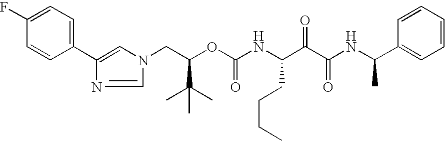

To 58 mg (0.105 mmol) of (1S)-1-{[4-(4-fluorophenyl)-1H-imidazol-1-yl]methyl}-2,2-dimethylpropyl(1S)-1-((1R)-1-hydroxy-2-oxo-2-{[(1R)-1-phenylethyl]amino}ethyl)pentylcarbamate Et(1S)-1-{[(4-fluorophenyl)-1H-imidazol-1-yl]methyl}-2,2-dimethylpropyl(1S)-1-((1S)-1-hydroxy-2-oxo-2-{[(1R)-1-phenylethyl]amino}ethyl)pentylcarbamate in 1.1 mL of dichloromethane at room temperature was added 89 mg (0.21 mmol) of Dess-Martin periodinane followed by sodium bicarbonate and the reaction mixture was stirred for 4 h. It was then partitioned between dichloromethane and saturated aqueous sodium bicarbonate. The extract was dried over anhydrous magnesium sulfate, filtered, and concentrated. The residue was purified by silica gel column chromatography eluting with an ethyl acetate:hexanes solution (1:9) to yield 29 mg (50%) of (1S)-1-{[4(4-fluorophenyl)-1H-imidazol-1-yl]methyl}-2,2-dimethylpropyl(1S)-1-(oxo{[(1R)-1-phenylethyl]amino}acetyl)pentyl carbamate. ¹H NMR (DMSO-d₆, Temp=80° C.): δ 8.86 (br m, 1H), 7.75 (m, 2H), 7.62 (s, 1H), 7.52 (s, 1H), 7.33-7.12 (m, 8H), 4.98 (m, 1H), 4.74-4.60 (m, 2H), 4.32 (d, J=14 Hz, 1H), 4.04 (m, 1H), 1.58 (m, 1H), 1.45 (m, overlapping d, J=7 Hz, 4H), 1.28-1.05 (m, 4H), 1.00 (s, 9H), 0.72 (m, 3H); HRMS C₃₁H₃₉N₄O₄F m/z 551.3033 (M+H)⁺_{Cal}; 551.3047 (M+H)⁺_{Obs.}

### Example 7

### Preparation of (1S)-2,2-dimethyl-1-({4-[4-(trifluoromethyl)phenyl]-1H-imidazol-1-yl}methyl)propyl(1S)-1-[oxo(1H-pyrazol-5-ylamino)acetyl]pentylcarbamate

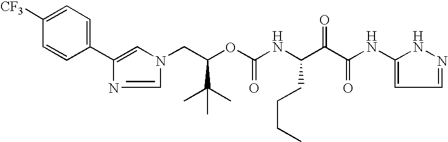

### Example 7a

### Preparation of 4-[4-(trifluoromethyl)phenyl]-1H-imidazole

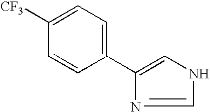

A mixture of 2.21 g (8.28 mmol) of 4-(trifluoromethyl)phenacylbromide and 9.3 mL of formamide was heated at 175° C. for 3 h. 30 mL of 1N hydrochloric acid was added to the resulting solution, which was then heated to reflux. The mixture was filtered and allowed to cool to room temperature before neutralization with concentrated ammonium hydroxide. The resulting mixture was partitioned between ethyl acetate and brine. The extract was dried over anhydrous magnesium sulfate, filtered, and evaporated. The residue was purified by silica gel column chromatography eluting with an ethyl acetate:methanol solution (9:1). Further purification by silica gel column chromatography eluting with a hexane:acetone solution (1:1) yielded 1.18 g (67%) of 4[4-(trifluoromethyl)phenyl]-1H-imidazole. ¹H NMR (DMSO-d₆): δ 12.34 (br s, 1H), 7.96 (d, J=8 Hz, 2H), 7.77 (s, 1H), 7.76 (s, 1H), 7.68 (d, J=8 Hz, 2H).

### Example 7b

### Preparation of (2S)-3,3-dimethyl-1-{4-[4-(trifluoromethyl)phenyl]-1H-imidazol-1-yl}-2-butanol

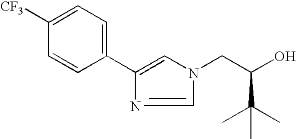

A solution 594 mg (5.94 mmol) of (S)-3,3-dimethyl-1,2-epoxybutane and 1.26 g (5.94 mmol) of 4′-trifluoromethyl-4-phenyl imidazole in 2.5 mL of ethanol was placed in a sealed tube and heated at 85° C. for 4 days. The mixture was cooled and concentrated, and the residue was purified by silica gel column chromatography eluting with ethyl acetate to yield 1.56 g (84%) of (2S)-3,3-dimethyl-1-{4-[4-(trifluoromethyl)phenyl]-1H-imidazol-1-yl}-2-butanol. ¹H NMR (DMSO-d₆): δ 7.93 (d, J=8 Hz, 2H), 7.84 (s, 1H), 7.68 (m, 3H), 5.01 (d, J=6 Hz, 1H), 4.15 (d, J=14 Hz, 1H), 3.73 (m, 1H), 3.31 (m, overlapping H₂O), 0.91 (s, 9H).

### Example 7c

### Preparation of (1S)-2,2-dimethyl-1-({4-[4-(trifluoromethyl)phenyl]-1H-imidazol-1-yl}methyl)propyl 4-nitrophenyl carbonate

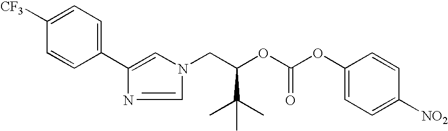

To solution of 1.91 g (6.12 mmol) of (2S)-3,3-dimethyl-1-{4-[4-(trifluoromethyl)phenyl]-1H-imidazol-1-yl}-2-butanol in 82 mL of tetrahydrofuran at 0° C. was added 4.2 mL (6.7 mmol) of 1.6M n-butyllithium in hexanes, and the resulting solution was stirred for 10 min. A solution of 1.85 g (9.19 mmol) of 4-nitrophenyl chloroformate in 38 mL of tetrahydrofuran was added, and the solution was stirred at room temperature for 19 h. Saturated aqueous sodium bicarbonate was added to the solution, and the mixture was extracted with ethyl acetate. The extract was washed with saturated aqueous sodium chloride, dried over anhydrous magnesium sulfate, filtered, and concentrated. The residue was purified by silica gel chromatography eluting with an ethyl acetate:hexanes solution (6:4) to give 1.83 g (sample contains •0.5 ethyl acetate by ¹H NMR for an effective weight of 1.68 g, 57%) of (1S)-2,2-dimethyl-1-({4-[4-(trifluoromethyl)phenyl]-1H-imidazol-1-yl}methyl)propyl 4-nitrophenyl carbonate. ¹H NMR (DMSO-d₆): δ 8.12 (d, J=7 Hz, 2H), 7.94 (d, J=8 Hz, 2H), 7.93 (s, 1H), 7.86 (s, 1H), 7.70 (d, J=8 Hz, 2H). 7.23 (d, J=9 Hz, 2H), 4.86 (d, J=8 Hz, 1H), 4.51 (d, J=13 Hz, 1H), 4.24 (dd, J=14 Hz, J=10 Hz, 1H). 1.05 (s, 9H).

### Example 7d

### Preparation of (1S)-2,2-dimethyl-1-{[4(4-methylphenyl)-1H-imidazol-1-yl]methyl}propyl(1S)-1-[(1R)-1-hydroxy-2-oxo-2-(1H-pyrazol-5-ylamino)ethyl]pentylcarbamate Et(1S)-2,2-dimethyl-1-{[4-(4-methylphenyl)-1H-imidazol-1-yl]methyl}propyl(1S)-1-[(1S)-1-hydroxy-2-oxo-2-(1H-pyrazol-5-ylamino)ethyl]pentylcarbamate

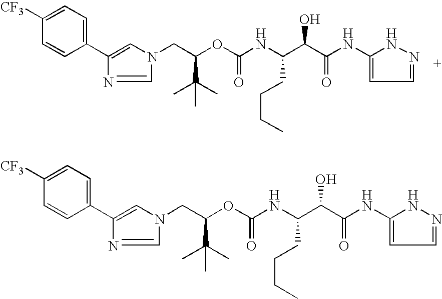

To 1.20 g (3.68 mmol) of tert-butyl(1S)-1-[(1R)-1-hydroxy-2-oxo-2-(1H-pyrazol-5-ylamino)ethyl]pentylcarbamate Et tert-butyl(1S)-1-[(1S)-1-hydroxy-2-oxo-2-(1H-pyrazol-5-ylamino)ethyl]pentylcarbamate in 11.8 mL of dioxane at room temperature was added 46 mL (184 mmol) of a 4M solution of hydrogen chloride in dioxane. The mixture was stirred for 1 h, concentrated, and dried under vacuum to afford an off-white solid, which was dissolved in 32 mL of N,N-dimethylformamide. One half of this solution was cooled to 0° C., and 0.792 g (sample contains •0.5 ethyl acetate by ¹H NMR for an effective weight of 0.725 g, 1.52 mmol) of (1S)-2,2-dimethyl-1-({[4-(trifluoromethyl)phenyl]-1H-imidazol-1-yl}methyl)propyl 4-nitrophenyl carbonate in 16 mL of N,N-dimethylformamide was added, followed by 1.3 mL (7.36 mmol) of N,N-diisopropylethylamine. The solution was stirred for 2 days at room temperature. It was concentrated, saturated sodium carbonate was added to the residue and the mixture was extracted with ethyl acetate. The extract was washed with saturated aqueous sodium chloride, dried over sodium sulfate, filtered, and concentrated. The residue was purified by silica gel column chromatography eluting with a methanol:chloroform solution (1:9) to give 0.64 g (75%) of (1S)-2,2-dimethyl-1-{[4-(4-methylphenyl)-1H-imidazol-1-yl]methyl}propyl(1S)-1-[(1R)-1-hydroxy-2-oxo-2-(1H-pyrazol-5-ylamino)ethyl]pentylcarbamate Et(1S)-2,2-dimethyl-1-{[4-(4-methylphenyl)-1H-imidazol-1-yl]methyl}propyl(1S)-1-[(1S)-1-hydroxy-2-oxo-2-(1H-pyrazol-5-ylamino)ethyl]pentylcarbamate. Isomer 1 ES-LCMS m/z 565 (M+H)⁺ retention time=3.70 min. Isomer 2 ES-LCMS m/z 565 (M+H)⁺ retention time=3.36 mm.

### Example 7e

### Preparation of (1S)-2,2-dimethyl-1-({4-[4-(trifluoromethyl)phenyl]-1H-imidazol-1-yl}methyl)propyl(1S)-1-[oxo(1H-pyrazol-5-ylamino)acetyl]pentylcarbamate

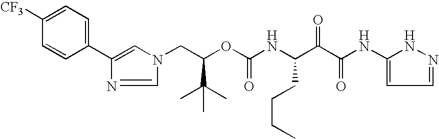

To 0.54 g (0.96 mmol) of (1S)-2,2-dimethyl-1-{[4-(4-methylphenyl)-1H-imidazol-1-yl]methyl}propyl(1S)-1-[(1R)-1-hydroxy-2-oxo-2-(1H-pyrazol-5-ylamino)ethyl]pentylcarbamate Et(1S)-2,2-dimethyl-1-{[4-(4-methylphenyl)-1H-imidazol-1-yl]methyl}propyl(1S)-1-[(1S)-1-hydroxy-2-oxo-2-(1H-pyrazol-5-ylamino)ethyl]pentylcarbamate in 25.5 mL of chloroform at room temperature was added 507 mg (1.20 mmol) of Dess-Martin periodinane, and the resulting mixture was stirred for 1 h. It was then poured into saturated aqueous sodium metabisulfite and subsequently neutralized with saturated aqueous sodium bicarbonate. The mixture was extracted with ethyl acetate. The extract was dried over sodium sulfate, filtered, and concentrated. The residue was purified by silica gel column chromatography eluting with an acetone:hexanes solution (1:1) to give 396 mg (73%) of (1S)-2,2-dimethyl-1-({4-[4-(trifluoromethyl)phenyl]-1H-imidazol-1-yl}methyl)propyl(1S)-1-[oxo(1H-pyrazol-5-ylamino)acetyl]pentylcarbamate ¹H NMR (300 MHz, DMSO-d₆, Temp=110° C.) δ 11.58 (br s, 1H), 10.32 (br s, 1H), 7.91 (d, J=8 Hz, 2H), 7.66 (s, 1H), 7.64 (s, 1H), 7.62 (d, J=8 Hz, 2H), 7.56 (s, 1H), 7.07 (br s, 1H), 6.46 (s, 1H), 4.75 (d, J=9 Hz, 1H), 4.74-4.64 (m, 1H), 4.33 (d, J=15 Hz, 1H), 4.02 (dd, J=14 Hz, J=10 Hz, 1H), 1.76-1.58 (m, 1H), 1.56 (m, 1H), 1.34-1.10 (m, 4H), 0.98 (s, 9H), 0.73 (t, J=7 Hz, 3H); HRMS C₂₇H₃₄N₆O₄F₃ m/z 563.2594 (M+H)⁺_{Cal}; 563.2594 (M+H)⁺_{Obs}Δ=0.0 mmu.

### Example 8

### Preparation of (1R)-2,2-dimethyl-1-[(5-phenyl-1,3,4-oxadiazol-2-yl)methyl]butyl (1S)-1-(oxo{[(1R)-1-phenylethyl]amino}acetyl)pentylcarbamate

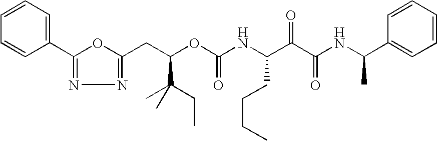

### Example 8a

### Preparation of (2R)-3,3-dimethyl-1-(5-phenyl-1,3,4-oxadiazol-2-yl)-2-pentanol Et(2S)-3,3-dimethyl-1-(5-phenyl-1,3,4-oxadiazol-2-yl)-2-pentanol

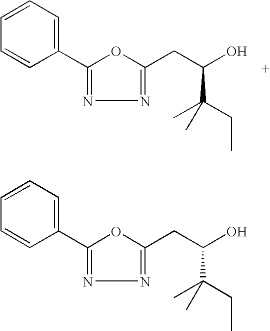

To a solution of 2.32 g (14.5 mmol) of 2-methyl-5-phenyl-1,3,4-oxadiazole in 38 mL of tetrahydrofuran at −78° C. was added 9 mL (14.4 mmol) of a 1.6M solution of n-butyllithium in hexanes, and the resulting red solution was stirred 5 min. A solution of 821 mg (8.21 mmol) of 2,2-dimethyl butyraldehyde in 2 mL of tetrahydrofuran was then added, and the resulting solution was stirred at −78° C. for 1 h, then at 0° C. for 1 h. The solution was allowed to warm to room temperature, and was stirred overnight. It was then partitioned between ethyl acetate and a saturated aqueous solution of sodium chloride. The extract was dried over anhydrous magnesium sulfate, filtered, and concentrated. The residue was purified by silica gel column chromatography eluting with an ethyl acetate:hexanes solution (3:7) to give 0.42 g (11%) of 3,3-dimethyl-1-(5-phenyl-1,3,4-oxadiazol-2-yl)-2-pentanol. ¹H NMR (300 MHz, DMSO-d₆) δ 8.02 (m, 2H), 7.62 (m, 3H), 4.95 (d, J=6 Hz, 1H), 3.74 (m, 1H), 3.08 (dd, J=15 Hz, J=2 Hz, 1H), 2.86 (dd, J=15 Hz, J=11 Hz, 1H), 1.42 (m, 1H), 1.29 (m, 1H), 0.89-0.86 (m, 9H). The enantiomers of the racemic alcohol were separated by supercritical fluid chromatography using a Chiralpak AD column, 27° C., 14 Mpa, 7% MeOH (3 mL/min methanol), 93% CO₂ (44 g/min CO₂). The separated enantiomers were analyzed by SFC chromatography using a Chiralpak AD column, 10 micron, 0.46×25 cm, 7% Methanol: 93% Carbon Dioxide, 2.0 mL/min, 2000 psi. Retention times: isomer 1, (2R)-3,3-dimethyl-1-(5-phenyl-1,3,4-oxadiazol-2-yl)-2-pentanol, 15.2 min; isomer 2, (2S)-3,3-dimethyl-1-(5-phenyl-1,3,4-oxadiazol-2-yl)-2-pentanol, 20.9 min. ¹H NMR and LC-MS data of each isomer were identical with that of the racemate above.

### Example 8b

### Preparation of methyl(2S)-2-{[({(1R)-2,2-dimethyl-1-[(5-phenyl-1,3,4-oxadiazol-2-yl)methyl]butyl}oxy)carbonyl]amino}hexanoate

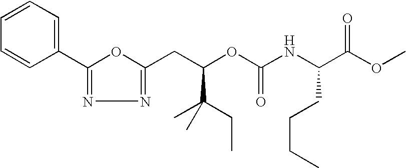

A solution of 155 mg (0.60 mmol) of (2R)-3,3-dimethyl-1-(5-phenyl-1,3,4-oxadiazol-2-yl)-2-pentanol and 113 mg (0.66 mmol) of methyl(2S)-2-isocyanatohexanoate in 2 mL of toluene was heated at 85° C. for 2 days. The solution was concentrated, and the residue was purified by silica gel column chromatography, eluting with an ethyl acetate:hexanes solution (4:6) to give 0.20 g (77%) of methyl(2S)-2-{[({(1R)-2,2-dimethyl-1-[(5-phenyl-1,3,4-oxadiazol-2-yl)methyl]butyl}oxy)carbonyl]amino}hexanoate. ¹H NMR (300 MHz, DMSO-d₆) δ 7.92 (m, 2H), 7.59 (m, 3H), 7.50 (d, J=8 Hz, 1H), 4.90 (m, 1H), 3.70 (m, 1H), 3.59 and 3.52 (s, 3H), 3.24 (m, overlapping H₂O), 3.04 (m, 1H), 1.65-1.20 (br m, 4H), 1.06 (br m, 4H), 0.91 and 0.90 (s, 6H), 0.83 (m, 3H), 0.71 (m, 3H); ES-LCMS m/z 432 (M+H)⁺ retention time=3.82 min.

### Example 8c

### Preparation of (1R)-2,2-dimethyl-1-[(5-phenyl-1,3,4-oxadiazol-2-yl)methyl]butyl(1S)-1-[cyano(triphenylphosphoranylidene)acetyl]pentylcarbamate

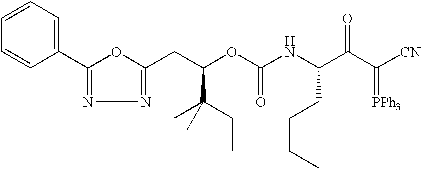

A solution of 26 mg (0.62 mmol) of lithium hydroxide monohydrate in 2.2 mL of water was added to a solution of 0.19 g (0.44 mmol) of ester methyl (2S)-2-{[({(1R)-2,2-dimethyl-1-[(5-phenyl-1,3,4-oxadiazol-2-yl)methyl)butyl}oxy)carbonyl]amino}hexanoate in 2.2 mL tetrahydrofuran. After 80 min, the solution was neutralized with 1N hydrochloric acid and extracted with ethyl acetate. The extract was dried over anhydrous magnesium sulfate, filtered, and concentrated. The residue was dissolved in 4.7 mL of dichloromethane and 159 mg (0.53 mmol) of (triphenylphosphoranylidene)acetonitrile was added. The solution was cooled to 0° C., and 4.0 mg (0.03 mmol) of 4-dimethylaminopyridine was added, followed by 102 mg (0.53 mmol) of 1-ethyl-3-(3-dimethyl-aminopropyl)carbodiimide. The solution was allowed to warm slowly to room temperature and was stirred overnight. The solution was partitioned between ethyl acetate and water. The extract was washed with 10% aqueous citric acid, saturated aqueous sodium bicarbonate, and brine before drying with magnesium sulfate. The mixture was filtered and concentrated. The residue was purified by silica gel column chromatography eluting with an ethyl acetate:hexanes solution (6:4) to yield 0.13 g (sample contains •1.7 ethyl acetate by ¹H NMR for an effective weight of 107 mg, 29%) of (1R)-2,2-dimethyl-1-[(5-phenyl-1,3,4-oxadiazol-2-yl)methyl]butyl(1S)-1-[cyano(triphenylphosphoranylidene)acetyl]pentylcarbamate. ¹H NMR (300 MHz, DMSO-d₆) δ 7.95 (d, J=6 Hz, 1H), 7.70 (m, 3H), 7.55 (m, 15H), 7.02 (d, J=8 Hz, 1H), 4.98 (d, J=9 Hz, 1H), 4.29 (m, 1H), 3.31 (m, overlapping H₂O), 3.04 (m, 1H), 1.62 (m, 1H), 1.50-1.40 (br m, 7H), 0.94-0.79 (m, 9H), 0.71 (t, J=7 Hz, 3H); ES-LCMS m/z 701 (M+H)⁺ retention time=4.02 min.

### Example 8d

### Preparation of (1R)-2,2-dimethyl-1-[(5-phenyl-1,3,4oxadiazol-2-yl)methyl]butyl(1S)-1-(oxo{[(1R)-1-phenylethyl]amino}acetyl)pentylcarbamate

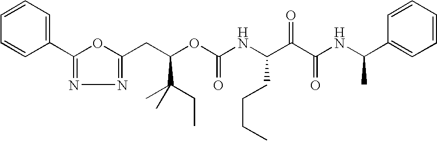

Ozone was bubbled through a −78° C. solution of 0.12 g (sample contains •1.7 EtOAc by ¹H NMR for an effective weight of 98 mg, 0.14 mmol) of (1R)-2,2-dimethyl-1-[(5-phenyl-1,3,4-oxadiazol-2-yl)methyl]butyl(1S)-1-[cyano(triphenyl phosphoranylidene)acetyl]pentylcarbamate in 6 mL of dichloromethane for 15 min. Nitrogen was then bubbled through the solution for 5 min before the addition of 22.5 μL (0.17 mmol) of (R)-(+)-α-methyl benzylamine. The solution was stirred at −78° C. for 2 h. The solution was concentrated, and the residue was diluted with 1.6 mL (1.6 mmol) of a 1M solution of silver nitrate in 4:1 tetrahydrofuran:water and allowed to stir for 3 days. The solution was partitioned between dichloromethane and water. The extract was dried over anhydrous magnesium sulfate, filtered, and evaporated. The residue was purified by silica gel column chromatography eluting with an ethyl acetate:hexanes solution (4:6) to yield 22 mg (29%) of (1R)-2,2-dimethyl-1-[(5-phenyl-1,3,4-oxadiazol-2-yl)methyl]butyl(1S)-1-(oxo{[(1R)-1-phenylethyl]amino}acetyl)pentylcarbamate. ¹H NMR (300 MHz, DMSO-d₆) δ 9.07 (d, J=8 Hz, 1H), 7.88 (d, J=7 Hz, 2H), 7.54 (m, 3H), 7.40 (d, J=8 Hz, 1H), 7.26-7.15 (m, 5H), 4.84 (m, 2H), 4.49 (m, 1H), 3.22 (m, overlapping H₂O), 3.02 (m, 1H), 1.6-0.6 (br m, 17H), 1.32 (d, J=7 Hz, 3H), 0.61 (m, 3H); ES-LCMS m/z 549 (M+H)⁺ retention time=4.16 minutes; HRMS C₃₁H₄₀N₄O₅ m/z 549.3077 (M+H)⁺_{Cal}; 549.3073 (M+H)⁺_{Obs}.

### Example 9

### Preparation of (1R)-2,2-dimethyl-1-[(5-phenyl-1,3,4-oxadiazol-2-yl)methyl]butyl (1S)-1-(oxo{[(1R)-1-phenylethyl]amino}acetyl)pentylcarbamate

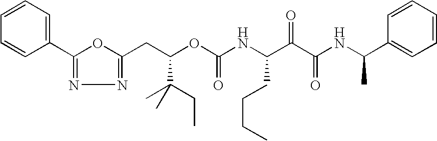

### Example 9a

### Preparation of methyl(2S)-2-{[({(1S)-2,2-dimethyl-1-[(5-phenyl-1,3,4-oxadiazol-2-yl)methyl]butyl}oxy)carbonyl]amino}hexanoate

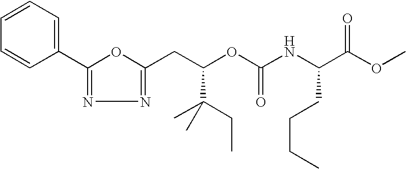

A solution of 126 mg (0.48 mmol) of (2S)-3,3-dimethyl-1-(5-phenyl-1,3,4-oxadiazol-2-yl)-2-pentanol and 99 mg (0.58 mmol) of methyl(2S)-2-isocyanatohexanoate in 2 mL of toluene was heated at 85° C. for 4 days. The solution was concentrated, and the residue was purified by silica gel column chromatography eluting with an ethyl acetate:hexanes solution (4:6) to give 0.17 g (76%) of methyl (2S)-2-{[({(1S)-2,2-dimethyl-1-[(5-phenyl-1,3,4-oxadiazol-2-yl)methyl]butyl}oxy)carbonyl]amino}hexanoate. ¹H NMR (300 MHz, DMSO-d₆) δ 7.96 (m, 2H), 7.62 (m, 3H), 7.25 (d, J=1 Hz, 1H), 4.95 (m, 1H), 4.0-3.8 (m, 1H), 3.64 (m, 3H), 3.31 (m, overlapping H₂O), 3.08 (dd, J=15 Hz, J=11 Hz, 1H), 1.55 (br m, 3H), 1.38 (m, 6H), 0.94 (m, 12H).

### Example 9b

### Preparation of (1S)-2,2-dimethyl-1-[(5-phenyl-1,3,4-oxadiazol-2-yl)methyl]butyl(1S)-1-butyl-3-cyano-2-oxo-3-butenylcarbamate

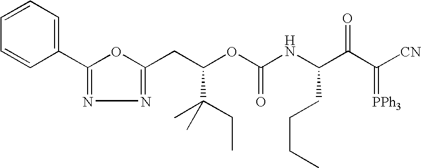

A solution of 23 mg (0.55 mmol) of lithium hydroxide monohydrate in 2.0 mL of water was added to a solution of 0.17 g (0.394 mmol) of methyl(2S)-2-{[({(1S)-2,2-dimethyl-1-[(5-phenyl-1,3,4-oxadiazol-2-yl)methyl]butyl}oxy)carbonyl]amino}hexanoate in 2.0 mL of tetrahydrofuran. After 70 min, the solution was neutralized with 1N hydrochloric acid, and then extracted with ethyl acetate. The extract was dried over anhydrous magnesium sulfate, filtered, and concentrated. The residue was dissolved in 3.0 mL of dichloromethane, and 102 mg (0.34 mmol) of (triphenylphosphoranylidene)acetonitrile was added. The resulting solution was cooled to 0° C. before 2.4 mg (0.02 mmol) of 4-dimethylaminopyridine was added, followed by 65 mg (0.34 mmol) of 1-ethyl-3-(3-dimethyl-aminopropyl)carbodiimide. The solution was allowed to warm slowly to room temperature and was stirred overnight. It was then partitioned between ethyl acetate and water. The extract was washed with 10% aqueous citric acid, saturated aqueous sodium bicarbonate, and then brine before drying with magnesium sulfate. The mixture was filtered and concentrated, and the residue was purified by silica gel column chromatography eluting with an ethyl acetate:hexanes solution (6:4) to yield 88.2 mg (37%) of (1S)-2,2-dimethyl-1-[(5-phenyl-1,3,4-oxadiazol-2-yl)methyl]butyl(1S)-1-[cyano(triphenylphosphoranylidene)acetyl]pentylcarbamate. ES-LCMS m/z 701 (M+H)⁺ retention time=4.08 min.

### Example 9c

### Preparation of (1S)-2,2-dimethyl-1-[(5-phenyl-1,3,4-oxadiazol-2-yl)methyl]butyl(1S)-1-(oxo{[(1R)-1-phenylethyl]amino}acetyl)pentylcarbamate

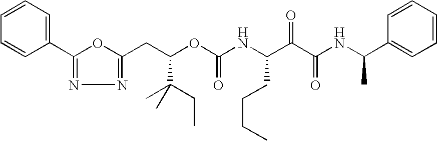

Ozone was bubbled through a −78° C. solution of 88 mg (0.13 mmol) of (1S)-2,2-dimethyl-1-[(5-phenyl-1,3,4-oxadiazol-2-yl)methyl]butyl(1S)-1-[cyano(triphenyl phosphoranylidene)acetyl]pentylcarbamate in 6 mL of dichloromethane for 15 min. Nitrogen was then bubbled through the solution for 5 min, before 17.2 μL (0.17 mmol) of (R)-(+)-α-methyl benzylamine was added. The solution was stirred at −78° C. for 2 h. It was concentrated, and the residue was diluted with 1.6 mL (1.6 mmol) of a 1M solution of silver nitrate in 4:1 tetrahydrofuran:water and allowed to stir for 1 day. The solution was then partitioned between dichloromethane and water. The extract was dried over anhydrous magnesium sulfate, filtered, and evaporated. The residue was purified by silica gel column chromatography eluting with an ethyl acetate:hexanes solution (4:6) to yield 14 mg (20%) of (1S)-2,2-dimethyl-1-[(5-phenyl-1,3,4-oxadiazol-2-yl)methyl]butyl(1S)-1-(oxo{[(1R)-1-phenylethyl]amino}acetyl)pentylcarbamate. ES-LCMS m/z 549 (M+H)⁺ retention time=4.12 minutes; HRMS C₃₁H₄₀N₄O₅ m/z 571.2896 (M+Na)⁺_{Cal}; 571.2906 (M+H)⁺_{Obs}.

### Example 10

### Preparation of (1R)-2-methyl-1-[(5-phenyl-1,3,4-oxadiazol-2-yl)methyl]propyl(1S)-1-(oxo{[(1R)-1-phenylethyl]amino}acetyl)pentylcarbamate

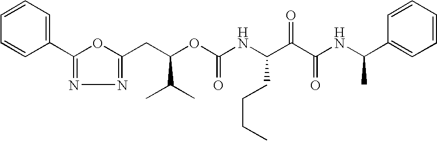

### Example 10a

### Preparation of 2-(2-isopropyl-1,3-dioxolan-2-yl)acetohydrazide

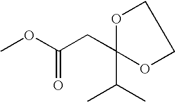

A flask containing a solution of 7.50 g (52 mmol) of methyl isobutyrlacetate, 90 mg (0.52 mmol) of p-toluene sulfonic acid, and 7.2 mL (130 mmol) ethylene glycol in 55 mL of benzene was fitted with a Dean Stark Trap and a condenser. The solution was heated at reflux for 3 days. It was then partitioned between saturated aqueous sodium bicarbonate and ether. The extract was dried over anhydrous magnesium sulfate, filtered, and evaporated. The residue was purified by silica gel column chromatography eluting with an ethyl acetate:hexanes solution (2:8) to yield 7.36 g (sample contains •0.25 ethyl acetate by ¹H NMR for an effective weight of 6.58 g, 67%) of 2-(2-isopropyl-1,3-dioxolan-2-yl)acetohydrazide. ¹H NMR (300 MHz, DMSO-d₆) δ 3.93 (m, 4H), 3.59 (s, 3H), 2.64 (s, 2H), 2.06 (septet, J=7 Hz, 1H), 0.90 (d, J=7 Hz, 6H).

### Example 10b

### Preparation of 2-(2-isopropyl-1,3-dioxolan-2-yl)acetohydrazide

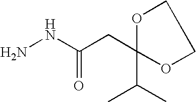

A solution of 4.50 g (23.9 mmol) of 2-(2-isopropyl-1,3-dioxolan-2-yl)acetohydrazide and 1.1 mL (35.9 mmol) of hydrazine in 10 mL of ethanol was stirred for 1 week at room temperature. The solution was evaporated, and the residue was purified by silica gel column chromatography eluting with an ethyl acetate:methanol solution (8:2) to yield 2.00 g (23%) of 2-(2-isopropyl-1,3-dioxolan-2-yl)acetohydrazide. ¹H NMR (300 MHz, DMSO-d₆) δ 8.82 (s, 1H), 4.19 (br s, 2H), 3.94 (m, 2H) 3.83 (m, 2H), 2.35 (s, 2H) 2.12 (septet, J=7 Hz, 1H), 0.89 (d, J=7 Hz, 6H).

### Example 10c

### Preparation of 2-[(2-isopropyl-1,3-dioxolan-2-yl)methyl]-5-phenyl-1,3,4-oxadiazole

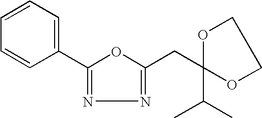

A solution of 2.00 g (8.92 mmol) of 2-(2-isopropyl-1,3-dioxolan-2-yl)acetohydrazide and 2.2 mL (7.5 mmol) of triethyl orthobenzoate in 23 mL of xylenes was heated to reflux in a flask fitted with a Dean Stark trap and a condenser. After 1 d, the solution was concentrated, and the residue was purified by silica gel column chromatography eluting with an ethyl acetate:hexane solution (2:8) to yield 2.50 g (sample contains 0.32 ethyl acetate by ¹H NMR for an effective weight of 2.26 g, 93%) of 2-[(2-isopropyl-1,3-dioxolan-2-yl)methyl]-5-phenyl-1,3,4-oxadiazole. ¹H NMR (300 MHz, DMSO-d₆) δ 7.92 (dd, J=8 Hz, J=2 Hz, 2H), 7.55 (m, 3H), 3.81 (m, 2H), 3.76 (m, 2H), 3.23 (s, 2H), 1.90 (septet, J=7 Hz, 1H), 0.90 (d, J=7 Hz, 6H).

### Example 10d

### Preparation of 3-methyl-1-(5-phenyl-1,3,4-oxadiazol-2-yl)-2-butanone

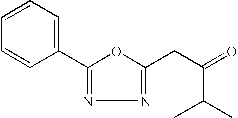

A solution of 2.5 g (sample contains 0.32 ethyl acetate by ¹H NMR for an effective weight of 2.26 g, 8.26 mmol) of 2-[(2-isopropyl-1,3-dioxolan-2-yl)methyl]-5-phenyl-1,3,4-oxadiazole in 17.5 mL of formic acid was stirred as 3 drops of concentrated sulfuric acid were added. The solution was heated in a 45° C. oil bath for 2 h. It was poured into a solution of ice and water, neutralized with bicarbonate, and then extracted with ethyl acetate. The extract was concentrated, and the residue was purified by silica gel column chromatography eluting with an ethyl acetate:hexane solution (2:8) to yield 1.65 g (87%) of 3-methyl-1-(5-phenyl-1,3,4-oxadiazol-2-yl)-2-butanone. ¹H NMR (300 MHz, DMSO-d₆) δ 8.00 (dd, J=8 Hz, J=2 Hz, 2H), 7.64 (m, 3H), 4.49 (s, 2H), 2.86 (septet, J=7 Hz, 1H), 1.13 (d, J=7 Hz, 6H).

### Example 10e

### Preparation of (2S)-3-methyl-1-(5-phenyl-1,3,4-oxadiazol-2-yl)-2-butanol Et(2R)-3-methyl-1-(5-phenyl-1,3,4-oxadiazol-2-yl)-2-butanol

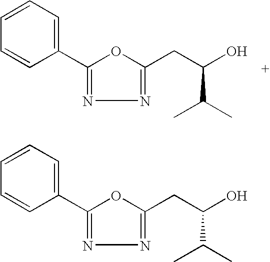

A solution of 1.64 g (7.152 mmol) of 3-methyl-1-(5-phenyl-1,3,4-oxadiazol-2-yl)-2-butanone in 24 mL of tetrahydrofuran and 2.4 mL of water was stirred as 352 mg (9.3 mmol) of sodium borohydride was added. The solution was stirred at room temperature for 17 h. Water was then added, and the mixture was extracted with ethyl acetate. The extract was dried over anhydrous magnesium sulfate, filtered, and concentrated. The residue was purified by silica gel column chromatography eluting with an ethyl acetate:hexane solution (4:6) to yield 1.47 g (89%) of (25)-3-methyl-1-(5-phenyl-1,3,4-oxadiazol-2-yl)-2-butanol Et(2R)-3-methyl-1-(5-phenyl-1,3,4-oxadiazol-2-yl)-2-butanol. ¹H NMR (300 MHz, DMSO-d₆) δ 8.01 (m, 2H), 7.63 (m, 3H), 4.94(br s, 1H), 3.77 (br m, 1H), 3.07 (dd, J=15 Hz, J=4 Hz, 1H), 2.93 (dd, J=15 Hz, J=1 Hz, 1H), 1.70 (m, 1H), 0.95 (m, 6H); ES-LCMS m/z233 (M+H)⁺.

### Example 10f

### Preparation of methyl(2S)-2-{[({(1S)-2-methyl-1-[(5-phenyl-1,3,4-oxadiazol-2-yl)methyl]propyl}oxy)carbonyl]amino}hexanoate Et methyl(2S)-2-{[({(1R)-2-methyl-1-[(5-phenyl-1,3,4-oxadiazol-2-yl)methyl]propyl}oxy)carbonyl]amino}hexanoate

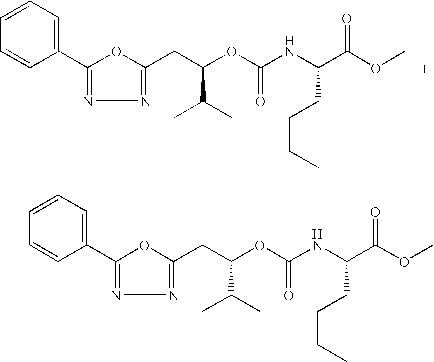

A solution of 0.92 g (4.0 mmol) of 3-methyl-1-(5-phenyl-1,3,4-oxadiazol-2-yl)-2-butanol and 0.82 g (4.8 mmol) of methyl(2S)-2-isocyanatohexanoate in toluene was stirred at 85° C. for 3 days. It was concentrated, and the residue was purified by silica gel column chromatography eluting with an ethyl acetate:hexane solution (4:6) to yield 1.47 g (89%) of methyl(2S)-2-{[({(1S)-2-methyl-1-[(5-phenyl-1,3,4-oxadiazol-2-yl)methyl]propyl}oxy)carbonyl]amino}hexanoate Et methyl(2S)-2-{[({(1R)-2-methyl-1-[(5-phenyl-1,3,4oxadiazol-2-yl)methyl]propyl}oxy)carbonyl]amino}hexanoate. ES-LCMS m/z 404 (M+H)⁺.

### Example 10g

### Preparation of (2S)-2-{[({(1R)-2-methyl-1-[(5-phenyl-1,3,4-oxadiazol-2-yl)methyl]propyl}oxy)carbonyl]amino}hexanoic acid Et(2S)-2-{[({(1S)-2-methyl-1-[(5-phenyl-1,3,4-oxadiazol-2-yl)methyl]propyl}oxy)carbonyl]amino}hexanoic acid

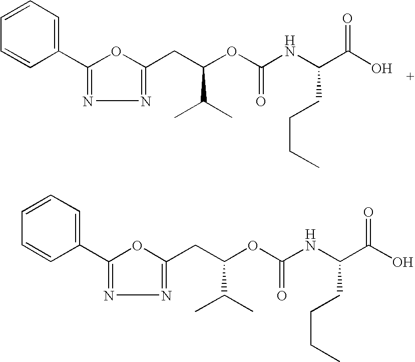

A solution of 186 mg (4.44 mmol) of lithium hydroxide monohydrate in 15.8 mL of water was added to a solution of 1.28 g (3.17 mmol) of methyl(2S)-2-[({2-methyl-1-[(5-phenyl-1,3,4-oxadiazol-2-yl)methyl]propoxy}carbonyl)amino]hexanoate in 15.8 mL of tetrahydrofuran. After 60 min, the solution was neutralized with 1N hydrochloric acid, and then extracted with ethyl acetate. The extract was dried over anhydrous magnesium sulfate, filtered, and concentrated to yield 0.83 g (67%) of (2S)-2-{[({(1R)-2-methyl-1-[(5-phenyl-1,3,4-oxadiazol-2-yl)methyl]propyl}carbonyl]amino}hexanoic acid Et(2S)-2-{[({(1S)-2-methyl-1-[(5-phenyl-1,3,4-oxadiazol-2-yl)methyl]propyl}oxy)carbonyl]amino}hexanoic acid. ¹H NMR (300 MHz, DMSO-d₆) δ 12.45 (br s, 1H), 7.99 (m, 2H), 7.63 (m, 3H), 7.48 and 7.42 (d, J=8 Hz, 1H), 4.92 (m, 1H), 3.90 and 3.77 (m, 1H), 3.44-3.11 (m, overlapping H₂O), 1.91 and 1.78 (m, 1H), 1.57 (br m, 2H), 1.40-1.05 (m, 6H), 1.01 -0.75 (m, 9H).

### Example 10h

### Preparation of (1S)-2-methyl-1-[(5-phenyl-1,3,4-oxadiazol-2-yl)methyl]propyl(1S)-1-[cyano(triphenylphosphoranylidene)acetyl]pentylcarbamate Et(1R)-2-methyl-1-[(5-phenyl-1,3,4-oxodiazol-2-yl)methyl]propyl(1S)-1-[cyano (triphenylphosphoranylidene)acetyl]pentylcarbamate

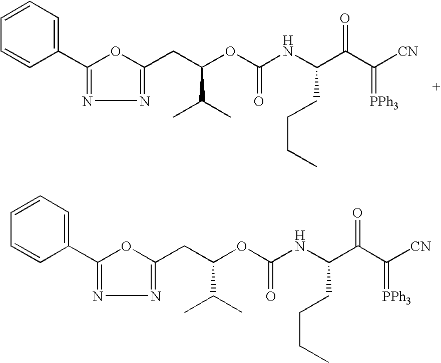

632 mg (2.1 mmol) of (triphenylphosphoranylidene)acetonitrile was added to a solution of 0.83 g (2.12 mmol) of (2S)-2-{[({(1R)-2-methyl-1-[(5-phenyl-1,3,4-oxadiazol-2-yl)methyl]propyl}oxy)carbonyl]amino}hexanoic acid Et(2S)-2-{[({(1S)-2-methyl-1-[(5-phenyl-1,3,4-oxadiazol-2-yl)methyl]propyl}oxy)carbonyl]amino}hexanoic acid in 18.5 mL of dichloromethane. The solution was cooled to 0° C. and 13.4 mg (0.11 mmol) of 4-dimethylaminopyridine was added followed by 403 mg (2.1 mmol) of 1-ethyl-3-(3-dimethyl-aminopropyl)carbodiimide. The solution was allowed to warm slowly to room temperature and was stirred overnight. It was then partitioned between ethyl acetate and water. The extract was washed with 10% aqueous citric acid, saturated aqueous sodium bicarbonate, and then brine before drying with magnesium sulfate. The mixture was filtered, and concentrated, and the resulting residue was purified by silica gel column chromatography eluting with an ethyl acetate:hexanes solution (7:3) to yield 0.44 g (35% ) of (1S)-2-methyl-1-[(5-phenyl-1,3,4-oxadiazol-2-yl)methyl]propyl(1S)-1-[cyano(triphenylphosphoranylidene)acetyl]pentylcarbamate Et(1R)-2-methyl-1-[(5-phenyl-1,3,4-oxadiazol-2-yl)methyl]propyl(1S)-1-[cyano(triphenylphosphoranylidene)acetyl]pentylcarbamate. ES-LCMS m/z 673 (M+H)⁺. The diastereomers were separated by supercritical fluid chromatography using a Kromasil DMB column at 40° C., 21 Mpa, 10% isopropanol, 41 g/min CO₂, 4 mL isopropanol. The separated diastereomers were analyzed by SFC chromatography using Kromasil DMB, 10 micron, 0.44×25 cm, 3000 psi, 10% isopropanol: 90% CO₂, 2 mL/min. retention times; isomer 1, (1S)-2-methyl-1-[(5-phenyl-1,3,4-oxadiazol-2-yl)methyl]propyl(1S)-1-[cyano(oxo)acetyl]pentylcarbamate, 22.3 min; isomer 2, (1R)-2-methyl-1-[(5-phenyl-1,3,4-oxadiazol-2-yl)methyl]propyl (1S)-1-[cyano(oxo)acetyl]pentylcarbamate, 25.7 min.

### Example 10i

### Preparation of (1R)-2-methyl-1-[(5-phenyl-1,3,4-oxadiazol-2-yl)methyl]propyl(1S)-1-(oxo{[(1R)-1-phenylethyl]amino}acetyl)pentylcarbamate

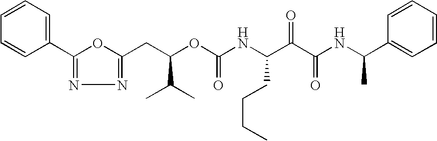

Ozone was bubbled through a −78° C. solution of 0.0641 g (0.095 mmol) of (1R)-2-methyl-1-[(5-phenyl-1,3,4-oxadiazol-2-yl)methyl]propyl(1S)-1-[cyano(oxo)acetyl]pentylcarbamate in 6 mL of dichloromethane for 15 min. The solution was then purged with a stream of nitrogen for 5 min before 12.6 μL (0.095 mmol) of (R)-(+)-α-methyl benzylamine were added. The solution was stirred at −78° C. for 1 h. It was concentrated, and the residue was diluted with 1.6 mL (1.6 mmol) of a 1M solution of silver nitrate in 4:1 tetrahydrofuran:water. The mixture was stirred for 2 days, and was then partitioned between dichloromethane and water. The extract was dried over anhydrous magnesium sulfate, filtered, and evaporated. The residue was purified by silica gel column chromatography eluting with an ethyl acetate:hexanes solution (4:6) to yield 9.8 mg (20%) of (1R)-2-methyl-1-[(5-phenyl-1,3,4-oxadiazol-2-yl)methyl]propyl(1S)-1-(oxo{[(1R)-1-phenylethyl]amino}acetyl)pentylcarbamate. ¹H NMR (300 MHz, DMSO-d₆) δ 8.07 (m, 2H), 7.55 (m, 3H), 7.34 (m, 5H), 7.34 (m, 5H), 7.12 (d, J=7 Hz, 1H), 5.30 (d, J=8 Hz, 1H), 5.10 (m, 3H), 3.23 (m, 2H), 2.10-1.85 (m, 2H), 1.57 (d, J=7 Hz, 3H), 1.40-1.15 (br m, 5H), 1.04 (m, 6H), 0.81 (m, 3H); HRMS C₂₉H₃₆N₄O₅ m/z 543.2583 (M+Na)⁺_{Cal}; 543.2574 (M+Na)⁺_{Obs}.

### Example 11

### Preparation of (1S)-2-methyl-1-[(5-phenyl-1,3,4-oxadiazol-2-yl)methyl]propyl(1S)-1-(oxo{[(1R)-1-phenylethyl]amino}acetyl)pentylcarbamate

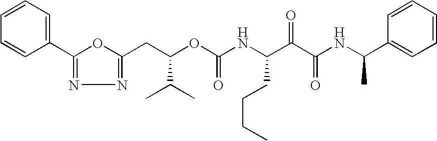

Ozone was bubbled through a −78° C. solution of 0.088 g (0.13 mmol) of (1S)-2-methyl-1-[(5-phenyl-1,3,4-oxadiazol-2-yl)methyl]propyl(1S)-1-[cyano(oxo)acetyl]pentylcarbamate in 6 mL of dichloromethane for 15 min. The solution was then purged with a stream of nitrogen for 5 min before 17.3 μL (0.13mmol) of (R)-(+)-α-methyl benzylamine were added. The solution was stirred at −78° C. for 1 h, and concentrated. The residue was diluted with 1.6 mL (1.6 mmol) of a 1M solution of silver nitrate in 4:1 tetrahydrofuran:water, and the solution was stirred for 1 day. The solution was partitioned between dichloromethane and water. The extract was dried over anhydrous magnesium sulfate, filtered, and evaporated. The residue was purified by silica gel column chromatography eluting with an ethyl acetate:hexanes solution (4:6) to yield 9.8 mg (20%) of (1S)-2-methyl-1-[(5-phenyl-1,3,4oxadiazol-2-yl)methyl]propyl(1S)-1-(oxo{[(1R)-1-phenylethyl]amino}acetyl)pentylcarbamate. ¹H NMR (300 MHz, DMSO-d₆) δ 9.20 and 9.05 (d, J=8 Hz, 1H), 7.93 (m, 2H), 7.58 (m, 4H), 7.30-7.15 (m, 5H), 4.87 (m, 2H), 4.70 (m, 1H), 3.30-3.08 (m, overlapping H₂O), 1.89 (m, 1H), 1.52 (br m, 1H) 1.40-1.09 (m, overlapping d at 1.37 ppm J=7 Hz, 8H), 0.94 and 0.83 (br m, 6H), 0.73 (m, 3H); HRMS C₂₉H₃₆N₄O₅ m/z 543.2583 (M+Na)⁺_{Cal}; 543.2581 (M+Na)⁺_{Obs}.

### Example 12

### Preparation of (1S)-2,2-dimethyl-1-[(4-phenyl-1H-imidazol-1-yl)methyl]propyl(1S)-1-(oxo{[(1R)-1-phenylethyl]amino}acetyl)pentylcarbamate

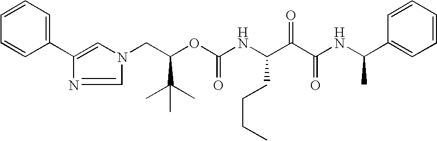

A solution of 100 mg (0.41 mmol) of (2S)-3,3-dimethyl-1-(4-phenyl-1H-imidazol-1-yl)-2-butanol in 2 mL of tetrahydrofuran was stirred as 0.26 mL (0.5 mmol) of a 1.92M solution of phosgene in toluene was added. The resulting solution was stirred for 17 h, and was then concentrated. The residue was dissolved in 2 mL of methanol, and this solution was added to a solution of 108 mg (0.41 mmol) of (3S-3-amino-2-hydroxy-N-[(1R)-1-phenylethyl]heptanamide and 71 μL (0.41 mmol) of diisopropylethylamine in 2.1 mL of methanol. The resulting solution was stirred for 24 h. It was concentrated and the residue was purified by silica gel column chromatography eluting with an ethyl acetate:hexanes solution (9:1) to yield 114 mg of the hydroxyamide which was dissolved in 1.9 mL of dichloromethane. 161 mg (0.38 mmol) of Dess-Martin periodinane was added to the solution followed by 32 mg (0.38 mmol) of sodium bicarbonate. The mixture was stirred for 195 min, before being partitioned between dichloromethane and water. The extract was dried over anhydrous magnesium sulfate, filtered, and concentrated. The residue was purified by silica gel column chromatography eluting with an ethyl acetate:hexanes solution (9:1) to yield 63 mg of impure product. This material was further purified by silica gel column chromatography eluting with an acetone:hexanes solution (6:4) to yield 21.8 mg (22%) of (1S)-2,2-dimethyl-1-[(4-phenyl-1H-imidazol-1-yl)methyl]propyl(1S)-1-(oxo{[(1R)-1-phenylethyl]amino}acetyl)pentylcarbamate ¹H NMR (300 MHz, DMSO-d₆) δ 9.08 (d, J=8 Hz, 1H), 7.64 (d, J=7 Hz, 2H), 7.57 (s, 1H), 7.51 (s, 1H), 7.44 (d, J=8 Hz, 1H), 7.24 (m, 6H), 7.17-7.08 (m, 2H), 4.86 (m, 1H), 4.61 (m, 1H), 4.54 (m, 1H), 4.26 (d, J=12 Hz, 1H), 3.94 (m, 1H), 1.42 (m, 1H) 1.33 (d, J=7 Hz, 3H), 1.23 (m, 1H), 1.20-0.70 (m, 13H), 0.61 (t, J=7 Hz, 3H); HRMS C₃₁H₄₀N₄O₄ m/z 533.3128 (M+H)⁺_{Cal}; 533.3119 (M+H)⁺_{Obs}.

### Example 13

### Preparation of (1R)-2,2-dimethyl-1-[(4-phenyl-1H-imidazol-1-yl)methyl]propyl(1S)-1-(oxo{[(1R)-1-phenylethyl]amino}acetyl)pentylcarbamate

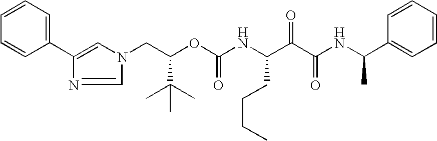

A solution of 100 mg (0.41 mmol) of (2R)-3,3-dimethyl-1-(4-phenyl-1H-imidazol-1-yl)-2-butanol in 2 mL of tetrahydrofuran was stirred as 0.26 mL (0.5 mmol) of a 1.92M solution of phosgene in toluene was added. The resulting solution was stirred for 17 h, and then concentrated. The residue was dissolved in 2 mL of methanol, and this solution was added to a solution of 108 mg (0.41 mmol) of (3S)-3-amino-2-hydroxy-N-[(1R)-1-phenylethyl]heptanamide and 71 μL (0.41 mmol) of diisopropylethylamine in 2.1 mL of methanol. The resulting solution was stirred for 24 h. It was concentrated and the residue was purified by silica gel column chromatography eluting with an ethyl acetate:hexanes solution (9:1) to yield 139.5 mg of the hydroxyamide, which was dissolved in 2.5 mL of dichloromethane. 212 mg (0.38 mmol) of Dess-Martin periodinane was added to the solution, followed by 42 mg (0.38 mmol) of sodium bicarbonate. The mixture was stirred for 3 h, and was then partitioned between dichloromethane and water. The extract was dried over anhydrous magnesium sulfate, filtered, and concentrated. The residue was purified by silica gel column chromatography eluting with an acetone:hexanes solution (4:6) to yield 67 mg of impure product. This material was further purified by silica gel column chromatography eluting with an ethyl acetate:hexanes solution (9:1) to yield 22.6 mg (17%) of (1S)-2,2-dimethyl-1-[(4-phenyl-1H-imidazol-1-yl)methyl]propyl(1S)-1-(oxo{[(1R)-1-phenylethyl]amino}acetyl)pentylcarbamate. HRMS C₃₁H₄₀N₄O₄ m/z 533.3128 (M+H)⁺_{Cal}; 533.3123 (M+H)⁺_{Obs}; APCI-LCMS m/z 533 (M+H)⁺ retention time=3.49 min.

### Example 14

### Preparation of (1S)-2,2-dimethyl-1-({4-[4-(trifluoromethyl)phenyl]-1H-pyrazol-1-yl}methyl)propyl(1S)-1-[oxo(1H-pyrazol-5-ylamino)acetyl]pentylcarbamate

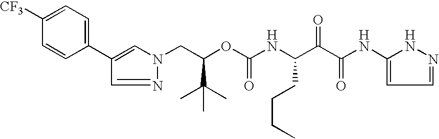

### Example 14a

### Preparation of 4-[4-(trifluoro)phenyl]-1H-pyrazole

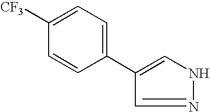

To 13.70 mL (146.95 mmol) of phosphorus oxychloride at 0° C. was added 13.92 mL (179.77 mmol) of anhydrous N,N-dimethylformamide dropwise. The resulting solution was stirred for 15 min at room temperature. Then, 10.00 g (48.98 mmol) of [4(trifluoromethyl)phenyl]acetic acid in 24 mL of anhydrous N,N-dimethylformamide was added dropwise. The resulting mixture was heated for 19 h at 70° C., poured into ice, and neutralized with potassium carbonate. To this solution was added 30 g of sodium hydroxide, and the resulting solution was heated at 50° C. for 15 min. The solution was then cooled to 0° C. and filtered. The filter cake was washed with water and dried under a vacuum to give 3-(dimethylamino)-2-[4-(trifluoromethyl)phenyl]-2-propenal. This dry solid was dissolved in 122 mL of methanol and 3.07 mL (97.97 mmol) of hydrazine was added to the solution, which was stirred for 6 h at room temperature. The solution was poured into water and the resulting mixture was filtered. The filter cake was washed with water, followed by hexanes, and dried under vacuum to give 8.35 g (80% yield) of 4-[4-(trifluoro)phenyl]-1H-pyrazole. R_{f}=0.26 (1:19 methanol:dichloromethane); ¹H NMR (300 MHz, DMSO-d₆) δ 13.09 (br s, 1H), 8.34 (s, 1H), 8.04 (s, 1H), 7.82 (d, J=8 Hz, 2H), 7.68 (d, J=8 Hz, 2H); ES-LCMS m/z 213 (M+H).

### Example 14b

### Preparation of (2S)-3,3-dimethyl-1-{4-[4-(trifluoromethyl)phenyl]-1H-pyrazol-1-yl}-2-butanol

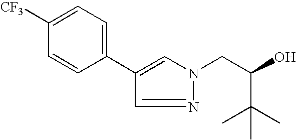

To 2.00 g (19.97 mmol) of (S)-3,3-dimethyl-1,2-epoxybutane in 5.0 mL of ethanol was added 5.08 g (23.96 mmol) of 4-[4-(trifluoro)phenyl]-1H-pyrazole. Then, 3.90 mL (27.96 mmol) of triethylamine was added to the solution, which was heated in a sealed tube at 85° C. for 16 h. The solution was cooled and concentrated, and the residue was purified by silica gel column chromatography eluting with an ethyl acetate:hexanes solution (3:7) to give 3.30 g (53%) of (2S)-3,3-dimethyl-1-{4-[4-(trifluoromethyl)phenyl]-1H-pyrazol-1-yl}-2-butanol. R_{f}=0.31 (3:7 ethyl acetate:hexanes); ¹H NMR (300 MHz, DMSO-d₆) δ 8.29 (s, 1H), 7.99 (s, 1H), 7.78 (d, J=8 Hz, 2H), 7.68 (d, J=8 Hz, 2H), 4.93 (d, J=6 Hz, 1H), 4.26 (dd, J=14 Hz, J=2 Hz, 1H), 3.89 (dd, J=14 Hz, J=10 Hz, 1H), 3.49 (dd, J=8 Hz, J=6=Hz, 1H), 0.91 (s, 9H); ES-LCMS m/z313 (M+H).

### Example 14c

### Preparation of (1S)-2,2-dimethyl-1-({4-[4-(trifluoromethyl)phenyl]-1H-pyrazol-1-yl}methyl)propyl 4-nitrophenyl carbonate

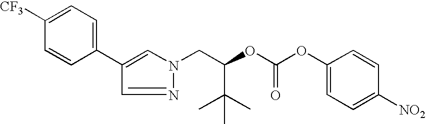

To 3.30 g (10.57 mmol) of (2S)-3,3-dimethyl-1-{4-[4-(trifluoromethyl)phenyl]-1H-pyrazol-1-yl}-2-butanol in 200 mL of tetrahydrofuran at 0° C. was added 7.26 mL (11.62 mmol) of 1.6 M n-butyllithium in hexanes, and the resulting solution was stirred for 10 min. Then, 3.19 g (15.84 mmol) of 4-nitrophenyl chloroformate in 11 mL of tetrahydrofuran was added, and the solution was stirred at room temperature for 75 min. Saturated aqueous sodium bicarbonate was added to the solution, and the resulting mixture was extracted with ethyl acetate. The extract was washed with saturated aqueous sodium chloride, and the extract was dried over anhydrous magnesium sulfate, filtered, and concentrated. The residue was purified by silica gel column chromatography eluting with an ethyl acetate:hexanes solution (3:7) to give 3.92 g (78%) of (1S)-2,2-dimethyl-1-({4-[4-(trifluoromethyl)phenyl]-1H-pyrazol-1-yl}methyl)propyl 4-nitrophenyl carbonate. R_{f}=0.19 (3:7 ethyl acetate:hexanes); ¹H NMR (300 MHz, DMSO-d₆) δ 8.44 (s, 1H), 8.14 (d, J=9 Hz, 2H), 8.08 (s, 1H), 7.79 (d, J=8 Hz, 2H), 7.70 (d, J=8 Hz, 2H), 7.27 (d, J=9 Hz, 2H), 4.88 (dd, J=10 Hz, J=2 Hz, 1H), 4.61 (d, J=13 Hz, 1H), 4.32 (dd, J=15 Hz, J=10 Hz, 1H), 1.05 (s, 9H); ES-LCMS m/z 478 (M+H).

### Example 14d

### Preparation of (1S)-2,2-dimethyl-1-({4-[4-(trifluoromethyl)phenyl]-1H-pyrazol-1-yl}methyl)propyl(1S)-1-[(1R)-1-hydroxy-2-oxo-2-(1H-pyrazol-5-ylamino)ethyl]pentylcarbamate Et(1S)-2,2-dimethyl-1-({4-[4-(trifluoromethyl)phenyl]-1H-pyrazol-1-yl}methyl)propyl(1S)-1-[(1S)-1-hydroxy-2-oxo-2-(1H-pyrazol-5-ylamino)ethyl]pentylcarbamate

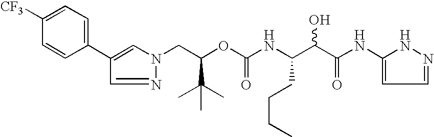

To 2.00 g (6.13 mmol) of tert-butyl(1S)-1-[(1R)-1-hydroxy-2-oxo-2-(1H-pyrazol-5-ylamino)ethyl]pentylcarbamate Et tert-butyl(1S)-1-[(1S)-1-hydroxy-2-oxo-2-(1H-pyrazol-5-ylamino)ethyl]pentylcarbamate in 20 mL of dioxane at room temperature was added 11.5 mL (45.96 mmol) of a 4M solution of hydrogen chloride in dioxane and the mixture was stirred for 1 h. It was concentrated and dried under vacuum, and then dissolved in 20 mL of N,N-dimethylformamide. This solution was cooled to 0° C. and 2.93 g (6.13 mmol) of (1S)-2,2-dimethyl-1-({4-[4-(trifluoromethyl)phenyl]-1H-pyrazol-1-yl}methyl)propyl 4-nitrophenyl carbonate in 11 mL of N,N-dimethylformamide was added. This was followed by 4.27 mL (24.51 mmol) of N,N-diisopropylethylamine, and the mixture was stirred for 18 h at room temperature. The solution was concentrated, saturated aqueous sodium bicarbonate was added and the mixture was extracted with ethyl acetate. The extract was washed with saturated aqueous sodium chloride, dried over anhydrous magnesium sulfate, filtered, and concentrated. The residue was purified by silica gel column chromatography eluting with a methanol:chloroform solution (1:9) to give two diastereomers. Isomer 1 (841.9 mg, 24%): R_{f}=0.28 (1:9 methanol:chloroform); ¹H NMR (300 MHz, DMSO-d₆) δ 12.33 (s, 1H), 9.56 (s, 1H). 8.23 (s, 1H), 7.94 (s, 1H), 7.75 (d, J=8 Hz, 2H), 7.63 (d, J=8 Hz, 2H), 7.55 (s, 1H), 6.66 (d, J=9 Hz, 1H), 6.42 (s, 1H), 5.86 (s, 1H), 4.76 (d, J=9 Hz, 1H), 4.39 (d, J=13 Hz, 1H), 4.12 (dd, J=14 Hz, J=11 Hz, 1H), 3.92 (d, J=7 Hz, 1H), 3.58 (p, J=5 Hz, 1H), 1.40-1.05 (m, 2H), 0.92 (s, 9H), 0.90-0.65 (m, 4H), 0.41 (t, J=6 Hz, 3H); ES-LCMS m/z 565 (M+H). Isomer 2 (1034.9 mg, 30%): R_{f}=0.24 (1:9 methanol:chloroform); ¹H NMR (300 MHz, DMSO-d₆) δ 12.26 (s, 1H), 9.67 (s, 1H), 8.24 (s, 1H), 7.94 (s, 1H), 7.76 (d, J=8 Hz, 2H), 7.64 (d, J=8 Hz, 2H), 7.54 (s, 1H), 6.40 (d, J=10 Hz, 1H), 6.38 (s, 1H), 5.60 (s, 1H), 4.64 (d, J=10 Hz, 1H), 4.35 (d, J=12 Hz, 1H), 4.07 (dd, J=14 Hz, J=11 Hz, 1H), 3.94 (s, 1H), 3.68-3.58 (m, 1H), 1.50-1.05 (m, 2H), 1.00-0.60 (m, 4H), 0.83 (s, 9H), 0.45 (t, J=7 Hz, 3H); ES-LCMS m/z 565 (M+H).

### Example 14e

### Preparation of (1S)-2,2-dimethyl-1-({4-[4-(trifluoromethyl)phenyl]-1H-pyrazol-1-yl}methyl)propyl(1S)-1-[oxo(1H-pyrazol-5-ylamino)acetyl]pentylcarbamate

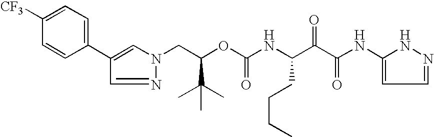

To 841.9 mg (1.49 mmol) of isomer 1 from example 14d in 15 mL of chloroform at room temperature was added 790.8 mg (1.86 mmol) of Dess-Martin periodinane, and the reaction mixture was stirred for 15 min. The mixture was poured into saturated aqueous sodium metabisulfite and subsequently neutralized with saturated aqueous sodium bicarbonate. The resulting mixture was extracted with ethyl acetate. The extract was dried over anhydrous magnesium sulfate, filtered, and concentrated. The residue was purified by silica gel column chromatography eluting with an ethyl acetate:hexanes solution (7:3) to give 690.0 mg (82%) of (1S)-2,2-dimethyl-1-({4-[4-(trifluoromethyl)phenyl]-1H-pyrazol-1-yl}methyl)propyl(1S)-1-[oxo(1H-pyrazol-5-ylamino)acetyl]pentylcarbamate. R_{f}=0.29 (7:3 ethyl acetate:hexanes); ¹H NMR (300 MHz, DMSO-d₆, Temp=110° C.) δ 10.32 (s, 1H), 8.11 (s, 1H), 7.86 (s, 1H), 7.72 (d, J=8 Hz, 2H), 7.62 (d, J=8 Hz, 2H), 7.56 (s, 1H), 6.98 (s, 1H), 6.45 (s, 1H), 4.82 (d, J=9 Hz, 1H), 4.80-4.66 (m, 1H), 4.41 (d, J=14 Hz, 1H), 4.14 (dd, J=14 Hz, 9 Hz, 1H), 1.76-1.58 (m, 1H), 1.54-1.36 (m, 1H), 1.34-1.08 (m, 4H), 0.97 (s, 9H), 0.70 (t, J=7 Hz, 3H); HRMS C₂₇H₃₄F₃N₆O₄ m/z 563.2594 (M+H)_{Cal}; 563.2589 (M+H)_{Obs}.

### Example 14f

### Preparation of (1S)-2,2-dimethyl-1-({4-[4-(trifluoromethyl)phenyl]-1H-pyrazol-1-yl}methyl)propyl(1S)-1-[oxo(1H-pyrazol-5-ylamino)acetyl]pentylcarbamate

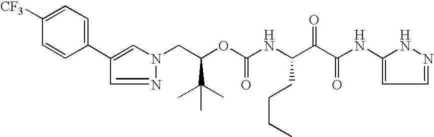

To 1.03 g (1.82 mmol) of isomer 2 from example 14d in 36 mL of chloroform:dimethyl sulfoxide (5:1) at room temperature was added 967.2 mg (2.28 mmol) of Dess-Martin periodinane, and the reaction mixture was stirred for 15 min. The mixture was poured into saturated aqueous sodium metabisulfite and subsequently neutralized with saturated aqueous sodium bicarbonate. The resulting mixture was extracted with ethyl acetate. The extract was dried over anhydrous magnesium sulfate, filtered, and concentrated. The residue was purified by silica gel column chromatography eluting with an ethyl acetate:hexanes solution (7:3) to give 889.0 mg (87%) of (1S)-2,2-dimethyl-1-({4-[4-(trifluoromethyl)phenyl]-1H-pyrazol-1-yl}methyl)propyl(1S)-1-[oxo(1H-pyrazol-5-ylamino)acetyl]pentylcarbamate. All spectral properties were identical to the title compound prepared from isomer 1 in example 14e.

### Example 15

### Preparation of (7R)-2,2-dimethyl-1-[(5-phenyl-1,3,4-oxadiazol-2-yl)methyl]propyl(1S)-1-(oxo{[(1R)-1-phenylethyl]amino}acetyl)pentylcarbamate

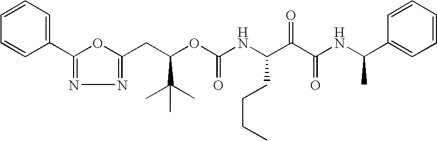

### Example 15a

### Preparation of methyl(2S)-2-isocyanatohexanoate

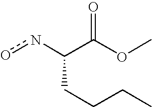

To 25.00 g (137.62 mmol) of methyl(2S)-2-aminohexanoate hydrochloride in 458 mL of dichloromethane at 0° C. was added 44.52 mL (550.49 mmol) of pyridine. Then, 85.57 mL (165.14 mmol) of a 1.93 M solution of phosgene in toluene was added to the solution, and the resulting mixture was stirred at 0° C. for 3 h. It was poured into 1N hydrochloric acid at 0° C., and the resulting mixture was extracted with dichloromethane. The extract was washed with 1N hydrochloric acid and saturated aqueous sodium chloride, dried over anhydrous magnesium sulfate, filtered, and concentrated. The residue was purified by vacuum distillation at 80° C. at 2 torr to give 21.32 g (91%) of methyl(2S)-2-isocyanatohexanoate. R_{f}=0.46 (1:4 ethyl acetate:hexanes); ¹H NMR (300 MHz, DMSO-d₆) δ 4.37 (dd, J=7 Hz, J=5 Hz, 1H), 3.73 (s, 3H), 1.78-1.60 (m, 2H), 1.36-1.16 (m, 4H), 0.85 (t, J=7 Hz, 3H); ES-MS m/z 172 (M+H).

### Example 75b

### Preparation of 2-methyl-5-phenyl-1,3,4-oxadiazole

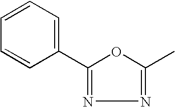

A solution of 10.10 mL (55.08 mmol) of triethyl orthoacetate and 5.00 g (36.72 mmol) of benzoic hydrazide in 122 mL of xylenes was heated at reflux for 18 h with water removal employing a Dean-Stark trap. The solution was cooled and concentrated. The residue was purified by silica gel column chromatography eluting with an ethyl acetate:hexanes solution (3:2) to give 5.75 g (98%) of 2-methyl-5-phenyl-1,3,4-oxadiazole. R_{f}=0.31 (2:3 ethyl acetate:hexanes); ¹H NMR (300 MHz, DMSO-d₆) δ 7.97 (dd, J=7 Hz, J=2 Hz, 2H), 7.61-7.56 (m, 3H), 2.58 (s, 3H); ES-MS m/z 161 (M+H).

### Example 15c

### Preparation of (2R)-3,3-dimethyl-1-(5-phenyl-1,3,4-oxadiazol-2-yl)-2-butanol Et(2S)-3,3-dimethyl-1-(5-phenyl-1,3,4-oxadiazol-2-yl)-2-butanol

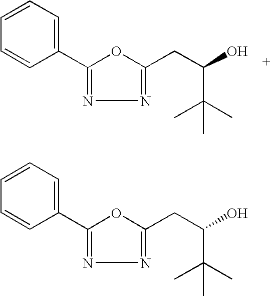

To 1.00 g (6.24 mmol) of 2-methyl-5-phenyl-1,3,4oxadiazole in 31 mL of tetrahydrofuran at −78° C. was added 4.29 mL (6.87 mmol) of 1.6 M n-butyllithium in hexanes, and the solution was stirred for 5 min. Then, 813.6 μL (7.49 mmol) of trimethylacetaldehyde were added. The reaction mixture was stirred for 15 min at −78° C., then allowed to warm to room temperature over 2 h in the bath. Water was added, and the mixture was extracted with ethyl acetate. The extract was washed with saturated aqueous sodium chloride, dried over anhydrous magnesium sulfate, filtered, and concentrated. The residue was purified by silica gel column chromatography eluting with an ethyl acetate:hexanes solution (1:1) to give 663.4 mg (440%) of a racemic mixture of (2R)-3,3-dimethyl-1-(5-phenyl-1,3,4-oxadiazol-2-yl)-2-butanol Et(2S)-3,3-dimethyl-1-(5-phenyl-1,3,4-oxadiazol-2-yl)-2-butanol. R_{f}=0.34 (2:3 ethyl acetate:hexanes); ¹H NMR (300 MHz, DMSO-d₆) δ 7.98 (dd, J=7 Hz, J=2 Hz, 2H), 7.61-7.58 (m, 3H), 4.96 (d, J=6 Hz, 1H), 3.65-3.55 (m, 1H), 3.11-2.76 (ABX, 2H), 0.91 (s, 9H); ES-LCMS m/z 247 (M+H). The enantiomers were separated by supercritical fluid chromatography utilizing a ChiralpaK AD column (20×250 mm) eluting with carbon dioxide:methanol (9:1 at 0.1 to 35.1 Mpa Et−10° C. to 100° C.).

### Example 15d

### Preparation of methyl(2S)-2-{[({(1R)-2,2-dimethyl-1-[(5-phenyl-1,3,4-oxadiazol-2-yl)methyl]propyl}oxy)carbonyl]amino}hexanoate

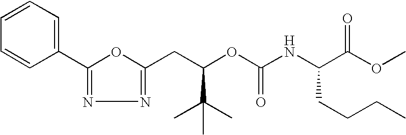

A solution of 232.2 mg (942.7 μmol) of (2R)-3,3-dimethyl-1-(5-phenyl-1,3,4-oxadiazol-2-yl)-2-butanol and 193.5 mg (1.13 mmol) of methyl(2S)-2-isocyanatohexanoate in 3.1 mL of toluene and the mixture was heated at 85° C. for 66 h. The solution was concentrated, and the residue was purified by silica gel column chromatography eluting with an ethyl acetate:hexanes solution (2:3) to give 393.1 mg (99%) of methyl(2S)-2-{[({(1R)-2,2-dimethyl-1-[(5-phenyl-1,3,4-oxadiazol-2-yl)methyl]propyl}oxy)carbonyl]amino}hexanoate. R_{f}=0.22 (2:3 ethyl acetate:hexanes); ¹H NMR (300 MHz, DMSO-d₆) δ 7.94 (d, J=8 Hz, 2H), 7.62-7.55 (m, 3H), 7.52 (d, J=8 Hz, 1H), 4.87 (d, J=8 Hz, 1H), 3.72 (dt, J=9 Hz, J=5 Hz, 1H), 3.53 (s, 3H), 3.32 (d, J=15 Hz, 1H), 3.04 (dd, J=15 Hz, J=11 Hz, 1H), 1.70-1.38 (m, 2H), 1.32-1.00 (m, 4H), 0.97 (s, 9H), 0.70 (t, J=7 Hz, 3H); ES-LCMS m/z 418 (M+H).

### Example 15e

### Preparation of (1R)-2,2-dimethyl-1-[(5-phenyl-1,3,4-oxadiazol-2-yl)methyl]propyl(1S)-1-[cyano(triphenylphosphoranylidene)acetyl]pentylcarbamate

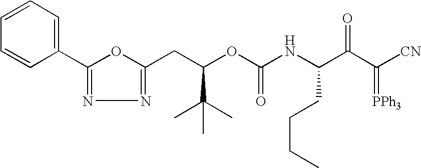

A solution of 393.1 mg (941.5 μmol) of (2S)-2-{[({(1R)-2,2-dimethyl-1-[(5-phenyl-1,3,4-oxadiazol-2-yl)methyl]propyl}oxy)carbonyl]amino}hexanoate and 55.3 mg (1.32 mmol) of lithium hydroxide monohydrate in 10 mL of tetrahydrofuran:water (1:1) was stirred at room temperature for 1 h. It was then concentrated. Water was added, and the resulting mixture was extracted with diethyl ether. The aqueous layer was acidified with 1 N hydrochloric acid and extracted with diethyl ether. The extract was dried over anhydrous magnesium sulfate, filtered, and concentrated. The residue was dissolved in 9.4 mL of dichloromethane, and 5.8 mg (47.1 μmol) of 4-dimethylaminopyridine was added. To this solution was added 297.9 mg (988.6 μmol) of (triphenylphosphoranylidene)acetonitrile, followed by 199.6 mg (1.03 mmol) of 1-ethyl-3-(3-dimethyl-aminopropyl)carbodiimide, and the mixture was stirred at room temperature for 17 h. Water was added and the resulting mixture was extracted with ethyl acetate. The extract was washed with 10% citric acid, followed by saturated aqueous sodium bicarbonate, and saturated aqueous sodium chloride. The extract was dried over anhydrous magnesium sulfate, filtered, and concentrated. The residue was purified by silica gel column chromatography eluting with an ethyl acetate:hexanes solution (7:3) to give 490.7 mg (76%) of (1R)-2,2-dimethyl-1-[(5-phenyl-1,3,4-oxadiazol-2-yl)methyl]propyl(1S)-1-[cyano(triphenylphosphoranylidene)acetyl]pentyl carbamate. R_{f}=0.33 (7:3 ethyl acetate:hexanes); ¹H NMR (300 MHz, DMSO-d₆) δ 7.96 (d, J=6 Hz, 2H), 7.71 (t, J=7 Hz, 3H), 7.58-7.48 (m, 15H), 7.06 (d, J=8 Hz, 1H), 4.95 (d, J=8 Hz, 1H), 4.36-4.26 (m, 1H), 3.30-2.96 (ABX, 2H), 1.70-1.35 (m, 2H), 1.20-1.00 (m, 4H), 0.95 (s, 9H), 0.70 (t, J=7 Hz, 3H); ES-LCMS m/z 687 (M+H).

### Example 15f

### Preparation of (1R)-2,2-dimethyl-1-[(5-phenyl-1,3,4-oxadiazol-2-yl)methyl]propyl(1S)-1-(oxo{[(1R)-1-phenylethyl]amino}acetyl)pentylcarbamate

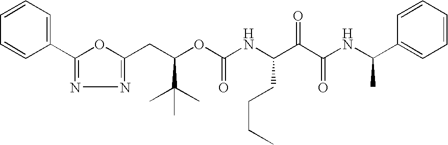

Ozone was bubbled through a solution of 176.8 mg (257.4 μmol) of (1R)-2,2-dimethyl-1-[(5-phenyl-1,3,4-oxadiazol-2-yl)methyl]propyl(1S)-1-[cyano(triphenyl phosphoranylidene)acetyl]pentylcarbamate in 12 mL of dichloromethane at −78° C. for 15 min. The solution was purged with a stream of nitrogen for 5 min, then 33.2 μL (257.4 μmol) of (S)-α-methylbenzylamine was added, and the solution was stirred at −78° C. for 15 min. It was then concentrated, and 5 mL of a 1 M solution of silver nitrate in tetrahydrofuran:water (4:1) was added. The mixture was stirred for 18 h at room temperature, and then was extracted with ethyl acetate. The extract was washed with 10% citric acid, followed by saturated aqueous sodium bicarbonate, and saturated aqueous sodium chloride. The extract was dried over anhydrous magnesium sulfate, filtered, and concentrated. The residue was purified by silica gel column chromatography eluting with an ethyl acetate:hexanes solution (2:3) to give 56.7 mg (41%) of (1R)-2,2-dimethyl-1-[(5-phenyl-1,3,4-oxadiazol-2-yl)methyl]propyl(1S)-1-(oxo{[(1R)-1-phenylethyl]amino}acetyl)pentylcarbamate. R_{f}=0.27 (2:3 ethyl acetate:hexanes); ¹H NMR (300 MHz, DMSO-d₆) δ 9.12 (d, J=8 Hz, 1H), 7.93 (d, J=8 Hz, 2H), 7.62-7.53 (m, 3H), 7.47 (d, J=8 Hz, 1H), 7.34-7.16 (m, 5H), 4.80 (p, J=7 Hz, 1H), 4.83 (d, J=8 Hz, 1H), 4.55 (m, 1H), 3.28 (d, J=8 Hz, 1H), 3.04 (dd, J=15 Hz, J=9 Hz, 1H), 1.54-1.00 (m, 6H), 1.37 (d, J=7 Hz, 3H), 0.97 (s, 9H), 0.65 (t, J=7 Hz, 3H); HRMS C₃₀H₃₉N₄O₅ m/z 535.2920 (M+H)_{Cal}; 535.2906 (M+H)_{Obs}.

### Example 16

### Preparation of (1S)-2,2-dimethyl-1-[(5-phenyl-1,3,4-oxadiazol-2-yl)methyl]propyl (1S)-1-(oxo{[(1R)-1-phenylethyl]amino}acetyl)pentylcarbamate

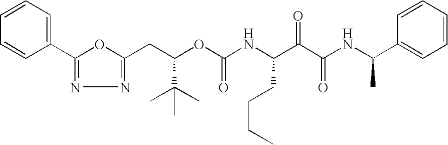

### Example 16a

### Preparation of methyl(2S)-2-{[({(2S)-2,2-dimethyl-1-[(5-phenyl-1,3,4-oxadiazol-2-yl)methyl]propyl}oxy)carbonyl]amino}hexanoate

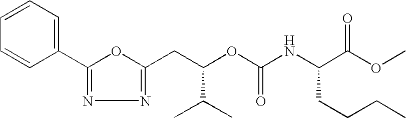

A solution of 259.1 mg (1.05 mmol) of (2S)-3,3-dimethyl-1-(5-phenyl-1,3,4-oxadiazol-2-yl)-2-butanol and 215.9 mg (1.26 mmol) of methyl(2S)-2-isocyanatohexanoate in 3.5 mL of toluene was heated at 85° C. for 70 h. It was concentrated, and the residue was purified by silica gel column chromatography eluting with an ethyl acetate:hexanes solution (2:3) to give 429.1 mg (98%) of methyl (2S)-2-{[({(1S)-2,2-dimethyl-1-[(5-phenyl-1,3,4-oxadiazol-2-yl)methyl]propyl}oxy)carbonyl]amino}hexanoate. R_{f}=0.24 (2:3 ethyl acetate:hexanes); ¹H NMR (300 MHz, DMSO-d₆) δ 7.93 (d, J=7 Hz, 2H), 7.62-7.54 (m, 3H), 7.22 (d, J=7 Hz, 1H), 4.89 (d, J=10 Hz, 1H), 3.94-3.82 (m, 1H), 3.61 (s, 3H), 3.28 (d, J=16 Hz, 1H), 3.03 (dd, J=16 Hz, J=11 Hz, 1H), 1.64-1.42 (m, 2H), 1.30-1.08 (m, 4H), 0.96 (s, 9H), 0.77 (t, J=7 Hz, 3H); ES-LCMS m/z 418 (M+H).

### Example 16b

### Preparation of (1S)-2,2-dimethyl-1-[(5-phenyl-1,3,4-oxodiazol-2-yl)methyl]propyl(1S)-1-[cyano(triphenylphosphoranylidene)acetyl]pentylcarbamate

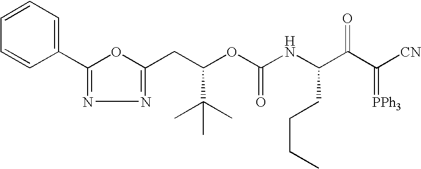

A solution of 429.1 mg (1.03 mmol) of (2S)-2-{[({(1S)-2,2-dimethyl-1-[(5-phenyl-1,3,4-oxadiazol-2-yl)methyl]propyl}oxy)carbonyl]amino}hexanoate and 60.4 mg (1.43 mmol) of lithium hydroxide monohydrate in 10 mL of tetrahydrofuran:water (1:1) was stirred at room temperature for 1 h. It was concentrated. Water was added, and the resulting mixture was extracted with diethyl ether. The aqueous layer was acidified with 1 N hydrochloric acid, and extracted with diethyl ether. The extract was dried over anhydrous magnesium sulfate, filtered, and concentrated. The residue was dissolved in 10 mL of dichloromethane, then 6.3 mg (51.4 μmol) of 4-dimethylaminopyridine was added. To this solution was added 355.2 mg (1.08 mmol) of (triphenylphosphoranylidene)acetonitrile, followed by 217.9 mg (1.13 mmol) of 1-ethyl-3-(3-dimethyl-aminopropyl) carbodiimide, and the mixture was stirred at room temperature for 14 h. Water was added to the solution, and the resulting mixture was extracted with ethyl acetate. The extract was washed with 10% citric acid, followed by saturated sodium aqueous bicarbonate and saturated aqueous sodium chloride. The extract was dried over anhydrous magnesium sulfate, filtered, and concentrated. The residue was purified by silica gel column chromatography eluting with an ethyl acetate:hexanes solution (7:3) to give 539.2 mg (76%) of (1S)-2,2-dimethyl-1-[(5-phenyl-1,3,4-oxadiazol-2-yl)methyl]propyl(1S)-1-[cyano(triphenyl phosphoranylidene)acetyl]pentylcarbamate. R_{f}=0.34 (7:3 ethyl acetate:hexanes); ¹H NMR (300 MHz, DMSO-d₆) δ 7.94 (d, J=7 Hz, 2H), 7.74-7.42 (m, 18H), 6.94 (d, J=8 Hz, 1H), 4.92 (m, 1H), 4.55 (m, 1H), 3.30-2.98 (ABX, 2H), 1.80-1.10 (m, 6H), 0.94 (s, 9H), 0.74 (t, J=7 Hz, 3H); ES-LCMS m/z 687 (M+H).

### Example 16c

### Preparation of (1S)-2,2-dimethyl-1-[(5-phenyl-1,3,4-oxadiazol-2-yl)methyl]propyl(1S)-1-(oxo{[(1R)-1-phenylethyl]amino}acetyl)pentylcarbamate

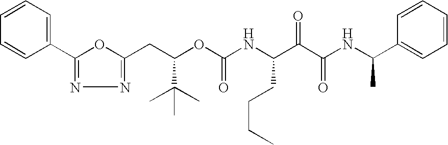

Ozone was bubbled through a solution of 250.2 mg (364.3 μmol) of (1S)-2,2-dimethyl-1-[(5-phenyl-1,3,4-oxadiazol-2-yl)methyl]propyl(1S)-1-[cyano(triphenylphosphoranylidene)acetyl]pentylcarbamate in 12 mL of dichloromethane at −78° C. for 15 min. The solution was purged with a stream of nitrogen for 5 min, and then 47.0 μL (364.3 μmol) of (S)-α-methylbenzylamine was added, and the solution was stirred at −78° C. for 15 min. The solution was concentrated and the residue was dissolved in 5 mL of a 1 M solution of silver nitrate in tetrahydrofuran:water (4:1). The mixture was stirred for 20 h at room temperature, and then extracted with ethyl acetate. The extract was washed with 10% citric acid, followed by saturated aqueous sodium bicarbonate and saturated aqueous sodium chloride. The extract was dried over anhydrous magnesium sulfate, filtered, and concentrated. The residue was purified by silica gel column chromatography eluting with an ethyl acetate:hexanes solution (2:3) to give 39.1 mg (20%) of (1S)-2,2-dimethyl-1-[(5-phenyl-1,3,4-oxadiazol-2-yl)methyl)propyl(1S)-1-(oxo{[(1R)-1-phenylethyl]amino}acetyl)pentylcarbamate. R_{f}=0.22 (2:3 ethyl acetate:hexanes); ¹H NMR (300 MHz, DMSO-d₆) δ 8.52 (br s, 1H). 7.93 (d, J=8 Hz, 2H), 7.60-7.50 (m, 3H), 7.34-7.16 (m, 5H), 6.96 (br s, 1H), 4.91 (p, J=7 Hz, 1H), 4.89 (dd, J=10 Hz, J=3 Hz, 1H), 4.84-4.74 (m, 1H), 3.26 (dd, J=15 Hz, J=3 Hz, 1H), 3.04 (dd, J=16 Hz, J=10 Hz, 1H), 1.66-1.52 (m, 1H), 1.50-1.36 (m, 1H), 1.42 (d, J=7 Hz, 3H), 1.30-1.10 (m, 4H), 0.96 (s, 9H), 0.76 (t, J=7 Hz, 3H); HRMS C₃₀H₃₉N₄O₅ m/z 535.2920 (M+H)_{Cal}; 535.2902 (M+H)_{Obs}.

### Example 17

### Preparation of (1R)-2,2-dimethyl-1-({5-[4-(trifluoromethyl)phenyl-1,3,4-oxadiazol-2-yl}methyl)propyl(1S)-1-(oxo{[(1R)-1-phenylethyl]amino}acetyl)pentylcarbamate

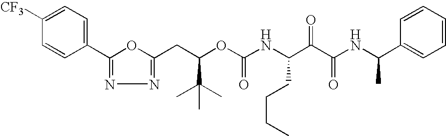

### Example 17a

### Preparation of (1R)-2,2-dimethyl-1-({5-[4-(trifluoromethyl)phenyl]-1,3,4-oxadiazol-2-yl}methyl)propyl(1S)-1-[cyano(triphenyl phosphoranylidene)acetyl]pentylcarbamate

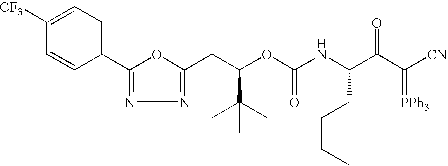

A solution of 357.5 mg (1.14 mmol) of (2R)-3,3-dimethyl-1-{5-[4-(trifluoromethyl)phenyl]-1,3,4-oxadiazol-2-yl}-2-butanol and 272.3 mg (1.59 mmol) of methyl(2S)-2-isocyanatohexanoate in 3.8 mL of toluene was heated at 85° C. for 44 h. The solution was concentrated, and the residue was purified by silica gel column chromatography eluting with an ethyl acetate:hexanes solution (2:3) to give methyl (2S)-2-[({[(1R)-2,2-dimethyl-1-({5-[4-(trifluoromethyl)phenyl]-1,3,4-oxadiazol-2-yl}methyl)propyl]oxy}carbonyl)amino]hexanoate, which was carried forward to the next reaction. R_{f}=0.22 (3:7 ethyl acetate:hexanes); ¹H NMR (300 MHz, DMSO-d₆) δ 8.15 (d, J=8 Hz, 2H), 7.97 (d, J 8 Hz, 2H), 7.52 (d, J=8 Hz, 1H), 4.88 (d, J=9 Hz, 1H), 3.71 (dt, J=8 Hz, J=5 Hz, 1H), 3.53 (s, 3H), 3.31 (dd, J=11 Hz, J=3 Hz, 1H), 3.08 (dd, J=15 Hz, J=11 Hz, 1H), 1.60-1.30 (m, 2H), 1.30-0.90 (m, 4H), 0.97 (s, 9H), 0.66 (t, J=7 Hz, 3H); ES-LCMS m/z 486 (M+H).

The above intermediate was dissolved in 11 mL of tetrahydrofuran:water (1:1) and 66.8 mg (1.59 mmol) of lithium hydroxide monohydrate was added. The mixture was stirred at room temperature for 1 h, before the tetrahydrofuran was removed under vacuum. The resulting aqueous mixture was washed with diethyl ether. The aqueous layer was acidified with 1 N hydrochloric acid, and extracted with diethyl ether. The extract was dried over anhydrous magnesium sulfate, filtered, and concentrated. The residue was dissolved in 11 mL of dichloromethane, and 6.9 mg (56.9 μmol) of 4-dimethylaminopyridine was added. To this solution was added 359.9 mg (1.20 mmol) of (triphenylphosphoranylidene)acetonitrile, followed by 241.1 mg (1.25 mmol) of 1-ethyl-3-(3-dimethyl-aminopropyl)carbodiimide, and the reaction mixture was stirred at room temperature for 17 h. Water was added to the solution, and the resulting mixture was extracted with ethyl acetate. The extract was washed with 10% citric acid, followed by saturated aqueous sodium bicarbonate, and saturated aqueous sodium chloride. It was then dried over anhydrous magnesium sulfate, filtered, and concentrated. The residue was purified by silica gel column chromatography eluting with an ethyl acetate:hexanes solution (3:2) to give 361.4 mg (42%) of (1R)-2,2-dimethyl-1-({5-[4-(trifluoromethyl)phenyl]-1,3,4-oxadiazol-2-yl}methyl)propyl(1S)-1-[cyano(triphenylphosphoranylidene)acetyl]pentylcarbamate. R_{f}=0.28 (3:2 ethyl acetate:hexanes); ¹H NMR (300 MHz, DMSO-d₆) δ 8.18 (d, J=8 Hz, 2H), 7.95 (d, J=8 Hz, 2H), 7.76-7.62 (m, 3H), 7.63-7.48 (m, 12H), 7.06 (d, J=8 Hz, 1H), 4.98 (d, J=9 Hz, 1H), 4.36-4.24 (m, 1H), 3.34-3.30 (m, 1H), 3.09 (dd, J=15 Hz, J=11 Hz, 1H), 1.70-1.46 (m, 1H), 1.44-1.26 (m, 1H), 1.16-0.90 (m, 4H), 0.96 (s, 9H), 0.64 (t, J=7 Hz, 3H); ES-LCMS m/z 755 (M+H).

### Example 17b

### Preparation of (1R)-2,2-dimethyl-1-({5-[4-(trifluoromethyl)phenyl]-1,3,4-oxadiazol-2-yl}methyl)propyl(1S)-1-(oxo{[(1R)-1-phenylethyl]amino}acetyl)pentylcarbamate

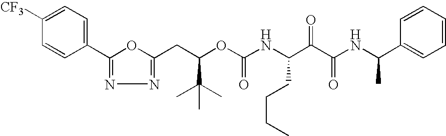

Ozone was bubbled through a solution of 159.1 mg (210.8 μmol) of (1R)-2,2-dimethyl-1-({5-[4-(trifluoromethyl)phenyl]-1,3,4-oxadiazol-2-yl}methyl)propyl(1S)-1-[cyano(triphenylphosphoranylidene)acetyl]pentylcarbamate in 7.0 mL of dichloromethane at −78° C for 15 min. The solution was purged with a stream of nitrogen for 5 min, and then 27.1 μL (210.8 μmol) of (S)-α-methylbenzylamine was added. The solution was stirred at −78° C. for 15 min. It was concentrated, and 5 mL of a 1 M solution of silver nitrate in tetrahydrofuran:water (4:1) was added. The resulting mixture was stirred for 20 h at room temperature, and then extracted with ethyl acetate. The extract was washed with 10% citric acid, followed by saturated aqueous sodium bicarbonate, and saturated aqueous sodium chloride. It was then dried over anhydrous magnesium sulfate, filtered, and concentrated. The residue was purified by silica gel column chromatography eluting with an ethyl acetate:hexanes solution (2:3) to give 55.0 mg (43%) of (1R)-2,2-dimethyl-1-({5-[4-(trifluoromethyl)phenyl]-1,3,4-oxadiazol-2-yl}methyl)propyl(1S)-1-(oxo{[(1R)-1-phenylethyl]amino}acetyl)pentylcarbamate. R_{f}=0.32 (2:3 ethyl acetate:hexanes); ¹H NMR (300 MHz, DMSO-d₆) δ 9.12 (d, J=8 Hz, 1H), 8.16 (d, J=8 Hz, 2H), 7.96 (d, J=8 Hz, 2H), 7.47 (d, J=8 Hz, 1H), 7.34-7.16 (m, 5H), 4.89 (p, J=8 Hz, 1H), 4.85 (d, J=8 Hz, 1H), 4.52 (dt, J=8 Hz, J=3 Hz, 1H), 3.31 (d, J=15 Hz, 1H), 3.08 (dd, J=15 Hz, J=11 Hz, 1H), 1.50-1.38 (m, 1H), 1.36 (d, J=7 Hz, 3H), 1.32-1.12 (m, 1H), 1.08-0.88 (m, 4H), 0.97 (s, 9H), 0.60 (t, J=7 Hz, 3H); HRMS C₃₁H₃₇F₃N₄O₅Na m/z 625.2614 (M+Na)_{Cal}; 625.2590 (M+Na)_{Obs}.

### Example 18

### Preparation of (1S)-2,2-dimethyl-1-({5-[4-(trifluoromethyl)phenyl]-1,3,4-oxadiazol-2-yl}methyl)propyl(1S)-1-(oxo{[(1R)-1-phenylethyl]amino}acetyl)pentylcarbamate

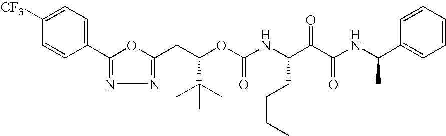

### Example 18a

### Preparation of (1S)-22-dimethyl-1-({5-[4-(trifluoromethyl)phenyl]-1,3,4-oxadiazol-2-yl}methyl)propyl(1S)-1-[cyano(triphenyl phosphoranylidene)acetyl]pentylcarbamate

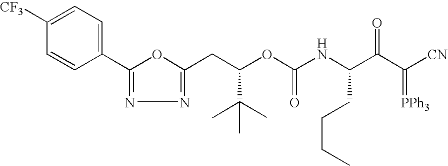

A solution of 347.5 mg (1.11 mmol) of (2S)-3,3-dimethyl-1-{5-[4-(trifluoromethyl)phenyl]-1,3,4-oxadiazol-2-yl}-2-butanol and 264.7 mg (1.55 mmol) of methyl(2S)-2-isocyanatohexanoate in 3.7 mL of toluene was heated at 85° C. for 40 h. The solution was concentrated and the residue was purified by silica gel column chromatography eluting with an ethyl acetate:hexanes solution (3:7) to give methyl (2S)-2-[({[(1S)-2,2-dimethyl-1-({5-[4-(trifluoromethyl)phenyl]-1,3,4-oxadiazol-2-yl}methyl)propyl]oxy}carbonyl)amino]hexanoate, which was carried forward to the next reaction. R_{f}=0.22 (3:7 ethyl acetate:hexanes); ¹H NMR (300 MHz, DMSO-d₆) δ 8.15 (d, J=8 Hz, 2H), 7.95 (d, J=8 Hz, 2H), 7.58 (d, J=8 Hz, 1H), 4.91 (d, J=11 Hz, 1H), 3.94-3.78 (m, 1H), 3.61 (s, 3H), 3.40-3.28 (m, 1H), 3.08 (dd, J=15 Hz, J=11 Hz, 1H), 1.62-1.44 (m, 2H), 1.34-1.06 (m, 4H), 0.96 (s, 9H), 0.77 (t, J=7 Hz, 3H); ES-LCMS m/z 486 (M+H).

The above intermediate was dissolved in 11 mL of tetrahydrofuran:water (1:1), and 64.9 mg (1.55 mmol) of lithium hydroxide monohydrate was added. The reaction mixture was stirred at room temperature for 1 h. Tetrahydrofuran was removed under vacuum and the resulting mixture was washed with diethyl ether. The aqueous layer was acidified with 1 N hydrochloric acid, and extracted with diethyl ether. The extract was dried over anhydrous magnesium sulfate, filtered, and concentrated. The residue was dissolved in 11 mL of dichloromethane, and 6.7 mg (55.3 μmol) of 4-dimethylaminopyridine was added, followed by 349.8 mg (1.16 mmol) of (triphenylphosphoranylidene)acetonitrile, and 234.4 mg (1.22 mmol) of 1-ethyl-3-(3-dimethyl-aminopropyl)carbodiimide. The reaction mixture was stirred at room temperature for 15 h. Water was added, and the mixture was extracted with ethyl acetate. The extract was washed with 10% citric acid, followed by saturated aqueous sodium bicarbonate, and saturated aqueous sodium chloride. The extract was dried over anhydrous magnesium sulfate, filtered, and concentrated. The residue was purified by silica gel column chromatography eluting with an ethyl acetate:hexanes solution (3:2) to give 185.3 mg (22%) of (1S)-2,2-dimethyl-1-({5-[4-(trifluoromethyl)phenyl]-1,3,4-oxadiazol-2-yl}methyl)propyl(1S)-1-[cyano(triphenyl phosphoranylidene)acetyl]pentylcarbamate. R_{f}=0.30 (3:2 ethyl acetate:hexanes); ¹H NMR (300 MHz, DMSO-d₆) δ 8.10 (d, J=8 Hz, 2H), 7.95 (d, J=8 Hz, 1H), 7.76-7.42 (m, 17H), 4.88 (d, J=7 Hz, 1H), 4.60-4.46 (m, 1H), 3.34-3.30 (m, 1H), 3.10-3.00 (m, 1H), 1.84-1.72 (m, 1H), 1.64-1.48 (m, 1H), 1.36-1.08 (m, 4H), 0.95 (s, 9H), 0.83 (t, J=7 Hz, 3H); ES-LCMS m/z 755 (M+H).

### Example 18b

### Preparation of (1S)-2,2-dimethyl-1-({5-[4-(trifluoromethyl)phenyl]-1,3,4-oxadiazol-2-yl}methyl)propyl(1S)-1-(oxo{[(1R)-1-phenylethyl]amino}acetyl)pentylcarbamate

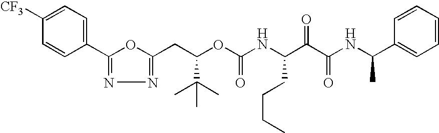

Ozone was bubbled through a solution of 185.3 mg (245.5 μmol) of (1S)-2,2-dimethyl-1-({5-[4-(trifluoromethyl)phenyl]-1,3,4-oxadiazol-2-yl}methyl)propyl(1S)-1-[cyano(triphenylphosphoranylidene)acetyl]pentylcarbamate in 8.1 mL of dichloromethane at −78° C. for 15 min. The solution was purged with a stream of nitrogen for 5 min, before 31.6 μL (245.5 μmol) of (S)-α-methylbenzylamine was added. The solution was stirred at −78° C. for 15 min, and then concentrated. The residue was dissolved in 5 mL of a 1 M solution of silver nitrate in tetrahydrofuran:water (4:1), and the reaction mixture was stirred for 17 h at room temperature. The solution was extracted with ethyl acetate. The extract was washed with 10% citric acid, followed by saturated aqueous sodium bicarbonate, and saturated aqueous sodium chloride. The extract was dried over anhydrous magnesium sulfate, filtered, and concentrated. The residue was purified by silica gel column chromatography eluting with an ethyl acetate:hexanes solution (2:3) to give 53.8 mg (36%) of (1S)-2,2-dimethyl-1-({5-[4-(trifluoromethyl)phenyl]-1,3,4-oxadiazol-2-yl}methyl)propyl(1S)-1-(oxo{[(1R)-1-phenylethyl]amino}acetyl)pentylcarbamate. R_{f}=0.25 (2:3 ethyl acetate:hexanes); ¹H NMR (300 MHz, DMSO-d₆, Temp=120° C.) δ 8.51 (br s, 1H), 8.15 (d, J=8 Hz, 2H), 7.90 (d, J=8 Hz, 2H), 7.32-7.16 (m, 5H), 6.98 (br s, 1H), 4.96-4.84 (m, 2H), 4.78-4.68 (m, 1H), 3.30 (dd, J=16 Hz, J=3 Hz, 1H), 3.08 (dd, J=16 Hz, J=10 Hz, 1H), 1.70-1.50 (m, 1H), 1.50-1.30 (m, 1H), 1.41 (d, J=7 Hz, 3H), 1.30-1.06 (m, 4H), 0.97 (s, 9H), 0.75 (t, J=7 Hz, 3H); HRMS C₃₁H₃₇F₃N₄O₅Na m/z 625.2614 (M+Na)_{Cal}; 625.2595 (M+Na)_{Obs}.

### Example 19

### Preparation of (1R)-1-{[5-(4-fluorophenyl)-1,3,4-oxadiazol-2-yl]methyl}-2,2-dimethylpropyl(1S)-1-(oxo{[(1R)-1-phenylethyl]amino}acetyl)pentylcarbamate

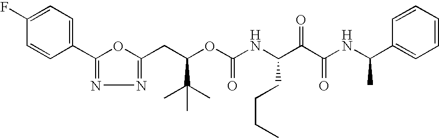

### Example 19a

### Preparation of (1R)-1-{[5-(4-fluorophenyl)-1,3,4-oxadiazol-2-yl]methyl}-2,2-dimethylpropyl(1S)-1-[cyano(triphenylphosphoranylidene)acetyl]pentylcarbamate

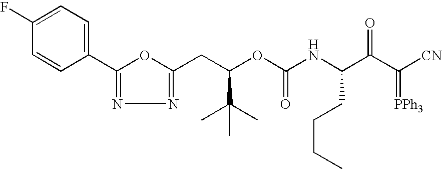

A solution of 328.8 mg (1.24 mmol) of (2R)-1-[5-(4fluorophenyl)-1,3,4-oxadiazol-2-yl]-3,3-dimethyl-2-butanol and 297.9 mg (1.74 mmol) of methyl(2S)-2-isocyanatohexanoate in 4.1 mL of toluene was heated at 85° C. for 42 h. The solution was concentrated and the residue was purified by silica gel column chromatography eluting with an ethyl acetate:hexanes solution (2:3) to give methyl(2S)-2-({[((1R)-1-{[5-(4-fluorophenyl)-1,3,4-oxadiazol-2-yl) methyl}-2,2-dimethylpropyl)oxy]carbonyl}amino)hexanoate, which was carried forward to the next reaction. R_{f}=0.27 (2:3 ethyl acetate:hexanes); ¹H NMR (300 MHz, DMSO-d₆) δ 7.99 (dd, J=9 Hz, J=5 Hz, 2H), 7.51 (d, J=8 Hz, 1H), 7.43 (t, J=9 Hz, 2H), 4.87 (dd, J=11 Hz, J=3 Hz, 1H), 3.72 (dt, J=8 Hz, J=5 Hz, 1H), 3.60 (s, 3H), 3.27 (dd, J=15 Hz, J=3 Hz, 1H), 3.04 (dd, J=15 Hz, J=11 Hz, 1H), 1.60-1.00 (m, 6H), 0.96 (s, 9H), 0.69 (t, J=7 Hz, 3H); ES-LCMS m/z 436 (M+H).

The above intermediate product was dissolved in 12 mL of tetrahydrofuran:water (1:1), and 73.1 mg (1.74 mmol) of lithium hydroxide monohydrate was added. The reaction mixture was stirred at room temperature for 2 h. Tetrahydrofuran was removed under vacuum and then the aqueous mixture was washed with diethyl ether. The aqueous layer was acidified with 1 N hydrochloric acid and extracted with diethyl ether. The extract was dried over anhydrous magnesium sulfate, filtered, and concentrated. The residue was dissolved in 12 mL of dichloromethane, and 7.6 mg (62.2 μmol) of 4-dimethylaminopyridine was added, followed by 393.6 mg (1.31 mmol) of (triphenylphosphoranylidene)acetonitrile, and 263.7 mg (1.37 mmol) of 1-ethyl-3-(3-dimethyl-aminopropyl)carbodiimide. The resulting mixture was stirred at room temperature for 16 h. Water was then added, and the mixture was extracted with ethyl acetate. The extract was washed with 10% citric acid, followed by saturated aqueous sodium bicarbonate, and saturated aqueous sodium chloride. The extract was dried over anhydrous magnesium sulfate, filtered, and concentrated. The residue was purified by silica gel column chromatography eluting with an ethyl acetate:hexanes solution (3:2) to give 598.6 mg (68%) of (1R)-1-{[5-(4-fluorophenyl)-1,3,4-oxadiazol-2-yl]methyl}-2,2-dimethylpropyl(1S)-1-[cyano(triphenylphosphoranylidene)acetyl]pentylcarbamate. R_{f}=0.21 (3:2 ethyl acetate:hexanes); ¹H NMR (300 MHz, DMSO-d₆) δ 8.02 (dd, J=9 Hz, J=5 Hz, 2H), 7.71 (t, J=7 Hz, 3H), 7.62-7.48 (m, 12H), 7.41 (t, J=9 Hz, 2H), 7.04 (d, J=8 Hz, 1H), 4.96 (dd, J=15 Hz, J=3 Hz, 1H), 4.36-4.28 (m, 1H), 3.27 (dd, J=8 Hz, J=3 Hz, 1H, 3.04 (dd, J=15 Hz, J=11 Hz, 1H), 1.68-1.52 (m, 1H), 1.44-1.24 (m, 1H), 1.20-1.00 (m, 4H), 0.95 (s, 9H), 0.69 (t, J=7 Hz, 3H); ES-LCMS m/z 705 (M+H).

### Example 19b

### Preparation of (1R)-1-{[5-(4-fluorophenyl)-1,3,4-oxadiazol-2-yl]methyl}-2,2-dimethylpropyl(1S)-1-(oxo{[(1R)-1-phenylethyl]amino}acetyl)pentylcarbamate

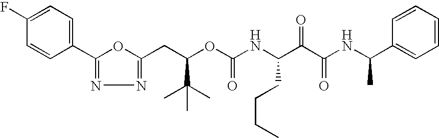

Ozone was bubbled through a solution of 177.7 mg (251.1 μmol) of (1R)-1-{[5-(4-fluorophenyl)-1,3,4oxadiazol-2-yl]methyl}-2,2-dimethylpropyl(1S)-1-[cyano (triphenylphosphoranylidene)acetyl]pentylcarbamate in 8.4 mL of dichloromethane at −78° C. for 15 min. The solution was purged with a stream of nitrogen for 5 min, and then 32.4 μL (251.1 μmol) of (S)-α-methylbenzylamine was added. The solution was stirred at −78° C. for 15 min, and then concentrated. The residue was dissolved in 5 mL of a 1 M solution of silver nitrate in tetrahydrofuran:water (4:1), and the reaction mixture was stirred for 16 h at room temperature. It was extracted with ethyl acetate. The extract was washed with 10% citric acid, followed by saturated aqueous sodium bicarbonate, and saturated aqueous sodium chloride. The extract was dried over anhydrous magnesium sulfate, filtered, and concentrated. The residue was purified by silica gel column chromatography eluting with an ethyl acetate:hexanes solution (2:3) to give 54.5 mg (39%) of (1R)-1-{[5-(4fluorophenyl)-1,3,4-oxadiazol-2-yl]methyl}-2,2-dimethylpropyl(1S)-1-(oxo{[(1R)-1-phenylethyl]amino}acetyl)pentylcarbamate. R_{f}=0.26 (2:3 ethyl acetate:hexanes); ¹H NMR (300 MHz, DMSO-d₆, Temp=120° C.) δ 8.56 (br s, 1H), 8.01 (dd, J=9 Hz, J=5 Hz, 2H), 7.37 (t, J=9 Hz, 2H), 7.34-7.16 (m, 5H), 6.93 (br s, 1H), 4.94 (p, J=7 Hz, 1H), 4.88 (dd, J=9 Hz, J=3 Hz, 1H), 4.74-4.58 (m, 1H), 3.25 (dd, J=5 Hz, J=3 Hz, 1H), 3.04 (dd, J=15 Hz, J=11 Hz, 1H), 1.62-1.48 (m, 1H), 1.43 (d, J=7 Hz, 3H), 1.42-1.32 (m, 1H), 1.18-1.06 (m, 4H), 0.98 (s, 9H), 0.72 (t, J=6 Hz, 3H); HRMS C₃₀H₃₇FN₄O₅Na m/z 575.2646 (M+Na)_{Cal}; 575.2650 (M+Na)_{Obs}.

### Example 20

### Preparation of (1S)-1-{[5-(4-fluorophenyl)-1,3,4-oxadiazol-2-yl]methyl}-2,2-dimethylpropyl(1S)-1-(oxo{[(1R)-1-phenylethyl]amino}acetyl)pentylcarbamate

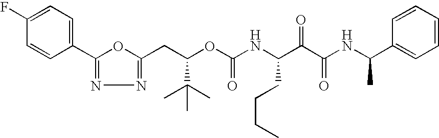

### Example 20a

### Preparation of (1S)-1-{[5-(4-fluorophenyl)-1,3,4-oxadiazol-2-yl]methyl}-2,2-dimethylpropyl(1S)-1-[cyano(triphenylphosphoranylidene)acetyl]pentylcarbamate

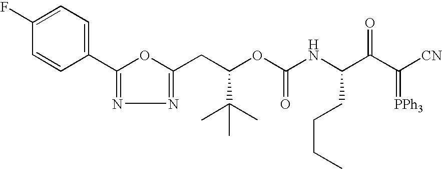

A solution of 331.2 mg (1.25 mmol) of (2S)-1-[5-(4-fluorophenyl)-1,3,4-oxadiazol-2-yl]-3,3-dimethyl-2-butanol and 300.0 mg (1.75 mmol) of methyl (2S)-2-isocyanatohexanoate in 4.1 mL of toluene was heated at 85° C. for 46 h. The solution was concentrated and the residue was purified by silica gel column chromatography eluting with an ethyl acetate:hexanes solution (2:3) to give methyl (2S)-2-({[((1S)-1-{[5-(4-fluorophenyl)-1,3,4-oxadiazol-2-yl]methyl}-2,2-dimethyl propyl)oxy]carbonyl}amino)hexanoate, which was carried forward to the next reaction. R_{f}=0.23 (2:3 ethyl acetate:hexanes); ¹H NMR (300 MHz, DMSO-d₆) δ 7.98 (dd, J=9 Hz, J=6 Hz, 2H), 7.57 (d, J=8 Hz, 1H), 7.42 (t, J=9 Hz, 2H), 4.89 (dd, J=11 Hz, J=3 Hz, 1H), 3.85 (dt, J=13 Hz, J=5 Hz, 1H), 3.61 (s, 3H), 3.27 (dd, J=16 Hz, J=3 Hz, 1H), 3.04 (dd, J=15 Hz, J=11 Hz, 1H), 1.68-1.40 (m, 2H), 1.36-1.06 (m, 4H), 0.95 (s, 9H), 0.78 (t, J=5 Hz, 3H); ES-LCMS m/z 436 (M+H).

The above intermediate was dissolved in 12 mL of tetrahydrofuran:water (1:1), and 73.6 mg (1.75 mmol) of lithium hydroxide monohydrate was added. The reaction mixture was stirred at room temperature for 2 h. Tetrahydrofuran was removed under vacuum, and the resulting mixture was washed with diethyl ether. The aqueous layer was then acidified with 1 N hydrochloric acid and extracted with diethyl ether. The extract was dried over anhydrous magnesium sulfate, filtered, and concentrated. The residue was dissolved in 12 mL of dichloromethane, and 7.7 mg (62.7 μmol) of 4-dimethylaminopyridine was added, followed by 396.5 mg (1.32 mmol) of (triphenylphosphoranylidene)acetonitrile, and 265.6 mg (1.38 mmol) of 1-ethyl-3-(3-dimethyl-aminopropyl)carbodiimide. The reaction mixture was stirred at room temperature for 20 h. Water was then added, and the resulting mixture was extracted with ethyl acetate. The extract was washed with 10% citric acid, followed by saturated aqueous sodium bicarbonate and saturated aqueous sodium chloride. The extract was dried over anhydrous magnesium sulfate, filtered, and concentrated. The residue was purified by silica gel column chromatography eluting with an ethyl acetate:hexanes solution (3:2) to give 537.4 mg (61%) of (1S)-1-{[5-(4-fluorophenyl)-1,3,4-oxadiazol-2-yl]methyl}-2,2-dimethylpropyl(1S)-1-[cyano(triphenylphosphoranylidene)acetyl]pentylcarbamate. R_{f}=0.30 (7:3 ethyl acetate:hexanes); ¹H NMR (300 MHz, DMSO-d₆, Temp=120° C.) δ 7.97 (dd, J=14 Hz, J=9 Hz, 2H), 7.74-7.64 (m, 3H), 7.62-7.50 (m, 12H), 7.26 (t, J=9 Hz, 2H), 6.31 (s, 1H), 4.93 (dd, J=9 Hz, J=3 Hz, 1H), 4.68-4.56 (m, 1H). 3.27 (dd, J=13 Hz, J=3 Hz, 1H). 3.06 (dd, J=15 Hz, J=6 Hz, 1H), 1.86-1.76 (m, 1H), 1.60-1.48 (m, 1H), 1.38-1.20 (m, 4H), 0.98 (s, 9H), 0.84 (t, J=7 Hz, 3H); ES-LCMS m/z 705 (M+H).

### Example 20b

### Preparation of (1S)-1-{[5-(4-fluorophenyl)-1,3,4-oxadiazol-2-yl]methyl}-2,2-dimethylpropyl(1S)-1-(oxo{[(1R)-1-phenylethyl]amino}acetyl)pentylcarbamate

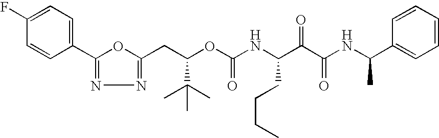

Ozone was bubbled through a solution of 166.6 mg (236.4 μmol) of (1S)-1-{[5-(4-fluorophenyl)-1,3,4-oxadiazol-2-yl]methyl}-2,2-dimethylpropyl(1S)-1-[cyano(triphenylphosphoranylidene)acetyl]pentylcarbamate in 8 mL of dichloromethane at −78° C. for 15 min. The solution was purged with a stream of nitrogen for 5 min. 30.5 μL (236.4 μmol) of (S)-α-methylbenzylamine was added and the solution was stirred at −78° C. for 15 min. The solution was concentrated, and the residue was dissolved in 5 mL of a 1 M solution of silver nitrate in tetrahydrofuran:water (4:1). The reaction mixture was stirred for 24 h at room temperature, and was then extracted with ethyl acetate. The extract was washed with 10% citric acid, followed by saturated aqueous sodium bicarbonate, and saturated aqueous sodium chloride. The extract was dried over anhydrous magnesium sulfate, filtered, and concentrated. The residue was purified by silica gel column chromatography eluting with an ethyl acetate:hexanes solution (2:3) to give 52.0 mg (40%) of (1S)-1-{[5-(4-fluorophenyl)-1,3,4-oxadiazol-2-yl]methyl}-2,2-dimethylpropyl(1S)-1-(oxo{[(1R)-1-phenylethyl]amino}acetyl)pentylcarbamate. R_{f}=0.25 (2:3 ethyl acetate:hexanes); ¹H NMR (300 MHz, DMSO-d₆, Temp=120° C.) δ 8.56 (br s, 1H), 7.99 (dd, J=9 Hz, J=5 Hz, 2H), 7.36 (t, J=10 Hz, 2H), 7.32-7.16 (m, 5H), 6.95 (br s, 1H), 4.96 (p, J=7 Hz, 1H), 4.88 (dd, J=10 Hz, J=6 Hz, 1H), 4.80-4.70 (m, 1H), 3.26 (dd, J=15 Hz, J=3 Hz, 1H), 3.04 (dd, J=15 Hz, J=10 Hz, 1H), 1.66-1.52 (m, 1H), 1.44-1.36 (m, 1H), 1.42 (d, J=7 Hz, 3H), 1.30-1.08 (m, 4H), 0.96 (s, 9H), 0.76 (t, J=7 Hz, 3H); HRMS C₃₀H₃₇FN₄O₅Na m/z 575.2646 (M+Na)_{Cal}; 575.2635 (M+Na)_{Obs}.

### Example 21

### Preparation of (1R)-1-{[5-(4-fluorophenyl)-1,3,4-oxadiazol-2-yl]methyl}-2,2-dimethylpropyl(1S)-1-[oxo(2-pyridinylamino)acetyl]pentylcarbamate

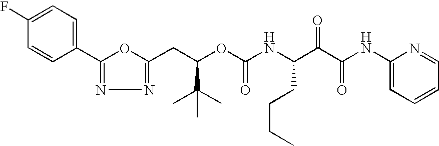

Ozone was bubbled through a solution of 176.9 mg (251.0 μmol) of (1R)-1-{[5-(4-fluorophenyl)-1,3,4-oxadiazol-2-yl]methyl}-2,2-dimethylpropyl(1S)-1-[cyano(triphenylphosphoranylidene)acetyl]pentylcarbamate in 8.4 mL of dichloromethane at −78° C. for 15 min. The solution was purged with a stream of nitrogen for 5 min, 23.6 mg (251.0 μmol) of 2-aminopyridine was added, and the resulting solution was stirred at −78° C. for 15 min. The solution was concentrated, and the residue was dissolved in 5 mL of a 1 M solution of silver nitrate in tetrahydrofuran:water (4:1). The reaction mixture was stirred for 24 h at room temperature, and was then extracted with ethyl acetate. The extract was washed with saturated aqueous sodium bicarbonate and saturated aqueous sodium chloride. The extract was dried over anhydrous magnesium sulfate, filtered, and concentrated. The residue was purified by silica gel column chromatography eluting with an ethyl acetate:hexanes solution (2:3) to give 5.1 mg (4%) of (1R)-1-{[5-(4-fluorophenyl)-1,3,4-oxadiazol-2-yl]methyl}-2,2-dimethylpropyl(1S)-1-[oxo(2-pyridinylamino)acetyl]pentylcarbamate. R_{f}=0.31 (4:1 ethyl acetate:hexanes); ¹H NMR (300 MHz, CDCL₃) δ 9.15 (br s, 1H), 8.39-8.29 (m, 1H), 8.19 (d, J=8 Hz, 1H), 8.11-7.94 (m, 2H), 7.73 (t, J=7 Hz, 1H), 7.18-7.04 (m, 3H), 5.30-5.18 (m, 1H), 5.06-5.04 (m, 2H), 3.24-3.12 (m, 2H), 1.96-0.80 (m, 6H), 1.29 (s, 9H), 0.84 (t, J=7 Hz, 3H); HRMS C₂₇H₃₃FN₅O₅ m/z 526.2465 (M+H)_{Cal}; 526.2482 (M+H)_{Obs}.

### Example 22

### Preparation of (1R)-1-{[5-(4-fluorophenyl)-1,3,4-oxadiazol-2-yl]methyl}-2,2-dimethylpropyl(1S)-1-[[(1-methyl-1H-pyrazol-5-yl)amino](oxo)acetyl]pentyl carbamate

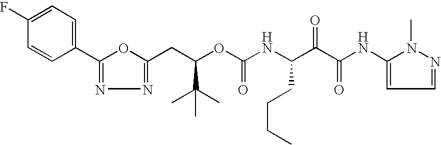

Ozone was bubbled through a solution of 205.1 mg (291.0 μmol) of (1S)-1-{[5-(4-fluorophenyl)-1,3,4-oxadiazol-2-yl]methyl}-2,2-dimethylpropyl(1S)-1-[cyano(triphenylphosphoranylidene)acetyl]pentylcarbamate in 10 mL of dichloromethane at −78° C. for 15 min. The solution was purged with a stream of nitrogen for 5 min, 28.3 mg (291.0 μmol) of 1-methyl-1H-pyrazol-5-amine in 2.9 mL of tetrahydrofuran was added and the solution was stirred at −78° C for 60 min. The solution was concentrated, 5 mL of a 1 M solution of silver nitrate in tetrahydrofuran:water (4:1) was added to the residue and the reaction mixture was stirred for 17 h at room temperature. It was then extracted with ethyl acetate. The extract was washed with saturated aqueous sodium bicarbonate, and saturated aqueous sodium chloride. The extract was dried over anhydrous magnesium sulfate, filtered, and concentrated. The residue was purified by silica gel column chromatography eluting with an ethyl acetate:hexanes solution (2:3) to give 43.1 mg (28%) of (1R)-1-{[5-(4-fluorophenyl)-1,3,4-oxadiazol-2-yl]methyl}-2,2-dimethylpropyl(1S)-1-[[(1-methyl-1H-pyrazol-5-yl)amino](oxo)acetyl]pentyl carbamate. R_{f}=0.28 (4:1 ethyl acetate:hexanes); ¹H NMR (300 MHz, DMSO-d₆, Temp=120° C.) δ 8.01 (dd, J=9 Hz, J=5 Hz, 2H), 7.52 (s, 1H), 7.38 (t, J=9 Hz, 2H), 7.13 (m, 1H), 6.83 (m, 2H), 4.83 (dd, J=11 Hz, J=3 Hz, 1H), 4.52-4.44 (m, 1H), 3.52 (s, 3H), 3.23 (dd, J=15 Hz, J=3 Hz, 1H), 3.03 (dd, J=15 Hz, J=11 Hz, 1H), 1.74-1.66 (m, 1H), 1.64-1.40 (m, 1H), 1.36-1.10 (m, 4H), 0.95 (s, 9H), 0.74 (t, J=7 Hz, 3H); HRMS C₂₆H₃₄FN₆O₅ m/z 529.2575 (M+H)_{Cal}; 529.2590 (M+H)_{Obs}.

### Example 23

### Preparation of (1R)-1-{[5-(4-fluorophenyl)-1,3,4-oxadiazol-2-yl]methyl}-2,2-dimethylpropyl(1S)-1-[oxo(1H-pyrazol-5-ylamino)acetyl]pentylcarbamate

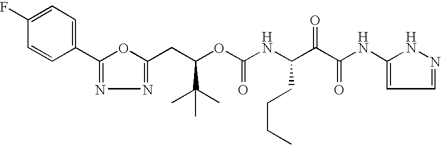

Ozone was bubbled through a solution of 175.4 mg (248.9 μmol) of (1S)-1-{[5-(4fluorophenyl)-1,3,4-oxadiazol-2-yl) methyl}-2,2-dimethylpropyl(1S)-1-[cyano(triphenylphosphoranylidene)acetyl]pentylcarbamate in 10 mL of dichloromethane at −78° C. for 15 min. The solution was purged with a stream of nitrogen for 5 min, 20.7 mg (248.9 μmol) of 1H-pyrazol-5-amine in 5 mL of tetrahydrofuran was added and the solution was stirred at −78° C for 60 min. The solution was concentrated, the residue was dissolved in 5 mL of a 1 M solution of silver nitrate in tetrahydrofuran:water (4:1). The reaction mixture was stirred for 64 h at room temperature, and then extracted with ethyl acetate. The extract was washed with saturated aqueous sodium bicarbonate, and saturated aqueous sodium chloride. The extract was dried over anhydrous magnesium sulfate, filtered, and concentrated. The residue was purified by silica gel column chromatography eluting with an ethyl acetate:hexanes solution (2:3) to give 45.6 mg (36%) of (1R)-1-{[5-(4-fluorophenyl)-1,3,4-oxadiazol-2-yl]methyl}-2,2-dimethylpropyl(1S)-1-[oxo(1H-pyrazol-5-ylamino)acetyl]pentylcarbamate. R_{f}=0.25 (7:3 ethyl acetate:hexanes); ¹H NMR (300 MHz, DMSO-d₆) δ 12.51 (s, 1H), 10.81 (s, 1H), 8.00 (dd, J=9 Hz, J=6 Hz, 2H), 7.63 (s, 1H), 7.52 (d, J=8 Hz, 1H), 7.44(t, J=9 Hz, 2H), 6.46 (s, 1H), 4.85 (dd, J=8 Hz, J=3 Hz, 1H), 4.66-4.56 (m, 1H), 3.28 (d, J=15 Hz, 1H), 3.05 (dd, J=15 Hz, J=11 Hz, 1H), 1.70-1.52 (m, 1H), 1.44-1.34 (m, 1H), 1.24-1.00 (m, 4H), 0.95 (s, 9H), 0.69 (t, J=7 Hz, 3H); HRMS C₂₅H₃₁FN₆O₅Na m/z 537.2238 (M+Na)_{Cal}; 537.2240 (M+Na)_{Obs}.

### Example 24

### Preparation of (1R)-1-{[5-(4-fluorophenyl)-1,3,4-oxadiazol-2-yl]methyl}-2,2-dimethylpropyl(1S)-1-{oxo[(4-pyridinylmethyl)amino]acetyl}pentylcarbamate

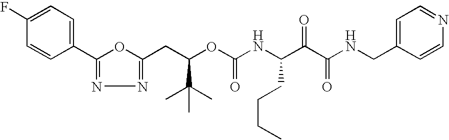

Ozone was bubbled through a solution of 164.4 mg (233.3 μmol) of (1S)-1-{[5-(4-fluorophenyl)-1,3,4-oxadiazol-2-yl]methyl}-2,2-dimethylpropyl(1S)-1-[cyano(triphenylphosphoranylidene)acetyl]pentylcarbamate in 12 mL of dichloromethane at −78° C. for 15 min. The solution was purged with a stream of nitrogen for 5 min, 23.7 μL (233.3 μmol) of 4-pyridinylmethanamine was added, and the solution was stirred at −78° C. for 60 min. The solution was concentrated, and the residue was dissolved in 5 mL of a 1 M solution of silver nitrate in tetrahydrofuran:water (4:1). The reaction mixture was stirred for 24 h at room temperature, and was then extracted with ethyl acetate. The extract was washed with saturated aqueous sodium bicarbonate and saturated aqueous sodium chloride. The extract was dried over anhydrous magnesium sulfate, filtered, and concentrated. The residue was purified by silica gel column chromatography eluting with an methanol:chloroform solution (1:19) to give 43.0 mg (340/%) of (1R)-1-{[5-(4-fluorophenyl)-1,3,4-oxadiazol-2-yl]methyl}-2,2-dimethylpropyl(1S)-1-{oxo[(4-pyridinylmethyl)amino]acetyl}pentylcarbamate. R_{f}=0.27 (1:9 methanol:chloroform); ¹H NMR (300 MHz, DMSO-d₆) δ 9.30 (t, J=6 Hz, 1H), 8.46 (d, J=5 Hz, 2H), 7.99 (dd, J=9 Hz, J=5 Hz, 2H), 7.51 (d, J=8 Hz, 1H), 7.43 (t, J=9 Hz, 2H), 7.19 (d, J=6 Hz, 2H), 4.85 (d, J=8 Hz, 1H), 4.62-4.52 (m, 1H), 4.28 (d, J=6 Hz, 2H), 3.27 (d, J=15 Hz, 1H), 3.04 (dd, J=15 Hz, J=11 Hz, 1H), 1.68-1.50 (m, 1H), 1.40-1.24 (m, 1H), 1.20-1.00 (m, 4H), 0.96 (s, 9H), 0.69 (t, J=6 Hz, 3H); HRMS C₂₈H₃₅FN₅O₅ m/z 540.2622 (M+H)_{Cal}; 540.2647 (M+H)_{Obs}.

### Example 25

### Preparation of (1R)-1-{[5-(4-fluorophenyl)-1,3,4-oxadiazol-2-yl]methyl}-2,2-dimethylpropyl(1S)-1-(oxo{[(3S)-2-oxopiperidinyl]amino}acetyl)pentylcarbamate

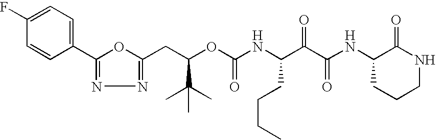

Ozone was bubbled through a solution of 160.0 mg (227.0 μmol) of (1S)-1-{[5-(4-fluorophenyl)-1,3,4-oxadiazol-2-yl]methyl}-2,2-dimethylpropyl(1S)-1-[cyano(triphenylphosphoranylidene)acetyl]pentylcarbamate in 12 mL of dichloromethane at −78° C. for 15 min. The solution was purged with a stream of nitrogen for 5 min, 25.9 mg (227.0 μmol) of (3S)-3-amino-2-piperidinone in 3 mL of tetrahydrofuran:dimethyl sulfoxide (1:1) was added, and the resulting solution was stirred at −78° C. for 60 min. It was concentrated, and the residue was dissolved in 5 mL of a 1 M solution of silver nitrate in tetrahydrofuran:water (4:1). The reaction mixture was stirred for 17 h at room temperature, and then extracted with ethyl acetate. The extract was washed with 10% citric acid, followed by saturated aqueous sodium bicarbonate and saturated aqueous sodium chloride. The extract was dried over anhydrous magnesium sulfate, filtered, and concentrated. The residue was purified by silica gel column chromatography eluting with an methanol:ethyl acetate solution (1:49) to give 14.1 mg (11%) of (1R)-1-{[5-(4-fluorophenyl)-1,3,4-oxadiazol-2-yl]methyl}-2,2-dimethylpropyl(1S)-1-(oxo{[(3S)-2-oxopiperidinyl]amino}acetyl)pentylcarbamate. R_{f}=0.23 (1:49 methanol:ethyl acetate); ¹H NMR (300 MHz, DMSO-d₆, Temp 110° C.) δ 8.31 (br s, 1H), 8.01 (dd, J=9 Hz, J=6 Hz, 2H), 7.38 (t, J=9 Hz, 2H), 7.33 (br s, 1H), 6.97 (br s, 1H), 4.87 (dd, J=8 Hz, J=3 Hz, 1H), 4.73-4.63 (m, 1H), 4.18-4.04 (m, 2H), 3.25 (dd, J=15 Hz, J=3 Hz, 1H), 3.20-3.10 (m, 1H), 3.05 (dd, J=15 Hz, J=11 Hz, 1H), 2.10-1.95 (m, 1H), 1.85-1.56 (m, 4H), 1.48-1.30 (m, 1H), 1.30-1.06 (m, 4H), 0.99 (s, 9H), 0.76 (t, J=7 Hz, 3H); HRMS C₂₇H₃₆FN₅O₆ m/z 568.2547 (M+Na)_{Cal}; 568.2557 (M+Na)_{Obs}.

### Example 26

### Preparation of (1R)-2,2-dimethyl-1-(2-{5-[4-(trifluoromethyl)phenyl]-1,3,4-oxadiazol-2-yl}ethyl)propyl(1S)-1-[oxo(1H-pyrazol-5-ylamino)acetyl]pentyl carbamate

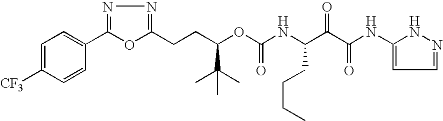

### Example 26a

### Preparation of (3R)-4,4-dimethyl-1-{5-[4-(trifluoromethyl)phenyl]-1,3,4-oxadiazol-2-yl}-3-pentanol

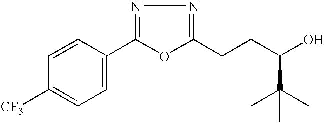

To 1.00 g (4.38 mmol) of 2-methyl-5-[4-(trifluoromethyl)phenyl]-1,3,4-oxadiazole in 40 mL of tetrahydrofuran at −78° C. was added 2.87 mL (4.60 mmol) of 1.6 M n-butyllithium in hexanes, and the solution was stirred for 5 min. Then, 526.8 mg (5.26 mmol) of (S)-3,3-dimethyl-1,2-epoxybutane in 4 mL of tetrahydrofuran was added, followed by 610.9 μL of boron trifluoride diethyl etherate, and the solution was stirred for 30 min at −78° C. Saturated aqueous sodium bicarbonate was added, and the mixture was extracted with ethyl acetate. The extract was dried over anhydrous magnesium sulfate, filtered, and concentrated. The residue was purified by silica gel column chromatography eluting with an methanol:chloroform solution (1:19) to give 182.0 mg (13%) of (3R)-4,4-dimethyl-1-{5-[4-(trifluoromethyl)phenyl]-1,3,4-oxadiazol-2-yl}-3-pentanol. R_{f}=0.21 (1:19 methanol:chloroform); ¹H NMR (300 MHz, DMSO-d₆) δ 8.19 (d, J=8 Hz, 2H), 7.96 (d, J=8 Hz, 2H), 4.65 (br s, 1H), 3.18-3.04 (m, 2H), 3.00-2.88 (1H), 2.02-1.86 (m, 1H), 1.72-1.54 (m, 1H), 0.84 (s, 9H); ES-LCMS m/z 351 (M+Na).

### Example 26b

### Preparation of (1R)-2,2-dimethyl-1-(2-{5-[4-(trifluoromethyl)phenyl]-1,3,4-oxadiazol-2-yl}ethyl)propyl(1S)-1-[cyano(triphenylphosphoranylidene)acetyl]pentylcarbamate

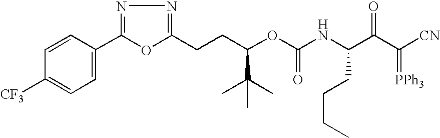

A solution of 182.0 mg (553.2 μmol) of (3R)-4,4-dimethyl-1-{5-[4-(trifluoromethyl)phenyl]-1,3,4-oxadiazol-2-yl}-3-pentanol and 132.7 mg (776.1 μmol) of methyl (2S)-2-isocyanatohexanoate in 2.8 mL of toluene was heated at 85° C. for 42 h. The solution was concentrated, and the residue was purified by silica gel column chromatography eluting with an ethyl acetate:hexanes solution (3:7). This residue was dissolved in 11 mL of tetrahydrofuran:water (1:1), and 32.6 mg (776.1 μmol) of lithium hydroxide monohydrate was added. The reaction mixture was stirred at room temperature for 3 h. Tetrahydrofuran was removed under vacuum, and the resulting aqueous mixture was washed with diethyl ether. The aqueous layer was acidified with 1 N hydrochloric acid and extracted with diethyl ether. The extract was dried over anhydrous magnesium sulfate, filtered, and concentrated. The residue was dissolved in 5.5 mL of dichloromethane, and 3.3 mg (27.7 μmol) of 4-dimethylaminopyridine was added, followed by 175.4 mg (582.0 μmol) of (triphenylphosphoranylidene)acetonitrile, and 117.5 mg (609.8 μmol) of 1-ethyl-3-(3-dimethyl-aminopropyl)carbodiimide. The reaction mixture was stirred at room temperature for 14 h. Water was added, and the resulting mixture was extracted with ethyl acetate. The extract was washed with 10% citric acid, followed by saturated aqueous sodium bicarbonate, and saturated aqueous sodium chloride. The extract was dried over anhydrous magnesium sulfate, filtered, and concentrated. The residue was purified by silica gel column chromatography eluting with an ethyl acetate:hexanes solution (3:2) to give 56.7 mg (13%) of (1R)-2,2-dimethyl-1-(2-{5-[4-(trifluoromethyl)phenyl]-1,3,4-oxadiazol-2-yl}ethyl)propyl(1S)-1-[cyano(triphenyl phosphoranylidene)acetyl]pentylcarbamate. R_{f}=0.28 (3:2 ethyl acetate:hexanes); ¹H NMR (300 MHz, DMSO-d₆) δ 8.19 (d, J=8 Hz, 2H), 7.95 (d, J=8 Hz, 2H), 7.71 (t, 3H, J=7 Hz), 7.66-7.50 (m, 12H), 7.06 (d, J=8 Hz, 1H), 4.61 (d, J=11 Hz, 1H), 4.54-4.42 (m, 1H), 2.93 (t, J=8 Hz, 2H), 2.20-2.08 (m, 1H), 1.96-1.78 (m, 1H), 1.78-1.62 (m, 1H), 1.62-1.42 (m, 1H), 1.40-1.16 (m, 4H), 0.88 (s, 9H), 0.84 (t, J=7 Hz, 3H); ES-LCMS m/z 769 (M+H).

### Example 26c

### Preparation of (1R)-2,2-dimethyl-1-(2-{5-[4-(trifluoromethyl)phenyl]-1,3,4-oxadiazol-2-yl}ethyl)propyl(1S)-1-[oxo(1H-pyrazol-5-ylamino)acetyl]pentylcarbamate

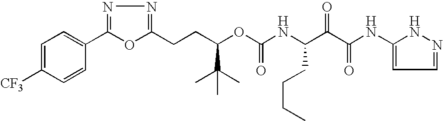

Ozone was bubbled through a solution of 52.8 mg (68.7 μmol) of (1R)-2,2-dimethyl-1-(2-{5-[4-(trifluoromethyl)phenyl]-1,3,4-oxadiazol-2-yl}ethyl)propyl(1S)-1-[cyano(triphenylphosphoranylidene)acetyl]pentylcarbamate in 7 mL of dichloromethane at −78° C. for 15 min. The solution was purged with a stream of nitrogen for 5 min, 5.7 mg (68.7 μmol) of 1H-pyrazol-5-amine in 2.5 mL of tetrahydrofuran was added, and the resulting solution was stirred at −78° C for 60 min. The solution was concentrated, and the residue was dissolved in 5 mL of a 1 M solution of silver nitrate in tetrahydrofuran:water (4:1). The reaction mixture was stirred for 24 h at room temperature, and then extracted with ethyl acetate. The extract was washed with saturated aqueous sodium bicarbonate, and saturated aqueous sodium chloride. The extract was dried over anhydrous magnesium sulfate, filtered, and concentrated. The residue was purified by silica gel column chromatography eluting with an ethyl acetate:hexanes solution (7:3) to give 12.5 mg (31%) of (1R)-2,2-dimethyl-1-(2-{5-[4-(trifluoromethyl)phenyl]-1,3,4-oxadiazol-2-yl}ethyl)propyl(1S)-1-[oxo(1H-pyrazol-5-ylamino)acetyl]pentylcarbamate. R_{f}=0.40 (1:9 methanol:chloroform); ¹H NMR (300 MHz, DMSO-d₆, Temp 110° C.) δ 10.28 (s, 1H), 8.17 (d, J=8 Hz, 2H), 7.92 (d, J=8 Hz, 2H), 7.54 (s, 1H), 7.12 (s, 1H), 6.47 (s, 1H), 4.92-4.74 (m, 1H), 4.58 (d, J=10 Hz, 1H), 2.88 (t, J=7 Hz, 2H), 2.24-2.08 (m, 1H), 2.00-1.86 (m, 1H), 1.84-1.72 (m, 1H), 1.70-1.48 (m, 1H), 1.44-1.24 (m, 4H), 0.92 (s, 9H), 0.86 (t, J=7 Hz, 3H); HRMS C₂₇H₃₄F₃N₆O₅ m/z 579.2542 (M+H)_{Cal}; 579.2523 (M+H)_{Obs}.

### Example 27

### Preparation of (1S)-1-(1H-benzimidazol-7-ylmethyl)-2,2-dimethylpropyl(1S)-1-[oxo(1H-pyrazol-5-ylamino)acetyl]pentylcarbamate

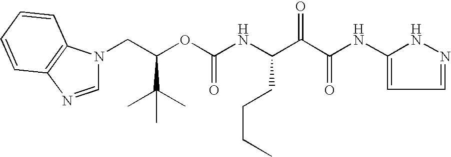

### Example 27a

### Preparation of (2S)-1-(1H-benzimidazol-1-yl)-3,3-dimethyl-2-butanol

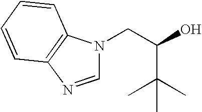

A solution of 847.8 mg (8.46 mmol) of (S)-3,3-dimethyl-1,2-epoxybutane and 1.00 g (8.46 mmol) of benzimidazole in 2.1 mL of ethanol was heated in a sealed tube at 85° C. for 17 h. The solution was cooled and concentrated, and the residue was purified by silica gel column chromatography eluting with a methanol:ethyl acetate solution (1:19) to give 1.23 g (67%) of (2S)-1-(1H-benzimidazol-1-yl)-3,3-dimethyl-2-butanol. R_{f}=0.27 (ethyl acetate); ¹H NMR (300 MHz, DMSO-d₆) δ 8.14 (s, 1H,), 7.63 (d, J=8 Hz, 1H), 7.49 (d, J=8 Hz, 1H), 7.24 (t, J=6 Hz, 1H), 7.19 (t, J=7 Hz, 1H), 4.95 (d, J=6 Hz, 1H), 4.38 (d, J=13 Hz, 1H), 3.96 (dd, J=14 Hz, J=10 Hz, 1H), 3.38-3.28 (m, 1H), 0.97 (s, 9H); ES-LCMS m/z 219 (M+H).

### Example 27b

### Preparation of 4-nitrophenyl(1S)-1-(1H-benzimidozol-1-ylmethyl)-2,2-dimethylpropyl carbonate

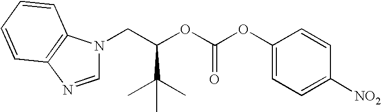

To 1.23 g (5.63 mmol) of (2S)-1-(1H-benzimidazol-1-yl)-3,3-dimethyl-2-butanol in 23 mL of tetrahydrofuran at 0° C. was added 3.70 mL (5.92 mmol) of 1.6 M n-butyllithium in hexanes, and the resulting solution was stirred for 10 min. Then, 1.48 g (7.32 mmol) of 4-nitrophenyl chloroformate in 5 mL of tetrahydrofuran was added and the solution was stirred at room temperature for 2 h. Saturated aqueous sodium bicarbonate was added to the solution, and the mixture was extracted with ethyl acetate. The extract was washed with saturated aqueous sodium chloride, dried over anhydrous magnesium sulfate, filtered, and concentrated. The residue was purified by silica gel column chromatography eluting with an ethyl acetate:hexanes solution (9:1) to give 379.8 mg (18%) of 4-nitrophenyl(1S)-1-(1H-benzimidazol-1-ylmethyl)-2,2-dimethylpropyl carbonate. R_{f}=0.37 (9:1 ethyl acetate:hexanes); ¹H NMR (300 MHz, DMSO-d₆) δ 8.30 (s, 1H), 8.16 (d, J=9 Hz, 2H), 7.67 (d, J=8 Hz, 1H), 7.61 (d, J=8 Hz, 1H), 7.28 (t, J=7 Hz, 1H), 7.21 (t, J=7 Hz, 1H), 6.84 (d, J=9 Hz, 2H), 4.85 (dd, J=10 Hz, J=2 Hz, 1H), 4.73 (dd, J=13 Hz, J=2 Hz, 1H), 4.51 (dd, J=15 Hz, 11=Hz, 1H), 1.11 (s, 9H); ES-LCMS m/z 384 (M+H).

### Example 27c

### Preparation of (1S)-1-(1H-benzimidazol-1-ylmethyl)-2,2-dimethylpropyl(1S)-1-[oxo(1H-pyrazol-5-ylamino)acetyl]pentylcarbamate

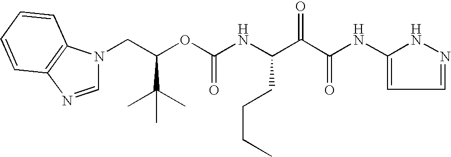

First, 83.4 mg (278.7 μmol) of (2R,3S)-3-amino-2-hydroxy-N-(1H-pyrazol-5-yl)heptanamide dihydrochloride Et(2S,3S)-3-amino-2-hydroxy-N-(1H-pyrazol-5-yl)heptanamide dihydrochloride in 2.6 mL of N,N-dimethylformamide was added to 106.9 mg (278.7 μmol) of 4-nitrophenyl (1S)-1-(1H-benzimidazol-1-ylmethyl)-2,2-dimethylpropyl carbonate in 3.0 mL of N,N-dimethylformamide, followed by 194.2 μL (1.11 mmol) of N,N-diisopropylethylamine. The reaction mixture was stirred for 14 h at room temperature, and then concentrated. Saturated aqueous sodium bicarbonate was added to the residue, and the resulting mixture was extracted with ethyl acetate. The extract was washed with saturated aqueous sodium chloride, dried over anhydrous magnesium sulfate, filtered, and concentrated. The residue was purified by silica gel column chromatography eluting with a methanol:ethyl acetate solution (1:9) to give 72.1 mg (55%) of a mixture of alcohols, which were dissolved in 5.1 mL of chloroform. Then, 81.2 mg (191.5 μmol) of Dess-Martin periodinane was added and the reaction mixture was stirred for 15 min. The mixture was poured into saturated aqueous sodium metabisulfite, and subsequently neutralized with saturated aqueous sodium bicarbonate. The mixture was extracted with ethyl acetate, and the extract was dried over anhydrous magnesium sulfate, filtered, and concentrated. The residue was purified by silica gel column chromatography eluting with an acetone:hexanes solution (3:2) to give 33.7 mg (47%) of (1S)-1-(1H-benzimidazol-1-ylmethyl)-2,2-dimethylpropyl(1S)-1-[oxo(1H-pyrazol-5-ylamino)acetyl]pentylcarbamate. R_{f}=0.42 (4:1 acetone:hexanes); ¹H NMR (300 MHz, DMSO-d₆, Temp=110° C.) δ 10.30 (s, 1H), 8.07 (s, 1H), 7.59 (d, J=8 Hz, 1H), 7.56 (s, 1H), 7.54 (d, J=8 Hz, 1H), 7.22 (t, J=7 Hz, 1H), 7.16 (t, J=7 Hz, 1H), 6.94 (br s, 1H), 6.44 (s, 1H), 4.78 (d, J=10 Hz, 1H), 4.68-4.56 (m, 1H), 4.52 (d, J=15 Hz, 1H), 4.24 (dd, J=15 Hz, J=10 Hz, 1H), 1.76-1.58 (m, 1H), 1.56-1.36 (m, 1H), 1.34-1.10 (m, 4H), 1.02 (s, 9H), 0.84 (t, J=7 Hz, 3H); HRMS C₂₄H₃₃N₆O₄ m/z 469.2563 (M+H)_{Cal}; 469.2546 (M+H)_{Obs}.

### Example 28

### Preparation of (1R)-2,2-dimethyl-1-({5-[4-(trifluoromethyl)phenyl]-1,3,4-oxadiazol-2-yl}methyl)propyl(1S)-1-{oxo[(2-oxo-1,3-oxazolidin-3-yl)amino]acetyl}pentyl carbamate

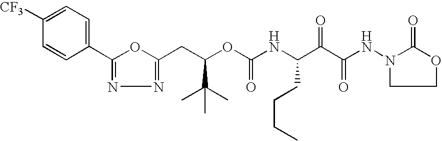

Ozone was bubbled through a solution of 156.4 mg (207.2 μmol) of (1R)-2,2-dimethyl-1-({5-[4-(trifluoromethyl)phenyl]-1,3,4-oxadiazol-2-yl}methyl)propyl(1S)-1-[cyano(triphenylphosphoranylidene)acetyl]pentylcarbamate in 6.9 mL of dichloromethane at −78° C. for 15 min. The solution was purged with a stream of nitrogen for 5 min. and then 21.2 mg (207.2 μmol) of 3-amino-1,3-oxazolidin-2-one in 4 mL of dimethyl sulfoxide:tetrahydrofuran (1:1) was added. The solution was stirred at −78° C. for 60 min, and then concentrated. The residue was dissolved in 5 mL of a 1 M solution of silver nitrate in tetrahydrofuran:water (4:1), and the mixture was stirred for 16 h at room temperature. It was extracted with ethyl acetate, and the extract was washed with 10% citric acid, followed by saturated aqueous sodium bicarbonate, and saturated aqueous sodium chloride. The extract was dried over anhydrous magnesium sulfate, filtered, and concentrated. The residue was purified by silica gel column chromatography eluting with an methanol:chloroform solution (1:19) to give 58.6 mg (48%) of (1R)-2,2-dimethyl-1-({5-[4-(trifluoromethyl)phenyl]-1,3,4-oxadiazol-2-yl}methyl)propyl(1S)-1-{oxo[(2-oxo-1,3-oxazolidin-3-yl)amino]acetyl}pentylcarbamate. R_{f}=0.21 (7:3 ethyl acetate:hexanes); ¹H NMR (300 MHz, DMSO-d₆, Temp=110° C.) δ 10.64 (s, 1H), 8.18 (d, J=8 Hz, 2H), 7.92 (d, J=8 Hz, 2H), 7.17 (s, 1H), 4.90 (dd, J=13 Hz, J=3 Hz, 1H), 4.76-4.54 (m, 1H), 4.37 (t, J=8 Hz, 2H), 3.65 (t, J=8 Hz, 2H), 3.30 (dd, J=15 Hz, J=3 Hz, 1H), 3.14 (dd, J=15 Hz, J=11 Hz, 1H), 1.76-1.56 (m, 1H), 1.52-1.32 (m, 1H), 1.30-1.04 (m, 4H), 1.00 (s, 9H), 0.74 (t, J=7 Hz, 3H); HRMS C₂₆H₃₃F₃N₅O₇ m/z 584.2332 (M+H)_{Cal}; 584.2327 (M+H)_{Obs}.

### Example 29

### Preparation of (1S)-2,2-dimethyl-1-{[3-(trifluoromethyl)-1H-pyrazol-1-yl]methyl}propyl(1S)-1-[oxo(1H-pyrazol-5-ylamino)acetyl]pentylcarbamate

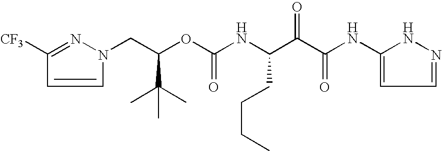

### Example 29a

### Preparation of (4S)-4-tert-butyl-1,3,2-dioxathiolane 2,2-dioxide

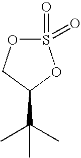

To 5.63 g (47.63 mmol) of (2S)-3,3-dimethyl-1,2-butanediol in 48 mL of carbon tetrachloride was added 3.47 mL (47.63 mmol) of thionyl chloride. The resulting mixture was heated at reflux for 1 h, and cooled to 0° C., before 48 mL of acetonitrile was added. Then, 1.0 mg (4.8 μmol) of ruthenium(III) chloride hydrate was added, followed by 15.28 g (71.45 mmol) of sodium periodate. The reaction mixture was diluted with 71 mL of water and stirred for 2 h. It was then extracted with diethyl ether. The extract was washed with saturated aqueous sodium bicarbonate, dried over anhydrous magnesium sulfate, filtered, and concentrated. The residue was purified by silica gel column chromatography eluting with an ethyl acetate:hexanes solution (3:7) to give 8.33 g (97%) of (4S)-4-tert-butyl-1,3,2-dioxathiolane 2,2-dioxide. R_{f}=0.30 (3:7 ethyl acetate:hexanes); ¹H NMR (300 MHz, DMSO-d₆) δ 4.99-4.88 (m, 2H), 4.78 (t, J=8 Hz, 1H), 0.94 (s, 9H).

### Example 29b

### Preparation of (2S)-3,3-dimethyl-1-[3-(trifluoromethyl)-1H-pyrazol-1-yl]-2-butanol

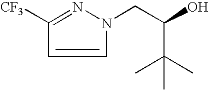

To 999.9 mg (5.55 mmol) of (4S)-4tert-butyl-1,3,2-dioxathiolane 2,2-dioxide in 18 mL of N,N-dimethylformamide was added 755.0 mg (5.55 mmol) of 3-(trifluoromethyl)-1H-pyrazole. Then, 805.2 mg (5.83 mmol) of potassium carbonate was added, and the resulting mixture was heated at 100° C. for 22 h. The solution was cooled and 20 mL of acetyl chloride:methanol (1:9) was added. The reaction mixture was stirred for 2 h, and then saturated aqueous sodium bicarbonate added. The mixture was extracted with ethyl acetate, and the extract was dried over anhydrous magnesium sulfate, filtered, and concentrated. The residue was purified by silica gel column chromatography eluting with an ethyl acetate:hexanes solution (1:4) to give 736.5 mg (56%) of (2S)-3,3-dimethyl-1-[3-(trifluoromethyl)-1H-pyrazol-1-yl]-2-butanol and a small amount of the other regioisomer. R_{f}=0.23 (1:4 ethyl acetate:hexanes); ¹H NMR (300 MHz, DMSO-d₆) δ 7.90 (s, 1H), 6.65 (d, J=2 Hz, 1H), 4.93 (br s, 1H), 4.30 (dd, J=14Hz, J=2 Hz, 1H), 3.96 (dd, J=14 Hz, 10 Hz, 1H), 3.45 (d, J=10 Hz, 1H), 0.90 (s, 9H), ES-LCMS m/z 237 (M+H).

### Example 29c

### Preparation of 4-nitrophenyl(1S)-2,2-dimethyl-1-{[3-(trifluoromethyl)-1H-pyrazol-1-ylmethyl}propyl carbonate

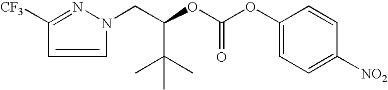

To 875.3 mg (3.71 mmol) of (2S)-3,3-dimethyl-1-[3-(trifluoromethyl)-1H-pyrazol-1-yl]-2-butanol in 12 mL of 1,2-dichloroethane at room temperature was added 746.8 g (3.71 mmol) of 4-nitrophenyl chloroformate. Then, 359.6 μL (4.45 mmol) of pyridine was added and the solution was heated at reflux for 22 h. Saturated aqueous sodium bicarbonate was added to the solution and the mixture was extracted with ethyl acetate. The extract was washed with saturated aqueous sodium chloride, dried over anhydrous magnesium sulfate, filtered, and concentrated. The residue was purified by silica gel column chromatography eluting with an ethyl acetate:hexanes solution (1:4) to give 1.39 g (93%) of 4-nitrophenyl (1S)-2,2-dimethyl-1-{[3-(trifluoromethyl)-1H-pyrazol-1-yl]methyl}propyl carbonate. R_{f}=0.29 (3:7 ethyl acetate:hexanes); ¹H NMR (300 MHz, DMSO-d₆) δ 8.28 (d, J=9 Hz, 2H), 8.07 (s, 1H), 7.31 (d, J=9 Hz, 2H), 6.74 (s, 1H), 4.87 (d, J=8 Hz, 1H), 4.69 (d, J=14 Hz 1H), 4.42 (dd, J=15 Hz, J=10 Hz, 1H), 1.03 (s, 9H); ES-LCMS m/z 424 (M+H).

### Example 29d

### Preparation of (7S)-2,2-dimethyl-1-{[3-(trifluoromethyl)-1H-pyrazol-1-yl]methyl}propyl(1S)-1-[oxo(1H-pyrazol-5-ylamino)acetyl]pentyl carbamate

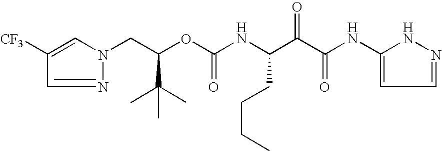

To 102.6 mg (314.3 μmol) of tert-butyl(1S)-1-[(1R)-1-hydroxy-2-oxo-2-(1H-pyrazol-5-ylamino)ethyl]pentylcarbamate Et tert-butyl(1S)-1-[(1S)-1-hydroxy-2-oxo-2-(1H-pyrazol-5-ylamino)ethyl]pentylcarbamate in 1.0 mL of dioxane at room temperature was added 3.9 mL (15.71 mmol) of a 4M solution of hydrogen chloride in dioxane. The mixture was stirred for 1 h, concentrated, and dried under vacuum. The residue was dissolved in 2.0 mL of N,N-dimethylformamide, and this solution was added to 126.2 mg (314.3 μmol) of 4-nitrophenyl(1S)-2,2-dimethyl-1-{[3-(trifluoromethyl)-1H-pyrazol-1-yl]methyl}propyl carbonate in 1.1 mL of N,N-dimethylformamide. This was followed by the addition of 219.0 μL (1.26 mmol) of N,N-diisopropylethylamine and the reaction mixture was stirred for 17 h at room temperature. The solution was concentrated, saturated aqueous sodium bicarbonate was added, and resulting the mixture was extracted with ethyl acetate. The extract was washed with saturated aqueous sodium chloride, dried over anhydrous magnesium sulfate, filtered, and concentrated. The residue was purified by silica gel column chromatography eluting with a methanol:ethyl acetate solution (1:19) to give 78.3 mg (51%) of a mixture of alcohols, which were dissolved in 3.2 mL of chloroform at room temperature. Then 85.0 mg (200.4 μmol) of Dess-Martin periodinane was added and the reaction mixture was stirred for 60 min. It was poured into saturated aqueous sodium metabisulfite, and the resulting mixture was subsequently neutralized with saturated aqueous sodium bicarbonate. The mixture was extracted with ethyl acetate. The extract was dried over anhydrous magnesium sulfate, filtered, and concentrated. The residue was purified by silica gel column chromatography eluting with an ethyl acetate:hexanes solution (4:1) to give 40.3 mg (52%) of (1S)-2,2-dimethyl-1-{[3-(trifluoromethyl)-1H-pyrazol-1-yl]methyl}propyl(1S)-1-[oxo(1H-pyrazol-5-ylamino)acetyl]pentylcarbamate. R_{f}=0.35 (4:1 ethyl acetate:hexanes); ¹H NMR (300 MHz, DMSO-d₆, Temp=110° C.) δ 10.39 (s, 1H), 7.79 (s, 1H), 7.58 (s, 1H), 7.06 (br s, 1H), 6.54 (s, 1H), 6.47 (s, 1H), 4.80 (d, J=9 Hz, 1H), 4.80-4.64 (m, 1H), 4.47 (d, J=14 Hz, 1H), 4.21 (dd, J=14 Hz, 9 Hz, 1H), 1.82-1.66 (m, 1H), 1.58-1.42 (m, 1H), 1.38-1.18 (m, 4H), 0.95 (s, 9H), 0.86 (t, J=7 Hz, 3H); HRMS C₂₁H₃₀F₃N₆O₄ m/z 487.2281 (M+H)_{Cal}; 487.2271 (M+H)_{Obs}.

### Example 30

### Preparation of (1S)-2,2-dimethyl-1-({S-[4-(trifluoromethyl)phenyl]-1H-pyrazol-1-yl}methyl)propyl(1S)-1-[oxo(1H-pyrazol-5-ylamino)acetyl]pentylcarbamate

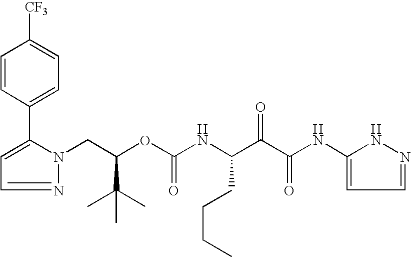

### Example 30a

### Preparation of 4-nitrophenyl(1S)-2,2-dimethyl-1-({5-[4-(trifluoromethyl)phenyl]-1H-pyrazol-1-yl}methyl)propyl carbonate

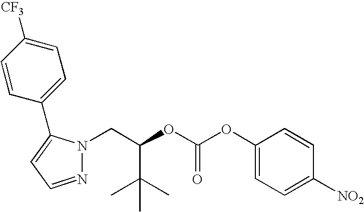

To 562.0 mg (1.80 mmol) of (2S)-3,3-dimethyl-1-{5-[4-(trifluoromethyl)phenyl]-1H-pyrazol-1-yl}-2-butanol in 18 mL of 1,2-dichloroethane at room temperature was added 544.1 g (2.70 mmol) of 4-nitrophenyl chloroformate. Then, 291.1 μL (3.60 mmol) of pyridine was added and the solution was heated at reflux for 16 h. Saturated aqueous sodium bicarbonate was added to the solution, and the resulting mixture was extracted with ethyl acetate. The extract was dried over anhydrous magnesium sulfate, filtered, and concentrated. The residue was purified by silica gel column chromatography eluting with an ethyl acetate:hexanes solution (3:7) to give 779.7 mg (91%) of 4-nitrophenyl (1S)-2,2-dimethyl-1-({5-[4-(trifluoromethyl)phenyl]-1H-pyrazol-1-yl}methyl)propyl carbonate. R_{f}=0.21 (3:7 ethyl acetate:hexanes); ¹H NMR (300 MHz, DMSO-d₆) δ 8.29 (d, J=8 Hz, 2H), 7.87 (d, J=8 Hz, 2H), 7.73 (d, J=8 Hz, 2H), 7.63 (s, 1H), 7.33 (d, J=9 Hz, 2H), 6.52 (s, 1H), 4.71 (d, J=9 Hz, 1H), 4.64 (d, J=15 Hz, 1H), 4.33 (dd, J=15 Hz, J=10 Hz, 1H), 0.89 (s, 9H); ES-LCMS m/z 478 (M+H).

### Example 30b

### Preparation of (1S)-2,2-dimethyl-1-({5-[4-(trifluoromethyl)phenyl]-1H-pyrazol-1-yl}methyl)propyl(1S)-1-[oxo(1H-pyrazol-5-ylamino)acetyl]pentylcarbamate

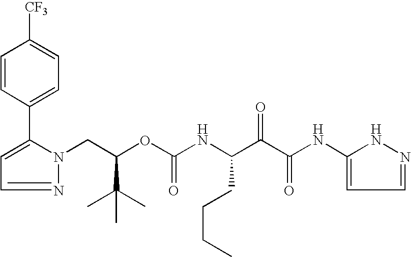

To 109.4 mg (335.0 μmol) of tert-butyl(1S)-1-[(1R)-1-hydroxy-2-oxo-2-(1H-pyrazol-5-ylamino)ethyl]pentylcarbamate Et tert-butyl(1S)-1-[(1S)-1-hydroxy-2-oxo-2-(1H-pyrazol-5-ylamino)ethyl]pentylcarbamate in 1.1 mL of dioxane at room temperature was added 4.2 mL (16.75 mmol) of a 4M solution of hydrogen chloride in dioxane. The mixture was stirred for 2 h, concentrated, dried under vacuum, and then dissolved in 2.0 mL of N,N-dimethylformamide. This solution was added to 133.3 mg (279.2 μmol) of 4-nitrophenyl(1S)-2,2-dimethyl-1-({5-[4-(trifluoromethyl)phenyl]-1H-pyrazol-1-yl}methyl)propyl carbonate in 3.6 mL of N,N-dimethylformamide, followed by 243.2 μL (1.40 mmol) of N,N-diisopropylethylamine. The resulting mixture was stirred for 66 h at room temperature. It was then concentrated, and the residue was partioned between saturated aqueous sodium bicarbonate and ethyl acetate. The organic layer was washed with saturated aqueous sodium chloride, dried over anhydrous magnesium sulfate, filtered, and concentrated. The residue was purified by silica gel column chromatography eluting with a methanol:ethyl acetate solution (1:19) to give 84.0 mg (53%) of a mixture of alcohols. The alcohols were dissolved in 3.0 mL of chloroform at room temperature, and 78.9 mg (186.0 μmol) of Dess-Martin periodinane was added. The reaction mixture was stirred for 60 min, and then poured into saturated aqueous sodium metabisulfite. The resulting mixture was subsequently neutralized with saturated aqueous sodium bicarbonate, and extracted with ethyl acetate. The extract was dried over anhydrous magnesium sulfate, filtered, and concentrated. The residue was purified by silica gel column chromatography eluting with ethyl acetate to give 48.3 mg (58%) of (1S)-2,2-dimethyl-1-({5-[4-(trifluoromethyl)phenyl]-1H-pyrazol-1-yl}methyl)propyl(1S)-1-[oxo(1H-pyrazol-5-ylamino)acetyl]pentylcarbamate. R_{f}=0.34 (ethyl acetate); ¹H NMR (300 MHz, DMSO-d₆, Temp=110° C.) δ 10.32 (s, 1H), 7.81 (d, J=8 Hz, 2H), 7.72 (d, J=8 Hz, 2H), 7.57 (s, 1H), 7.45 (s, 1H), 6.92 (br s, 1H), 6.46 (s, 1H), 6.35 (s, 1H), 4.81 (d, J=10 Hz, 1H), 4.80-4.62 (m, 1H), 4.38 (d, J=14 Hz, 1H), 4.15 (dd, J=14 Hz, J=11 Hz, 1H), 1.78-1.64 (m, 1H), 1.58-1.40 (m, 1H), 1.38-1.18 (m, 4H), 0.84 (s, 9H), 0.81 (t, J=7 Hz, 3H); HRMS C₂₇H₃₄F₃N₆O₄ m/z 563.2594 (M+H)_{Cal}; 563.2620 (M+H)_{Obs}.

### Example 31

### Preparation of (1S)-1-(1,3-benzothiazol-2-yl)-2,2-dimethylpropyl(1S)-1-[oxo(1H-pyrazol-5-ylamino)acetyl]pentylcarbamate

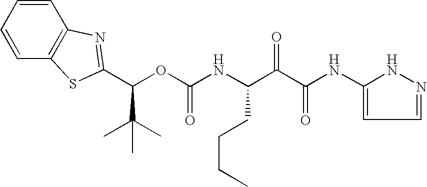

### Example 31a

### Preparation of 1-(1,3-benzothiazol-2-yl)-2,2-dimethyl-1-propanol

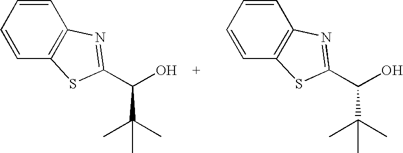

To a stirred solution of 2.0 g (14.8 mmol) of benzothiazole in 20 mL of tetrahydrofuran at −78° C. was added 10.0 mL (16.0 mmol) of a 1.6 M solution of n-butyllithium in hexane over 45 min. Then 1.4 g (16.3 mmol) of trimethylacetaldehyde in 10.0 mL of tetrahydrofuran was added dropwise over 30 min. The reaction mixture was stirred for 1 h at −78° C., and was then allowed to warm to 0° C. Then 25 mL of water was added, and the mixture was extracted with 80 mL of ether. The extract was washed with water, dried over anhydrous magnesium sulfate, filtered, and concentrated in vacuo. The residue was crystallized from ether/hexane to afford 2.24 g (69%) of 1-(1,3-benzothiazol-2-yl)-2,2-dimethyl-1-propanol as an off-white solid. The enantiomers were separated in 98.8% ee using a Chiralpak AD, 10 micron column with a mobile phase of 90% CO₂: 10% methanol and a flow rate of 2.0 mL/min. ¹H NMR (400MHz, DMSO-d₆): δ 8.01 (d, J=8 Hz, 1H), 7.90 (d, J=8 Hz, 1H), 7.44-7.33 (m, 2H), 6.41 (d, J=5 Hz, 1H), 4.53 (d, J=5 Hz, 1H), 0.93 (s, 9H).

### Example 31b

### Preparation of (1S)-1-(1,3-benzothiazol-2-yl)-2,2-dimethylpropyl-4-nitrophenyl carbonate

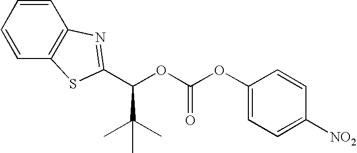

Treatment of (1S)-1-(1,3-benzothiazol-2-yl)-2,2-dimethyl-1-propanol with 4-nitrophenylchloroformate as described in example 1g provided the title compound as a pale yellow glass in 43% yield. ¹H NMR (400 MHZ, DMSO-d₆): δ 8.26 (d, J=9 Hz, 2H), 8.11 (d, J=8 Hz, 1H), 8.03 (d, J=8 Hz, 1H), 7.55-7.43 (m, 4H), 5.77 (s, 9H).

### Example 31c

### Preparation of (1S)-1-(1,3-benzothiazol-2-yl)-2,2-dimethylpropyl (1S)-1-[(1R)-1-hydroxy-2-oxo-2-(1H-pyrazol-5-ylamino)ethyl]pentyl carbamate Et(1S)-1-(1,3-benzothiazol-2-yl)-2,2-dimethylpropyl(1S)-1-[(1S)-1-hydroxy-2-oxo-2-(1H-pyrazol-5-ylamino)ethyl]pentylcarbamate

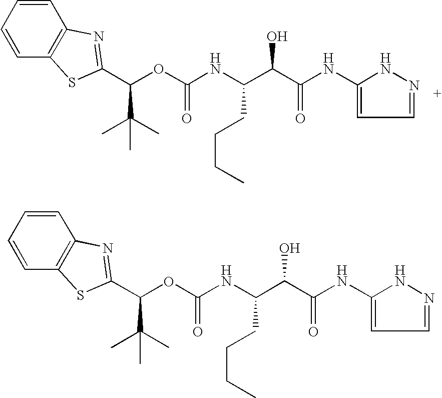

Treatment of tert-butyl(1S)-1-[hydroxy-2-oxo-2(1H-pyrazol-5-ylamino)ethyl]pentylcarbamate with (1S)-1-(1,3-benzothiazol-2-yl)-2,2-dimethylpropyl-4-nitrophenyl carbonate as described in example in provided the title compound as an off-white solid in 33% yield. ¹H NMR (400MHz, DMSO-d₆): δ 12.30 (br s, 1H), 9.70, 9.50 (2 br, 1H), 8.08-7.04 (m, 6H), 6.51-6.28 (m, 1H), 5.98-5.70 (m, 1H), 5.50-5.40 (m, 1H), 4.18-3.72 (m, 2H), 1.64-1.10 (m, 6H), 0.99, 0.97 (2s, 9H), 0.92-0.76 (m, 3H); ES-LCMS m/z 474 (M+H).

### Example 31d

### Preparation of (1S)-1-(1,3-benzothiazol-2-yl)-2,2-dimethylpropyl (1S)-1-[oxo(1H-pyrazol-5-ylamino)acetyl]pentylcarbamate

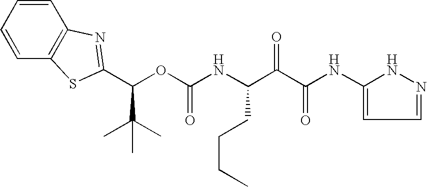

Treatment of (1S)-1-(1,3-benzothiazol-2-yl)-2,2-dimethylpropyl(1S)-1-[(1R)-1-hydroxy-2-oxo-2-(1H-pyrazol-5-ylamino)ethyl]pentylcarbamate Et(1S)-1-(1,3-benzothiazol-2-yl)-2,2-dimethylpropyl(1S)-1-[(1S)-1-hydroxy-2-oxo-2-(1H-pyrazol-5-ylamino)ethyl]pentylcarbamate with Dess-Martin reagent as described in example 1o provided the title compound as a white foam in 96% yield. ¹H NMR (400 MHz, DMSO-d₆): δ 12.51 (br s, 1H), 10.88 (br s, 1H), 8.07-7.38 (m, 6H), 6.47 (br s, 1H), 5.54 (s, 1H), 4.82-4.76 (m, 1H), 1.74-1.16 (m, 6H), 1.00 (s, 9H), 0.82-0.75 (m, 3H); ES-LCMS m/z 472 (M+H).

### Example 32

### Preparation of (1R)-1-(1,3-benzothiazol-2-yl)-2,2-dimethylpropyl(1S)-1-[oxo(1H-pyrazol-5-ylamino)acetyl]pentylcarbamate

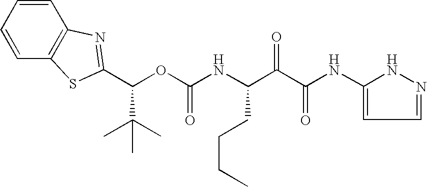

The title compound was obtained as in example 31 using (1R)-1-(1,3-benzothiazol-2-yl)-2,2-dimethyl-1-propanol from example 31a. ¹H NMR (400 MHz, DMSO-d₆): δ 12.49 (br s, 1H), 10.83 (br s, 1H), 8.08-7.37 (m, 6H), 6.45 (br s, 1H), 5.56 (s, 1H), 4.82-4.77 (m, 1H), 1.76-1.18 (m, 6H), 0.99 (s, 9H), 0.87-0.76 (m, 3H); ES-LCMS m/z 472 (M+H).

### Example 33

### Preparation of (1S)-2,2-dimethyl-1-{[3-(3-pyridinyl)-1H-pyrazol-1-yl]methyl}propyl(1S)-1-[oxo(1,3-thiazol-2-ylamino)acetyl]pentylcarbamate

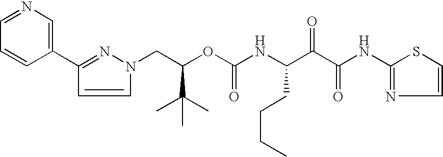

### Example 33a

### Preparation of tert-butyl(1S)-1-[(1R)-1-hydroxy-2-oxo-2-(1,3-thiazol-2-ylamino)ethyl]pentylcarbamate Et tert-butyl(1S)-1-[(1S)-1-hydroxy-2-oxo-2-(1,3-thiazol-2-ylamino)ethyl]pentylcarbamate

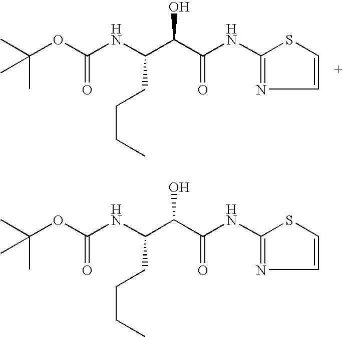

The title compound was obtained as in example 4d using (2R, 3S)-3-[(tert-butoxycarbonyl)amino]-2-hydroxyheptanoic acid Et(2S, 3S)-3-[(tert-butoxycarbonyl)amino]-2-hydroxyheptanoic acid from example 1l and 2-aminothiazole as a foam in 53% yield. ¹H NMR (400 MHz, DMSO-d₆): δ 11.67, 11.43 (2s, 1H), 7.46-7.43 (m, 1H), 7.20-7.17 (m, 1H), 6.43, 6.27 (2d, J=9 Hz, J=10 Hz, 1H), 5.84, 5.46 (2d, J=6 Hz, J=7 Hz, 1H), 4.17-4.03 (m, 1H), 3.85-3.67 (m, 1H), 1.44-1.08 (m, 6H), 1.26, 1.23 (2s, 9H), 0.85-0.78 (m, 3H); ES-LCMS m/z 344 (M+H).

### Example 33b

### Preparation of (1S)-2,2-dimethyl-1-{[3-(3-pyridinyl)-1H-pyrazol-1-yl]methyl}propyl(1S)-1-[(1R)-1-hydroxy-2-oxo-2-(1,3-thiazol-2-ylamino)ethyl]pentylcarbamate Et(1S)-2,2-dimethyl-1-{[3-(3-pyridinyl)-1H-pyrazol-1-yl]methyl}propyl(1S)-1-[(1S)-1-hydroxy-2-oxo-2-(1,3-thiazol-2-ylamino)ethyl]pentylcarbamate

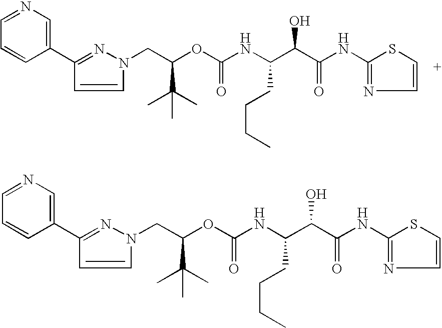

Treatment of tert-butyl(1S)-1-[(1R)-1-hydroxy-2-oxo-2-( 1,3-thiazol-2-ylamino)ethyl]pentylcarbamate Et tert-butyl(1S)-1-[(1S)-1-hydroxy-2-oxo-2-(1,3-thiazol-2-ylamino)ethyl]pentylcarbamate with (1S)-2,2-Dimethyl-1-{[3-(3-pyridinyl)-1H-pyrazol-1-yl]methyl}propyl 4-nitrophenyl carbonate from example 44c as described in example in provided the title compound as a foam in 69% yield. ¹H NMR (400 MHz, DMSO-d₆): δ 11.56, 11.47 (2s, 1H), 8.93 (s, 1H), 8.44 (d, J=4 Hz, 1H), 8.07 (d, J=8 Hz, 1H), 7.70-7.68 (m, 1H), 7.43-7.35 (m, 2H), 7.17-7.14 (m, 1H), 6.69 (s, 1H), 6.61, 6.46 (2d, J=10 Hz, J=10 Hz, 1H), 5.79, 5.46 (2d, J=6 Hz, J=7 Hz, 1H), 4.73, 4.66 (2d, J=10 Hz, J=10 Hz, 1H), 4.37 (t, 1H), 4.14-3.92 (m, 2H), 3.73-3.53 (m, 1H), 1.38-0.85 (m, 6H), 0.80, 0.78 (2s, 9H), 0.75-0.60 (m, 3H); ES- LCMS m/z 515 (M+H).

### Example 33c

### Preparation of (1S)-2,2-dimethyl-1-{[3-(3-pyridinyl)-1H-pyrazol-1-yl]methyl}propyl(1S)-1-[oxo(1,3-thiazol-2-ylamino)acetyl]pentyl carbamate

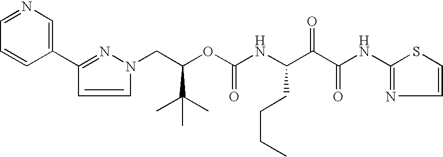

Treatment of (1S)-2,2-dimethyl-1-{[3-(3-pyridinyl)-1H-pyrazol-1-yl]methyl}propyl(1S)-1-[(1R)-1-hydroxy-2-oxo-2-(1,3-thiazol-2-ylamino)ethyl]pentyl carbamate Et(1S)-2,2-dimethyl-1-{[3-(3-pyridinyl)-1H-pyrazol-1-yl]methyl}propyl(1S)-1-[(1S)-1hydroxy-2-oxo-2-(1,3-thiazol-2-ylamino)ethyl]pentylcarbamate with Dess-Martin periodinane as described in example 1o provided the title compound as a pale yellow foam in 21% yield. ¹H NMR (400 MHz, DMSO-d₆): δ 12.75 (br s, 1H), 9.00-8.88 (m, 1H), 8.48-8.41 (m, 1H), 8.14-8.02 (m, 1H), 7.82-7.52 (m, 3H), 7.41-7.31 (m, 2H), 6.78-6.62 (m, 1H), 4.88-4.72 (m, 1H), 4.60-4.34 (m, 1H), 4.19-4.10 (m, 1H), 1.70-0.81 (m, 6H), 0.90 (s, 9H), 0.80-0.67 (m, 3H); ES-LCMS m/z 513 (M+H).

### Example 34

### Preparation of (1S)-1-[(4-benzyl-1H-imidazol-1-yl)methyl]-2,2-dimethylpropyl(1R)-1-[oxo(1H-pyrazol-5-ylamino)acetyl]pentylcarbamate

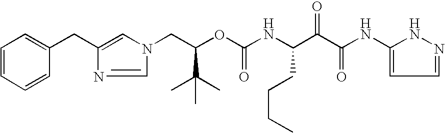

### Example 34a

### Preparation of (1-trityl-1H-imidazol-4-yl)methanol

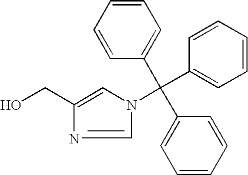

To a stirred solution of 4.99 g (37 mmol) of 4-(hydroxymethyl)imidazole hydrochloride in 38 mL of N,N-dimethylformamide was added 12.4 mL (89 mmol) of triethylamine. The resulting mixture was diluted with 140 mL of N,N-dimethylformamide, and 11.3 g (41 mmol) of triphenylmethyl chloride was added. The reaction mixture was stirred at room temperature for 18 h, and then poured into 400 g of ice. The ice was allowed to melt, and the white precipitate was collected by filtration. The filter cake was dissolved in hot dioxane and the resulting solution was cooled. The resulting mixture was diluted with diethyl ether, and the precipitate was triturated for 30 min. The solid was collected by filtration, washed with diethyl ether, and air dried to afford 10.05 g (80%) of (1-trityl-1H-imidazol-4-yl)methanol. ¹H NMR (300 MHz, DMSO-d₆ ) δ 7.37-7.42 (m, 9H), 7.27 (s, 1H), 7.08 (d, J=8 Hz, 6H), 6.70 (s, 1H), 4.86 (t, J=6 Hz, 1H), 4.31 (d, J=5 Hz, 2H); ES-LCMS m/z 363 (M+Na).

### Example 34b

### Preparation of 1-trityl-1H-imidazole-4-carbaldehyde

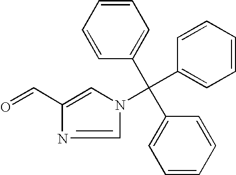

To a suspension of 10.05 g (29.5 mmol) of (1-trityl-1H-imidazol-4-yl)methanol in 200 mL of dichloromethane was added 13.8 g (32.5 mmol) of Dess-Martin periodinane. The reaction mixture was stirred at room temperature for 1 h and then filtered through a celite plug. The filter pad was washed with 50 mL of dichloromethane, and the filtrate was concentrated. The residue was taken up in dichloromethane and passed through a silica plug with 1:9 acetone:dichloromethane. The filtrate was concentrated, and the residue was further purified by silica chromatography eluting with 1:19 acetone:chloroform to yield 8.1 g (810/0) of 1-trityl-1H-imidazole-4-carbaldehyde. ¹H NMR (300 MHz, DMSO-d₆) δ 9.70 (s, 1H), 7.78 (s, 1H), 7.65 (s, 1H), 7.38-7.45 (m, 9H), 7.11 (d, J=8 Hz, 6H); ES-LCMS m/z 361 (M+Na).

### Example 34c

### Preparation of phenyl(1-trityl-1H-imidazol-4-yl)methanol

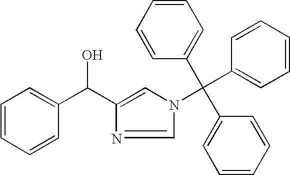

To a solution of 5.00 g (14.8 mmol) of 1-trityl-1H-imidazole-4-carbaldehyde in 100 mL of tetrahydrofuran was added a solution of 44.4 mL (44.4 mmol) of 1M phenylmagnesium bromide in 65 mL of tetrahydrofuran. The reaction mixture was stirred at room temperature for 1.5 h and cooled in an ice-bath before 1N hydrochloric acid was added dropwise. The resulting mixture was extracted with 40 mL of ethyl acetate (2×), and the combined extracts dried over anhydrous magnesium sulfate and concentrated. The residue was purified by silica chromatography eluting with 1:9 acetone:chloroform to afford 1.64 g (27%) of phenyl(1-trityl-1H-imidazol-4-yl)methanol. ¹H NMR (300 MHz, DMSO-d₆) δ 7.17-7.41 (m, 15H), 7.06 (d, J=8 Hz, 6H), 6.59 (s, 1H), 5.56 (dd, J=17 Hz, J=5 Hz, 2H); ES-LCMS m/z 439 (M+Na).

### Example 34d

### Preparation of 4-benzyl-1H-imidazole

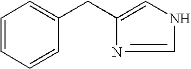

To a solution of 1.64 g (3.9 mmol) of phenyl(1-trityl-1H-imidazol-4-yl)methanol in 37 mL of dichloromethane was added 5.0 mL (31.5 mmol) of triethylsilane followed by 9.6 mL (12.5 mmol) of trifluoroacetic acid. The reaction mixture was stirred at room temperature for 48 h, and then 25 mL of water was added. The resulting mixture was extracted with 25 mL (2×) of dichloromethane. The combined extracts were dried over anhydrous magnesium sulfate and concentrated. The residue was purified by silica gel chromatography eluting with 2M ammonia in methanol:ethyl acetate (1:19) to afford 0.57 g (95%) of 4-benzyl-1H-imidazole. ¹H NMR (300 MHz, DMSO-d₆) δ 11.81 (br s, 1H), 7.49 (s, 1H), 7.13-7.26 (m, 5H), 6.71 (s, 1H), 3.80 (s, 2H). ES-LCMS m/z 159 (M+H).

### Example 34e

### Preparation of (2S)-1-(4-benzyl-1H-imidazol-1-yl)-3,3-dimethyl-2-butanol

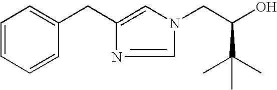

To a stirred solution of 0.57 g (3.6 mmol) of 4-benzyl-1H-imidazole in 10 mL of N,N-dimethylformamide was added 0.16 g (4.0 mmol) of 60% sodium hydride in mineral oil portionwise. Gas evolution was noted. The reaction mixture was stirred for 1 h at room temperature before 0.65 g (3.6 mmol) of (4S)-4tert-butyl-1,3,2-dioxathiolane 2,2-dioxide was added. The reaction mixture was stirred at 100° C. for 18 h, allowed to cool to room temperature, and diluted with methanol. Then 4 mL of acetyl chloridewas carefully added. The reaction mixture was stirred for 3 h, and then made basic with saturated aqueous sodium bicarbonate. It was extracted with ethyl acetate, and the extract was dried over anhydrous magnesium sulfate and concentrated. The residue was purified by silica gel chromatography eluting with 2M ammonia in methanol:chloroform (1:19) to afford 0.51 g (55%) of (2S)-1-(4-benzyl-1H-imidazol-1-yl)-3,3-dimethyl-2-butanol. ¹H NMR (300 MHz, DMSO-d₆) δ 7.51 (s, 1H), 7.16-7.29 (m, 5H), 6.84 (s, 1H), 4.92 (d, J=6 Hz, 1H), 4.05 (d, J=14 Hz, 1H), 3.78 (s, 2H), 3.62 (dd, J=14 Hz, J=10 Hz, 1H), 3.20-3.26 (m, 1H), 0.89 (s, 9H). ES-LCMS m/z 259 (M+H).

### Example 34f

### Preparation of (1S)-1-[(4-benzyl-1H-imidazol-1-yl)methyl]-2,2-dimethylpropyl 4-nitrophenyl carbonate

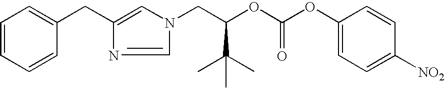

To a stirred solution of 0.20 g (0.78 mmol) of (2S)-1-(4-benzyl-1H-imidazol-1-yl)-3,3-dimethyl-2-butanol in 5 mL of 1,2-dichloroethane was added 0.27 mL (1.55 mmol) of N,N-diisopropylethylamine, and 0.31 g (1.55 mmol) of 4-nitrophenyl chloroformate. The reaction mixture was stirred for 16 h at 85° C. After cooling, the reaction mixture was diluted with ethyl acetate, and washed with 1M sodium hydroxide. The organic phase was washed with brine, dried over anhydrous magnesium sulfate, and concentrated. The residue was purified by silica gel chromatography eluting with ethyl acetate to afford 0.13 g (40%) of (1S)-1-[(4-benzyl-1H-imidazol-1-yl)methyl]-2,2-dimethylpropyl 4nitrophenyl carbonate. ES-LCMS m/z 424 (M+H).

### Example 34g

### Preparation of (1S)-1-[(4-benzyl-1H-imidazol-1-yl)methyl]-2,2-dimethylpropyl(1S)-1-[(1R)-1-hydroxy-2-oxo-2-(1H-pyrazol-5-ylamino)ethyl]pentyl carbamate Et(1S)-1-[(4-benzyl-1H-imidazol-1-yl)methyl]-2,2-dimethylpropyl(1S)-1-[(1S)-1-hydroxy-2-oxo-2-(1H-pyrazol-5-ylamino)ethyl]pentylcarbamate

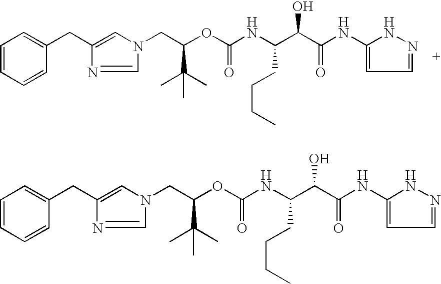

To a solution of 0.21 mmol of (3S)-3-amino-2-hydroxy-N-(1H-pyrazol-5-yl)heptanamide and 0.11 mL (0.64 mmol) of N,N-diisopropylethylamine in 1 mL of N,N-dimethylformamide was added 0.09 g (0.21 mmol) of (1S)-1-[(4-benzyl-1H-imidazol-1-yl)methyl]-2,2-dimethylpropyl 4nitrophenyl carbonate. The reaction mixture was stirred at room temperature for 16 h, and diluted with ethyl acetate. The resulting solution was washed with 1M sodium hydroxide and brine, dried over anhydrous magnesium sulfate, and concentrated. The residue was purified by silica gel chromatography, eluting with methanol:dichloromethane (1:9) to afford 0.03 g (27%, 2 steps) of (1S)-1-[(4-benzyl-1H-imidazol-1-yl)methyl]-2,2-dimethylpropyl(1S)-1-[(1R)-1-hydroxy-2-oxo-2-(1H-pyrazol-5-ylamino)ethyl]pentylcarbamate Et(1S)-1-[(4-benzyl-1H-imidazol-1-yl)methyl]-2,2-dimethylpropyl(1S)-1-[(1S)-1-hydroxy-2-oxo-2-(1H-pyrazol-5-ylamino)ethyl]pentylcarbamate. ES-LCMS m/z 511 (M+H).

### Example 34h

### Preparation of (1S)-1-[(4-benzyl-1H-imidazol-1-yl)methyl]-2,2-dimethylpropyl(1S)-1-[oxo(1H-pyrazol-5-ylamino)acetyl]pentylcarbamate

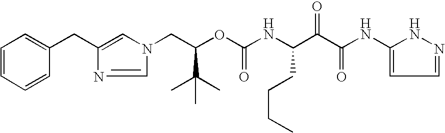

To a solution of 0.03 g (0.06 mmol) of (1S)-1-[(4-benzyl-1H-imidazol-1-yl)methyl]-2,2-dimethylpropyl(1S)-1-[(1R)-1-hydroxy-2-oxo-2-(1H-pyrazol-5-ylamino)ethyl]pentylcarbamate Et(1S)-1-[(4-benzyl-1H-imidazol-1-yl)methyl]-2,2-dimethylpropyl(1S)-1-[(1S)-1-hydroxy-2-oxo-2-(1H-pyrazol-5-ylamino)ethyl]pentyl carbamate in 1 mL of dichloromethane was added 0.03 g (0.07 mmol) of Dess-Martin periodinane. The reaction mixture was stirred for 30 min at room temperature before being filtered through a celite plug. The wash was concentrated, and the residue was purified by silica gel chromatography eluting with methanol:dichloromethane (1:9) to afford 13.2 mg (43%) of (1S)-1-[(4-benzyl-1H-imidazol-1-yl)methyl]-2,2-dimethylpropyl(1S)-1-[oxo(1H-pyrazol-5-ylamino)acetyl]pentylcarbamate. ¹H NMR (300 MHz, DMSO-d₆, 100° C.) δ 10.43 (br s, 1H), 7.62 (s, 1H), 7.48 (s, 1H), 7.14-7.29 (m, 6H), 6.81 (s, 1H), 6.52 (s, 1H), 4.82 (m, 1H), 4.68 (d, J=7 Hz, 1H), 4.24 (d, J=15 Hz, 1H), 3.68-3.98 (m, 1H), 3.79 (s, 2H), 1.76 (m, 1H), 1.56 (m, 1H), 1.22-1.40 (m, 4H), 0.82-0.96 (m, 12H); ES-LCMS m/z 509 (M+H).

### Example 35

### Preparation of (1S)-1-[(4-benzyl-1H-imidazol-1-yl)methyl]-2,2-dimethylpropyl(1R)-1-[oxo(1H-pyrazol-5-ylamino)acetyl]pentylcarbamate

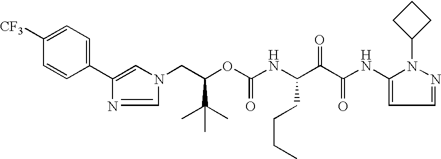

### Example 35a

### Preparation of 1-cyclobutyl-1H-pyrazol-5-ylamine

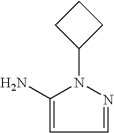

14.3 mL (456 mmol) of hydrazine was added slowly to 30 mL (456 mmol) of acrylonitrile at a rate that the reaction temperature was kept below 50° C. by cooling with an ice bath. After addition, the reaction mixture was stirred at roomtemperature for another 1.5 h, and then concentrated to give a quantitative yield of 3-hydrazinopropanenitrile.

A solution of 10.2 g (120 mmol) of 3-hydrazinopropanenitrile and 10.0 g (143 mmol) of cyclobutanone in 30 ml of ethanol was heated at reflux for 5 h. Volatiles were removed, and the residue was dissolved in 20 mL of t-butanol, and 11.5 g (120 mmol) of sodium t-butoxide was added. The resulting mixture was heated at reflux, overnight, and then concentrated. Water was added and the mixture was extracted with ethyl acetate (4×). The combined extracts were washed with brine and dried over anhydrous magnesium sulfate. After concentration, purification by column chromatography with dichloromethane:methanol (40:1) as eluant gave 2.8 g (17%) of 1-cyclobutyl-1H-pyrazol-5-ylamine. ¹H NMR (400 MHz, DMSO-d₆): δ 7.06 (s, 1H), 5.21 (s, 1H), 5.07 (br s, 2H), 4.64 (m, 1H), 2.43 (m, 2H), 2.24 (m, 2H), 1.70 (m, 2H).

### Example 35b

### Preparation of tert-butyl(1S)-1-[cyano(triphenylphosphoranylidene)acetyl]pentylcarbamate

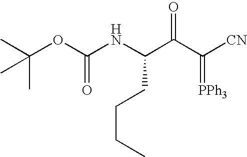

To a solution of 15.36 g (66.4 mmol) of (2S)-2-[(tert-butoxycarbonyl)amino]hexanoic acid in 150 mL of dichloromethane was added 20.0 g (66.4 mmol) of (triphenylphosphoranylidene)acetonitrile, 13.38 g (69.72 mmol) of 1-ethyl-3-(3-dimethyl-aminopropyl)carbodiimide, and 0.81 g (6.6 mmol) of 4-dimethylaminopyridine. The solution was stirred for 16 h and then concentrated under vacuum. The residue was purified by column chromatography eluting with hexane:ethyl acetate (1:1) to afford 34.0 g (99%) of tert-butyl(1s)-1-[cyano(triphenylphosphoranylidene)acetyl]pentylcarbamate as a yellow oil. ¹H NMR (300 MHz, CDCl₃-d₆) δ 5.19 (d, J=8 Hz, 1H), 4.86 (m, 1H), 2.08-2.02 (m, 1H), 1.73-1.66 (m, 1H), 1.47-1.27 (m, 13H), 0.95 (t, J=7.2 Hz, 3H); ES-LCMS m/z 515 (M+H).

### Example 35c

### Preparation of tert-butyl(1S)-1-[[(1-cyclobutyl-1H-pyrazol-5-yl)amino](oxo)acetyl]pentylcarbamate

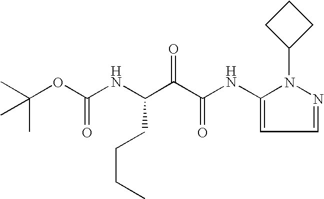

Ozone was bubbled through a solution of 2.25 g (4.38 mmol) of tert-butyl(1S)-1-[cyano(triphenylphosphoranylidene)acetyl]pentylcarbamate in 50 mL of dichloromethane at −78° C. for 30 min. Excess ozone was purged with a stream of nitrogen, and 0.54 g (3.94 mmol) of 1-cyclobutyl-1H-pyrazol-5-ylamine in 10 mL of tetrahydrofuran was added. The reaction mixture was stirred at room temperature for 1 h, and then concentrated. The residue was dissolved in 20 mL of tetrahydrofuran and 4.4 mL (4.4 mmol) of 1M silver nitrate was added. The reaction mixture was stirred at room temperature overnight. Water was added and the mixture was extracted with ethyl acetate (4×). The combined extracts were washed with brine (3×) and dried over anhydrous magnesium sulfate. After removal of solvent, purification by silica gel column chromatography with hexane:ethyl acetate (2:1) gave 1 g (67%) of tert-butyl(1S)-1-[[(1-cyclobutyl-1H-pyrazol-5-yl)amino](oxo)acetyl]pentyl carbamate. ¹H NMR (300 MHz, CDCl₃): 7.99 (s, 1H), 7.26 (s, 1H), 5.84 (br s, 1H), 5.09 (br s, 1H), 4.61-4.35 (m, 1H), 2.68 (m, 2H), 2.40 (m, 2H), 1.91 (m, 2H), 1.45 (s, 3H), 1.44 (s, 3H), 1.42 (s, 3H), 1.38 (m. 6H), 0.89 (m, 3H); ES-LCMS: 379 (M+H).

### Example 35d

### Preparation of tert-butyl(1S)-1-{(1R)-2-[(1-cyclobutyl-1H-pyrazol-5-yl)amino]-1-hydroxy-2-oxoethyl}pentylcarbamate Et tert-butyl(1S)-1-{(1R)-2-[(1-cyclobutyl-1H-pyrazol-5-yl)amino]-1-hydroxy-2-oxoethyl}pentylcarbamate

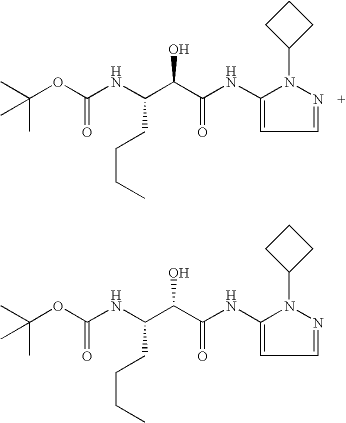

First, 101 mg (2.67 mmol) of sodium borohydride was added to a solution of 780 mg (2.06 mmol) of tert-butyl(1S)-1-[[(1-cyclobutyl-1H-pyrazol-5-yl)amino](oxo)acetyl]pentylcarbamate in 20 mL of tetrahydrofuran at 0° C. The reaction mixture was stirred for 30 min, and then saturated aqueous ammonium chloride was added. The resulting mixture was extracted with ethyl acetate. The extract was washed with brine, and dried over anhydrous magnesium sulfate, and concentrated. The residue was purified by silica gel column chromatography with hexane:ethyl acetate [(2:1) then (1:1)] gave 200 mg (26%) of tert-butyl(1S)-1-{(1R)-2-[(1-cyclobutyl-1H-pyrazol-5-yl)amino]-1-hydroxy-2-oxoethyl}pentylcarbamate Et tert-butyl(1S)-1-{(1R)-2-[(1-cyclobutyl-1H-pyrazol-5-yl)amino]-1-hydroxy-2-oxoethyl}pentylcarbamate. ¹H NMR (400 MHz CDCl₃): δ 8.14 (s, 1H), 5.63 (br s, 1H), 4.86 (d, J=9 Hz, 1H), 4.77 (s, 1H), 4.45 (m, 1H), 3.96 (t, J=9 Hz, 1H (m, 2H), 1.90 (m, 2H), 1.45 (s, 9H), 1.2-1.4 (m, 6H), 0.80 (t, J=7 Hz, 3H); ES-LCMS: 381 (M+H).

### Example 35e

### Preparation of (1S)-2,2-dimethyl-1-({4-[4-(trifluoromethyl)phenyl]-1H-imidazol-1-yl}methyl)propyl(1S)-1-{(1R)-2-[(1-cyclobutyl-1H-pyrazol-5-yl)amino]-1-hydroxy-2-oxoethyl}pentylcarbamate Et(1S)-2,2-dimethyl-1-({4-[4-(trifluoromethyl)phenyl]-1H-imidazol-1-yl}methyl)propyl(1S)-1-{(1S)-2-[(1-cyclobutyl-1H-pyrazol-5-yl)amino]-1-hydroxy-2-oxoethyl}pentylcarbamate

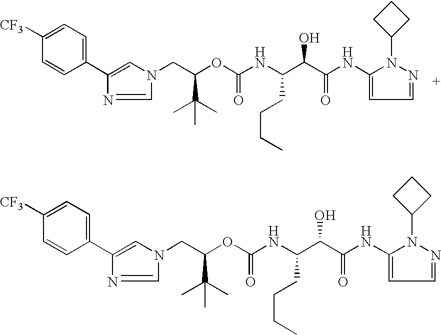

First, 5.5 mL of 4 N hydrochloric acid in 1,4-dioxane was added to a solution of 170 mg (0.447 mmol) of tert-butyl(1S)-1-{(1R)-2-[(1-cyclobutyl-1H-pyrazol-5-yl)amino]-1-hydroxy-2-oxoethyl}pentylcarbamate Et tert-butyl(1S)-1-{(1R)-2-[(1-cyclobutyl-1H-pyrazol-5-yl)amino]-1-hydroxy-2-oxoethyl}pentylcarbamate in 5 mL of 1,4-dioxane. The reaction mixture was stirred at room temperature for 30 min. Volatiles were removed, the residue was dissolved in 5 mL of N,N-dimethylformamide, and 192 mg (0.403 mmol) of (1S)-2,2-dimethyl-1-({4-[4-(trifluoromethyl)phenyl]-1H-imidazol-1-yl}methyl)propyl 4-nitrophenyl carbonate and 390 μL (2.24 mmol) of N,N-diethylisopropylamine were added. The reaction mixture was stirred at room temperature overnight. After removal of solvent, the residue was purified by silica gel column chromatography. Elution with hexane:ethyl acetate (1:1) followed by ethyl acetate afforded 150 mg (60%) of (1S)-2,2-dimethyl-1-({4-[4-(trifluoromethyl)phenyl]-1H-imidazol-1-yl}methyl)propyl(1S)-1-{(1R)-2-[(1-cyclobutyl-1H-pyrazol-5-yl)amino]-1-hydroxy-2-oxoethyl}pentylcarbamate Et(1S)-2,2-dimethyl-1-({4-[4-(trifluoromethyl)phenyl]-1H-imidazol-1-yl }methyl)propyl(1S)-1-{(1S)-2-[(1-cyclobutyl-1H-pyrazol-5-yl)amino]-1-hydroxy-2-oxoethyl}pentylcarbamate. ¹H NMR (400 MHz CDCl₃) δ 7.93 (s, 1H), 7.84 (d, J=8 Hz, 2H), 7.58 (d, J=8 Hz, 2H), 7.51 (s, 1H), 5.52 (s, 2H), 5.01 (m, 2H), 4.68 (m, 1H), 4.41 (m, 1H), 4.22 (dd, J=4 Hz, J=3 Hz, 1H), 4.00 (dd, J=14 Hz, J=10 Hz, 1H), 3.88 (d, J=12 Hz, 2H), 2.66 (m, 2H), 2.04 (m, 2H), 1.90 (m, 2H), 1.04 (s, 9H), 0.88-1.06 (m, 6H), 0.60 (t, J=7 Hz, 3H).

### Example 35f

### Preparation of (1S)-2,2-dimethyl-1-({4-[4-(trifluoromethyl)phenyl]-1H-imidazol-1-yl}methyl)propyl(1S)-1-[[(1-cyclobutyl-1H-pyrazol-5-yl)amino](oxo)acetyl]pentylcarbamate

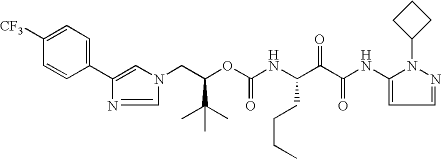

To 2 mL of dichloromethane were added 142 μL (0.3 mmol) of 2 M oxalyl chloride in dichloromethane followed by 50 μL (0.705 mmol) of dimethylsulfoxide at −60° C. After 2 min, 70 mg (0.113 mmol) of (1)-2,2-dimethyl-1-({4-[4-(trifluoromethyl)phenyl]-1H-imidazol-1-yl}methyl)propyl(1S)-1-{(1R)-2-[(1-cyclobutyl-1H-pyrazol-5-yl)amino]-1-hydroxy-2-oxoethyl}pentylcarbamate Et(1S)-2,2-dimethyl-1-({4-[4-(trifluoromethyl)phenyl]-1H-imidazol-1-yl}methyl)propyl(1S)-1-{(1S)-2-[(1-cyclobutyl-1H-pyrazol-5-yl)amino]-1-hydroxy-2-oxoethyl}pentylcarbamate in 2.5 mL of dichloromethane were added. After 5 min, 158 μL (1.13 mmol) of triethylamine was added. The reaction mixture was stirred at room temperature for 1 h. After removal of solvent, the residue was purified by silica gel column chromatography. Elution with ethyl acetate followed by ethyl acetate:acetone (2:1) afforded 16 mg (23%) of (1S)-2,2-dimethyl-1-({4-[4-(trifluoromethyl)phenyl]-1H-imidazol-1-yl}methyl)propyl(1S)-1-[[(1-cyclobutyl-1H-pyrazol-5-yl)amino](oxo)acetyl]pentylcarbamate. ¹H NMR (400 MHz, CDCl₃): δ 7.97 and 7.95 (s, 1H), 7.84 and 7.78 (d, J=8 Hz, 2H), 7.58 (d, J=8 Hz, 2H), 7.33 and 7.29 (s, 1H), 7.27 (s, 1H), 5.81 and 5.76 (s, 1H), 5.41 and 5.35 (d, J=8 Hz, 1H), 5.04 (m, 1H), 4.93 and 4.87 (d, J=7 Hz, 1H), 4.41 (m, 1H), 4.20 (d, J=14 Hz, 1H), 4.00 (m, 1H), 2.66 (m, 2H), 2.39 (m, 2H), 1.02 (s, 9H), 0.65 (t, J=7 Hz, 3H). ¹H NMR (300 Mz, DMSO-d₆, Temp=100° C.): δ 7.90 (d, 2H), 7.62 (m, 4H), 6.83 (s, 1H); HRMS C₃₁H₃₉F₃N₆O₄ m/z 617.3063 (M+H)_{Cal}; 617.3070 (M+H)_{Obs}.

### Example 36

### Preparation of (1S)-2,2-dimethyl-1-({3-[4-(trifluoromethyl)phenyl]-1H-pyrazol-1-yl}methyl)propyl(1S)-1-[[(5-isoxazolylmethyl)amino](oxo)acetyl]pentylcarbamate

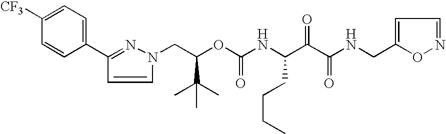

### Example 36a

### Preparation of 5-(bromomethyl)isoxazole

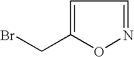

To a solution of 3.92 g (48 mmol) of 5-methylisoxazole in 120 mL of carbon tetrachloride were added 8.56 g (48 mmol) of N-bromosuccinimide and 1.16 g (4.8 mmol) of benzoyl peroxide. The resulting mixture was heated at reflux for a period of 6 h, cooled, and filtered. The filtrate was concentrated under vacuum and distilled (3 mm/70° C.) to afford 4.47 g (58%) of 5-(bromomethyl)isoxazole. ¹H NMR (300 MHz, CDCl₃) δ 8.26 (s, 1H), 6.36 (s, 1H), 4.53 (s, 2H).

### Example 36b

### Preparation of 5-(azidomethyl)isoxazole

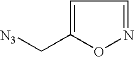

To a solution of 4.47 g (27.7 mmol) of 5-(bromomethyl)isoxazole in 50 mL methanol/water (9:1) was added 2.34 g (36.01 mmol) of sodium azide. The reaction mixture was stirred at room temperature for 16 h. Then, 100 mL of ethyl acetate and 50 mL of saturated aqueous sodium bicarbonate were added, and the layers were separated. The organic phase was dried over anhydrous magnesium sulfate, and concentrated. The residue was purified by silica gel column chromatography eluting with ethyl acetate:hexanes (3:7) to afford 1.5 g (44%) of 5-(azidomethyl)isoxazole. ¹H NMR (300 MHz, CDCl₃) δ 8.21 (s, 1H), 6.26 (s, 1H), 4.45 (s, 2H).

### Example 36c

### Preparation of 5-isoxazolylmethanamine

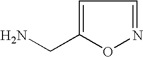

To a solution of 4.66 g (38.0 mmol) of 5-(azidomethyl)isoxazole in 50 ml of tetrahydrofuran were added 9.96 g (38.0 mmol) of triphenylphosphine and 5 mL of water. The reaction mixture was stirred at room temperature for 16 h, and concentrated under vacuum. The residue was purified by silica gel column chromatography eluting with dichloromethane:methanol (8:2) to afford 2.6 g (72%) of 5-isoxazolylmethanamine. ¹H NMR (300 MHz, CDCl₃) δ 8.15 (s, 1H), 6.10 (s, 1H), 3.97 (s, 2H), 1.60 (s, 2H); MS m/z 99 (M+H).

### Example 36d

### Preparation of tert-butyl(1S)-1-{(1R)-1-hydroxy-2-[(5-isoxazolylmethyl)amino]-2-oxoethyl}pentylcarbamate Et tert-butyl(1S)-1-{(1S)-1-hydroxy-2-[(5-isoxazolylmethyl)amino]-2-oxoethyl}pentylcarbamate

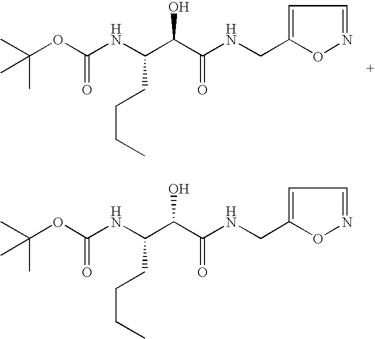

To a stirred solution of 0.450 g (1.7 mmol) of (2R,3S)-3-[(tert-butoxycarbonyl)amino]-2-hydroxyheptanoic acid Et(2S,3S)-3-[(tert-butoxycarbonyl)amino]-2hydroxyheptanoic acid and 0.416 ml (3.74 mmol) of 1-methylmorpholine in 5 mL of dichloromethane at 0° C. was added 3.57 mL (3.57 mmol) of a 1.0 M solution of isopropyl chloroformate in toluene over 20 min. The reaction mixture was stirred at 0° C. for 1 h. A solution of 0.249 g (2.55 mmol) of 5-aminomethyl isoxazole in 3.0 mL of dichloromethane was then added, and the reaction mixture was allowed to warm to room temperature. After stirring for 16 h, the reaction mixture was partitioned between 20 mL of dichloromethane and 10 mL of saturated aqueous sodium bicarbonate. The organic phase was dried over anhydrous magnesium sulfate and concentrated under vacuum. The residue was dissolved in 5 mL of methanol, 5 mL of 10% aqueous potassium carbonate was added and the reaction mixture was stirred at room temperature for 2 h. The methanol was removed in vacuo, and 20 mL of ethyl acetate was added. The organic phase was washed with water, dried over anhydrous magnesium sulfate, and concentrated in vacuo. The residue was purified by silica gel column chromatography eluting with hexane:ethyl acetate (3:7) to afford 0.381 g (65%) of tert-butyl(1S)-1-{(1R)-1-hydroxy-2-[(5-isoxazolylmethyl)amino]-2-oxoethyl}pentylcarbamate Et tert-butyl(1S)-1-{(1S)-1-hydroxy-2-[(5-isoxazolylmethyl)amino]-2-oxoethyl}pentylcarbamate as a colorless oil. ¹H NMR (300 MHz, CDCl₃) δ 8.16 (s, 1H), 7.26 (br s, 1H), 6.18 (s, 1H), 5.16, 4.88 (d, J=6 Hz, 1H), 4.71-4.50 (m, 3H), 4.25, 4.16 (2s, 1H), 3.76-3.60 (m, 1H), 1.71-1.44 (m, 6H), 1.41, 1.39 (2s, 9H), 0.88 (m, 3H); ES-LCMS m/z 342 (M+H).

### Example 36e

### Preparation of (1S)-2,2-dimethyl-1-({3-[4-(trifluoromethyl)phenyl]-1H-pyrazol-1-yl}methyl)propyl(1S)-1-{(1R)-1-hydroxy-2-[(5-isoxazolylmethyl)amino]-2-oxoethyl}pentylcarbamate Et(1S)-2,2-dimethyl-1-({3-[4-(trifluoromethyl)phenyl]-1H-pyrazol-1-yl}methyl)propyl(1S)-1-{(1S)-1-hydroxy-2-[(5-isoxazolylmethyl)amino]-2-oxoethyl}pentylcarbamate

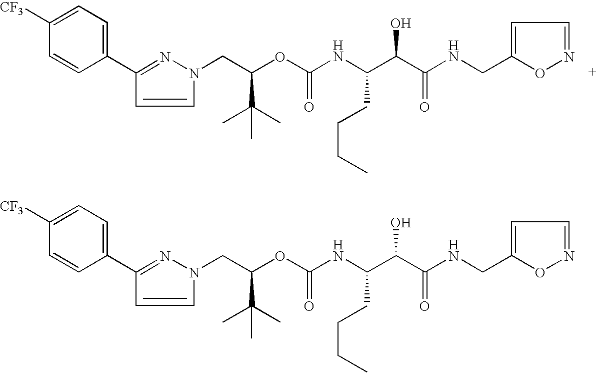

First, 2.0 mL of 4 N hydrochloric acid in dioxane were added to a solution of 0.1 g (0.293 mmol) of tert-butyl(1S)-1-{(1R)-1-hydroxy-2-[(5-isoxazolylmethyl)amino]-2-oxoethyl}pentylcarbamate Et tert-butyl(1S)-1-{(1S)-1-hydroxy-2-[(5-isoxazolylmethyl)amino]-2-oxoethyl}pentylcarbamate in 2.0 mL of dioxane. The reaction mixture was stirred for 30 min, and then concentrated. The residue was dissolved in 3.0 mL of N,N-dimethylformamide and 0.26 mL (1.46 mmol) of N,N-diisopropylethylamine was added. The solution was cooled to 0° C. and 0.139 g (0.293 mmol) of 4-nitrophenyl(1S)-2,2-dimethyl-1-{3-[4-(trifluoromethyl)phenyl]-1H-pyrazol-1-yl}propyl carbonate was added. The reaction mixture was stirred for 16 h. It was then partitioned between 50 mL of diethyl ether and 10 mL of saturated aqueous sodium bicarbonate. The aqueous layer was then extracted with 10 mL of diethyl ether (3×). The combined organic extracts were dried over anhydrous magnesium sulfate and concentrated to afford a yellow oil, which was further purified by silica gel column chromatography eluting with ethyl acetate to afford 0.1 g (59%) of (1S)-2,2-dimethyl-1-({3-[4-(trifluoromethyl)phenyl]-1H-pyrazol-1-yl}methyl)propyl(1)-1-{(1R)-1-hydroxy-2-[(5-isoxazolylmethyl)amino]-2-oxoethyl}pentyl carbamate Et(1S)-2,2-dimethyl-1-({3-[4-(trifluoromethyl)phenyl]-1H-pyrazol-1-yl}methyl)propyl(1S)-1-{(1S)-1-hydroxy-2-[(5-isoxazolylmethyl)amino]-2-oxoethyl}pentylcarbamate as a yellow oil. ¹H NMR (300 MHz, CDCl₃) δ 8.14, 8.13 (2s, 1H), 7.84 (d, J=5 Hz, 2H), 7.57 (d, J=6 Hz, 2H), 7.39, 7.38 (2s, 1H), 7.14 (t, J=4 Hz, 1H), 6.54, 6.51 (2s, 1H), 6.11, 6.09 (2s, 1H), 5.31, 5.03 (2d, J=7 Hz, J=6 Hz, 1H), 4.99-4.95 (m, 1H), 4.52-4.33 (m, 3H), 4.18-4.04 (m, 2H), 3.67-3.65 (m, 1H), 1.47-1.01 (m, 6H), 0.98 (s, 9H), 0.78-0.72 (m, 3H).

### Example 36f

### Preparation of (1S)-2,2-dimethyl-1-({3-[4-(trifluoromethyl)phenyl]-1H-pyrazol-1-yl}methyl)propyl(1S)-1-[[(5-isoxazolylmethyl)amino](oxo)acetyl]pentylcarbamate

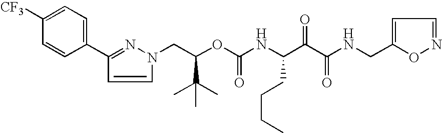

To a solution of 0.1 g (0.173 mmol) of (1S)-2,2-dimethyl-1-({3-[4-(trifluoromethyl)phenyl]-1H-pyrazol-1-yl}methyl)propyl(1S)-1-{(1R)-1-hydroxy-2-[(5-isoxazolylmethyl)amino]-2-oxoethyl}pentylcarbamate Et(1S)-2,2-dimethyl-1-({3-[4-(trifluoromethyl)phenyl]-1H-pyrazol-1-yl}methyl)propyl(1S)-1-{(1S)-1-hydroxy-2-[(5-isoxazolylmethyl)amino]-2-oxoethyl}pentylcarbamate in dichloromethane was added 0.088 g (0.207 mmol) of Dess-Martin periodinane. The reaction mixture was stirred at room temperature for 15 min, and then subjected directly to silica gel column chromatography eluting with hexane:ethyl acetate (1:1) to afford 0.026 g (26%) of (1S)-2,2-dimethyl-1-({3-[4-(trifluoromethyl)phenyl]-1H-pyrazol-1-yl}methyl)propyl(1S)-1-[[(5-isoxazolylmethyl)amino](oxo)acetyl]pentylcarbamate as a white solid. ¹H NMR (300 MHz, CDCl₃) δ 8.22 (s, 1H), 7.90 (d, J=8 Hz, 2H), 7.63 (d, J=8 Hz, 2H), 7.49 (s, 1H), 7.35 (br s, 1H), 6.59 (s, 1H), 6.23 (s, 1H), 5.22 (d, J=8 Hz, 1H), 5.05-4.96 (m, 2H), 4.63 (d, J=6 Hz, 2H), 4.44 (d, J=9 Hz, 1H), 4.29-4.21 (m, 1H), 1.49-1.10 (m, 6H), 1.05 (s, 9H), 0.84 (t, J=7 Hz, 3H); ES-LCMS m/z 578 (M+H).

### Example 37

### Preparation of (1S)-1-[(5,6-dichloro-1H-benzimidazol-1-yl)methyl]-2,2-dimethylpropyl(1S)-1-[oxo(1H-pyrazol-5-ylamino)acetyl]pentylcarbamate

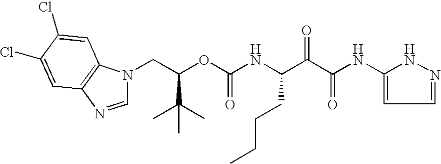

### Example 37a

### Preparation of (2S)-1-(5,6-dichloro-1H-benzimidazol-1-yl)-3,3-dimethyl-2-butanol

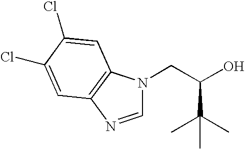

A solution of 2.08 g (11.1 mmol) of 5,6-dichloro-1H-benzimidazole and 2.00 g (11.1 mmol) of (4S)-4-tert-butyl-1,3,2-dioxathiolane 2,2-dioxide in 28 mL of N,N-dimethylformamide was stirred as 1.57 g (11.4 mmol) of potassium carbonate was added. The mixture was stirred at 60° C. for 18 h, and then cooled in an ice bath. A solution of 12 mL of acetyl chloride in 120 mL of methanol was then added. The reaction mixture was stirred for one day, concentrated, and then partitioned between ethyl acetate and saturated aqueous sodium bicarbonate. The organic phase was concentrated, and the residue was slurried in ethyl acetate. The solid was isolated by filtration to yield 1.88 g of (2S)-1-(5,6-dichloro-1H-benzimidazol-1-yl)-3,3-dimethyl-2-butanol. The filtrate was concentrated, then the residue was purified by silica gel column chromatography eluting with ethyl acetate to yield an additional 0.50 g (total yield 2.38 g, 74%) of the title compound. ¹H NMR (300 MHz, DMSO-d₆): δ 8.29 (d, J=8 Hz, 2H), 7.90 (s, 1H), 7.86 (s, 1H), 4.96 (d, J=6 Hz, 1H), 4.39 (dd, J=14 Hz, J=2 Hz, 1H), 3.98 (dd, J=14 Hz, J=10 Hz, 1H), 3.32 (m, overlapping H₂O), 0.95 (s, 9H).

### Example 37b

### Preparation of (1S)-1-[(5,6-dichloro-1H-benzimidazol-1-yl)methyl]-2,2-dimethylpropyl 4-nitrophenyl carbonate

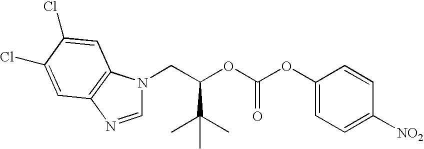

A solution of 2.38 g (8.29 mmol) of (2S)-1-(5,6-dichloro-1H-benzimidazol-1-yl)-3,3-dimethyl-2-butanol and 1.36 mL (16.6 mmol) of pyridine in 83 mL of 1,2-dichloroethane was stirred as 3.34 g (16.6 mmol) of 4-nitrophenylchloroformate was added. The solution was heated at 95° C. for one day, and then partitioned between ethyl acetate and saturated aqueous sodium bicarbonate. The organic phase was washed with brine, then concentrated. The residue was purified by silica gel column chromatography eluting with ethyl acetate to yield 1.68 g (sample contains 0.33 EtOAc by ¹H NMR for an effective weight of 1.58 g. 42%) of (1S)-1-[(5,6-dichloro-1H-benzimidazol-1-yl)methyl]-2,2-dimethylpropyl 4-nitrophenyl carbonate. ¹H NMR (300 MHz, DMSO-d₆): δ 8.44 (s, 1H), 8.20 (d, J=9 Hz, 2H), 8.01 (s,1H), 7.96 (s, 1H), 6.94 (d, J=9 Hz, 2H), 4.85 (m, 1H), 4.74 (m, 1H), 4.57 (m, 1H), 1.09 (s, 9H); ES-LCMS m/z 452 (M+H)⁺ retention time=4.33 min.

### Example 37c

### Preparation of (1S)-1-[(5,6-dichloro-1H-benzimidazol-1-yl)methyl]-2,2-dimethylpropyl(1S)-1-[(1R)-1-hydroxy-2-oxo-2-(1H-pyrazol-5-ylamino)ethyl]pentylcarbamate and (1S)-1-[(5,6-dichloro-1H-benzimidazol-1-yl)methyl]-2,2-dimethylpropyl(1S)-1-[(1S)-1-hydroxy-2-oxo-2-(1H-pyrazol-5-ylamino)ethyl]pentylcarbamate

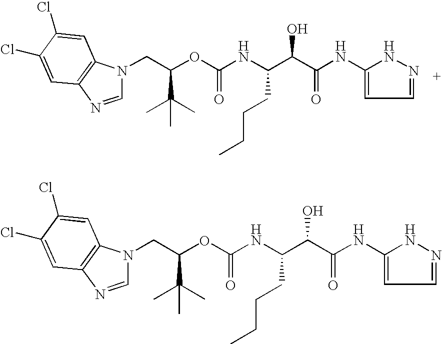

To 111 mg (0.34 mmol) of tert-butyl(1S)-1-[(1R)-1-hydroxy-2-oxo-2-(1H-pyrazol-5-ylamino)ethyl)pentylcarbamate Et tert-butyl(1S)-1-[(1S)-1-hydroxy-2-oxo-2-(1H-pyrazol-5-ylamino)ethyl]pentylcarbamate in 1 mL of dioxane at room temperature was added 4 mL (16 mmol) of a 4 M solution of hydrogen chloride in dioxane. The reaction mixture was stirred for 1 h, and then concentrated. The residue was dried under vacuum, and then dissolved in 6 mL of N,N-dimethylformamide. To this solution was added 0.14 g (0.31 mmol) of (1S)-1-[(5,6-dichloro-1H-benzimidazol-1-yl)methyl]-2,2-dimethylpropyl 4-nitrophenyl carbonate, followed by 0.24 mL (1.36 mmol) of diisopropylethylamine. The resulting yellow solution was stirred for 18 h at room temperature, and then concentrated. The residue was partitioned between ethyl acetate and saturated aqueous sodium bicarbonate. The organic phase was concentrated and the residue was purified by silica gel column chromatography eluting with 10% methanol in chloroform to yield 25 mg of diastereomer 1 and 22 mg of diastereomer 2. Combined yield 28%. diastereomer 1 ES-LCMS m/z 539 (M+H)⁺, retention time=3.95 min; diastereomer 2 ES-LCMS m/z 539 (M+H)⁺, retention time=3.86 min.

### Example 37d

### Preparation of (1S)-1-[(5,6-dichloro-1H-benzimidazol-1-yl)methyl]-2,2-dimethylpropyl(1S)-1-[oxo(1H-pyrazol-5-ylamino)acetyl]pentyl carbamate

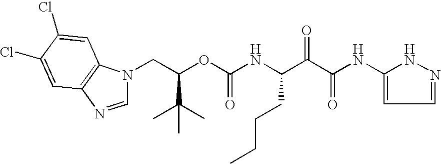

To 40 mg (0.074 mmol) of (1s)-1-[(5,6-dichloro-1H-benzimidazol-1-yl)methyl]-2,2-dimethylpropyl(1S)-1-[(1R)-1-hydroxy-2-oxo-2-(1H-pyrazol-5-ylamino)ethyl]pentylcarbamate Et(1S)-1-[(5,6-dichloro-1H-benzimidazol-1-yl)methyl]-2,2-dimethylpropyl(1S)-1-[(1S)-1-hydroxy-2-oxo-2-(1H-pyrazol-5-ylamino)ethyl]pentylcarbamate in 2 mL of chloroform at room temperature was added 39 mg (0.093 mmol) of Dess-Martin periodinane. The reaction mixture was stirred for 75 min, and then poured into saturated aqueous sodium metabisulfite solution. The mixture was subsequently neutralized with saturated aqueous sodium bicarbonate solution, and extracted with ethyl acetate. The extract was dried over anhydrous magnesium sulfate, filtered, and concentrated. The residue was purified by silica gel column chromatography eluting with a methanol:chloroform solution (1:9) to give 17.1 mg (43%). HRMS C₂₄H₃₂Cl₂N₆O₄ m/z 537.1784 (M+H)⁺_{Cal}; 537.1795 (M+H)⁺_{Obs}.

### Example 38

### Preparation of (1S)-2,2-dimethyl-1-{5-[4-(trifluoromethyl)phenyl]-1,3,4-oxadiazol-2-yl}propyl(1S)-1-[oxo(1H-pyrazol-5-ylamino)acetyl]pentylcarbamate

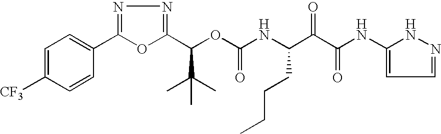

### Example 38a

### Preparation of 2-[4-(trifluoromethyl)phenyl-1,3,4-oxadiazole

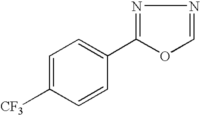

A flask containing 1.35 g (6.6 mmol) of 4-(trifluoromethyl)benzhydrazide, 1.1 mL (6.6 mmol) of triethylorthoformate and 6 mL of xylenes was fitted with a Dean-Stark trap and a condenser. The apparatus was placed in a 175° C. oil bath for 6 h and 15 min. The solution was then concentrated, and the residue was purified by silica gel column chromatography eluting with an ethyl acetate:hexane solution (1:1) to give 1.30 g (920/%) of the title compound. ¹H NMR (300 MHz, DMSO-d₆): δ 9.44 (s, 1H), 8.23 (d, J=8 Hz, 2H), 7.98 (d, J=8 Hz, 2H).

### Example 38b

### Preparation of (1S)-2,2-dimethyl-1-{5-[4-(trifluoromethyl)phenyl]-1,3,4-oxadiazol-2-yl}-1-propanol Et(1R)-2,2-dimethyl-1-{5-[4-(trifluoromethyl)phenyl]-1,3,4-oxadiazol-2-yl}-1-propanol

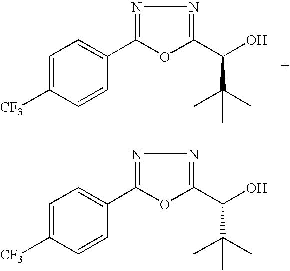

A solution of 8.1 mL (13.0 mmol) of a 1.6 M solution of butyllithium in hexanes was added to 2.2 mL (13.0 mmol) of neat 2,2,6,6-tetramethylpiperidine at 0° C. under nitrogen. After 1 h, the resulting yellow slurry was dissolved in 18 mL of anhydrous tetrahydrofuran, and the solution was added over 20 min to a solution of 1.39 g (6.50 mmol) of 2-[4-(trifluoromethyl)phenyl]-1,3,4-oxadiazole and 23.46 mL (216 mmol) of trimethylacetaldehyde in 150 mL of anhydrous tetrahydrofuran in a dry ice-acetonitrile bath. After the addition was complete, the resulting light orange solution was stirred for 2 h, and then allowed to warm slowly to room temperature. After 3 d, the yellow solution was partitioned between 10% aqueous citric acid and ethyl acetate. The extract was washed with brine, dried over anhydrous magnesium sulfate, and concentrated under vacuum. The resulting oil was further purified by silica gel column chromatography eluting with hexane:ethyl acetate (3:2) to afford 1.86 g (sample contains •0.32 EtOAc by ¹H NMR for an effective weight of 1.70g, 87%) of (1S)-2,2-dimethyl-1-{5-[4-(trifluoromethyl)phenyl]-1,3,4-oxadiazol-2-yl}-1-propanol Et(1R)-2,2-dimethyl-1-{5-[4-(trifluoromethyl)phenyl]-1,3,4-oxadiazol-2-yl}-1-propanol. ¹H NMR (300 MHz, DMSO-d₆) δ 8.19 (d, J=8 Hz, 2H), 7.97 (d, J=8 Hz, 2H), 6.11 (d, J=5 Hz, 1H), 4.58 (d, J=5 Hz, 1), •0.97 (s, 9H); ES-LCMS m/z 300.78 (M+H)⁺ retention time=4.11 minutes. The enantiomers were separated by supercritical fluid chromatography utilizing a ChiralpaK AD column (20×250 mm) eluting with carbon dioxide:methanol (93:7 @ 0.1 to 35.1 Mpa Et −10° C. to 100° C.).

### Example 38c

### Preparation of (1S)-2,2-dimethyl-1-{5-[4-(trifluoromethyl)phenyl]-1,3,4-oxadiazol-2-yl}propyl(1S)-1-[cyano(triphenylphosphoranylidene)acetyl]pentylcarbamate

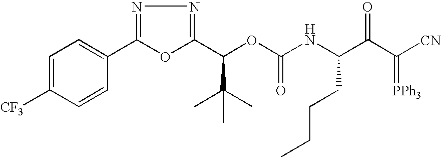

To 0.24 g (0.8 mmol) of (1S)-2,2-dimethyl-1-{5-[4-(trifluoromethyl)phenyl]-1,3,4-oxadiazol-2-yl}-1-propanol in 2.7 mL of toluene was added 246 mg (1.44 mmol) of methyl(2S)-2-isocyanatohexanoate. The reaction mixture was stirred at 85° C. for 2 days, and then concentrated. The residue was purified by silica gel column chromatography eluting with an ethyl acetate:hexanes solution (1:4) to give methyl (2S)-2-({[((1S)-2,2-dimethyl-1-{5-[4-(trifluoromethyl)phenyl]-1,3,4-oxadiazol-2-yl}propyl)oxy]carbonyl}amino)hexanoate. ES-LCMS m/z 472 (M+H)⁺ retention time=4.1 min.

This material was dissolved in 8 mL of tetrahydrofuran:water (1:1), and 47 mg (1.12 mmol) of lithium hydroxide monohydrate was added. The reaction mixture was stirred at room temperature for 1.5 h, acidified by the addition of 1 N hydrochloric acid, and extracted with diethyl ether, followed by ethyl acetate. The combined extracts were dried over anhydrous magnesium sulfate, filtered, and concentrated. The residue was dissolved in 7 mL of dichloromethane, and 5 mg (0.04 mmol) of 4-dimethylaminopyridine was added. To this solution was added 238 mg (0.79 mmol) of (triphenylphosphoranylidene)acetonitrile, and 152 mg (0.79 mmol) of 1-ethyl-3-(3-dimethyl-aminopropyl)carbodiimide. The reaction mixture was stirred at room temperature for 17 h, and then partitioned between water and ethyl acetate. The organic phase was washed with 10% citric acid, saturated aqueous sodium bicarbonate, and saturated aqueous sodium chloride. It was then dried over anhydrous magnesium sulfate, filtered, and concentrated. The residue was purified by silica gel column chromatography eluting with an ethyl acetate:hexanes solution (3:2) to give 0.16 g (27%) of (1S)-2,2-dimethyl-1-{5-[4-(trifluoromethyl)phenyl]-1,3,4-oxadiazol-2-yl}propyl(1S)-1-[cyano(triphenylphosphoranylidene)acetyl]pentyl carbamate. ES-LCMS m/z 741 (M+H)⁺ retention time=4.58 min.

### Example 38d

### Preparation of (1S)-2,2-dimethyl-1-{5-[4-(trifluoromethyl)phenyl]-1,3,4-oxadiazol-2-yl}propyl(1S)-1-[oxo(1H-pyrazol-5-ylamino)acetyl]pentylcarbamate

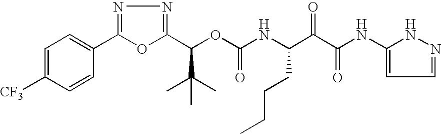

Ozone was bubbled through a solution of 0.16 g (0.22 mmol) of (1S)-2,2-dimethyl-1-{5-[4-(trifluoromethyl)phenyl]-1,3,4-oxadiazol-2-yl}propyl(1S)-1-[cyano(triphenylphosphoranylidene)acetyl]pentylcarbamate in 6 mL of dichloromethane at −78° C. for 15 min. The solution was purged with a stream of nitrogen for 5 min, 22 mg (0.26 mmol) of 3-amino pyrazole was added, and the resulting solution was stirred at −78° C. for 1.5 h. It was then concentrated, and 2.6 mL of a 1 M solution of silver nitrate in tetrahydrofuran:water (4:1) was added to the residue. The mixture was stirred for 15.75 h at room temperature, and then partitioned betwee dichloromethane and 10% citric acid. The organic phase was washed with saturated aqueous sodium bicarbonate and saturated aqueous sodium chloride, dried over anhydrous magnesium sulfate, filtered, and concentrated. The residue was purified by silica gel column chromatography eluting with methanol:chloroform solution (1:9) to give 36.0 mg (30%) of (1S)-2,2-dimethyl-1-{5-[4-(trifluoromethyl)phenyl]-1,3,4-oxadiazol-2-yl}propyl(1S)-1-[oxo(1H-pyrazol-5-ylamino)acetyl]pentylcarbamate. HRMS C₂₅H₃₀F₃N₆O₅m/z 551.2229 (M+Na)_{Cal}; 551.2235; ES-LCMS m/z 551 (M+H)⁺ retention time=4.30 min.

### Example 39

### Preparation of (1S)-2,2-dimethyl-1-{5-[4-(trifluoromethyl)phenyl]-1,3,4-oxadiazol-2-yl}propyl(1S)-1-{oxo[(2-oxo-1,3-oxazolidin-3-yl)amino]acetyl}pentylcarbamate

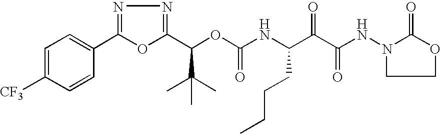

Ozone was bubbled through a solution of 0.11 g (0.15 mmol) of (1S)-2,2-dimethyl-1-{5-[4-(trifluoromethyl)phenyl]-1,3,4-oxadiazol-2-yl}propyl(1S)-1-[cyano(triphenylphosphoranylidene)acetyl]pentylcarbamate in 6 mL of dichloromethane at −78° C. for 15 min. The solution was purged with a stream of nitrogen for 5 min, a solution of 11 mg (0.18 mmol) of 3-amino-2-oxazolidinone in 2 mL of 1:1 tetrahydrofuran:dimethylsulfoxide was added and the solution was stirred at −78° C. for 1.5 h. It was concentrated, and 2.6 mL of a 1 M solution of silver nitrate in tetrahydrofuran:water (4:1) was added to the residue. The reaction mixture was stirred for 17 h at room temperature, and then partitioned between dichloromethane and 10% citric acid. The organic phase was washed with saturated aqueous sodium bicarbonate and saturated aqueous sodium chloride, dried over anhydrous magnesium sulfate, filtered, and concentrated. The residue was purified by silica gel column chromatography, eluting with methanol:chloroform solution (1:9) to give a sample that was further purified by silica gel column chromatography eluting with hexane:acetone solution (6:4) to give 20.0 mg (23%) of (1S)-2,2-dimethyl-1-{5-[4-(trifluoromethyl)phenyl]-1,3,4-oxadiazol-2-yl}propyl(1S)-1-{oxo[(2-oxo-1,3-oxazolidin-3-yl)amino]acetyl}pentylcarbamate. HRMS C₂₅H₃₀F₃N₆O₅ m/Z 551.2229(M+Na)_{Cal}; 551.2235 (M+Na)_{Obs}. Δ=0.5 mmu; ES-LCMS m/z 624 (M+MeOH+Na)⁺ retention time=4.18 min.

### Example 40

### Preparation of (1R)-2,2-dimethyl-1-{5-[4-(trifluoromethyl)phenyl]-1,3,4-oxadiazol-2-yl}propyl(1S)-1-[oxo(1H-pyrazol-5-ylamino)acetyl]pentylcarbamate

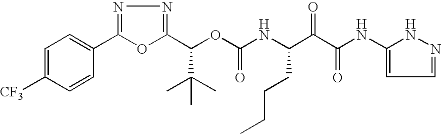

### Example 40a

### Preparation of (1R)-2,2-dimethyl-1-{5-[4-(trifluoromethyl)phenyl]-1,3,4-oxadiazol-2-yl}propyl 4-nitrophenyl carbonate

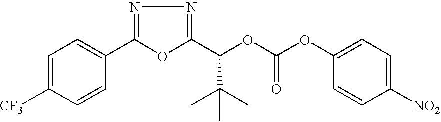

A solution of 0.30 g (1.0 mmol) of (1R)-2,2-dimethyl-1-{5-[4-(trifluoromethyl)phenyl]-1,3,4-oxadiazol-2-yl}-1-propanol in 10 mL of 1,2-dichloroethane was stirred as 241 mg (1.2mmol) of 4-nitrophenylcarbonate was added, followed by 0.1 mL (1.2 mmol) of pyridine. The resulting solution was stirred at room temperature for 1 d, and then at reflux for 3 d. The solution was then cooled, and partitioned between ethyl acetate and saturated aqueous sodium bicarbonate. The organic phase was dried over anhydrous magnesium sulfate, filtered, and concentrated. The residue was purified by silica gel column chromatography eluting with hexane:ethyl acetate solution (7:3) to give 0.39 g (sample contains *0.8 EtOAc by ¹H NMR for an effective weight of 0.34 g. 73%) ¹H NMR (300 MHz, DMSO-d₆) δ 8.34 (d, J=9 Hz, 2H), 8.26 (d, J=8 Hz, 2H), 8.04 (d, J=8 Hz, 2H), 7.63 (d, J=9 Hz, 2H), 5.83 (s, 1H), 1.13 (s, 9H).

### Example 40b

### Preparation of (1R)-2,2-dimethyl-1-{5-[4-(trifluoromethyl)phenyl]-1,3,4-oxadiazol-2-yl}propyl(1S)-1-[(1R)-1-hydroxy-2-oxo-2-(1H-pyrazol-5-ylamino)ethyl]pentylcarbamate Et(1R)-2,2-dimethyl-1-{5-[4-(trifluoromethyl)phenyl]-1,3,4-oxadiazol-2-yl}propyl(1S)-1-[(1S)-1-hydroxy-2-oxo-2-(1H-pyrazol-5-ylamino)ethyl]pentylcarbamate

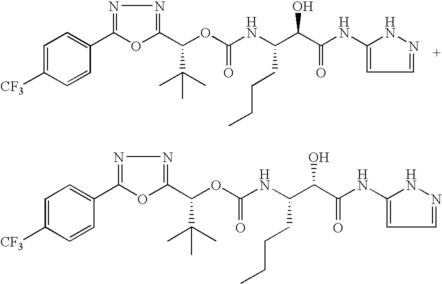

To 200 mg (0.61 mmol) of tert-butyl(1S)-1-[(1R)-1-hydroxy-2-oxo-2-(1H-pyrazol-5-ylamino)ethyl]pentylcarbamate Et tert-butyl(1S)-1-[(1S)-1-hydroxy-2-oxo-2-(1H-pyrazol-5-ylamino)ethyl]pentylcarbamate in 1.8 mL of dioxane at room temperature was added 7.2 mL (28.8 mmol) of a 4 M solution of hydrogen chloride in dioxane. The resulting mixture was stirred for 1 h, and then concentrated. The residue was dried under vacuum, and then dissolved in 12 mL of N,N-dimethylformamide. Then, 332 mg (0.62 mmol as •0.25 ethyl acetate) of (1R)-2,2-dimethyl-1-{5-[4-(trifluoromethyl)phenyl]-1,3,4-oxadiazol-2-yl}propyl 4-nitrophenyl carbonate and 0.5 mL (2.24 mmol) of diisopropylethylamine were added and the resulting yellow solution was stirred for 18 h at room temperature. The solution was concentrated, and the residue was partitioned between ethyl acetate and saturated aqueous sodium bicarbonate. The organic phase was concentrated, and the residue was purified by silica gel column chromatography eluting with methanol:chloroform solution (1:9), to afford 0.19 g (56%) of the title compound after drying under vacuum. ES-LCMS m/z 553 (M+H)⁺ retention time=4.26 min.

### Example 40c

### Preparation of (1R)-2,2-dimethyl-1-{5-[4-(trifluoromethyl)phenyl]-1,3,4-oxadiazol-2-yl}propyl(1S)-1-[oxo(1H-pyrazol-5-ylamino)acetyl]pentylcarbamate

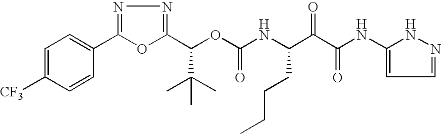

To 0.19 g (0.34 mmol) of (1R)-2,2-dimethyl-1-{5-[4-(trifluoromethyl)phenyl]-1,3,4oxadiazol-2-yl}propyl(1S)-1-[(1R)-1-hydroxy-2-oxo-2-(1H-pyrazol-5-ylamino)ethyl]pentylcarbamate Et(1R)-2,2-dimethyl-1-{5-[4-(trifluoromethyl)phenyl]-1,3,4-oxadiazol-2-yl}propyl(1S)-1-[(1S)-1-hydroxy-2-oxo-2-(1H-pyrazol-5-ylamino)ethyl]pentylcarbamate in 9 mL of chloroform at room temperature was added 180 mg (0.43 mmol) of Dess-Martin periodinane. The reaction mixture was stirred for 2.25 h, and then diluted with ethyl acetate. The solution was poured into saturated aqueous sodium metabisulfite solution, and the mixture was subsequently neutralized with saturated aqueous sodium bicarbonate solution. The organic phase was dried over anhydrous magnesium sulfate, filtered, and concentrated. The residue was purified by silica gel column chromatography eluting with a methanol:chloroform solution (1:9) to give 76.3 mg (41%). HRMS (C₂₅H₂₉F₃N₆O₅ +Na+CH₃OH) m/z 605.2311 (M+Na+CH₃OH)⁺_{Cal}; 605.2337 (M+H)⁺_{Obs}; ES-LCMS m/z 551 (M+H)⁺ retention time=4.23 min.

### Example 41

### Preparation of (1R)-1-[1,1′-biphenyl]-3-yl-2,2-dimethylpropyl(1S)-1-[oxo(1H-pyrazol-5-ylamino)acetyl]pentylcarbamate

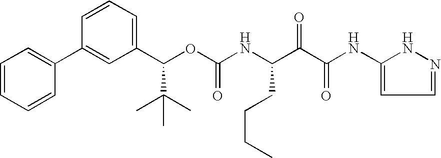

### Example 41a

### Preparation of (1S)-1-[1,1′-biphenyl]-3-yl-2,2-dimethyl-1-propanol Et(1R)-1-[1,1′-biphenyl]-3-yl-2,2-dimethyl-1-propanol

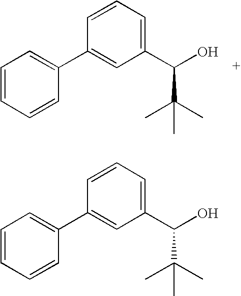

To a solution of 3.00 g (13.2 mmol) of 3-bromo-1,1′-biphenyl in 45 mL of tetrahydrofuran cooled to −78° C. was added 9.89 mL (15.84 mmol) of a 1.6 M solution of n-butyllithium in hexanes. After 30 min, 1.57 g (14.52 mmol) of 2,2-dimethyl-1-propanal was added, and the resulting mixture was warmed to room temperature and left to stir for 16 h. Then 20 mL of saturated aqueous sodium bicarbonate was added and the resulting mixture was extracted with 100 mL of ethyl acetate. The extract was dried over anhydrous magnesium sulfate, and concentrated under vacuum. The residue was purified by silica gel column chromatography eluting with hexane:ethyl acetate (9:1) to afford 3.01 g (97%) of (1S)-1-[1,1′-biphenyl]-3-yl-2,2-dimethyl-1-propanol Et(1R)-1-[1,1′-biphenyl]-3-yl-2,2-dimethyl-1-propanol as a yellow oil.¹H NMR (300 MHz, CDCl₃) δ 7.65-7.30 (m, 9H), 4.51 (s, 1H), 1.01 (s, 9H). The enantiomers were separated by supercritical fluid chromatography utilizing a Chiralpak AD column (20×250 mm) eluting with carbon dioxide:methanol (95:5 @ 0.1 to 35.1 Mpa Et 27° C.).

### Example 41b

### Preparation of 3-((1R)-1-{[(4-aminophenoxy)carbonyl]oxy}-2,2-dimethylpropyl)-1,1′-biphenyl

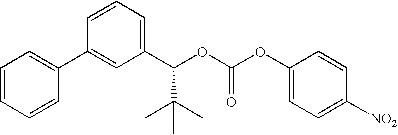

A solution of 0.97 g (4.04 mmol) of 1-[1,1′-biphenyl]-3-yl-2,2-dimethyl-1-propanol, 1.62 g (8.08 mmol) of 4nitrophenylchloroformate, and 0.66 mL (8.08 mmol) of pyridine in 40 mL of 1,2-dichloroethane was stirred at 95° C for 16 h. The solution was partitioned between ethyl acetate and saturated aqueous sodium bicarbonate solution. The organic phase was dried over anhydrous magnesium sulfate, filtered, and concentrated. The residue was purified by silica gel column chromatography eluting with a hexane:ethyl acetate solution (19:1) to give 1.20 g (73%) of the title compound. ¹H NMR (300 MHz, DMSO-d₆) δ 8.32 (d, J=9 Hz, 2H), 7.68 (m, 3H), 7.61-7.46 (m, 6H), 7.44-7.34 (m, 2H), 5.59 (s, 1H), 1.00 (s, 9H); ES-LCMS m/z 428 (M+Na)⁺ retention time=4.6 min.

### Example 41c

### Preparation of 1-[1,1′-biphenyl]-3-yl-2,2-dimethylpropyl(1S)-1-[(1S)-1-hydroxy-2-oxo-2-(1H-pyrazol-5-ylamino)ethyl]pentylcarbamate Et 1-[1,1′-biphenyl]-3-yl-2,2-dimethylpropyl(1S)-1-[(1R)-1-hydroxy-2-oxo-2-(1H-pyrazol-5-ylamino)ethyl]pentylcarbamate

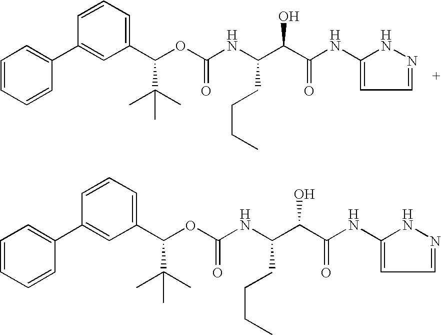

To a solution of 134 mg (0.41 mmol) of tert-butyl(1S)-1-[(1R)-1-hydroxy-2-oxo-2-(1H-pyrazol-5-ylamino)ethyl]pentylcarbamate Et tert-butyl(1S)-1-[(1S)-1-hydroxy-2-oxo-2-(1H-pyrazol-5-ylamino)ethyl]pentylcarbamate in 1.1 mL of dioxane at room temperature was added 4.4 mL (17.6 mmol) of a 4 M solution of hydrogen chloride in dioxane. The mixture was stirred for 1 h, and then concentrated. The residue was dried under vacuum, and dissolved in 3.7 mL of N,N-dimethylformamide. Then, 150 mg (0.37 mmol) of 3-((1R)-1-{[(4-aminophenoxy)carbonyl]oxy}-2,2-dimethylpropyl)-1,1′-biphenyl was added, followed by 0.3 mL (1.64 mmol) of diisopropylethylamine. The resulting yellow solution was stirred for 18 h at 60° C., and then concentrated. The residue was partitioned between ethyl acetate and saturated aqueous sodium bicarbonate. The organic phase was concentrated and the residue was purified by silica gel column chromatography eluting with methanol:chloroform solution (1:9) to afford 0.19 g (56%) of the title compound after drying under vacuum. ES-LCMS m/z 553 (M+H)⁺ retention time=4.26 min.

### Example 41d

### Preparation of (1R)-1-[1,1′-biphenyl]-3-yl-2,2-dimethylpropyl(1S)-1-[oxo(1H-pyrazol-5-ylamino)acetyl]pentylcarbamate

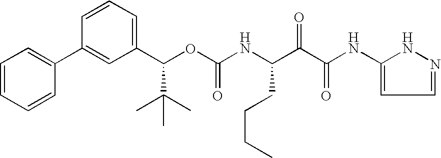

To 0.11 g (0.34 mmol) of 1-[1,1′-biphenyl]-3-yl-2,2-dimethylpropyl(1S)-1-[(1S)-1-hydroxy-2-oxo-2-(1H-pyrazol-5-ylamino)ethyl]pentylcarbamate Et 1-[1,1′-biphenyl]-3-yl-2,2-dimethylpropyl(1S)-1-[(1R)-1-hydroxy-2-oxo-2-(1H-pyrazol-5-ylamino)ethyl]pentylcarbamate in 2.2 mL of chloroform at room temperature was added 112 mg (0.43 mmol) of Dess-Martin periodinane. The reaction mixture was stirred for 1.25 h, diluted with ethyl acetate, and poured into saturated aqueous sodium metabisulfite solution. The mixture was subsequently neutralized with saturated aqueous sodium bicarbonate solution, and the layers were separated. The organic phase was dried over anhydrous magnesium sulfate, filtered, and concentrated. The residue was purified by silica gel column chromatography eluting with a methanol:chloroform solution (1:9) to give 56 mg (52%) of the title compound. HRMS (C₂₈H₃₅N₄O₄) m/z 491.2658 (M+H)⁺_{Cal}; 491.2685 (M+H)⁺_{Obs}; ES-LCMS m/z 491 (M+H)⁺ retention time=4.3 min.

### Example 42

### Preparation of (1S)-1-[1,1′-biphenyl]-3-yl-2,2-dimethylpropyl(1S)-1-[oxo(1H-pyrazol-5-ylamino)acetyl]pentylcarbamate

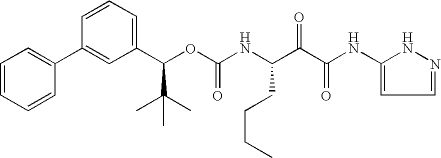

### Example 42a

### Preparation of 3-((1S)-1-{[(4-aminophenoxy)carbonyl]oxy}-2,2-dimethylpropyl)-1,1′-biphenyl

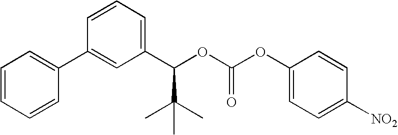

A solution of 1.02 g (4.24 mmol) of 1-[1,1′-biphenyl]-3-yl-2,2-dimethyl-1-propanol (isomer 1), 1.62 g (8.49 mmol) of 4-nitrophenylchloroformate, and 0.69 mL (8.49 mmol) of pyridine in 42 mL of 1,2-dichloroethane was stirred at 95° C. for 16 h. The solution was diluted with ethyl acetate and washed with a saturated aqueous sodium bicarbonate solution. The organic phase was dried over anhydrous magnesium sulfate, filtered, and concentrated. The residue was purified by silica gel column chromatography eluting with a hexane:ethyl acetate solution (19:1) to give 0.85 g (51%) of the title compound. ES-LCMS m/z 428 (M+Na)⁺ retention time=4.6 min; ¹H NMR (300 MHz, DMSO-d₆) δ 8.32 (d, J=9 Hz, 2H), 7.68 (m, 3H), 7.61-7.46 (m, 6H), 7.44-7.34 (m, 2H), 5.59 (s, 1H), 1.00 (s, 9H).

### Example 42b

### Preparation of 1-[1,1′-biphenyl]-3-yl-2,2-dimethylpropyl(1S)-1-[(1S)-1-hydroxy-2-oxo-2-(1H-pyrazol-5-ylamino)ethyl]pentylcarbamate and 1-[1,1′-biphenyl]-3-yl-2,2-dimethylpropyl(1S)-1-[(1R)-1-hydroxy-2-oxo-2-(1H-pyrazol-5-ylamino)ethyl]pentylcarbamate

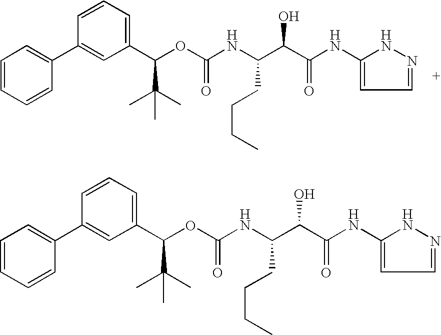

To a solution of 268 mg (0.82 mmol) of tert-butyl(1S)-1-[(1R)-1-hydroxy-2-oxo-2-(1H-pyrazol-5-ylamino)ethyl]pentylcarbamate Et tert-butyl(1S)-1-[(1S)-1-hydroxy-2-oxo-2-(1H-pyrazol-5-ylamino)ethyl]pentylcarbamate in 2.2 mL of dioxane at room temperature was added 8.8 mL (35.2 mmol) of a 4 M solution of hydrogen chloride in dioxane. The resulting mixture was stirred for 1 h, and then concentrated. The residue was dried under vacuum, and dissolved in 7.4 mL of N,N-dimethylformamide. The resulting solution was divided into two equal portions. Then, 150 mg (0.37 mmol) of 3-((1S)-1-{[(4-aminophenoxy)carbonyl]oxy}-2,2-dimethylpropyl)-1,1′-biphenyl was added to one of these portions, followed by 0.3 mL (1.64 mmol) of diisopropylethylamine. The resulting yellow solution was stirred for 18 h at 60° C., and then concentrated. The residue was partitioned between ethyl acetate and saturated aqueous sodium bicarbonate. The organic phase was concentrated, and the residue was purified by silica gel column chromatography eluting with methanol:chloroform solution (1:9) to afford 81 mg (44%) of the title compound after drying under vacuum. ES-LCMS m/z 553 (M+H)⁺ retention time=4.3 min.

### Example 42c

### Preparation of (1S)-1-[1,1′-biphenyl]-3-yl-2,2-dimethylpropyl(1S)-1-[oxo(1H-pyrazol-5-ylamino)acetyl]pentylcarbamate

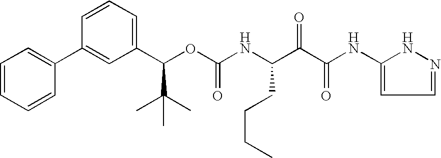

To a solution of 0.11 g (0.34 mmol) of 1-[1,1′-biphenyl]-3-yl-2,2-dimethylpropyl(1S)-1-[(1S)-1-hydroxy-2-oxo-2-(1H-pyrazol-5-ylamino)ethyl]pentylcarbamate Et 1-[1,1′-biphenyl]-3-yl-2,2-dimethylpropyl(1S)-1-[(1R)-1-hydroxy-2-oxo-2-(1H-pyrazol-5-ylamino)ethyl]pentylcarbamate in 2.2 mL of chloroform at room temperature was added 112 mg (0.43 mmol) of Dess-Martin periodinane. The reaction mixture was stirred for 1.25 h, diluted with ethyl acetate, and poured into saturated aqueous sodium metabisulfite solution. The resulting mixture was subsequently neutralized with saturated aqueous sodium bicarbonate solution, and the layers were separated. The organic phase was dried over anhydrous magnesium sulfate, filtered, and concentrated, and the residue was purified by silica gel column chromatography eluting with a methanol:chloroform solution (1:9) to give 20.6 mg (28%) of the title compound. HRMS (C₂₈H₃₅N₄O₄) m/z 491.2658 (M+H)⁺_{Cal}; 491.2653 (M+H)⁺_{Obs}; ES-LCMS m/z 491 (M+H)⁺ retention time=4.3 min.

### Example 43

### Preparation of 1-(4,7-diethoxy-1-methyl-1H-benzimidazol-2-yl)-2,2-dimethylpropyl(1S)-1-[(1H-pyrazol-5-ylamino)carbonyl]pentylcarbamate

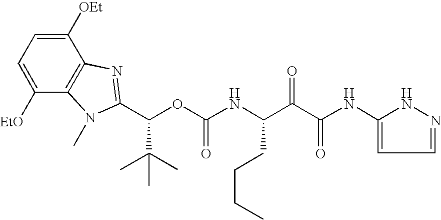

### Example 43a

### Preparation of N-1-methyl-4,7-diethoxy-benzoimidazole

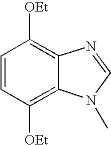

To a suspension of 2.00 g (9.71 mmol) of 4,7-diethoxy-benzimidazole in 10 mL of N,N-dimethylformamide were added 21 mL (10.7 mmol) of 0.5 M sodium methoxide in methanol and 1.01 mL (10.7 mmol) of dimethyl sulfate at room temperature. The reaction mixture was stirred at 70° C. overnight Solvent was removed and dichloromethane was added. The extract was washed with brine (3×) and dried over anhydrous magnesium sulfate. After removal of solvent, purification by silica gel column chromatography with dichloromethane:methanol (30:1) as eluant gave 1.5 g (70%) of the title compound as a yellow solid. ¹H NMR(300 MHz, DMSO-d₆): δ 7.96 (s, 1H), 6.63 (d, J=10 Hz, 1H), 6.54 (d, J=10 Hz, 1H), 4.21-4.01 (m, 4H), 3.99 (s, 3H), 1.43-1.35 (m, 6H).

### Example 43b

### Preparation of (1S)-1-(4,7-diethoxy-1-methyl-1H-benzimidazol-2-yl)-2,2-dimethyl-1-propanol Et(1R)-1-(4,7-diethoxy-1-methyl-1H-benzimidazol-2-yl)-2,2-dimethyl-1-propanol

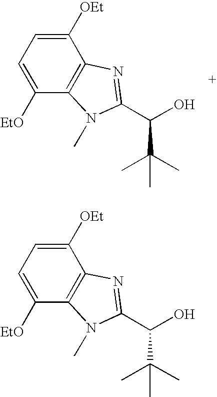

To a solution of 2.5 g (11.4 mmol) of N-1-methyl-4,7-diethoxy-benzimidazole in 60 mL of tetrahydrofuran was added 5.5 mL (13.75 mmol) of 2.5 M n-butyllithium in hexanes at −78° C. The reaction mixture was stirred at −78° C. to −35° C. for 1 h. It was cooled to −78° C. and 3.8 mL (34.2 mmol) of trimethylacetaldehyde was added. The reaction was stirred at room temperature overnight. Water was added, and the mixture was extracted with diethyl ether. The extract was washed with brine (3×) and dried over anhydrous magnesium sulfate. After removal of solvent, purification by silca gel column chromatography eluting with hexane:ethyl acetate (2:1) gave 1.6 g (48%) of (1S)-1-(4,7-diethoxy-1-methyl-1H-benzimidazol-2-yl)-2,2-dimethyl-1-propanol Et(1R)-1-(4,7-diethoxy-1-methyl-1H-benzimidazol-2-yl)-2,2-dimethyl-1-propanol as a yellow solid. ¹H NMR(300 MHz, DMSO-d₆): δ 6.63 (d, J=8 Hz, 1H), 6.54 (d, J=8 Hz, 1H), 5.46 (d, J=6 Hz, 1H), 4.60 (d, J=6 Hz, 1H), 4.24-4.06 (m, 4H), 4.09 (s, 3H), 1.45-1.37 (m, 6H), 1.05 (s, 9H); ES-LCMS: 307 (M+H). The enantiomers were separated by supercritical fluid chromatography utilizing a Chiralpak AD column (20×250 mm) eluting with carbon dioxide:methanol (95:5 @ 0.1 to 35.1 Mpa).

### Example 43c

### Preparation of 4-aminophenyl(1R)-1-(4,7-diethoxy-1-methyl-1H-benzimidazol-2-yl)-2,2-dimethylpropyl carbonate

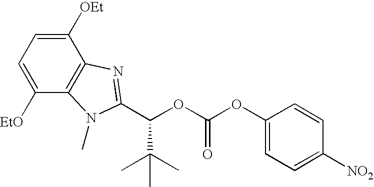

To a solution of 120 mg (0.392 mmol) of (1R)-1-(4,7-diethoxy-1-methyl-1H-benzimidazol-2-yl)-2,2-dimethyl-1-propanol in 10 mL of dichloromethane were added 79 mg (0.471 mmol) of 4-nitrophenylchloroformate and 60.0 mg (0.590 mmol) of 4-dimethylaminopyridine. The reaction mixture was stirred at room temperature for 3 h. After removal of solvent, purification by silica gel column chromatography eluting with hexane:ethyl acetate (4:1 then 2:1) gave 150 mg (81%) of 4-aminophenyl(1R)-1-(4,7-diethoxy-1-methyl-1H-benzimidazol-2-yl)-2,2-dimethylpropyl carbonate as an oil. ¹H NMR (300 MHz, DMSO-d₆): δ 8.29 (d, J=9 Hz, 2H), 7.55 (d, J=9 Hz, 2H), 6.67 (d, J=9 Hz, 1H), 6.57 (d, J=9 Hz, 1H), 5.70 (s, 1H), 4.19 (q, J=7 Hz, 2H), 4.08-4.05 (m, 2H), 4.14 (s, 3H), 1.36 (m, 6H), 1.12 (s, 9H); ES-LCMS: 472 (M+H).

### Example 43d

### Preparation of 1-(4,7-diethoxy-1-methyl-1H-benzimidazol-2-yl)-2,2-dimethylpropyl(1S)-1-[(S)-hydroxy(1H-pyrazol-5-ylamino)methyl]pentyl carbamate Et 1-(4,7-diethoxy-1-methyl-1H-benzimidazol-2-yl)-2,2-dimethyl propyl(1S)-1-[(R)-hydroxy(1H-pyrazol-5-ylamino)methyl]pentylcarbamate

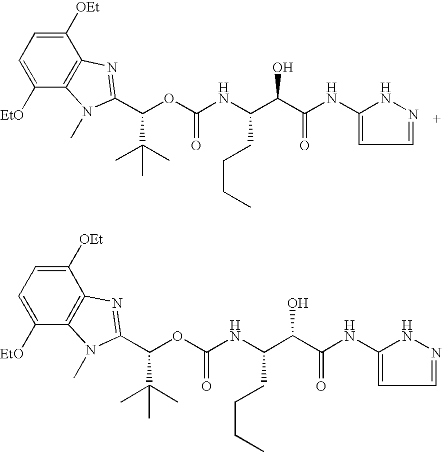

To a solution of 268 mg (0.82 mmol) of tert-butyl(1S)-1-[(1R)-1-hydroxy-2-oxo-2-(1H-pyrazol-5-ylamino)ethyl]pentylcarbamate Et tert-butyl(1S)-1-[(1S)-1-hydroxy-2-oxo-2-(1H-pyrazol-5-ylamino)ethyl]pentylcarbamate in 2.2 mL of dioxane at room temperature was added 8.8 mL (35.2 mmol) of a 4 M solution of hydrogen chloride in dioxane. The mixture was stirred for 1 h, and then concentrated. The residue was dried under vacuum, and dissolved in 7.4 mL of N,N-dimethylformamide. The resulting solution was divided into two equal portions. Then, 193 mg (0.41 mmol) of 4-aminophenyl(1R)-1-(4,7-diethoxy-1-methyl-1H-benzimidazol-2-yl)-2,2-dimethylpropyl carbonate and 0.4 mL of N,N-dimethylformamide was added to one of these portions, followed by 0.3 mL (1.64 mmol) of diisopropylethylamine. The resulting yellow solution was stirred for 1 d at 60° C. It was concentrated, and the residue was partitioned between ethyl acetate and saturated aqueous sodium bicarbonate. The organic phase was concentrated, and the residue was purified by silica gel column chromatography eluting with methanol:chloroform solution (1:9). The sample was purified further by silica gel column chromatography eluting with ethyl acetate:chloroform solution (1:4) to yield 28 mg (12%) of the title compound. ES-LCMS m/z 559 (M+H)⁺ retention time=3.9 min.

### Example 43e

### Preparation of 1-(4,7-diethoxy-1-methyl-1H-benzimidazol-2-yl)-2,2-dimethylpropyl(1S)-1-[(1H-pyrazol-5-ylamino)carbonyl]pentylcarbamate

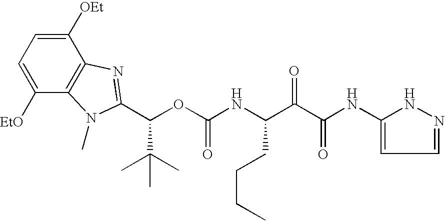

To a solution of 26 mg (0.047 mmol) of 1-(4,7-diethoxy-1-methyl-1H-benzimidazol-2-yl)-2,2-dimethylpropyl(1S)-1-[(S)-hydroxy(1H-pyrazol-5-ylamino)methyl]pentylcarbamate Et 1-(4,7-diethoxy-1-methyl-1H-benzimidazol-2-yl)-2,2-dimethylpropyl(1S)-1-[(R)-hydroxy(1H-pyrazol-5-ylamino)methyl]pentylcarbamate in 1 mL of chloroform at room temperature was added 25 mg (0.058 mmol) of Dess-Martin periodinane. The reaction mixture was stirred for 1 h, and then an additional 25 mg (0.058 mmol) of Dess-Martin periodinane was added. The reaction mixture was stirred for 1 h, diluted with ethyl acetate, and then poured into saturated aqueous sodium metabisulfite solution. The resulting mixture was subsequently neutralized with saturated aqueous sodium bicarbonate solution, and the two layers were separated. The organic phase was dried over anhydrous magnesium sulfate, filtered, and concentrated. The residue was purified by silica gel column chromatography eluting with a methanol:chloroform solution (1:9) to give 6.5 mg (25%) of the title compound. HRMS (C₂₈H₄₀N₆O₆) m/z 557.3088 (M+H)⁺_{Cal}; 557.3096 (M+H)⁺_{Obs}; ES-LCMS m/z 589 (M+MeOH+H)⁺ retention time=3.8 min.

### Example 44

### Preparation of (1S)-2,2-dimethyl-1-{[3-(3-pyridinyl)-1H-pyrazol-1-yl]methyl}propyl (1S)-1-[oxo(1H-pyrazol-5-ylamino)acetyl]pentylcarbamate

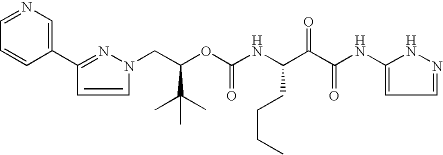

### Example 44a

### Preparation of 3-(1H-Pyrazol-3-yl)pyridine

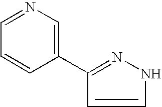

A solution of 5.0 g (41.3 mmol) of 3-acetylpyridine and 5.4 g (45.4 mmol) of dimethyl formamide dimethylacetal in 40 mL of anhydrous dimethylformamide was stirred at 130° C. for 4 h. Solvent was evaporated, and the residue was triturated with diethyl ether to obtain 5.7 g of a yellow solid. To a solution of the solid in 50 mL of methanol was added 1.59 g (32 mmol) of hydrazine monohydrate. After 48 h at room temperature, solvent was removed and portions of acetonitrile were distilled from the residue to provide 4.8 g (81%) of 3-(1H-Pyrazol-3-yl)pyridine as a tan oil. ¹H NMR (DMSO-d₆) δ 11.5 (br s, 1H), 9.05 (d, J=2 Hz, 1H), 8.56 (dd, J=5 Hz, J=2 Hz, 1H), 8.10 (dt, J=8 Hz, J=2 Hz, 1H), 7.65 (d, J=2 Hz, 1H), 7.33 (dd, J=8 Hz, J=5 Hz, 1H), 6.66 (d, J=2 Hz, 1H); ES-LCMS m/z 2146 (M+H).

### Example 44b

### Preparation of (2S)-3,3-Dimethyl-1-[3-(3-pyridinyl)-1H-pyrazol-1-yl]-2-butanol

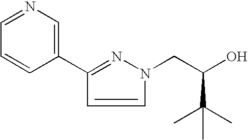

To a solution of 0.38 g (2.62 mmol) of 3-(1H-pyrazol-3-yl)pyridine and 0.47 g (2.62 mmol) of (4S)-4-tert-butyl-1,3,2-dioxathiolane 2,2-dioxide in 15 mL of acetonitrile was added 1.3 g of a potassium fluoride/alumina mixture [prepared by mixing 10 g of potassium fluoride, 200 mL of water, and 15 g of activated neutral alumina (Brockmann I, 150 mesh) and concentrating at 55 ° C.]. The mixture was stirred at ambient temperature for 4 h. Then, 2 mL of acetyl chloride was slowly added to 10 mL of methanol, and this solution was added to the reaction mixture. After 18 h, saturated aqueous sodium bicarbonate/water was added, the mixture was filtered, and the filter cake was rinsed with methanol. The filtrate was concentrated, and the residue was partitioned between saturated aqueous sodium bicarbonate and dichloromethane. The organic phase was dried over sodium sulfate, and concentrated to provide 0.6 g (94%) of (2S)-3,3-Dimethyl-1-[3-(3-pyridinyl)-1H-pyrazol-1-yl]-2-butanol as a yellow solid. ¹H NMR (DMSO-d₆) δ 8.97 (s, 1H), 8.45 (d, J=4 Hz, 1H), 8.1 (t, J=8 Hz, 1H), 7.76 (d, J=2 Hz, 1H), 7.38 (dd, J=8 Hz, J=5 Hz, 1H), 4.86 (d, J=6 Hz, 1H), 4.27 (dd, J=13 Hz, J=2 Hz, 1H), 3.90 (dd, J=14 Hz, J=10 Hz, 1H), 3.5-3.4 (m, 1H), 0.90 (s, 9H).

### Example 44c

### (1S)-2,2-Dimethyl-1-{[3-(3-pyridinyl)-1H-pyrazol-1-yl]methyl}propyl 4-nitrophenyl carbonate

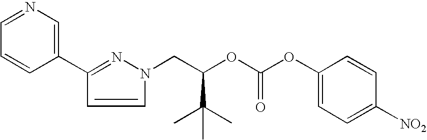

(2S)-3,3-Dimethyl-1-[3-(3-pyridinyl)-1H-pyrazol-1-yl]-2-butanol was treated with p-nitrophenyl chloroformate as described in example 1 g to provide the title compound as a solid foam. ¹H NMR (DMSO-d₆) δ 8.97 (s, 1H), 8.46 (d, J=4 Hz, 1H), 8.13 (d, J=9 Hz, 2H), 8.10 (d, J=8 Hz, 1H), 7.9 (d, J=2 Hz, 1H), 7.38 (dd, J=7 Hz, J=4 Hz, 1H), 7.22 (d, J=9 Hz, 2H), 6.83 (d, J=2 Hz, 1H), 4.90 (d, J=9 Hz, 1H), 4.62 (d, J=14 Hz, 1H), 4.35 (dd, J=14 Hz, J=9 Hz, 1H), 1.03 (s, 9H).

### Example 44d

### Preparation of (1S)-2,2-Dimethyl-1-{[3-(3-pyridinyl)-1H-pyrazol-1-yl]methyl}propyl(1S)-1-[oxo(1H-pyrazol-5-ylamino)acetyl]pentylcarbamate

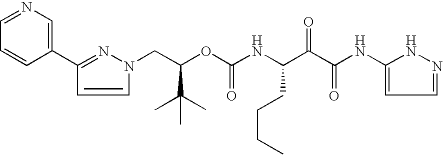

(1S)-2,2-Dimethyl-1-{[3-(3-pyridinyl)-1H-pyrazol-1-yl]methyl}propyl 4nitrophenyl carbonate was subjected sequentially to coupling with tert-butyl(1S)-1-[1-hydroxy-2-oxo-2-(1H-pyrazole-5-ylamino)ethyl]pentylcarbamate and oxidation with Dess-Martin periodinane as described in examples 1n Et 1o to provide the title compound as a solid foam. ¹H NMR (DMSO-d₆) δ 12.5 (br s, 1H), 10.8 (br s, 1H), 8.96 (s, 1H), 8.46 (d, J=4 Hz, 1H), 8.1 (d, J=8 Hz, 1H), 7.7 (d, J=2 Hz, 1H), 7.65-7.60 (m, 1H), 7.41 (d, J=8 Hz, 1H), 7.38 (dd, J=8 Hz, J=4 Hz, 1H), 6.72 (d, J=2 Hz, 1H), 4.79 (d, J=8 Hz, 1H), 4.7-4.6 (m, 1H), 4.43 (d, J=12 Hz, 1H), 4.16 (dd, J=14 Hz, J=10 Hz, 1H), 1.7-1.6 (br m, 2H), 1.4-1.1 (br m, 4H), 0.9 (s, 9H), 0.86 (t, 3H); ES-LCMS m/z 496 (M+H); Analysis calculated for C₂₅H₃₃N₇O₄•0.5 H₂O: C, 59.51; H, 6.79; N, 19.43. Found: C, 59.55; H, 6.62; N, 19.20.

### Example 45

### Preparation of (1S)-2,2-dimethyl-1-{[3-(4-pyridinyl)-1H-pyrazol-1-yl]methyl}propyl(1S)-1-[oxo(1H-pyrazol-5-ylamino)acetyl]pentylcarbamate

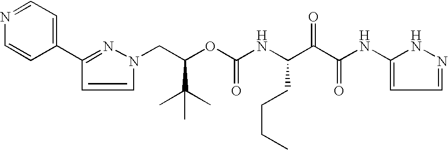

### Example 45a

### (1S)-2,2-Dimethyl-1-{[3-(4-pyridinyl)-1H-pyrazol-1-yl]methyl}propyl 4-nitrophenyl carbonate

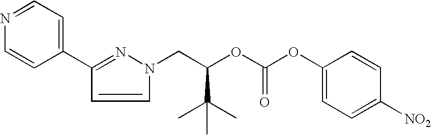

4Acetylpyridine was subjected sequentially to the procedures described in examples 44a, 44b, Et 44c to provide the title compound as a solid foam. ¹H NMR (DMSO-d₆) δ 8.53 (d, J=6 Hz, 2H), 8.14 (d, J=9 Hz, 2H), 7.93 (d, J=2 Hz, 1H), 7.71 (d, J=6 Hz, 2H), 7.23 (d, J=9 Hz, 2H), 6.9 (d, J=2 Hz, 1H), 4.89 (d, 10 Hz, 1H), 4.65 (br d, J=14 Hz, 1H), 4.37 (dd, J=14 Hz, J=10 Hz, 1H), 1.04 (s, 9H).

### Example 45b

### (1S)-2,2-Dimethyl-1-{[3-(4-pyridinyl)-1H-pyrazol-1-yl]methyl}propyl(1S)-1-[oxo(1H-pyrazol-5-ylamino)acetyl]pentylcarbamate

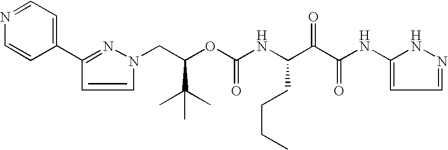

(1S)-2,2-Dimethyl-1-{[3-(4-pyridinyl)-1H-pyrazol-1-yl]methyl}propyl 4-nitrophenyl carbonate was subjected sequentially to coupling with tert-butyl(1S)-1-[1-hydroxy-2-oxo-2-(1H-pyrazole-5-ylamino)ethyl]pentylcarbamate and oxidation with Dess-Martin periodinane as described in example 44d to provide the title compound as a solid foam. ¹H NMR (DMSO-d₆) δ 12.5 (br s, 1H), 10.8 (br s, 1H), 8.6-8.5 (m, 2H), 7.8-7.6 (m, 4H), 7.44 (d, J=8 Hz, 1H), 6.85-6.75 (m, 1H), 6.45 (br s, 1H), 4.82-4.78 (m, 1H), 4.62-4.58 (m, 1H), 4.5-4.4 (m, 1H), 4.20-4.15 (m, 1H), 1.4-1.0 (m, 6H), 0.95 (s, 9H), 0.88-0.80 (m, 3H); ES-LCMS m/z 528 (M+H+MeOH); Analysis calculated for C₂₅H₃₃N₇O₄•0.1 H₂O: C, 59.81; H, 6.64; N, 19.45. Found: C, 59.79; H, 6.72; N, 19.26.

### Example 46

### Preparation of (1S)-2,2-dimethyl-1-({3-[4-(trifluoromethyl)phenyl]-1H-pyrazol-1-yl}methyl)propyl(1S)-1-[oxo(1,3-thiazol-2-ylamino)acetyl]pentylcarbamate

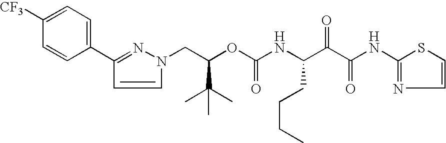

(1S)-2,2-Dimethyl-1-({3-[4-(trifluoromethyl)phenyl]-1H-pyrazol-1-yl}methyl)propyl 4-nitrophenyl carbonate was coupled with tert-butyl(1S)-1-[1-hydroxy-2-oxo-2-(1,3-thiazol-2-ylamino)ethyl]pentylcarbamate, and the resulting product was oxidized with Dess-Martin periodinane as described in example 1o to provide the title compound as a solid foam. ¹H NMR (DMSO-d₆) δ 12.60 (br s, 1H), 8.00-7.92 (m, 2H), 7.74-7.68 (m, 2H), 7.60-7.52 (m, 2H), 7.44-7.32 (m, 2H), 6.70 (s, 1H), 4.8-4.4 (m, 3H), 4.2-4.1 (m, 1H), 1.8-1.5 (m, 2H), 1.4, 0.95 (2s, 9H), 1.38-1.30 (m, 4H), 0.92, 0.75 (dt, 3H); ES-LCMS m/z 580 (M+H); Analysis calculated for C₂₇H₃₂F₃N₅O₄S•0.61 H2O: C, 54.91; H, 5.67; N, 11.86. Found: C, 54.90; H, 6.03; N, 12.15.

### Example 47

### Preparation of (1S)-1-{[4-(benzyloxy)phenoxy]methyl}-2,2-dimethylpropyl(1S)-1-[oxo(1H-pyrazol-5-ylamino)acetyl]pentylcarbamate

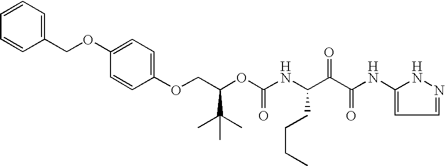

### Example 47a

### Preparation of (2S)-1-[4-(Benzyloxy)phenoxy]-3,3-dimethyl-2-butanol

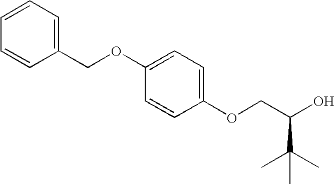

A mixture of 0.10 g (0.50 mmol) of 4-(benzyloxy)-phenol, 0.09 g (0.50 mmol) of (4S)-4-tert-Butyl-1,3,2-dioxathiolane 2,2-dioxide, and 0.20 g (1.5 mmol) of potassium carbonate in 3 mL of acetonitrile was stirred at 85 ° C. for 2.5 h. The mixture was allowed to cool to room temperature, and a solution of 1 mL of acetyl chloride and 10 mL of methanol was added. After 2 h, the volatiles were removed, and the residue was partitioned between saturated aqueous sodium bicarbonate and dichloromethane. The organic phase was dried over sodium sulfate, evaporated, and purified by silica gel chromatography eluting with hexanes:ethyl acetate (9:1) to afford 0.124 g (83%) of (2S)-1-[4-(Benzyloxy)phenoxy]-3,3-dimethyl-2-butanol as a white solid. ¹H NMR (DMSO-d₆) δ 7.40 (d, J=7 Hz, 2H), 7.35 (t, J=7 Hz, 2H), 7.29 (t, J=7 Hz, 1H), 6.90 (d, J=9 Hz, 2H), 6.85 (d, J=9 Hz, 2H), 5.00 (s, 2H), 4.8 (br s, 1H), 3.97 (dd, J=10 Hz, J=3 Hz, 1H), 3.67 (dd, J=10 Hz, J=8 Hz, 1H), 3.37 (dd, J=8 Hz, J=7 Hz, 1H), 0.88 (s, 9H); ES-LCMS m/z 301 (M+H).

### Example 47b

### Preparation of (1S)-1-{[4-(Benzyloxy)phenoxy]methyl}-2,2-dimethylpropyl 4-nitrophenyl carbonate

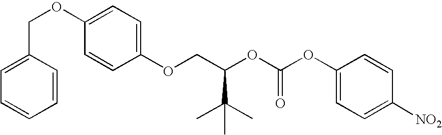

(2S)-1-[4-(Benzyloxy)phenoxy]-3,3-dimethyl-2-butanol was treated with p-nitrophenyl chloroformate as described in example 1 g to provide the title compound as a yellow oil. ¹H NMR (DMSO-d₆) δ 8.29 (d, J=9 Hz, 2H), 7.48 (d, J=9 Hz, 2H), 7.4 (d, J=7 Hz, 2H), 7.35 (t, J=7 Hz, 2H), 7.29 (t, J=7 Hz, 1H), 6.93 (d, J=9 Hz, 2H), 6.89 (d, J=9 Hz, 2H), 5.02 (s, 2H), 4.86 (dd, J=9 Hz, J=3 Hz, 1H), 4.29 (dd, J=11 Hz, J=3 Hz, 1H), 4.02 (dd, J=11 Hz, J=9Hz, 1H), 1.02(s, 9H).

### Example 47c

### Preparation of (1S)-1-{[4-(Benzyloxy)phenoxy]methyl}-2,2-dimethylpropyl(1S)-1-[oxo(1H-pyrazol-5-ylamino)acetyl]pentylcarbamate

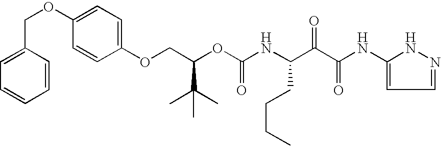

(1S)-1-{[4-(Benzyloxy)phenoxy]methyl}-2,2-dimethylpropyl 4-nitrophenyl carbonate was coupled with tert-butyl(1S)-1-[1-hydroxy-2-oxo-2-(1H-pyrazole-5-ylamino)ethyl]pentylcarbamate and oxidized with Dess-Martin periodinane as described in examples 1n Et 1o to afford the title compound as a solid foam. ¹H NMR (DMSO-d₆) δ 12.5 (br s, 1H), 10.8 (br s, 1H), 7.7-7.5 (m, 2H), 7.41 (d, J=7 Hz, 2H), 7.36 (t, J=7 Hz, 2H), 7.29 (t, J=7 Hz, 1H), 6.89 (d, J=9 Hz, 2H), 6.82 (d, J=9 Hz, 2H), 6.6-6.4 (m, 1H), 5.02 (s, 2H), 4.85-4.80 (m, 1H), 4.70-4.75 (m, 1H), 4.20-4.15 (m, 1H), 3.9-3.8 (m, 1H), 1.80-1.75 (m, 1H), 1.5-1.1 (m, 5H), 0.94 (s, 9H), 0.86 (t, 3H); ES-LCMS m/z 551 (M+H); Analysis calculated for C₃₀H₃₈N₄O₆•0.5 H₂O: C, 64.38; H, 7.02; N, 10.01. Found: C, 64.35; H, 6.94; N, 9.66.

### Example 48

### Preparation of (1S)-1-{[4-(aminocarbonyl)phenoxy]methyl}-2,2-dimethylpropyl(1S)-1-[oxo(1H-pyrazol-5-ylamino)acetyl]pentylcarbamate

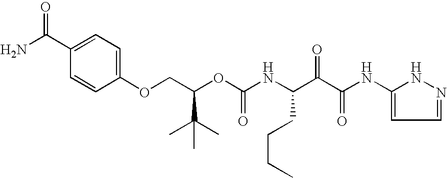

### Example 48a: (1S)-1-{[4-(aminocarbonyl)phenoxy]methyl}-2,2-dimethylpropyl 4-nitrophenyl carbonate

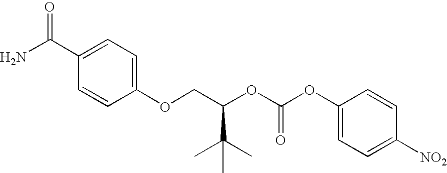

4-Hydroxybenzamide was subjected to the procedure described in example 47a and then treated with p-nitrophenyl chloroformate as described in example 1g to provide the title compound as a solid foam. ¹H NMR (DMSO-d₆) δ 8.3 (d, J=9 Hz, 2H), 7.84 (d, J=9 Hz, 3H), 7.5 (d, J=9 Hz, 2H), 7.19 (br s, 1H), 7.02 (d, J=9 Hz, 2H), 4.93 (dd, J=9 Hz, J=2Hz, 1H), 4.43 (dd, J=11 Hz, J=2Hz, 1H), 4.18 (dd, J=11 Hz, J=9 Hz, 1H), 1.03 (s, 9H); ES-LCMS m/z 403 (M+H).

### Example 48b

### (1S)-1-{[4-(Aminocarbonyl)phenoxy]methyl}-2,2-dimethylpropyl(1S)-1-[oxo(1H-pyrazol-5-ylamino)acetyl]pentylcarbamate

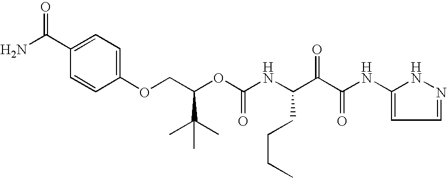

(1S)-1-{[4-(Aminocarbonyl)phenoxy]methyl}-2,2-dimethylpropyl 4-nitrophenyl carbonate was coupled with tert-butyl(1S)-1-[1-hydroxy-2-oxo-2-(1H-pyrazole-5-ylamino)ethyl]pentylcarbamate and oxidized with Dess-Martin periodinane as described in examples 1n Et 1o to afford the title compound as a solid foam. ¹H NMR (DMSO-d₆) δ 12.5 (br s, 1H), 10.8 (br s, 1H), 7.79 (d, J=8 Hz, 3H), 7.64 (br s, 1H), 7.54 (d, J=8 Hz, 1H), 7.14 (br s, 1H), 6.92 (d, J=8 Hz, 2H), 6.50 (br s, 1H), 4.87-4.81 (m, 1H), 4.80-4.76 (m, 1H), 4.25 (d, J=9 Hz, 1H), 3.97 (br t, J=9 Hz, 1H), 1.8-1.7 (m, 1 H), 1.5-1.4 (m, 1H), 1.35-1.10 (m, 4H), 0.96 (s, 9H), 0.85 (t, 3H); ES-LCMS m/z 488 (M+H).

### Example 49

### Preparation of (1S)-1-{[4-(1H-imidazol-1-yl)phenoxy]methyl}-2,2-dimethylpropyl(1S)-1-[oxo(1H-pyrazol-5-ylamino)acetyl]pentylcarbamate

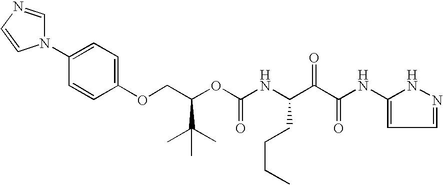

### Example 49a

### Preparation of (1S)-1-{[4-(1H-Imidazol-1-yl)phenoxy]methyl}-2,2-dimethylpropyl 4nitrophenyl carbonate

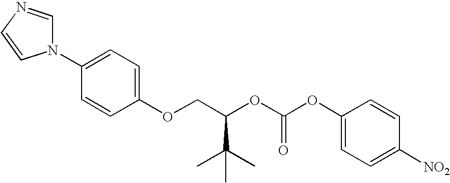

4(Imidazol-1-yl)phenol was subjected to the procedure described in example 47a, and then treated with p-nitrophenyl chloroformate as described in example 1g to afford the title compound as a solid foam. ¹H NMR (DMSO-d₆) δ 8.31 (d, J=9 Hz, 2H), 8.14 (s, 1H), 7.65 (s, 1H), 7.55 (d, J=9 Hz, 2H), 7.51 (d, J=9 Hz, 2H), 7.11 (d, J=9 Hz, 2H), 7.06 (s, 1H), 4.93 (dd, J=9 Hz, J=2 Hz, 1H), 4.43 (dd, J=11 Hz, J=2 Hz, 1H), 4.16 (dd, J=11 Hz, J=9 Hz, 1H), 1.04 (s, 9H).

### Example 49b

### Preparation of (1S)-1-{[4-(1H-Imidazol-1-yl)phenoxy]methyl}-2,2-dimethylpropyl(1S)-1-[oxo(1H-pyrazol-5-ylamino)acetyl]pentylcarbamate

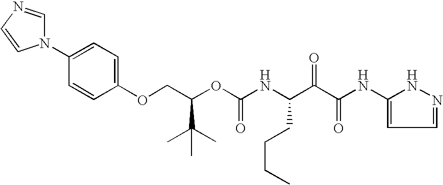

(1)-1-{[4(1H-Imidazol-1-yl)phenoxy]methyl}-2,2-dimethylpropyl 4-nitrophenyl carbonate was coupled with tert-butyl(1S)-1-[1-hydroxy-2-oxo-2-(1H-pyrazole-5-ylamino)ethyl]pentylcarbamate and oxidized with Dess-Martin periodinane as described in examples 1n Et 1o to afford the title compound as a solid foam. ¹H NMR (DMSO-d₆) δ 12.50 (br s, 1H), 10.80 (br s, 1H), 8.10 (s, 1H), 7.80-7.40 (m, 5H), 7.10-6.90 (m, 3H), 6.50 (br s, 1H), 4.90-4.70 (m, 2H), 4.25 (br d, J=10 Hz, 1H), 3.97 (br t, J=9 Hz, 1H). 1.80-1.70 (m, 1H), 1.50-1.20 (m, 5H), 0.94 (s, 9H), 0.82 (t, 3H); ES-LCMS m/z 511.5 (M+H).

### Example 50

### Preparation of (1S)-1-({4-[3,5-bis(trifluoromethyl)phenyl]-7H-imidazol-1-yl}methyl)-2,2-dimethylpropyl(1S)-1-[oxo(1H-pyrazol-5-ylamino)acetyl]butylcarbamate

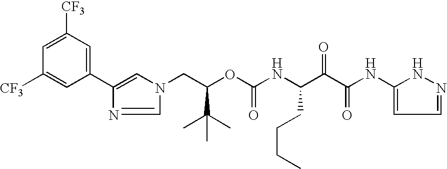

### Example 50a

### Preparation of 4-[3,5-bis(trifluoromethyl)phenyl]-1H-imidazole

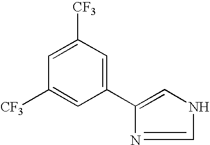

A solution of 1.20 g (3.6 mmol) of 3,5-bis(trifluoromethyl)phenyacylbromide in 10 mL of formamide was stirred at 185° C. for 1.25 h. The reaction mixture was allowed to cool to room temperature, and was stirred for 1 h before being diluted with 35 mL of saturated aqueous sodium bicarbonate. The resulting mixture was extracted with three 35 mL portions of ethyl acetate, and the combined extracts were washed with 30 mL portions of saturated aqueous sodium bicarbonate, and saturated aqueous sodium chloride, dried over anhydrous magnesium sulfate, and concentrated under vacuum. The resulting yellow solid was further purified by silica gel chromatography, eluting with 20% hexane in ethyl acetate to afford 1.04 g (quantitative yield) of 4-[3,5-bis(trifluoromethyl)phenyl]-1H-imidazole as an off-white solid. ¹H NMR (300 MHz, DMSO-d₆) δ 12.48 (br s, 1H), 8.40 (s, 2H), 8.05 (s, 1H), 7.84 (s, 1H), 7.81 (s, 1H); ES-LCMS m/z 279 (M−H).

### Example 50b

### Preparation of (2S)-1-{4-[3,5-bis(trifluoromethyl)phenyl]-1H-imidazol-1-yl}-3,3-dimethyl-2-butanol

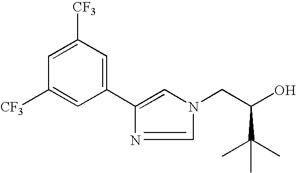

First, 147 mg (3.68 mmol) of sodium hydride was added to a solution of 940 mg (3.35 mmol) of 4-[3,5-bis(trifluoromethyl)phenyl]-1H-imidazole in 12 mL of anhydrous N,N-dimethylformamide in a thick-walled sealable tube. After gas evolution had ceased, a solution of 911 mg (3.35 mmol) of (2S)-2-hydroxy-3,3-dimethylbutyl 4-methylbenzenesulfonate in 3 mL of anhydrous N,N-dimethylformamide was added. The tube was sealed and heated at 80° C. for 25 h. When at this time no product was detected by thin-layer silica gel chromatography, eluting with 20% ethyl acetate in hexane, 0.50 mL (3.35 mmol) of 1,8-diazabicyclo[5.4.0]undec-7-ene was added, and the reaction mixture was again heated in the sealed tube at 80° C. for 17 h. When no reaction was detected, the reaction temperature was raised to 130° C. After 24 h at this temperature, the reaction mixture was cooled, and diluted with 50 mL of ethyl acetate, causing a solid to precipitate. The mixture was then washed with three 50 mL portions of water, and the aqueous washes were back-extracted with 30 mL of ethyl acetate. The ethyl acetate layers were combined, washed with 30 mL of saturated aqueous sodium chloride, dried over anhydrous magnesium sulfate, and concentrated to an oil, which was separated into it's components by column chromatography on silica gel. Elution with 25% ethyl acetate in hexane resulted in the recovery of 380 mg (30%) of unreacted 4-[3,5-bis(trifluoromethyl)phenyl]-1H-imidazole, and afforded 559 mg (44%) of (2S)-1-{4-[3,5-bis(trifluoromethyl)phenyl]-1H-imidazol-1-yl}-3,3-dimethyl-2-butanol as an off-white solid. ¹H NMR (300 MHz, DMSO-d₆) δ 8.36 (s, 2H), 8.14 (s, 1H), 7.83 (s, 1H), 7.76 (s, 1H), 5.03 (d, J=7 Hz, 1H), 4.15 (app dd, J=14 Hz, J=2 Hz, 1H), 3.75 (app dd, J=14 Hz, J=10 Hz, 1H), 3.34-3.36 (m, 1H), 0.92 (s, 9H); ES-LCMS m/z 381 (M+H).

### Example 50c

### Preparation of (1S)-1-({4-[3,5-bis(trifluoromethyl)phenyl]-1H-imidazol-1-yl}methyl)-2,2-dimethylpropyl 4-nitrophenyl carbonate

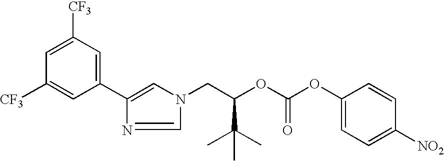

A solution of 0.940 mL (1.50 mmol) of 1.6 M butyllithium in hexanes was added dropwise to a solution of 543 mg (1.43 mmol) of (2S)-1-{4-[3,5-bis(trifluoromethyl)phenyl]-1H-imidazol-1-yl}-3,3-dimethyl-2-butanol in 15 mL of anhydrous tetrahydrofuran at 0° C. The resulting yellow solution was stirred for 12 min at 0° C., before a solution of 430 mg (2.14 mmol) of 4-nitrophenyl chloroformate in 5 mL of anhydrous tetrahydrofuran was added. The resulting solution was stirred and allowed to warm to room temperature. After 18 h, the solution was diluted with 60 mL of ethyl acetate, and washed with two 30 mL aliquots of water. The aqueous washes were then back-extracted with 30 mL of ethyl acetate, and this extract was combined with the original ethyl acetate phase. After washing with 31 mL of saturated aqueous sodium chloride and drying over magnesium sulfate, the volatiles were removed under vacuum to afford a yellow oil that crystallized on standing. This mixture was partially purified by column chromatography on silica gel. Elution with 17% ethyl acetate in hexane followed by 33% ethyl acetate in hexane resulted in the recovery of 270 mg (50%) of (2S)-1-{4-[3,5-bis(trifluoromethyl)phenyl]-1H-imidazol-1-yl}-3,3-dimethyl-2-butanol, and 386 mg of a 3:1 mixture of starting material and desired product. This mixture was dissolved in 5 mL of anhydrous dichloromethane under nitrogen, and 332 mg (1.65 mmol) of 4-nitrophenyl chloroformate was added, followed by 0.230 mL (1.65 mmol) of triethylamine. The resulting mixture was stirred for 6 d. Volatiles were then removed under vacuum, and the resulting residue was further purified by column chromatography on silica gel. Elution with dichloromethane provided 340 mg of a yellow oil, which was dissolved in 30 mL of ethyl acetate. This solution was washed with two 20 mL aliqouts of 1 N sodium hydroxide, followed by 20 mL of saturated aqueous sodium chloride, dried over magnesium sulfate, and concentrated under vacuum to provide 170 mg (22%) of (1S)-1-({4-[3,5-bis(trifluoromethyl)phenyl]-1H-imidazol-1-yl}methyl)-2,2-dimethylpropyl 4-nitrophenyl carbonate as a white solid. ¹H NMR (300 MHz, DMSO-d₆) δ 8.34 (s, 1H), 8.22 (s, 1H), 8.09 (d, J=9 Hz, 2H), 7.91 (s, 1H), 7.87 (s,1H), 7.26 (d, J=9 Hz, 2H), 4.85 (app dd, J=10 Hz, J=2 Hz, 1H), 4.53 (app dd, J=14 Hz, J=2 Hz, 1H), 4.24 (app dd, J=14 Hz, J=10 Hz, 1H), 1.06 (s, 9H); ES-LCMS m/z 546 (M+H).

### Example 50d

### Preparation of (1S)-1-({4-[3,5-bis(trifluoromethyl)phenyl]-1H-imidazol-1-yl}methyl)-2,2-dimethylpropyl(1S)-1-[(1R)-1-hydroxy-2-oxo-2-(1H-pyrazol-5-ylamino)ethyl]pentylcarbamate Et(1S)-1-({4-[3,5-bis(trifluoromethyl)phenyl]-1H-imidazol-1-yl}methyl)-2,2-dimethylpropyl(1S)-1-[(1S)-1-hydroxy-2-oxo-2-(1H-pyrazol-5-ylamino)ethyl]pentylcarbamate

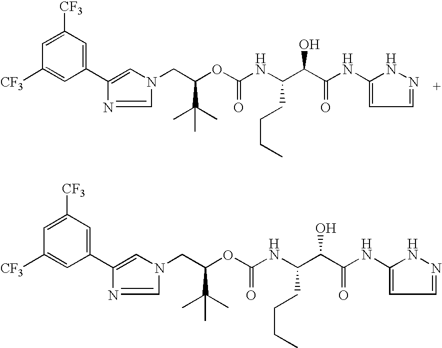

First, 98 mg (0.30 mmol) of tert-butyl(1S)-1-[(1R)-1-hydroxy-2-oxo-2-(1H-pyrazol-5-ylamino)ethyl]pentylcarbamate Et tert-butyl(1S)-1-[(1S)-1-hydroxy-2-oxo-2-(1H-pyrazol-5-ylamino)ethyl]pentylcarbamate were dissolved in 1.5 mL of anhydrous dioxane. Then, 1.5 mL of a 4 N solution of hydrogen chloride in dioxane was added, and the resulting solution was stirred for 20 min, during which a white precipitate formed. The mixture was concentrated under vacuum. The resulting white solid was dried under vacuum, and then slurried in 1 mL of anhydrous N,N-dimethylformamide. Addition of 160 μL (0.90 mmol) of diisopropylethylamine resulted in a light yellow solution, to which a solution of 165 mg (0.30 mmol) of (1S)-1-({4-[3,5-bis(trifluoromethyl)phenyl]-1H-imidazol-1-yl}methyl)-2,2-dimethylpropyl 4-nitrophenyl carbonate in 1 mL of N,N-dimethylformamide was added. The resulting solution was stirred for 40 h. It was then diluted with 50 mL of ethyl acetate, and washed with three 30 ml aliquots of saturated aqueous sodium bicarbonate, followed by two 30 mL portions of saturated aqueous sodium chloride. After drying over magnesium sulfate, volatiles were removed under vacuum to afford a yellow oil, which was further purified by column chromatography on silica gel. Elution with 5% methanol in chloroform afforded 100 mg (53%) of (1S)-1-({4-[3,5-bis(trifluoromethyl)phenyl]-1H-imidazol-1-yl}methyl)-2,2-dimethylpropyl(1S)-1-[(1R)-1-hydroxy-2-oxo-2-(1H-pyrazol-5-ylamino)ethyl]pentylcarbamate Et(1S)-1-({4-[3,5-bis(trifluoromethyl)phenyl]-1H-imidazol-1-yl}methyl)-2,2-dimethylpropyl(1S)-1-[(1S)-1-hydroxy-2-oxo-2-(1H-pyrazol-5-ylamino)ethyl]pentylcarbamate as a white solid. ¹H NMR (300 MHz, DMSO-d₆) δ 12.40-12.23 (m, 1H), 9.65 (br s) and 9.63 (br s) total 1H, 8.83 (s, 1H), 8.07 (s, 1H), 7.82 (s, 1H), 7.74 (s) and 7.72 (s) total 1H, 7.54 (br s, 1H), 6.77 (d, J=10 Hz) and 6.50 (d, J=10 Hz) total 1H, 6.46-6.35 (m, 1H), 5.88 (br s) and 5.61 (br s) total 1H, 4.68 (app dd, J=11 Hz, J=3 Hz) total 1H, 4.37-4.23 (m, 1H), 4.07-3.88 (m, 2H), 3.72-3.52 (m, 1H), 1.32-0.70 (m, 15H), 0.42 (m, 3H); ES-LCMS m/z 633 (M+H).

### Example 50e

### Preparation of (1S)-1-({4-[3,5-bis(trifluoromethyl)phenyl]-1H-imidazol-1-yl}methyl)-2,2-dimethylpropyl(1S)-1-[oxo(1H-pyrazol-5-ylamino)acetyl]pentylcarbamate

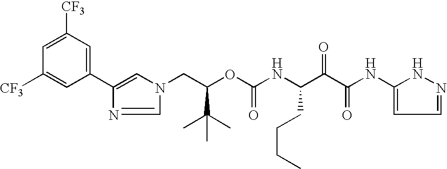

First, 705 mg (0.19 mmol) of Dess Martin periodinane was added to a stirred solution of 100 mg (0.16 mmol) of (1S)-1-({4-[3,5-bis(trifluoromethyl)phenyl]-1H-imidazol-1-yl}methyl)-2,2-dimethylpropyl(1S)-1-[(1R)-1-hydroxy-2-oxo-2-(1H-pyrazol-5-ylamino)ethyl]pentylcarbamate Et(1S)-1-({4-[3,5-bis(trifluoromethyl)phenyl]-1H-imidazol-1-yl}methyl)-2,2-dimethylpropyl(1S)-1-[(1S)-1-hydroxy-2-oxo-2-(1H-pyrazol-5-ylamino)ethyl]pentylcarbamate in 2 mL of dichloromethane. After 45 min, the cloudy reaction mixture was applied directly to a silica gel column, which was eluted with 20% acetone in chloroform, to afford 109 mg of the desired product as a white solid. The solid was dissolved in 50 mL of ethyl acetate, and washed with two 20 mL portions of saturated aqueous sodium thiosulfate. After drying over magnesium sulfate, volatiles were removed to provide (1S)-1-({4-[3,5-bis(trifluoromethyl)phenyl]-1H-imidazol-1-yl}methyl)-2,2-dimethylpropyl(1S)-1-[oxo(1H-pyrazol-5-ylamino)acetyl]pentylcarbamate as a light pink solid, which was further dried under vacuum to afford 56 mg (60%). ¹H NMR (300 MHz, DMSO-d₆) δ 12.35-12.20 (br m, 1H), 10.32 (br s, 1H), 8.81 (s, 2H), 7.93 (s, 1H), 7.75 (s, 1H), 7.68 (s, 1H), 7.55 (d, J=2 Hz, 1H), 7.15-7.06 (m, 1H), 6.44 (br s, 1H), 4.80-4.67 (m, 2H), 4.36-4.30 (m, 1H), 4.06-3.98 (m, 1H), 1.74-1.58 (m, 1H), 1.51-1.33 (m, 1H), 1.22-1.03 (m, 4H), 0.98 (s, 9H), 0.71-0.61 (m, 3H); ES-LCMS m/z 631 (M+H); HRMS C₂₈H₃₂₀N₆O₄F₆ m/z 631.2467 (M+H)_{Cal}. 631.2466 (M+H)_{Obs}.

### Example 51

### Preparation of (1S)-2,2-dimethyl-1-({4-[4-(trifluoromethyl)phenyl]-1H-imidazol-1-yl}methyl)propyl(1S)-1-[oxo(1,3-thiazol-2-ylamino)acetyl]pentylcarbamate

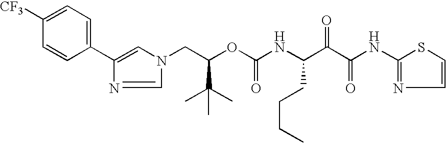

### Example 51a

### Preparation of (1S)-2,2-dimethyl-1-({4-[4-(trifluoromethyl)phenyl]-1H-imidazol-1-yl}methyl)propyl(1S)-1-[(1S)-1-hydroxy-2-oxo-2-(1,3-thiazol-2-ylamino)ethyl]pentylcarbamate Et(1S)-2,2-dimethyl-1-({4-[4-(trifluoromethyl)phenyl]-1H-imidazol-1-yl}methyl)propyl(1S)-1-[(1R)-1-hydroxy-2-oxo-2-(1,3-thiazol-2-ylamino)ethyl]pentylcarbamate

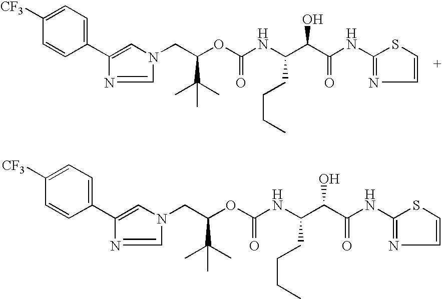

First, 490 mg (1.4 mmol) of tert-butyl(1s)-1-[(1S)-1-hydroxy-2-oxo-2-(1,3-thiazol-2-ylamino)ethyl]pentylcarbamate Et tert-butyl(1S)-1-[(1R)-1-hydroxy-2-oxo-2-(1,3-thiazol-2-ylamino)ethyl]pentylcarbamate were slurried in 2 mL of anhydrous dioxane. Then, 5 mL of a 4 N solution of hydrogen chloride in dioxane was added, and the resulting solution was stirred for 1 h, during which a white precipitate formed. The mixture was concentrated under vacuum, and then the residue was slurried in 4 mL of anhydrous N,N-dimethylformamide. Addition of 694 μL (3.90 mmol) of diisopropylethylamine resulted in a light yellow solution, to which a solution of 660 mg (1.30 mmol) of (1S)-2,2-dimethyl-1-({4-[4-(trifluoromethyl)phenyl]-1H-imidazol-1-yl}methyl)propyl 4-nitrophenyl carbonate in 4 mL of N,N-dimethylformamide was added. The resulting solution was stirred for 15 h at room temperature, and then for 5.5 h at 55° C. Upon cooling to room temperature, it was diluted with 150 mL of ethyl acetate, and washed with two 40 ml aliquots of saturated aqueous sodium bicarbonate. The aqueous washes were then back-extracted with ethyl acetate, and the combined ethyl acetate layers were washed with saturated aqueous sodium chloride, dried over anhydrous magnesium sulfate, and concentrated. The resulting yellow oil was further purified by column chromatography on silica gel. Elution with 2.5-5% methanol in dichloromethane afforded 629 mg (83%) of (1S)-2,2-dimethyl-1-({4-[4-(trifluoromethyl)phenyl]-1H-imidazol-1-yl}methyl)propyl(1S)-1-[(1S)-1-hydroxy-2-oxo-2-(1,3-thiazol-2-ylamino)ethyl]pentylcarbamate Et(1S)-2,2-dimethyl-1-({4-[4-(trifluoromethyl)phenyl]-1H-imidazol-1-yl}methyl)propyl(1S)-1-[(1R)-1-hydroxy-2-oxo-2-(1,3-thiazol-2-ylamino)ethyl]pentylcarbamate as a light yellow foam. ¹H NMR (300 MHz, DMSO-d₆) δ 11.67-11.55 (br m, 1H), 7.97-7.91 (m, 2H), 7.79 (s, 1H), 7.73-7.63 (m, 3H), 7.18 (d, J=4Hz) and 7.16 (d, J=4Hz) total 1H, 7.22 (d, J=4Hz) and 7.20 (d, J=4 Hz) total 1H. 6.78 (d, J=10 Hz) and 6.65 (d, J=10 Hz) total 1H. 5.88 (d, J=7 Hz) and 5.56 (d, J=7 Hz) total 1H. 4.67 (m) and 4.58 (m) total 1H. 4.34-4.26 (m, 1H), 4.20-3.90 (m, 1H), 3.81-3.71 (m) and 3.68-3.55 (m) total 1H, 1.45-1.13 (m, 2H), 1.04-0.90 (m, 4H), 0.86 (s) and 0.82 (s) total 3H; ES-LCMS m/z 582 (M+H).

### Example 51b

### Preparation of (1S)-2,2-dimethyl-1-({4-[4-(trifluoromethyl)phenyl]-1H-imidazol-1-yl}methyl)propyl(1S)-1-[oxo(1,3-thiazol-2-ylamino)acetyl]pentylcarbamate

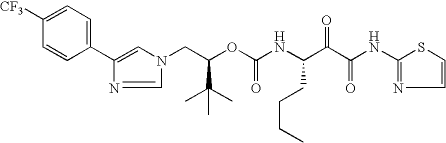

488 mg (1.30 mmol) of Dess Martin periodinane was added to a stirred solution of 629 mg (1.08 mmol) of (1S)-2,2-dimethyl-1-({[4-(trifluoromethyl)phenyl]-1H-imidazol-1-yl}methyl)propyl(1S)-1-[(1S)-1-hydroxy-2-oxo-2-(1,3-thiazol-2-2-ylamino)ethyl]pentylcarbamate Et(1S)-2,2-dimethyl-1-({4-[4(trifluoromethyl) phenyl]-1H-imidazol-1-yl}methyl)propyl(1S)-1-[(1R)-1-hydroxy-2-oxo-2-(1,3-thiazol-2-ylamino)ethyl]pentylcarbamate in 20 ml of dichloromethane. The mixture was sonicated briefly, and then stirred for 20 min. It was then diluted with 20 mL of saturated aqueous sodium thiosulfate. Then, 100 mL of saturated aqueous sodium bicarbonate was added, and the pH of the mixture was adjusted to a value of approximately 10 by the addition of 1 N aqueous sodium hydroxide. The two layers were then separated, and the aqueous phase was extracted with three 40 mL portions of dichloromethane. The extracts were combined with the original dichloromethane layer and washed with two 50 mL portions of saturated aqueous sodium chloride. They were then dried over anhydrous magnesium sulfate, and concentrated to an oily solid, which was further purified by column chromatography on silica gel. Elution with 2.5% methanol in dichloromethane afforded (1S)-2,2-dimethyl-1-({4-[4-(trifluoromethyl)phenyl]-1H-imidazol-1-yl}methyl)propyl(1S)-1-[oxo(1,3-thiazol-2-ylamino)acetyl]pentylcarbamate as a yellow foam, which was further dried under vacuum to provide 497 mg (83%). ¹H NMR (300 MHz, DMSO-d₆, Temp=100° C.) δ 7.94 (d, J=8 Hz, 1H), 7.70-7.65 (m, 4H), 7.56 (d, J=4 Hz, 1H), 7.33 (d, J=4 Hz, 1H), 7.34-7.26 (br m, 1H), 4.79-4.75 (m, 1H), 4.76-4.65 (m, 1H), 4.38-4.32 (m, 1H), 4.08-4.00 (m, 1H), 1.81-1.65 (m, 1H), 1.59-1.44 (m, 1H), 1.33-1.09 (m, 4H), 0.98 (s, 9H), 0.75 (m, 3H); ES-LCMS m/z 580 (M+H). HRMS C₂₇H₃₂N₅O₄SF₃ m/z 580.2205 (M+H)_{Cal}. 580.2197 (M+H)_{Obs}.

### Example 52

### Preparation of (1S)-2,2-dimethyl-1-[5-(3-pyridinyl)-1,3,4-oxadiazol-2-yl]propyl(1S)-1-[oxo(1H-pyrazol-5-ylamino)acetyl]pentylcarbamate

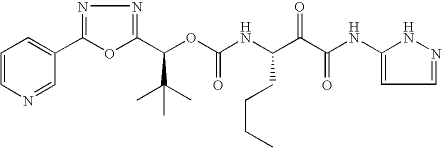

### Example 52a

### Preparation of 3-(1,3,4-oxadiazol-2-yl)pyridine

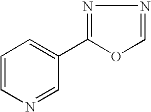

A slurry of 9.10 g (66.0 mmol) of nicotinic hydrazide in 120 mL of triethylorthoacetate was heated at reflux for 24 h. The mixture was allowed to cool to room temperature, and volatiles were removed under vacuum. The resulting solid was slurried in 100 mL of boiling ethyl acetate and 10 mL of ethanol. Volatiles were then removed. The residue was dissolved in a minimal amount of boiling ethanol and allowed to stand overnight. Boiling ethyl acetate was then added to the resulting mixture, which was filtered hot, and then concentrated under vacuum to a light tan solid, which was further dried under vacuum to afford 9.41 g (87%) of 3-(1,3,4-oxadiazol-2-yl)pyridine. ¹H NMR (300 MHz, DMSO-d₆) δ 9.47 (s, 1H), 9.22 (dd, J=2 Hz, J=1 Hz, 1H), 8.84 (dd, J=5 Hz, J=2 Hz, 1H), 8.43 (ddd, J=8 Hz, J=2.3 Hz, J=1.8 Hz, 1H) 7.68 (ddd, J=8 Hz, J=5 Hz, J=1 Hz, 1H); ES-LCMS m/z 148 (M+H).

### Example 52b

### Preparation of (1S)-2,2-dimethyl-1-[5-(3-pyridinyl)-1,3,4-oxadiazol-2-yl-1-propanol and (1R)-2,2-dimethyl-1-[5-(3-pyridinyl)-1,3,4-oxadiazol-2-y]-1-propanol

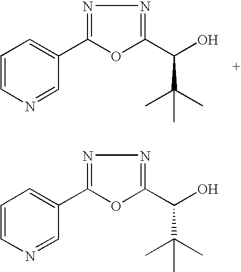

First, 12.50 mL (20.0 mmol) of 1.6 M butyllithium in hexanes was added to 3.38 mL (20.0 mmol) of 2,2,6,6-tetramethylpiperidine at 0° C. in a heat-dried flask, and the resulting slurry was stirred for 1 h. Then, 30 mL of anhydrous tetrahydrofuran was added to afford a tan solution, which was added dropwise to a stirred solution of 1.47 g (10.0 mmol) of 3-(1,3,4-oxadiazol-2-yl)pyridine and 6.52 mL (60.0 mmol) of trimethylacetaldehyde in 40 mL of anhydrous tetrahydrofuran at −42° C. in a heat-dried flask. After 1 h, 20 mL of water was added dropwise, and the mixture was allowed to warm to room temperature. It was then diluted with 80 mL of ethyl acetate and 50 mL of 1 M Tris-HCl buffer (pH 8). After separation of the layers, the upper phase was further washed with two 50 mL portions of 1 M Tris-HCl buffer (pH 8). The washes were combined and back-extracted with 30 mL of ethyl acetate. This extract was combined with the other ethyl acetate layer, and these were washed once more with 50 mL of 1 M Tris-HCl buffer (pH 8), dried over anhydrous magnesium sulfate, and concentrated under vacuum to provide a tan oil. This oil was further purified by column chromatography on silica gel. Elution with 50% methanol containing ammonia (2M) in chloroform, followed by drying under vacuum afforded 2.07 g (89%) of 2,2-dimethyl-1-[5-(3-pyridinyl)-1,3,4-oxadiazol-2-yl]-1-propanol as an off-white solid. ¹H NMR (300 MHz, DMSO-d₆) δ 9.18 (dd, J=2 Hz, J=1 Hz, 1H), 8.83 (dd, J=5 Hz, J=1 Hz, 1H), 8.37 (ca dt, J=8 Hz, J=2 Hz, 1H), 7.67 (dd, J=8 Hz, J=4 Hz, 1H), 6.14 (d, J=5 Hz, 1H), 4.62 (d, J=5 Hz, 1H), 1.00 (s, 9H); APCI-LCMS m/z 234 (M+H). The individual enantiomers were obtained via preparative supercritical fluid chromatography on a Chiralpak AD column (20×250 mm) using a Super C-20 supercritical fluid chromatograph equipped with a carbon dioxide pump, a modifier pump, an automated injector, a column oven, and a UV detector (Novasep, France). 8.55 g of 2,2-dimethyl-1-[5-(3-pyridinyl)-1,3,4-oxadiazol-2-yl]-1-propanol was dissolved in 15 mL of chloroform and 10 mL of methanol. Aliquots of this solution (0.4 mL) were injected onto the Chiralpak AD column, which was eluted with carbon dioxide (45 g/min) and methanol (5 mL/min) at a pressure of 210 bar. The column was maintained at 40° C., and compounds were detected at 290 nm. In this manner, 3.46 g of (1R)-2,2-dimethyl-1-[5-(3-pyridinyl)-1,3,4-oxadiazol-2-yl]-1-propanol was obtained as a colorless crystalline solid in >99% ee and 3.64 g of (1S)-2,2-dimethyl-1-[5-(3-pyridinyl)-1,3,4-oxadiazol-2-yl]-1-propanol was also obtained as a colorless crystalline solid in 94% ee.

### Example 52c: Preparation of (1S)-2,2-dimethyl-1-[5-(3-pyridinyl)-1,3,4-oxadiazol-2-yl]propyl 4-nitrophenyl carbonate

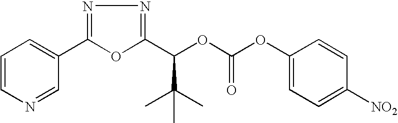

First, 2.01 g (10.0 mmol) of p-nitrophenylchloroformate was added to a slurry of 1.16 g (5.0 mmol) of (1S)-2,2-dimethyl-1-[5-(3-pyridinyl)-1,3,4-oxadiazol-2-yl]-1-propanol and 1.8 mL (10.0 mmol) of diisopropylethylamine in 25 mL of 1,2-dichloroethane under nitrogen. The mixture was then stirred at 80° C. for 24 h. After cooling to room temperature, the mixture was diluted with 50 mL of dichloromethane and 50 mL of 1 M Tris-HCl buffer (pH 8). The layers were separated, and the aqueous phase was extracted with three 50 mL portions of dichloromethane. The dichloromethane extracts and original layer were combined, washed with three 30 mL aliquots of 1 M Tris-HCl buffer (pH 8), dried over anhydrous magnesium sulfate, and concentrated to a red oil. This oil was further purified by column chromatography on silica gel. Elution with 2:1 hexane:ethyl acetate afforded 1.40 g (70%) of (1S)-2,2-dimethyl-1-[5-(3-pyridinyl)-1,3,4-oxadiazol-2-yl]propyl 4-nitrophenyl carbonate as a viscous yellow oil. ¹H NMR (300 MHz, DMSO-d₆) δ 9.22 (dd, J=2 Hz, J=1 Hz, 1H), 8.86 (dd, J=5 Hz, J=1 Hz, 1H), 8.42 (ca dt, J=8 Hz, J=2 Hz, 1H), 8.35 (d, J=9 Hz, 2H), 7.69 (dd, J=8 Hz, J=5 Hz, 1H), 7.62 (d, J=9 Hz, 2H), 5.82 (s, 1H), 1.13 (s, 9H); APCI-LCMS m/z 399 (M+H).

### Example 52d

### Preparation of (1S)-2,2-dimethyl-7-[5-(3-pyridinyl)-1,3,4-oxadiazol-2-yl]propyl(1S)-1-[oxo(1H-pyrazol-5-ylamino)acetyl]pentylcarbamate

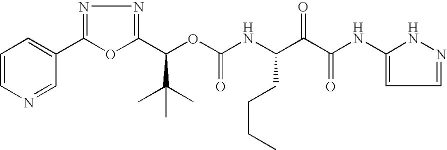

First, 98 mg (0.30 mmol) of tert-Butyl(1S)-1-[(1R)-1-hydroxy-2-oxo-2-(1H-pyrazol-5-ylamino)ethyl]pentylcarbamate Et tert-butyl(1S)-1-[(1S)-1-hydroxy-2-oxo-2-(1H-pyrazol-5-ylamino)ethyl]pentylcarbamate were slurried in 1.0 mL of anhydrous dioxane. Then, 2.5 mL of a 4 N solution of hydrogen chloride in dioxane were added, and the resulting solution was stirred for 40 min, during which a white precipitate formed. The mixture was concentrated under vacuum, and the resulting white solid was then slurried in 1.0 mL of anhydrous N,N-dimethylformamide. Then, 160 μL (0.90 mmol) of diisopropylethylamine was added, followed by a solution of 120 mg (0.30 mmol) of (1S)-2,2-dimethyl-1-[5-(3-pyridinyl)-1,3,4-oxadiazol-2-yl]propyl 4-nitrophenyl carbonate in 1.0 mL of N,N-dimethylformamide. The resulting solution was stirred at 50° C. for 24 h, and then for 18 h at room temperature. It was then concentrated under vacuum to afford a yellow oil, which was further purified by column chromatography on silica gel. Elution with 1:1 hexane:ethyl acetate, followed by 8% methanol in chloroform afforded 111 mg (76%) of (1S)-2,2-dimethyl-1-[5-(3-pyridinyl)-1,3,4-oxadiazol-2-yl]propyl(1S)-1-[(1R)-1-hydroxy-2-oxo-2-(1H-pyrazol-5-ylamino)ethyl]pentylcarbamate Et(1S)-2,2-dimethyl-1-[5-(3-pyridinyl)-1,3,4-oxadiazol-2-yl]propyl(1S)-1-[(1S)-1-hydroxy-2-oxo-2-(1H-pyrazol-5-ylamino)ethyl]pentylcarbamate as a white solid foam.

Then, 97 mg (0.26 mmol) of Dess Martin periodinane was added to a stirred solution of 100 mg (0.21 mmol) of (1S)-2,2-dimethyl-1-[5-(3-pyridinyl)-1,3,4-oxadiazol-2-yl]propyl(1S)-1-[(1R)-1-hydroxy-2-oxo-2-(1H-pyrazol-5-ylamino)ethyl]pentylcarbamate Et(1S)-2,2-dimethyl-1-[5-(3-pyridinyl)-1,3,4-oxadiazol-2-yl]propyl (1S)-1-[(1S)-1-hydroxy-2-oxo-2-(1H-pyrazol-5-ylamino)ethyl]pentylcarbamate in 3 mL of dichloromethane. After 27 min, the cloudy reaction mixture was applied directly to a silica gel column, which was eluted with 3% methanol in dichloromethane, followed by 4% methanol in dichloromethane to afford a 62 mg of a yellow foam. The foam was dissolved in 10 mL of dichloromethane, and washed with 3 mL of saturated aqueous sodium thiosulfate. Aqueous saturated aqueous sodium bicarbonate was added to the mixture until the pH of the resulting aqueous layer was 10. The two layers were then separated, and the aqueous phase was extracted with three 5 mL portions of dichloromethane. The dichloromethane layers were combined, dried over anhydrous magnesium sulfate, concentrated, and further dried under vacuum to afford 49 mg (48%) of (1S)-2,2-dimethyl-1-[5-(3-pyridinyl)-1,3,4-oxadiazol-2-yl]propyl(1S)-1-[oxo(1H-pyrazol-5-ylamino)acetyl]pentylcarbamate as a light yellow solid foam. ¹H NMR (300 MHz, DMSO-d₆, Temp=100° C.) δ 10.46 (br s, 1H), 9.17 (m, 1H), 8.83 (m, 1H), 8.33 (m, 1H), 7.85-7.61 (m, 3H), 6.60 (br s, 1H), 5.60 (s, 1H), 5.03-4.81 (m, 2H), 1.89-1.78 (m, 1H), 1.71-1.56 (m, 1H), 1.47-1.24 (m, 4H), 1.09 (s, 9H), 0.92-0.76 (m, 3H); ES-LCMS m/z 484 (M+H). HRMS C₂₃H₂₉N₇O₅ m/z 484.2308 (M+H)_{Cal}. 484.2309 (M+H)_{Obs}.

### Example 53

### Preparation of (1R)-2,2-dimethyl-1-[5-(3-pyridinyl)-1,3,4-oxadiazol-2-yl]propyl(1S)-1-[oxo(1H-pyrazol-5-ylamino)acetyl]pentylcarbamate

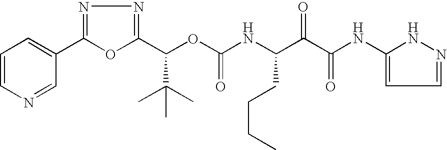

### Example 53a

### Preparation of (1R)-2,2-dimethyl-1-[5-(3-pyridinyl)-1,3,4-oxadiazol-2-yl]propyl 4-nitrophenyl carbonate

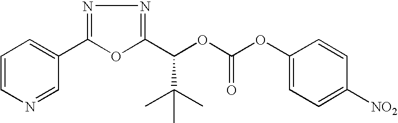

Following the procedure outlined in example 52c, 1.014 g (49%) of (1R)-2,2-dimethyl-1-[5-(3-pyridinyl)-1,3,4-oxadiazol-2-yl]propyl 4-nitrophenyl carbonate was obtained as a viscous yellow oil from 1.16 g (5.0 mmol) of (1R)-2,2-dimethyl-1-[5-(3-pyridinyl)-1,3,4-oxadiazol-2-yl]-1-propanol. Spectral properties were identical to those given for (1S)-2,2-dimethyl-1-[5-(3-pyridinyl)-1,3,4-oxadiazol-2-yl]propyl 4-nitrophenyl carbonate in Example 52c.

### Example 53b

### Preparation of (1R)-2,2-dimethyl-1-[5-(3-pyridinyl)-1,3,4-oxadiazol-2-yl]propyl(1s)-1-[oxo(1H-pyrazol-5-ylamino)acetyl]pentylcarbamate

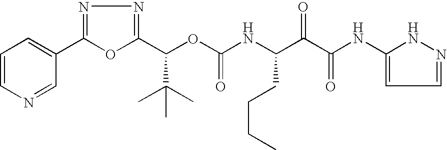

Following the procedure outlined in example 52d, 54 mg (39%) of (1R)-2,2-dimethyl-1-[5-(3-pyridinyl)-1,3,4oxadiazol-2-yl]propyl(1S)-1-[oxo(1H-pyrazol-5-ylamino)acetyl]pentylcarbamate was obtained as a off-white solid foam in two steps from 120 mg (5.0 mmol) of (1R)-2,2-dimethyl-1-[5-(3-pyridinyl)-1,3,4-oxadiazol-2-yl]propyl 4nitrophenyl carbonate. ¹H NMR (300 MHz, DMSO-d₆, Temp=100° C.) δ 10.42 (br s, 1H), 9.17 (br s, 1H), 8.83 (d, J=4 Hz, 1H), 8.35 (m, 1H), 7.70-7.59 (m, 3H), 6.48 (br s, 1H), 5.61 (s, 1H), 5.10-4.85 (m, 1H), 1.89-1.80 (m, 1H), 1.70-1.60 (m, 1H), 1.50-1.20 (m, 4H), 1.09 (s, 9H), 0.92-0.83 (m, 3H); ES-LCMS m/z 484 (M+H); HRMS C₂₃H₂₉N₇O₅ m/z 484.2308 (M+H)_{Cal}. 484.2307 (M+H)_{Obs}.

### EXAMPLE 54

### Preparation of (1S)-2,2-dimethyl-1-[5-(4-pyridinyl)-1,3,4-oxadiazol-2-yl]propyl(1S)-1-[oxo(1H-pyrazol-5-ylamino)acetyl]pentylcarbamate

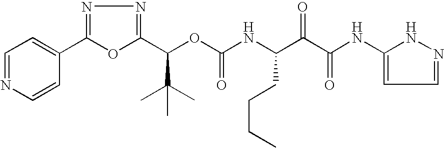

### Example 54a

### Preparation of 4-(1,3,4-oxadiazol-2-yl)pyridine

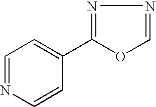

A slurry of 9.60 g (70.0 mmol) of isonicotinic hydrazide in 100 mL of triethylorthoacetate was heated at reflux for 16 h. The mixture was allowed to cool to room temperature, volatiles were removed under vacuum, and the resulting solid was recrystallized twice from ethanol to afford 6.23 g (60%) of 4-(1,3,4oxadiazol-2-yl)pyridine contaminated with ca 5% ethyl isonicotinoylhydrazonoformate. ¹H NMR (300 MHz, DMSO-d₆) δ 9.51 (s, 1H), 8.86 (m, 2H), 7.99 (m, 2H); APCI-LCMS m/z 148 (M+H).

### Example 54b

### Preparation of (1S)-22-dimethyl-1-[5-(4-pyridinyl)-1,3,4-oxadiazol-2-yl]-1-propanol and (1R)-2,2-dimethyl-1-[5-(4-pyridinyl)-1,3,4-oxadiazol-2-yl]-1-propanol

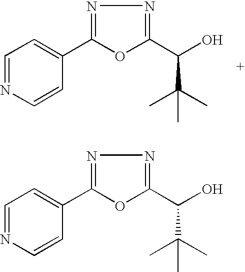

First, 51.3 mL (82.0 mmol) of 1.6 M butyllithium in hexanes was added to 13.8 mL (82.0 mmol) of 2,2,6,6-tetramethylpiperidine at 0° C. in a heat-dried flask, and the resulting slurry was stirred for 1 h. Then, 80 mL of anhydrous tetrahydrofuran was added to afford a tan solution, which was added dropwise to a stirred solution of 6.03 g (41.0 mmol) of 4-(1,3,4-oxadiazol-2-yl)pyridine and 17.8 mL (164 mmol) of trimethylacetaldehyde in 160 mL of anhydrous tetrahydrofuran at −42° C. in a heat-dried flask. After 2.5 h, 50 mL of 1 M Tris-HCl buffer (pH 8) was added dropwise, and the mixture was allowed to warm to room temperature. It was then extracted with 250 mL of ethylacetate. The extract was further washed with three 50 mL portions of 1 M Tris-HCl buffer (pH 8). The washes were combined and back-extracted with 50 mL of ethyl acetate. The extracts were combined and washed once more with 50 mL of 1 M Tris-HCl buffer (pH 8), dried over anhydrous magnesium sulfate, and concentrated under vacuum to provide a yellow slurry. The slurry was triturated with 100 mL of hexane, and the solids were isolated by filtration and recrystallized from ethyl acetate to afford 4.28 g (45%) of 2,2-dimethyl-1-[5-(4-pyridinyl)-1,3,4-oxadiazol-2-yl]-1-propanol as a colorless crystalline solid. ¹H NMR (300 MHz, DMSO-d₆) δ 8.86 (m, 2H), 7.94 (m, 2H), 6.16 (d, J=5 Hz, 1H), 4.63 (d, J=5 Hz, 1H), 1.00 (s, 9H); APCI-LCMS m/z 234 (M+H). The individual enantiomers were obtained via preparative supercritical fluid chromatography on a Chiralpak AD column (20×250 mm) using a Super C-20 supercritical fluid chromatograph equipped with a carbon dioxide pump, a modifier pump, an automated injector, a column oven, and a UV detector (Novasep, France). 4.28 g of 2,2-dimethyl-1-[5-(4-pyridinyl)-1,3,4-oxadiazol-2-yl]-1-propanol was dissolved in 12 mL of chloroform and 23 mL of methanol, and syringe filtered. Aliquots of this solution (0.75 mL) were injected onto the Chiralpak AD column, which was eluted with carbon dioxide (45 g/min) and methanol (5 mL/min) at a pressure of 210 bar. The column was maintained at 40° C., and compounds were detected at 290 nm. In this manner, 1.88 g of (1R)-2,2-dimethyl-1-[5-(4pyridinyl)-1,3,4oxadiazol-2-yl]-1-propanol was obtained as a colorless crystalline solid in >99% ee and 1.95 g of (1S)-2,2-dimethyl-1-[5-(4pyridinyl)-1,3,4-oxadiazol-2-yl]-1-propanol was also obtained as a colorless crystalline solid in 95% ee.

### Example 54c

### Preparation of (1S)-2,2-dimethyl-1-[5-(4-pyridinyl)-1,3,4-oxadiazol-2-yl]propyl 4-nitrophenyl carbonate

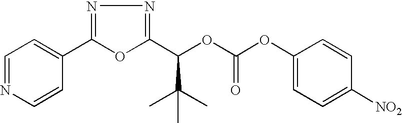

Following the procedure outlined in example 52c, 1.227 g (62%) of (1S)-2,2-dimethyl-1-[5-(4-pyridinyl)-1,3,4-oxadiazol-2-yl]propyl 4-nitrophenyl carbonate was obtained as an off-white solid from 1.16 g (5 mmol) of (1S)-2,2-dimethyl-1-[5-(4-pyridinyl)-1,3,4-oxadiazol-2-yl]-1-propanol. ¹H NMR (300 MHz, DMSO-d₆) δ 8.88 (m, 2H), 8.33 (m, 2H), 7.98 (m, 2H), 7.62 (m, 2H), 5.83 (s, 1 H), 1.00 (s, 9H); APCI-LCMS m/z 399 (M+H).

### Example 54d

### Preparation of (1S)-2,2-dimethyl-1-[5-(4-pyridinyl)-1,3,4-oxadiazol-2-yl]propyl (1S)-1-[oxo(1H-pyrazol-5-ylamino)acetyl]pentylcarbamate

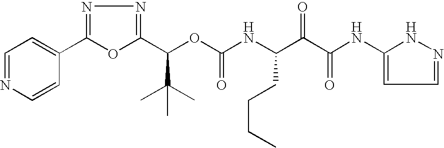

Following the procedure outlined in example 52d, 19 mg (22%) of (1S)2,2-dimethyl-1-[5-(4-pyridinyl)-1,3,4-oxadiazol-2-yl]propyl(1S)-1-[oxo(1H-pyrazol-5-ylamino)acetyl]pentylcarbamate was obtained as an off-white solid foam in two steps from 120 mg (5.0 mmol) of (1S)-2,2-dimethyl-1-[5-(4-pyridinyl)-1,3,4-oxadiazol-2-yl]propyl 4-nitrophenyl carbonate. ¹H NMR (300 MHz, DMSO-d₆, Temp=100° C.) δ 10.48 (br s, 1H), 8.84 (m, 2H), 7.89 (m, 2H), 7.80-7.65 (m, 1H), 7.61 (br s, 1H), 6.51 (br s, 1H), 5.60 (s, 1H), 5.03-4.81 (m, 1H), 1.90-1.78 (m, 1H), 1.65-1.55 (m, 1H), 1.40-1.23 (m, 4H), 1.09 (s, 9H), 0.91-0.81 (m, 3H); ES-LCMS m/z 484 (M+H). HRMS C₂₃H₂₉N₇O₅ m/z 484.2308 (M+H)_{Cal.} 484.2309 (M+H)_{Obs.}

### Example 55

### Preparation of (1R)-2,2-dimethyl-1-[5-(4-pyridinyl)-1,3,4-oxadiazol-2-yl]propyl (1S)-1-[oxo(1H-pyrazol-5-ylamino)acetyl]pentylcarbamate

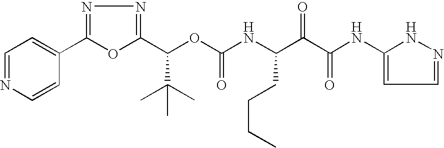

### Example 55a

### Preparation of (1R)-2,2-dimethyl-1-[5-(4-pyridinyl)-1,3,4-oxadiazol-2-yl]propyl 4-nitrophenyl carbonate

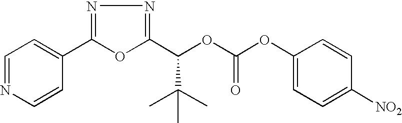

First, 1.21 g (6.0 mmol) of p-nitrophenylchloroformate was added to a slurry of 1.16 g (5.0 mmol) of (1R)-2,2-dimethyl-1-[5-(4-pyridinyl)-1,3,4-oxadiazol-2-yl]-1-propanol and 1.8 mL (10.0 mmol) of diisopropylethylamine in 25 mL of 1,2-dichloroethane under nitrogen. The mixture was then stirred at 80° C. for 5 h, at which point 0.804 g (4.0 mmol) of p-nitrophenylchloroformate was added. The reaction mixture was then stirred at 80° C. for an additional 17 h. Upon cooling to room temperature, the mixture was diluted with 30 mL of 1 M Tris-HCl buffer (pH 8). The layers were separated, and the aqueous phase was extracted with three 50 mL portions of dichloromethane. The dichloromethane extracts and original layer were combined, washed with three 30 mL aliquots of 1 M Tris-HCl buffer (pH 8), dried over anhydrous magnesium sulfate, and concentrated to a brown oil. This oil was further purified by column chromatography on silica gel. Elution with hexane:ethyl acetate (2:1) afforded 1.84 g (91%) of (1R)-2,2-dimethyl-1-[5-(4-pyridinyl)-1,3,4-oxadiazol-2-yl]propyl 4-nitrophenyl carbonate as a viscous yellow oil. Spectral properties were identical to those given in example 54c for (1S)-2,2-dimethyl-1-[5-(4-pyridinyl)-1,3,4-oxadiazol-2-yl]propyl 4-nitrophenyl carbonate, except that the product was shown to contain 0.18 equivalents of ethyl acetate.

### Example 55b

### Preparation of (1R)-2,2-dimethyl-1-[5-(4-pyridinyl)-1,3,4-oxadiazol-2-yl]propyl(1S)-1-[oxo(1H-pyrazol-5-ylamino)acetyl]pentylcarbamate

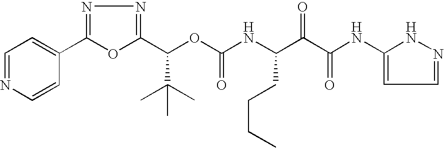

Following the procedure outlined in example 52d, 26 mg (19%) of (1R)-2,2-dimethyl-1-[5-(4-pyridinyl)-1,3,4-oxadiazol-2-yl]propyl(1S)-1-[oxo(1H-pyrazol-5-ylamino)acetyl]pentylcarbamate was obtained as an off-white solid foam in two steps from 124 mg (5.0 mmol) of (1R)-2,2-dimethyl-1-[5-(4-pyridinyl)-1,3,4-oxadiazol-2-yl]propyl 4-nitrophenyl carbonate. ¹H NMR (300 MHz, DMSO-d₆, Temp=100° C.) δ 10.45 (br s, 1H), 8.86 (m, 2H), 7.91 (m, 2H), 7.80-7.65 (m, 1H), 7.60 (m, 1H), 6.48 (br s, 1H), 5.61 (s, 1H), 5.05-4.85 (m, 1H), 1.91-1.76 (m, 1H), 1.73-1.55 (m, 1H), 1.47-1.27 (m, 4H), 1.09 (s, 9H), 0.95-0.81 (m, 3H); ES-LCMS m/z 484 (M+H); HRMS C₂₃H₂₉N₇O₅ m/z 484.2308 (M+H)_{Cal.} 484.2298 (M+H)_{Obs.}

### Example 56

### Preparation of (1S)-2,2-dimethyl-1-(3-thien-2-ylphenyl)propyl(1S)-1-[oxo(1H-pyrazol-5-ylamino)acetyl]pentylcarbamate

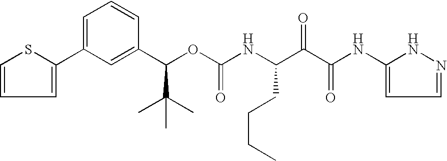

### Example 56a

### Preparation of 1-(3-bromophenyl)-2,2-dimethylpropan-1-ol

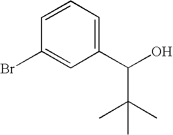

First, 11.89 mL (11.89 mmol) of 1 M tert-butylmagnesium chloride in tetrahydrofuran was added to a solution of 2.00 g (10.81 mmol) of 3-bromobenzaldehyde in 54 mL of tetrahydrofuran at −78° C. and the reaction was allowed to warm to rt and stirred for 4 h. Then, saturated sodium carbonate was added and the resulting mixture was extracted with ethyl acetate. The organic extracts were washed with saturated sodium chloride, dried over anhydrous magnesium sulfate, and concentrated. The residue was purified by silica chromatography eluting with 1:4 ethyl acetate:hexanes to afford 1.11 g (42%) of 1-(3-bromophenyl)-2,2-dimethylpropan-1-ol. R_{f}=0.34 (1:4 ethyl acetate:hexanes); ¹H NMR (300 MHz, DMSO-d₆) δ 7.44 (s, 1H), 7.42 (t, J=5 Hz, 1H), 7.25 (s, 1H), 7.24 (d, J=6 Hz, 1H), 5.30 (s, 1H), 4.22 (s, 1H), 0.81 (s, 9H); ES-LCMS m/z243 (M+H).

### Example 56b

### Preparation of (1S)-2,2-dimethyl-1-(3-thien-2-ylphenyl)propan-1-ol Et (1R)-2,2-dimethyl-1-(3-thien-2-ylphenyl)propan-1-ol

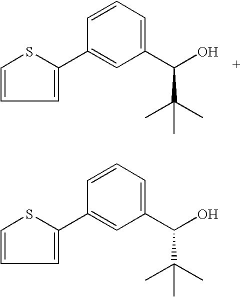

A solution of 1.45 g (13.70 mmol) of sodium carbonate in 9 mL of water was added to a mixture of 1.11 g (4.56 mmol) of 1-(3-bromophenyl)-2,2-dimethylpropan-1-ol, 817.8 mg (6.39 mmol) of thiophene-2-boronic acid, and 320.4 mg (456.5 μmol) of palladium dichlorobis(triphenylphosphine) in 46 mL of N,N-dimethylforamide and the mixture was heated at 90° C. for 16 h. The mixture was allowed to cool to rt and extracted with ethyl acetate. The combined organic extracts were dried over anhydrous magnesium sulfate, and concentrated. The residue was purified by silica chromatography eluting with 1:9 ethyl acetate:hexanes to afford 695.6 mg (62%)) of (1 S)-2,2-dimethyl-1-(3-thien-2-ylphenyl)propan-1-ol Et (1R)-2,2-dimethyl-1-(3-thien-2-ylphenyl)propan-1-ol. R_{f}=0.19 (1:9 ethyl acetate:hexanes); ¹H NMR (300 MHz, DMSO-d₆) δ 7.53 (s, 2H), 7.52 (d, J=5 Hz, 1H), 7.46 (d, J=4 Hz, 1H), 7.32 (t, J=7 Hz, 1H), 7.19 (d, J=7 Hz, 1H), 7.12 (t, J=4 Hz, 1H), 5.24 (br s, 1H), 4.26 (s, 1H), 0.84 (s, 9H); ES-LCMS m/z 247 (M+H). The enantiomers were separated by supercritical fluid chromatography utilizing a Chiralcel OD column (4.6×250 mm) eluting with carbon dioxide:methanol (90:10@21 Mpa).

### Example 56c

### Preparation of (1S)-22-dimethyl-1-(3-thien-2-ylphenyl)propyl 4-nitrophenyl carbonate

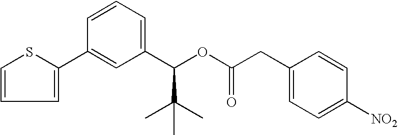

First, 296.4 mg (1.47 mmol) of p-nitrophenylchloroformate was added to a mixture of 241.5 mg (980.2 μmol) of (1S)-2,2-dimethyl-1-(3-thien-2-ylphenyl)propan-1-ol and 158.6 μL (1.96 mmol) of pyridine in 9.8 mL of 1,2-dichloroethane under argon. The mixture was then stirred at 83° C. for 17 h, then allowed to cool to rt Saturated sodium bicarbonate was added and the resulting solution was extracted with ethyl acetate. The combined organic extracts were dried over anhydrous magnesium sulfate, and concentrated. The residue was purified by silica chromatography eluting with 1:9 ethyl acetate:hexanes to afford 231.5 mg (57%) of (1S)-2,2-dimethyl-1-(3-thien-2-ylphenyl)propyl 4-nitrophenyl carbonate. R_{f}=0.21 (1:9 ethyl acetate:hexanes); 1H NMR (300 MHz, DMSO-d6) δ 8.29 (d, J=9 Hz, 2H), 7.65 (d, J=8 Hz, 1H), 7.57 (s, 1H), 7.56 (d, J=5 Hz, 1H), 7.52 (d, J=9 Hz, 2H), 7.52 (m, 1H), 7.44 (t, J=8 Hz, 1H), 7.28 (d, J=8 Hz, 1H), 7.14 (dd, J=5, 4 Hz, 1H), 5.52 (s, 1H), 0.96 (s, 9H); ES-LCMS m/z 434 (M+Na).

### Example 56d

### Preparation of (1S)-2,2-dimethyl-1-(3-thien-2-ylphenyl)propyl(1S)-1-[oxo(1H-pyrazol-5-ylamino)acetyl]pentylcarbamate

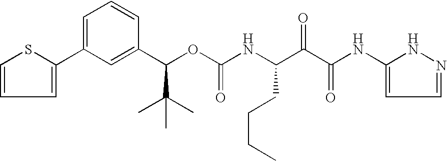

To 202.0 mg (618.9 μmol) of tert-butyl (1)-1-[(1R)-1-hydroxy-2-oxo-2-(1H-pyrazol-5-ylamino)ethyl]pentylcarbamate Et tert-butyl (1S)-1-[(1S)-i -hydroxy-2-oxo-2-(1H-pyrazol-5-ylamino)ethyl]pentylcarbamate in 2.0 mL of dioxane at room temperature was added 7.7 mL (30.94 mmol) of a 4M solution of hydrogen chloride in dioxane. The mixture was stirred for 1 h, concentrated, dried under vacuum, and then dissolved in 2.6 mL of N,N-dimethylformamide. This solution was added to 231.5 mg (562.6 μmol) of (1S)-2,2-dimethyl-1-(3-thien-2-ylphenyl)propyl 4-nitrophenyl carbonate in 3.0 mL of N,N-dimethylformamide, followed by 490.0 μL (2.81 mmol) of N,N-diisopropylethylamine. The resulting mixture was stirred for 23 h at 60° C. It was then concentrated, and the residue was partioned between saturated aqueous sodium bicarbonate and ethyl acetate. The organic layer was washed with saturated aqueous sodium chloride, dried over anhydrous magnesium sulfate, filtered, and concentrated. The residue was purified by silica gel column chromatography eluting with ethyl acetate to give 198.5 mg (71%) of a mixture of alcohols. The alcohols were dissolved in 4.0 mL of dichloromethane at room temperature, and 211.1 mg (497.6 μmol) of Dess-Martin periodinane was added. The reaction mixture was stirred for 2 h, and then poured into saturated aqueous sodium metabisulfite. The resulting mixture was subsequently neutralized with saturated aqueous sodium bicarbonate, and extracted with ethyl acetate. The organic extract was dried over anhydrous magnesium sulfate, filtered, and concentrated. The residue was purified by silica gel column chromatography eluting with 3:2 ethyl acetate:hexanes to give 65.6 mg (33%) of (1S)-2,2-dimethyl-1-(3-thien-2-ylphenyl)propyl(1S)-1-[oxo(1H-pyrazol-5-ylamino)acetyl]pentylcarbamate. R_{f}=0.33 (3:2 ethyl acetate:hexanes); ¹H NMR (300 MHz, DMSO-d₆, Temp=110° C.) δ 10.35 (s, 1H), 7.58 (s, 1H), 7.55-7.40 (m, 5H), 7.33 (t, J=7 Hz, 1H), 7.18 (d, J=7 Hz, 1H), 7.09 (br s, 1H), 6.48 (s, 1H), 5.32 (s, 1), 4.98-4.84 (m, 1H), 1.88-1.68 (m, 1H), 1.64-1.44 (m, 1H), 1.40-1.18 (m, 4H), 0.93 (s, 9H), 0.80 (t, J=7 Hz, 3H); HRMS C₂₆H₃₃N₄O₄S m/z497.2223 (M+H)_{Cal}; 497.2249 (M+H)_{Obs.}

### Example 57

### Preparation of (1R)-2,2-dimethyl-1-(3-thien-2-ylphenyl)propyl(1S)-1-[oxo(1H-pyrazol-5-ylamino)acetyl]pentylcarbamate

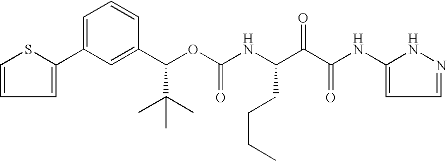

### Example 57a

### Preparation of (1R)-2,2-dimethyl-1-(3-thien-2-ylphenyl)propyl 4-nitrophenyl carbonate

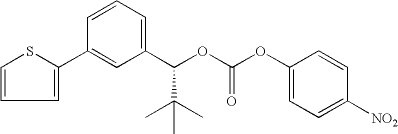

First, 328.9 mg (1.63 mmol) of p-nitrophenyichloroformate was added to a mixture of 268.0 mg (1.09 mmol) of (1R)-2,2-dimethyl-1-(3-thien-2-ylphenyl)propan-1-ol and 176.0 μL (2.18 mmol) of pyridine in 11 mL of 1,2-dichloroethane under argon. The mixture was then stirred at 83° C. for 20 h, then allowed to cool to rt. Saturated sodium bicarbonate was added and the resulting solution was extracted with ethyl acetate. The combined organic extracts were dried over anhydrous magnesium sulfate, and concentrated. The residue was purified by silica chromatography eluting with 1:9 ethyl acetate:hexanes to afford 240.7 mg (54%) of (1R)-2,2-dimethyl-1-(3-thien-2-ylphenyl)propyl 4-nitrophenyl carbonate. R_{f}=0.18 (1:9 ethyl acetate:hexanes); 1H NMR (300 MHz, DMSO-d6) δ 8.29 (d, J=9 Hz, 2H), 7.64 (d, J=8 Hz, 1H), 7.57 (s, 1H), 7.56 (d, J=5 Hz, 1H), 7.52 (d, J=9 Hz, 2H), 7.52 (m, 1H), 7.44 (t. J=8 Hz, 1H), 7.28 )d, J=8 Hz, 1H), 7.14 (dd, J=5, 4 Hz, 1H), 5.52 (s, 1H), 0.96 (s, 9H); ES-LCMS m/z 434 (M+Na).

### Example 57b

### Preparation of (1R)-2,2-dimethyl-1-(3-thien-2-ylphenyl)propyl(1S)-1-[oxo(1H-pyrazol-5-ylamino)acetyl]pentylcarbamate

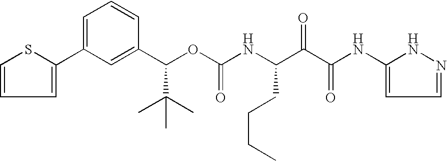

To 210.0 mg (643.5 μmol) of tert-butyl (1S)-1-[(1R)-1-hydroxy-2-oxo-2-(1H-pyrazol-5-ylamino)ethyl]pentylcarbamate Et tert-butyl (1S)-1-[(1S)-1-hydroxy-2-oxo-2-(1H-pyrazol-5-ylamino)ethyl]pentylcarbamate in 2.1 mL of dioxane at room temperature was added 8.0 mL (32.17 mmol) of a 4M solution of hydrogen chloride in dioxane. The mixture was stirred for 1 h, concentrated, dried under vacuum, and then dissolved in 2.8 mL of N,N-dimethylformamide. This solution was added to 240.7 mg (558.0 μmol) of (1R)-2,2-dimethyl-1-(3-thien-2-ylphenyl)propyl 4-nitrophenyl carbonate in 3.0 mL of N,N-dimethylformamide, followed by 509.5 μL (2.92 mmol) of N,N-diisopropylethylamine. The resulting mixture was stirred for 21 h at 60° C. It was then concentrated, and the residue was partioned between saturated aqueous sodium bicarbonate and ethyl acetate. The organic layer was washed with saturated aqueous sodium chloride, dried over anhydrous magnesium sulfate, filtered, and concentrated. The residue was purified by silica gel column chromatography eluting with 9:1 ethyl acetate:hexanes to give 143.3 mg (49%) of a mixture of alcohols. The alcohols were dissolved in 2.9 mL of dichloromethane at room temperature, and 152.3 mg (359.2 μmol) of Dess-Martin periodinane was added. The reaction mixture was stirred for 1 h, and then poured into saturated aqueous sodium metabisulfite. The resulting mixture was subsequently neutralized with saturated aqueous sodium bicarbonate, and extracted with ethyl acetate. The organic extract was dried over anhydrous magnesium sulfate, filtered, and concentrated. The residue was purified by silica gel column chromatography eluting with 3:2 ethyl acetate:hexanes to give 38.1 mg (27%) of (1R)-2,2-dimethyl-1-(3-thien-2-ylphenyl)propyl(1S)-1-[oxo(1H-pyrazol-5-ylamino)acetyl]pentylcarbamate. R_{f}=0.30 (3:2 ethyl acetate:hexanes); ¹H NMR (300 MHz, DMSO-d₆, Temp=110° C.) δ 10.31 (s, 1H), 7.56 (s, 1H), 7.52-7.42 (m, 5H), 7.34 (t, J=8 Hz, 1H), 7.20 (d,J=7 Hz, 1H), 7.12 (t, J=4 Hz, 1H), 6.45 (s, 1H), 5.34 (s, 1H), 5.04-4.84 (m, 1H), 1.86-1.68 (m, 1H), 1.64-1.48 (m, 1H), 1.44-1.22 (m, 4H), 0.90 (s, 9H), 0.86 (t, J=6 Hz, 3H); HRMS C₂₆H₃₃N₄O₄S m/z 497.2223 (M+H)_{Cal}; 497.2246 (M+H)_{Obs.}

### Example 58

### Preparation of (1S)-1-[(5,6-Dichloro-1H-benzimidazol-1-yl)methyl]-2,2-dimethylpropyl(1S)-1-[oxo(pyridin-2-ylamino)acetyl]pentylcarbamate

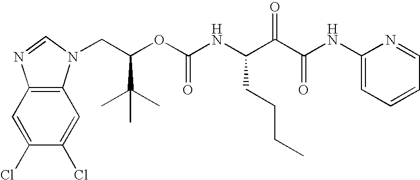

### Example 58a

### Preparation of (1S)-1-[(5,6-Dichloro-1H-benzimidazol-1-yl)methyl]-2,2-dimethylpropyl(1S)-1-[(1R)-1-hydroxy-2-oxo-2-(pyridin-2-ylamino)ethyl]pentylcarbamate Et (1S)-1-[(5,6-Dichloro-1H-benzimidazol-1-yl)methyl]-2,2-dimethylpropyl (1S)-1-[(1S)-1-hydroxy-2-oxo-2-(pyridin-2-ylamino)ethyl]pentylcarbamate

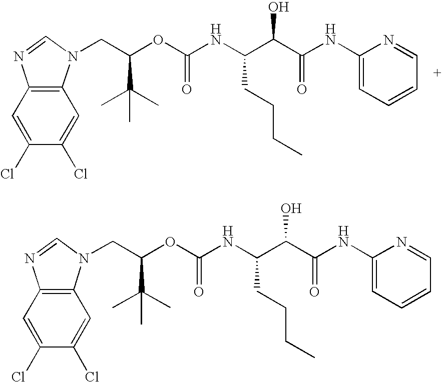

First, 0.41 g (1.2 mmol) of tert-butyl (1S)-1-[(1R)-1-hydroxy-2-oxo-2-(2-pyridinylamino)ethyl]pentylcarbamate Et tert-butyl (1S)-1-[(1S)-1-hydroxy-2-oxo-2-(2-pyridinylamino)ethyl]pentylcarbamate were dissolved in 7 mL of 4N hydrogen chloride in 1,4-dioxane. White solids precipitated within 5 min. The resulting mixture was stirred at ambient temperature for 45 min. Volatiles were removed under vacuum, and the residue was dissolved in 4 mL of N,N-dimethylformamide. Then, 0.86 mL (4.8 mmol) of diisopropylethylamine was added, followed by a solution of 0.48 g (1.1 mmol) of (1S)-1-[(5,6-dichloro-1H-benzimidazol-1-yl)methyl]-2,2-dimethyl propyl 4-nitrophenyl carbonate in 4 mL of N,N-dimethylformamide. The reaction mixture was stirred at 60° C. for 15 h. Volatiles were removed under vacuum, and the resulting oil was purified by silica gel chromatography eluting with ethyl acetate:hexanes (1:1), followed by 40% of a 2M ammonia in methanol solution in chloroform to afford a yellow oil. This material was purified further by silica gel chromatography. Elution with 2.5-4% of a 2M ammonia in methanol solution in chloroform afforded 0.43 g (70%) of the title compounds as a white foam (˜1:1 mixture of diastereomers). ¹H NMR (DMSO-d₆): δ 9.59, 9.53 (2s, 1H), 8.35-8.27 (m, 2H), 8.07-7.71 (m, 4H), 7.17-7.06 (m, 1H), 6.68 (d, J=9 Hz) and 6.50 (d, J=10 Hz) total 1H, 6.00 (d, J=6 Hz) and 5.86 (d, J=7 Hz) total 1 H, 4.75-4.67 and 4.59-4.48 (2m, 2H), 4.33-4.16 (m, 1H), 4.04-3.98 and 3.95-3.90 (2m, 1H), 3.65-3.54 (m, 1H), 1.38-0.63 (m, 18H); ES-LCMS m/z 550, 552 (M+H).

### Example 58b

### Preparation of (1S)-1-[(5,6-dichloro-1H-benzimidazol-1-yl)methyl]-2,2-dimethylpropyl(1S)-1-[oxo(pyridin-2-ylamino)acetyl]pentylcarbamate

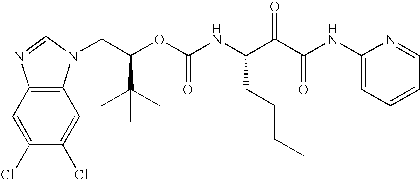

First, 0.36 g (0.94 mmol) of Dess Martin periodinane was added to a solution of 0.41 g (0.75 mmol) of (1S)-1-[(5,6-dichloro-1H-benzimidazol-1-yl)methyl]-2,2-dimethylpropyl(1S)-1-[(1R)-1-hydroxy-2-oxo-2-(pyridin-2-ylamino)ethyl]pentylcarbamate Et (1S)-1-[(5,6-dichloro-1H-benzimidazol-1-yl)methyl]-2,2-dimethylpropyl(1S)-1-[(1S)-1-hydroxy-2-oxo-2-(pyridin-2-ylamino)ethyl]pentylcarbamate in dichloromethane. Then the mixture was sonicated for ca 30 s, and stirred for 30 min. The reaction mixture was then applied directly to a silica gel column, which was eluted with 2% methanol in chloroform to afford a colorless film, which was dissolved in dichloromethane. Saturated aqueous sodium thiosulfate was added, and the two phases were mixed well. The pH of the mixture was adjusted to a value of 10 by the addition of aqueous sodium bicarbonate and sodium hydroxide. The two layers were then separated, and the aqueous layer was extracted with dichloromethane. The combined dichloromethane layers were then washed with saturated aqueous sodium chloride, dried over magnesium sulfate, and concentrated to a yellow oil, which was further purified by silica gel chromatography. Elution with 3% methanol in chloroform afforded 0.15 g (35%) of the title compound as a pale yellow solid foam that contained dichloromethane (0.23 eq. based on integration of signals in the ¹H NMR spectrum). ¹H NMR (DMSO-d₆, Temp=100° C.): δ 10.14 (br s, 1H), 8.36 (br s, 1H), 8.31-8.20 (m, 1H), 8.02-7.78 (m, 4H), 7.31-7.00 (m, 2H), 4.94-4.18 (m, 4H), 1.76-1.60 (m, 1H), 1.57-1.38 (m, 1H), 1.38-0.78 (m, 16H); ES-LCMS m/z 548, 550 (M+H); HRMS C₂₆H₃₁N₅O₄Cl₂ m/z 548.1831 (M+H)_{Cal.} 548.1837 (M+H)_{Obs.}.

### Example 59

### Preparation of (1S)-1-[5-(2,6-dichloropyridin-4-yl)-1,3,4-oxadiazol-2-yl]-2,2-dimethylpropyl(1S)-1-[oxo(1H-pyrazol-5-ylamino)acetyl]pentylcarbamate

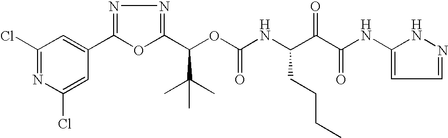

### Example 59a

### Preparation of 2,6-dichloroisonicotinohydrazide

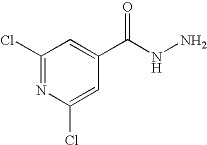

To a suspension of 3.0 g (14.5 mmol) of methyl 2,6-dichloroisonicotinate in 5 mL of 2-propanol was added 0.46 g (14.5 mmol) of hydrazine. The mixture was heated to 70° C. for 2 h. An additional 0.46 g (14.5 mmol) of hydrazine was added and the reaction stirred at 70° C. for 2 h. The reaction was allowed to cool to room temperature and diluted with 2-propanol. The solids and were collected by filtration and vacuum dried to yield 2.0 g (67%) of 2,6-dichloroisonicotinohydrazide. ¹H NMR (DMSO-d₆) δ 10.27 (br s, ₁ H), 7.88 (s, 2H), 4.77 (br s, 2H); ES-LCMS m/z 206, 208 (M+H)⁺.

### Example 59b

### Preparation of 2,6-dichloro-4-(1,3,4-oxadiazol-2-yl)pyridine

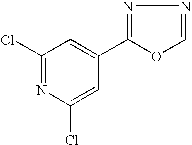

To a flask containing 25 mL of triethylorthoformate was added 2.0 g (9.7 mmol) of 2,6-dichloroisonicotinohydrazide. The reaction was heated to reflux and stirred for 48 h. The mixture was allowed to cool to rt and concentrated. The residue was purified by silica chromatography, eluting with (1:1) ethyl acetate:hexanes to yield 1.63 g (78%) of 2,6-dichloro-4-(1,3,4-oxadiazol-2-yl)pyridine. ¹H NMR (DMSO-d₆) δ 9.56 (s, 1H), 8.09 (s, 2H); ES-LCMS m/z 216, 218 (M+H)⁺.

### Example 59c

### Preparation of (1S)-1-[5-(2,6-dichloropyridin-4-yl)-1,3,4-oxadiazol-2-yl]-2,2-dimethylpropan-1-ol and (1R)-1-[5-(2,6-dichloropyridin-4-yl)-1,3,4-oxadiazol-2-yl]-2,2-dimethylpropan-1-ol

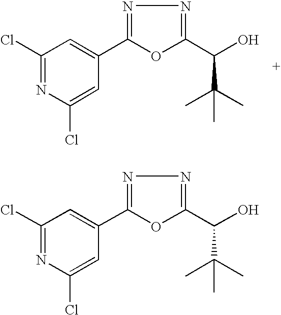

To 2.5 mL (15 mmol) of 2,2,6,6-tetramethylpiperidine at 0C was added 9.4 mL (15 mmol) of 1.6 M n-butyllithium in hexanes, dropwise. The mixture was stirred for 1.5 h and placed in a 4° C. freezer for 18 h. The tan slurry was dissolved in 40 mL of tetrahydrofuran at 0° C. This solution was transferred via canula to a −55° C. solution of 1.63 g (7.5 mmol) of 2,6-dichloro-4-(1,3,4-oxadiazol-2-yl)pyridine and 3.9 g (45 mmol) of 2,2-dimethylpropanal in 30 mL of tetrahydrofuran. The reaction was stirred for 2 h and was then allowed to warm to rt. The mixture was stirred an additional 2 h before being quenched with 20 mL of water and 50 mL of 1 M Tris-HCl buffer (pH 8), and extracted with ethyl acetate. The organic layers were combined, washed with 50 mL 1 M Tris-HCl buffer (pH 8), dried over magnesium sulfate, and concentrated. The residue was purified by silica chromatography, eluting with (1:2) ethyl acetate:hexanes to yield 1.98 g (85%) of (1S)-1-[5-(2,6-dichloropyridin-4-yl)-1,3,4-oxadiazol-2-yl]-2,2-dimethylpropan-1-ol and (1R)-1-[5-(2,6-dichloropyridin-4-yl)-1,3,4-oxadiazol-2-yl]-2,2-dimethylpropan-1-ol. ¹H NMR (DMSO-d₆) δ 8.06 (s, 2H), 6.18 (d, J=5 Hz, 1H), 4.83 (d, J=5 Hz, 1H), 1.00 (s, 9H); ES-LCMS m/z 302, 304 (M+H)⁺. The enantiomers were separated by supercritical fluid chromatography utilizing a Chiralcel OD column (4.6×250 mm) eluting with carbon dioxide:methanol (90:10@21 Mpa).

### Example 59d

### Preparation of (1S)-1-[5-(2,6-dichloropyridin-4-yl)-1,3,4-oxadiazol-2-yl-2,2-dimethylpropyl 4-nitrophenyl carbonate

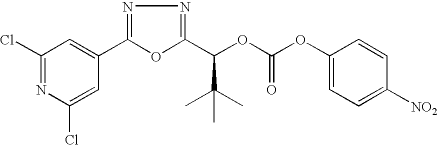

To a solution of 0.83 g (2.7 mmol) of (1S)-1-[5-(2,6-dichloropyridin-4-yl)-1,3,4-oxadiazol-2-yl]-2,2-dimethylpropan-1-ol in 10 mL of dichloroethane was added 0.95 mL (5.5 mmol) of diisopropylethylamine and 1.1 g (5.5 mmol) of p-nitrophenylchloroformate. The mixture was heated at 80° C. for 16 h and allowed to cool before being diluted with ethyl acetate, washed with brine, and concentrated. The residue was purified by silica chromatography, eluting with (3:7) ethyl acetate:hexanes to yield 0.90 g (70%) of (1S)-1-[5-(2,6-dichloropyridin-4-yl)-1,3,4-oxadiazol-2-yl]-2,2-dimethylpropyl 4-nitrophenyl carbonate. ¹H NMR (DMSO-d₆) δ 8.33 (m, 4H), 7.45 (m, 2H), 4.20 (m, 1H), 2.52 (m, 9H).

### Example 59e

### Preparation of (1S)-1-[5-(2,6-dichloropyridin-4-yl)-1,3,4-oxadiazol-2-yl]-2,2-dimethylpropyl(1S)-1-[oxo(1H-pyrazol-5-ylamino)acetyl]pentylcarbamate

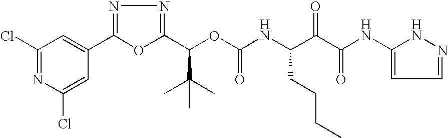

Following the procedure outlined in example 52d, 62 mg (8%) of (1S)-1-[5-(2,6-dichloropyridin-4-yl)-1,3,4-oxadiazol-2-yl]-2,2-dimethyl propyl(1S)-1-[oxo(1H-pyrazol-5-ylamino)acetyl]pentylcarbamate was obtained in two steps from 650 mg (14 mmol) of (1S)-1-[5-(2,6-dichloropyridin-4-yl)-1,3,4-oxadiazol-2-yl]-2,2-dimethylpropyl 4-nitrophenyl carbonate. ¹H NMR (DMSO-d₆) δ 12.58 (m, 1H), 10.93 (m, 1H), 8.11 (d, J=8 Hz, 1H), 8.03 (s, 1H), 7.67 (br s, 2H), 6.52 (br s, 1H), 5.55 (s, 1H), 4.90 (m, 1H), 1.76 (m, 1H), 1.53 (m, 1H), 1.27 (m, 4H), 0.80-1.07 (m. 12H); ES-LCMS m/z 552, 554 (M+H)⁺.

### Example 60

### Preparation of (1R)-1-[5-(2,6-dichloropyridin-4-yl)-1,3,4-oxadiazol-2-yl]-2,2-(1S)-1-[oxo(1H-pyrazol-5-ylamino)acetyl]pentylcarbamate

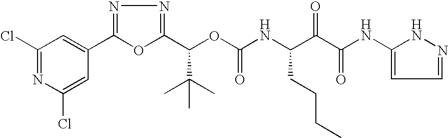

Following the procedures outlined in example 59, (1R)-1-[5-(2,6-dichloropyridin-4yl)-1,3,4-oxadiazol-2-yl]-2,2-dimethylpropyl (1S)-1-[oxo(1H-pyrazol-5-ylamino)acetyl]pentylcarbamate was obtained. ¹H NMR (DMSO-d₆) δ 12.57 (m, 1H), 10.84 (m, 1H), 8.16 (d, J=8 Hz, 1H), 8.05 (s, 2H), 7.65 (m, 1H), 6.54 (m, 1H), 5.55 (s, 1H), 4.89 (m, 1H), 1.78 (m, 1H), 1.23-1.60 (m, 5H), 0.82-1.07 (m, 0.82-1.07 (m, 12H); ES-LCMS m/z 552, 554 (M+H)⁺.

### Example 61

### Preparation of (1S)-1-(4,7-diethoxy-1-methyl-1H-benzimidazol-2-yl)-2,2-dimethylpropyl(1S)-1-[oxo(1H-pyrazol-5-ylamino)acetyl]pentylcarbamate

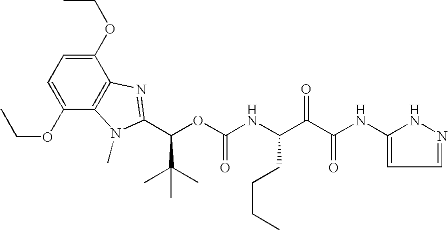

### Example 61a

### Preparation of (1S)-1-(4,7-diethoxy-1-methyl-1H-benzimidazol-2-yl)-2,2-dimethylpropyl 4-nitrophenyl carbonate

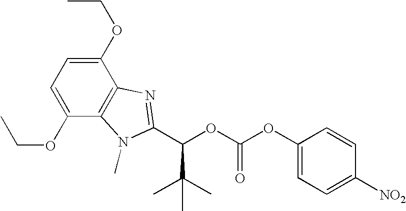

To a solution of 120 mg (0.392 mmol) of (1S)-1-(4,7-diethoxy-1-methyl-1H-benzimidazol-2-yl)-2,2-dimethylpropan-1-ol in 10 mL of dichloromethane was added 79 mg (0.471 mmol) of 4-nitrophenylchloroformate and 60 mg (0.590 mmol) of 4-dimethylaminopyridine. The reaction was stirred at rt for 3 h. The mixture was concentrated and purified by column chromatography with hexane:ethyl acetate ((4:1) then (2:1)) to give 150 mg of product as an oil (81%). ¹H NMR (300 MHz, DMSO-d₆): δ 8.29 (d, J=9 Hz, 2H), 7.55 (d, J=9 Hz, 2H), 6.67 (d, J=9 Hz, 1H), 6.57 (d, J=89 Hz, 1H), 5.70 (s, 1H), 4.19 (q, J=7 Hz, 2H), 4.08-4.05 (m, 2H), 4.14 (s, 3H), 1.36 (m, 6H), 1.12 (s, 9H). ES-LCMS m/z 472 (M+H).

### Example 61b

### (1S)-1-(4,7-diethoxy-1-methyl-1H-benzimidazol-2-yl)-2,2-dimethylpropyl(1S)-1-[(1S)-1-hydroxy-2-oxo-2-(1H-pyrazol-5-ylamino)ethyl]pentylcarbamate Et (1S)-1-(4,7-diethoxy-1-methyl-1H-benzimidazol-2-yl)-2,2-dimethylpropyl(1S)-1-[(1R)-1-hydroxy-2-oxo-2-(1H-pyrazol-5-ylamino)ethyl]pentylcarbamate

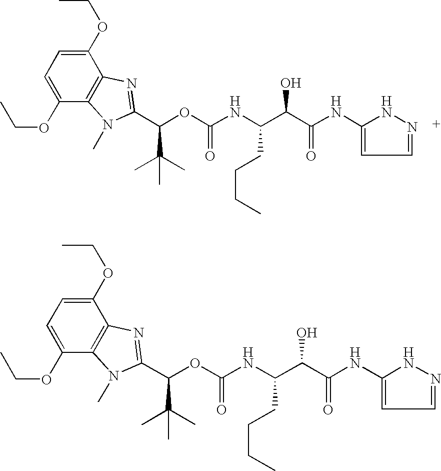

To a solution of 0.31 g (0.95 mmol) of tert-butyl (1S)-1-[(1R)-1-hydroxy-2-oxo-2-(1H-pyrazol-5-ylamino)ethyl]pentylcarbamate Et tert-butyl (1S)-1(1S)-1-hydroxy-2-oxo-2-(1H-pyrazol-5-ylamino)ethyl]pentylcarbamate in 2.5 mL of dioxane at rt was added 10.2 mL (40.8 mmol) of a 4 M solution of hydrogen chloride in dioxane. The mixture was stirred for 1 h, and then concentrated. The residue was dried under vacuum, and dissolved in 8.6 mL of N,N-dimethylformamide. The resulting solution was divided into two equal portions. Then, 0.21 g (0.45 mmol) of (1S)-1-(4,7-diethoxy-1-methyl-1H-benzimidazol-2-yl)-2,2-dimethylpropyl 4nitrophenyl carbonate was added to one of these portions, followed by 0.4 mL (2 mmol) of diisopropylethylamine. The resulting yellow solution was stirred for 1 d at 60° C. It was concentrated, and the residue was partitioned between ethyl acetate and saturated aqueous sodium bicarbonate. The organic phase was concentrated, and the residue was purified by silica gel column chromatography eluting with methanol:chloroform solution (1:9) to yield 63 mg (25%) of the title compound. ES-LCMS m/z 559 (M+H)⁺ retention time=3.7 min.

### Example 61c

### (1S)-1-(4,7-diethoxy-1-methyl-1H-benzimidazol-2-yl)-2,2-dimethylpropyl(1S)-1-[oxo(1H-pyrazol-5-ylamino)acetyl]pentylcarbamate

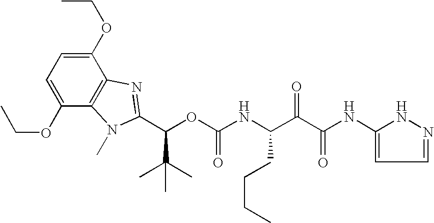

To a solution of 61 mg (0.11 mmol) of (1S)-(4,7-diethoxy-1-methyl-1H-benzimidazol-2-yl)-2,2-dimethylpropyl(1S)-1-[(1S)-1-hydroxy-2-oxo-2-(1H-pyrazol-5-ylamino)ethyl]pentylcarbamate Et (1S)-1-(4,7-diethoxy-1-methyl-1H-benzimidazol-2-yl)-2,2-dimethylpropyl(1S)-1-[(1R)-1-hydroxy-2-oxo-2-(1H-pyrazol-5-ylamino)ethyl]pentylcarbamate in 1.1 mL of chloroform at rt was added 59 mg (0.14 mmol) of Dess-Martin periodinane. The reaction mixture was stirred for 1 h, diluted with ethyl acetate, and then poured into saturated aqueous sodium metabisulfite solution. The resulting mixture was subsequently neutralized with saturated aqueous sodium bicarbonate solution, and the two layers were separated. The organic phase was dried over anhydrous magnesium sulfate, filtered, and concentrated. The residue was purified by silica gel column chromatography eluting with a methanol:chloroform solution (1:9). Fractions containing the product were combined and concentrated to yield 26.2 mg (43%) of the title compound. HRMS (C₂₈H₄₀N₆O₆) m/z 557.3088 (M+H)⁺_{Cal}; 557.3070 (M+H)⁺_{Obs}; ES-LCMS m/z 589 (M+MeOH+H)⁺ retention time=3.8 min.

### Example 62

### Preparation of (1R)-1-{5-[3,5-bis(trifluoromethyl)phenyl]-1,3,4-oxadiazol-2-yl}-2,2-dimethylpropyl(1S)-1-[oxo(1H-pyrazol-5-ylamino)acetyl]pentylcarbamate

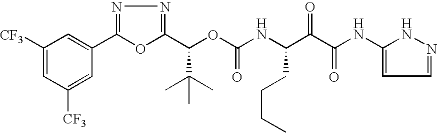

### Example 62a

### Preparation of (1S)-1-{5-[3,5-bis(trifluoromethyl)phenyl]-1,3,4-oxadiazol-2-yl}-butan-1-ol, and (1R)-1-{5-[3,5-bis(trifluoromethyl)phenyl]-1,3,4-oxadiazol-2-yl}-butan-1-ol

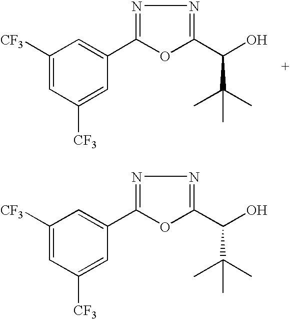

To a flask containing 2.2 mL (13 mmol) of tetramethylpiperdine at 0° C. was added 8.1 mL (13 mmol) of a 1.6 M solution of n-butyllithium in hexanes, and the resulting mixture was stirred 1 h. The mixture was diluted with 18 mL of tetrahydrofuran and added to a solution of 4.2 mL (39 mmol) of trimethylacetaldehyde and 1.83 g (6.5 mmol) of 2-[4-(trifluoromethyl)phenyl]-1,3,4-oxadiazole in 27 mL of tetrahydrofuran at −42° C. The resulting solution was stirred at −42° C. for 2 h. The solution was allowed to warm to rt, and was stirred overnight. It was diluted with ethyl acetate and washed with 10% aqueous citric acid followed by saturated aqueous solution of sodium chloride. The organic layer was dried over anhydrous magnesium sulfate, filtered, and concentrated. The residue was purified by silica gel column chromatography eluting with a chloroform:ethyl acetate solution (4:1) to give racemic 2,2-dimethyl-1-{5-[4-(trifluoromethyl)phenyl]-1,3,4-oxadiazol-2-yl}butan-1-ol. ¹H NMR (300 MHz, DMSO-d₆) δ 8.53 (s, 2H), 8.48 (s, 1H), 6.11 (d, J=5 Hz, 1H), 4.64 (d, J=5 Hz, 1 H), 1.13 and 1.01 (s, 9H). The enantiomers of the racemic alcohol were separated by supercritical fluid chromatography using a Chiralcel OD column, 27° C., 10.5 Mpa, 50% MeOH (1.1 mL/min methanol), 95% CO₂ (20 g/min CO₂). The separated enantiomers were analyzed by SFC chromatography using a Chiralpak AD column, 10 micron, 0.46×25 cm, 5% Methanol:95% Carbon Dioxide, 1.0 mL/min, 1250 psi. Retention times: isomer 1, (1S)-1-{5-[3,5-bis(trifluoromethyl)phenyl]-1,3,4oxadiazol-2-yl}-butan-1-ol, 4.9 min; isomer 2, (1R)-1-{5-[3,5-bis(trifluoromethyl)phenyl]-1,3,4-oxadiazol-2-yl}-butan-1-ol, 6.9 min. ¹H NMR and LC-MS data of each isomer were identical with that of the racemate above.

### Example 62b

### Preparation of (1R)-1-{5-[3,5-bis(trifluoromethyl)phenyl]-1,3,4-oxadiazol-2-yl}-22-dimethylpropyl 4-nitrophenyl carbonate

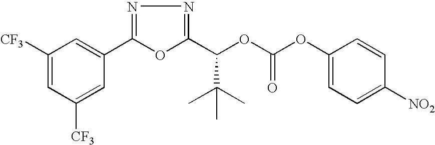

A solution of 0.89 g (2.42 mmol) of (1R)-2,2-dimethyl-1-{5-[4-(trifluoromethyl)phenyl]-1,3,4-oxadiazol-2-yl}butan-1-ol, 0.98 g (4.88 mmol) of 4-nitrophenylchloroformate, and 0.39 mL (4.84 mmol) of pyridine in 20 mL of 1,2-dichloroethane was stirred at 95° C. for 16 h. The solution was partitioned between ethyl acetate and saturated aqueous sodium bicarbonate solution. The organic phase was dried over anhydrous magnesium sulfate, filtered, and concentrated. The residue was purified by silica gel column chromatography eluting with a hexanes:ethyl acetate solution (7:3) to give 0.44 g (34%) of the title compound. ¹H NMR (300 MHz, DMSO-d₆) δ 8.57 (s, 2H), 8.51 (s, 1H), 8.34 (d, J=9 Hz, 2H), 7.62 (d, J=9 Hz, 2), 5.84 (s, 1H), 1.03 (s, 9H).

### Example 62c

### Preparation of (1R)-1-{5-[3,5-bis(trifluoromethyl)phenyl]-1,3,4-oxadiazol-2-yl}-2,2-dimethylpropyl(1S)-1-[(1S)-1-hydroxy-2-oxo-2-(1H-pyrazol-5-ylamino)ethyl]pentylcarbamate and (1R)-1-{5-[3,5-bis(trifluoromethyl)phenyl]-1,3,4-oxadiazol-2-yl}-2,2-dimethylpropyl(1S)-1-[(1R)-1-hydroxy-2-oxo-2-(1H-pyrazol-5-ylamino)ethyl]pentylcarbamate

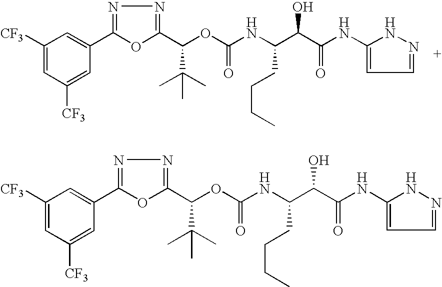

To a solution of 0.31 g (0.95 mmol) of tert-butyl (15)-1-[(1R)-1-hydroxy-2-oxo-2-(1H-pyrazol-5-ylamino)ethyl]pentylcarbamate Et tert-butyl (1s)-1-[(1S)-1-hydroxy-2-oxo-2-(1H-pyrazol-5-ylamino)ethyl]pentylcarbamate in 2.5 mL of dioxane at rt was added 10.2 mL (40.8 mmol) of a 4 M solution of hydrogen chloride in dioxane. The mixture was stirred for 1 h, and then concentrated. The residue was dried under vacuum, and dissolved in 8.6 mL of N,N-dimethylformamide. The resulting solution was divided into two equal portions. Then, 218 mg (1R)-1-{5-[3,5-bis(trifluoromethyl)phenyl]-1,3,4-oxadiazol-2-yl}-2,2-dimethylpropyl 4nitrophenyl carbonate was added, followed by 0.33 mL (1.64 mmol) of diisopropylethylamine. The resulting yellow solution was stirred for 23 h at 60° C. It was concentrated, and the residue was partitioned between ethyl acetate and saturated aqueous sodium bicarbonate. The organic phase was concentrated, and the residue was purified by silica gel column chromatography eluting with methanol:chloroform solution (1:9) to yield 0.09 g (35%) of the title compound. ES-LCMS m/z 621 (M+H)⁺ retention time=4.3 min.

### Example 62d

### Preparation of (1R)-1-{5-[3,5-bis(trifluoromethyl)phenyl]-1,3,4-oxadiazol-2-yl}-2,2-dimethylpropyl(1S)-1-[oxo(1H-pyrazol-5-ylamino)acetyl]pentylcarbamate

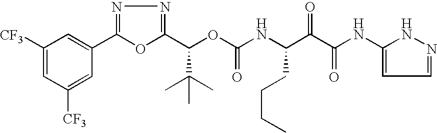

To a solution of 0.084 g (0.135 mmol) of (1R)-1-{5-[3,5-bis(trifluoromethyl)phenyl]-1,3,4oxadiazol-2-yl}-2,2-dimethyl propyl(1S)-1-[(1S)-1-hydroxy-2-oxo-2-(1H-pyrazol-5-ylamino)ethyl]pentylcarbamate and (1R)-1-{5-[3,5-bis(trifluoromethyl)phenyl]-1,3,4-oxadiazol-2-yl }-2,2-dimethylpropyl(1S)-1-[(1R)-1-hydroxy-2-oxo-2-(1H-pyrazol-5-ylamino)ethyl]pentylcarbamate in 1.4 mL of chloroform at rt was added 72 mg (0.17 mmol) of Dess-Martin periodinane. The reaction mixture was stirred for 1 h, diluted with ethyl acetate, and then poured into saturated aqueous sodium metabisulfite solution. The resulting mixture was subsequently neutralized with saturated aqueous sodium bicarbonate solution, and the two layers were separated. The organic phase was washed with brine then dried over anhydrous magnesium sulfate, filtered, and concentrated. The residue was purified by silica gel column chromatography eluting with a methanol:chloroform solution (1:9). Fractions containing the product were combined and concentrated to yield 21.7 mg (26%) of the title compound. HRMS (C₂₆H₂₈F₆N₆O₅) m/z 619.2104 (M+H)⁺_{Cal}; 619.2092 (M+H)⁺_{Obs}; ES-LCMS m/z 619 (M+H)⁺ retention time=4.4 min.

### Example 63

### Preparation of (1S)-1-{5-[3,5-bis(trifluoromethyl)phenyl]-1,3,4-oxadiazol-2-yl}-2,2-dimethylpropyl (1S)-1-[oxo(1H-pyrazol-5-ylamino)acetyl]pentylcarbamate

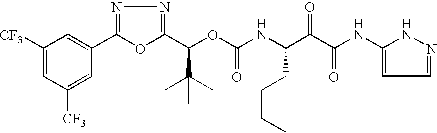

### Example 63a

### Preparation of (1S)-1-{5-[3,5-bis(trifluoromethyl)phenyl]-1,3,4-oxadiazol-2-yl}-2,2-dimethylpropyl 4-nitrophenyl carbonate

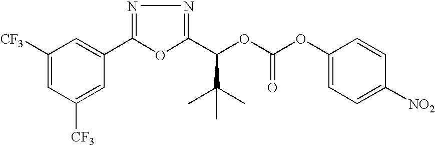

A solution of 0.95 g (2.6 mmol) of (1S)-1-{5-[3,5-bis(trifluoromethyl)phenyl]-1,3,4-oxadiazol-2-yl}-2,2-dimethylpropan-1-ol, 0.98 g (4.88 mmol) of 4-nitrophenylchloroformate, and 0.39 mL (4.84 mmol) of pyridine in 20 mL of 1,2-dichloroethane was stirred at 95° C. for 16 h. The solution was partitioned between ethyl acetate and saturated aqueous sodium bicarbonate solution. The organic phase was dried over anhydrous magnesium sulfate, filtered, and concentrated. The residue was purified by silica gel column chromatography eluting with a hexanes:ethyl acetate solution (7:3) to give 0.807 g (58%) of the title compound. ¹H NMR (300 MHz, DMSO-d₆) δ 8.57 (s, 2H), 8.51 (s, 1H), 8.34 (d, J=9 Hz, 2H), 7.62 (d, J=9 Hz, 2H), 5.84 (s, 1H), 1.14 (s, 9H).

### Example 63b

### Preparation of (1S)-1-{5-[3,5-bis(trifluoromethyl)phenyl]-1,3,4-oxadiazol-2-yl}-2,2-dimethylpropyl(1S)-1-[(1S)-1-hydroxy-2-oxo-2-(1H-pyrazol-5-ylamino)ethyl]pentylcarbamate and (1S)-1-{5-[3,5-bis(trifluoromethyl)phenyl]-1,3,4-oxadiazol-2-yl}-2,2-dimethylpropyl(1S)-1-[(1R)-1-hydroxy-2-oxo-2-(1H-pyrazol-5-ylamino)ethyl]pentylcarbamate

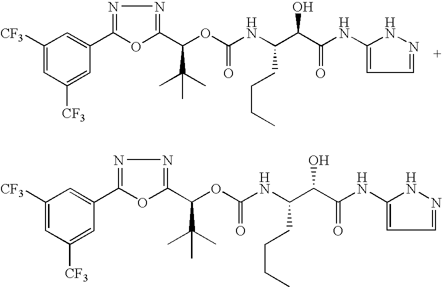

To a solution of 155 mg (0.47 mmol) of tert-butyl (1)-1-[(1R)-1-hydroxy-2-oxo-2-(1H-pyrazol-5-ylamino)ethyl]pentylcarbamate Et tert-butyl (1S)-1-[(1S)-1-hydroxy-2-oxo-2-(1 H-pyrazol-5-ylamino)ethyl]pentylcarbamate in 1.25 mL of dioxane at rt was added 5.1 mL (20.4 mmol) of a 4 M solution of hydrogen chloride in dioxane. The mixture was stirred for 1 h, and then concentrated. The residue was dried under vacuum, and dissolved in 8.6 mL of N,N-dimethylformamide. Then, 218 mg (0.41 mmol) of (1S)-1-{5-[3,5-bis(trifluoromethyl)phenyl]-1,3,4-oxadiazol-2-yl}-2,2-dimethylpropyl 4-nitrophenyl carbonate was added, followed by 0.33 mL (1.64 mmol) of diisopropylethylamine. The resulting yellow solution was stirred for 23 h at 60° C. It was concentrated, and the residue was partitioned between ethyl acetate and saturated aqueous sodium bicarbonate. The organic phase was concentrated, and the residue was purified by silica gel column chromatography eluting with methanol:chloroform solution (1:9) to yield 113.4 mg (45%) of the title compound. ES-LCMS m/z 621 (M+H)⁺ retention time=4.3 min.

### Example 63c

### Preparation of (1S)-1-{5-[3,5-bis(trifluoromethyl)phenyl]-1,3,4-oxadiazol-2-yl}-2,2-dimethylpropyl(1S)-1-[oxo(1H-pyrazol-5-ylamino)acetyl]pentylcarbamate

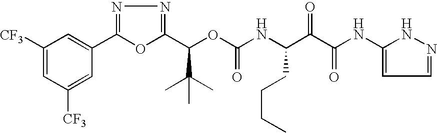

To a solution of 102 mg (0.16 mmol) of (1S)-1-{5-[3,5-bis(trifluoromethyl)phenyl]-1,3,4-oxadiazol-2-yl}-2,2-dimethylpropyl(1S)-1-[(1S)-1-hydroxy-2-oxo-2-(1H-pyrazol-5-ylamino)ethyl]pentylcarbamate and (1S)-1-{5-[3,5-bis(trifluoromethyl)phenyl]-1,3,4-oxadiazol-2-yl }-2,2-dimethylpropyl(1S)-1-[(1R)-1-hydroxy-2-oxo-2-(1H-pyrazol-5-ylamino)ethyl]pentylcarbamate in 1.6 mL of chloroform at rt was added 87 mg (0.17 mmol) of Dess-Martin periodinane. The reaction mixture was stirred for 1 h, diluted with ethyl acetate, and then poured into saturated aqueous sodium metabisulfite solution. The resulting mixture was subsequently neutralized with saturated aqueous sodium bicarbonate solution, and the two layers were separated. The organic phase was washed with brine then dried over anhydrous magnesium sulfate, filtered, and concentrated. The residue was purified by silica gel column chromatography eluting with a methanol:chloroform solution (1:9). Fractions containing the product were combined and concentrated to yield 57 mg (58%) of the title compound. HRMS (C₂₆H₂₈F₆N₆O₅) m/z 619.2104 (M+H)⁺_{Cal}; 619.2123 (M+H)⁺_{Obs}; ES-LCMS m/z 619 (M+H)⁺ retention time=4.3 min.

### Example 64

### Preparation of (1S)-2,2-dimethyl-1-{5-[4-(trifluoromethyl)phenyl]-1,3,4-oxadiazol-2-yl}butyl(1S)-1-[oxo(1H-pyrazol-5-ylamino)acetyl]pentylcarbamate

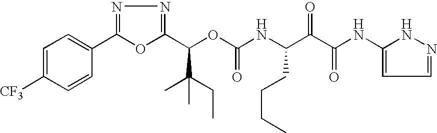

### Example 64a

### Preparation of (1S)-2,2-dimethyl-1-{5-[4-(trifluoromethyl)phenyl]-1,3,4-oxadiazol-2-yl}butan-1-ol, and (1S)-2,2-dimethyl-1-{5-[4-(trifluoromethyl)phenyl]-1,3,4-oxadiazol-2-yl}butan-1-ol

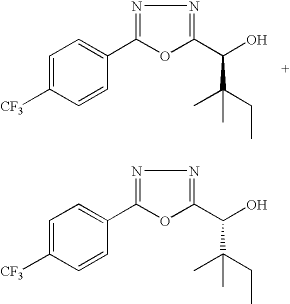

To a flask containing 2 mL (11.8 mmol) of tetramethylpiperdine at 0° C. was added 7.4 mL (11.8 mmol) of a 1.6 M solution of n-butyllithium in hexanes, and the resulting mixture was stirred 1 h. The mixture was diluted with 16 mL of tetrahydrofuran and added to a solution of 2.86 g (22.3 mmol) of 2,2-dimethyl butyraldehyde and 1.26 g (5.9 mmol) of 2-[4-(trifluoromethyl)phenyl]-1,3,4-oxadiazole in 24 mL of tetrahydrofuran at −42° C. The resulting solution was stirred at −42° C. for 2 h. The solution was allowed to warm to rt, and was stirred overnight. It was diluted with ethyl acetate and washed with 100% aqueous citric acid, then saturated aqueous solution of sodium chloride. The organic layer was dried over anhydrous magnesium sulfate, filtered, and concentrated. The residue was purified by silica gel column chromatography eluting with an ethyl chloroform:ethyl acetate solution (3:7) to give 1.36 g (74%) of 2,2-dimethyl-1-{5-[4-(trifluoromethyl)phenyl]-1,3,4-oxadiazol-2-yl}butan-1-ol. ¹H NMR (300 MHz, DMSO-d₆) δ 8.22 (d, J=8 Hz, 2H), 8.01 (d, J=8 Hz, 2H), 6.11 (d, J=5 Hz, 1H), 4.68 (d, J=5 Hz, 1H), 1.5-1.2 (m, 2H), 1.00 (s, 3H), 0.95-0.80 (m, 6H). The enantiomers of the racemic alcohol were separated by supercritical fluid chromatography using a Chiralpak AD column, 27° C., 14 Mpa, 5% MeOH (2.3 mL/min methanol), 95% CO₂ (41 g/min CO₂). The separated enantiomers were analyzed by SFC chromatography using a Chiralpak AD column, 10 micron, 0.46×25 cm, 5% Methanol:95% Carbon Dioxide, 2.0 mL/min, 2000 psi. Retention times: isomer 1, (1S)-2,2-dimethyl-1-{5-[4-(trifluoromethyl)phenyl]-1,3,4-oxadiazol-2-yl}butan-1-ol, 6.6 min; isomer 2, (1S)-2,2-dimethyl-1-{5-[4-(trifluoromethyl)phenyl]-1,3,4-oxadiazol-2-yl}butan-1-ol, 11.3 min. ¹H NMR and LC-MS data of each isomer were identical with that of the racemate above.

### Example 64b

### Preparation of (1S)-2,2-dimethyl-1-{5-[4-(trifluoromethyl)phenyl]-1,3,4-oxadiazol-2-yl}butyl 4-nitrophenyl carbonate

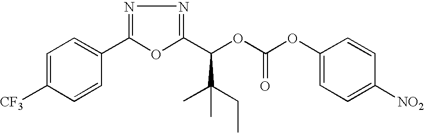

A solution of 0.44 g (sample contains 0.5 toluene by ¹H NMR for an effective weight of 0.38 g, 1.22 mmol) of (1S)-2,2-dimethyl-1-{5-[4-(trifluoromethyl)phenyl]-1,3,4oxadiazol-2-yl}butan-1-ol, 490 mg (2.44 mmol) of 4-nitrophenylchloroformate, and 0.2 mL (2.44 mmol) of pyridine in 12.2 mL of 1,2-dichloroethane was stirred at 95° C. for 16 h. The solution was partitioned between ethyl acetate and saturated aqueous sodium bicarbonate solution. The organic phase was dried over anhydrous magnesium sulfate, filtered, and concentrated. The residue was purified by silica gel column chromatography eluting with a hexanes:ethyl acetate solution (7:3) to give 0.39 g (sample contains 0.67 EtOAc by ¹H NMR for an effective weight of 0.35 g, 1.31 mmol, 59%) of the title compound. ¹H NMR (300 MHz, DMSO-d₆) δ 8.32 (d, J=9 Hz, 2H), 8.21 (d, J=8 Hz, 2H), 8.00 (d, J=8 Hz, 2H), 7.58 (d, J=8 Hz, 2H), 5.81 (s, 1H), 1.43 (q, J=7 Hz, 2H), 1.08 (s, 3H), 1.01 (s, 3H), 0.90 (t, J=7 Hz, 3H).

### Example 64c

### Preparation of (1S)-2,2-dimethyl-1-{5-[4-(trifluoromethyl)phenyl]-1,3,4-oxadiazol-2-yl}butyl (1S)-1-[(1S)-1-hydroxy-2-oxo-2-(1H-pyrazol-5-ylamino)ethyl]pentylcarbamate and (1S)-2,2-dimethyl-1-{5-[4-(trifluoromethyl)phenyl]-1,3,4-oxadiazol-2-yl}butyl(1S)-1-[(1R)-1-hydroxy-2-oxo-2-(1H-pyrazol-5-ylamino)ethyl]pentylcarbamate

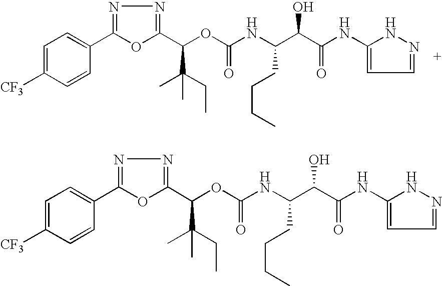

To a solution of 155 mg (0.47 mmol) of tert-butyl(1S)-1-[(1R)-1-hydroxy-2-oxo-2-(1H-pyrazol-5-ylamino)ethyl]pentylcarbamate Et tert-butyl (1S)-1-[(1S)-1-hydroxy-2-oxo-2-(1H-pyrazol-5-ylamino)ethyl]pentylcarbamate in 1.25 mL of dioxane at rt was added 5.1 mL (20.4 mmol) of a 4 M solution of hydrogen chloride in dioxane. The mixture was stirred for 1.25 h, and then concentrated. The residue was dried under vacuum, and dissolved in 4.1 mL of N,N-dimethylformamide. Then, 197 mg (0.41 mmol) of (1S)-2,2-dimethyl-1-{5-[4-(trifluoromethyl)phenyl]-1,3,4-oxadiazol-2-yl}butyl 4-nitrophenyl carbonate was added to followed by 0.33 mL (1.64 mmol) of diisopropylethylamine. The resulting yellow solution was stirred for 1 d at 60° C. It was concentrated, and the residue was partitioned between ethyl acetate and saturated aqueous sodium bicarbonate. The organic phase was concentrated, and the residue was purified by silica gel column chromatography eluting with methanol:chloroform solution (1:9) to yield 0.14 g (60%) of the title compound. ES-LCMS m/z 567 (M+H)⁺ retention time=4.1 min.

### Example 64d

### Preparation of (1S)-2,2-dimethyl-1-{5-[4-(trifluoromethyl)phenyl]-1,3,4-oxadiazol-2-yl}butyl(1S)-1-[oxo(1H-pyrazol-5-ylamino)acetyl]pentylcarbamate

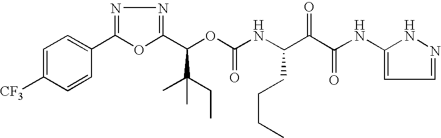

To a solution of 0.14 g (0.25 mmol) of (1S)-2,2-dimethyl-1-{5-[4-(trifluoromethyl)phenyl]-1,3,4-oxadiazol-2-yl}butyl (1S)-1-[(1S)-1-hydroxy-2-oxo-2-(1H-pyrazol-5-ylamino)ethyl]pentylcarbamate and (1S)-2,2-dimethyl-1-{5-[4-(trifluoromethyl)phenyl]-1,3,4-oxadiazol-2-yl}butyl(1S)-1-[(1R)-1-hydroxy-2-oxo-2-(1H-pyrazol-5-ylamino)ethyl]pentylcarbamate in 2.5 mL of chloroform at rt was added 131 mg (0.31 mmol) of Dess-Martin periodinane. The reaction mixture was stirred for 1.25 h, diluted with ethyl acetate, and then poured into saturated aqueous sodium metabisulfite solution. The resulting mixture was subsequently neutralized with saturated aqueous sodium bicarbonate solution, and the two layers were separated. The organic phase was washed with brine, then dried over anhydrous magnesium sulfate, filtered, and concentrated. The residue was purified by silica gel column chromatography eluting with a methanol:chloroform solution (1:9). Fractions containing the product were combined and concentrated to yield 82.2 mg (59%) of the title compound. HRMS (C₂₆H₃₁F₃N₆O₅) m/z 565.2386 (M+H)⁺_{Cal}; 565.2366 (M+H)⁺_{Obs}; ES-LCMS m/z 565 (M+H)⁺ retention time=4.2 min.

### Example 65

### Preparation of (1R)-2,2-dimethyl-1-{5-[4-(trifluoromethyl)phenyl]-1,3,4-oxadiazol-2-yl}butyl(1S)-1-[oxo(1H-pyrazol-5-ylamino)acetyl]pentylcarbamate

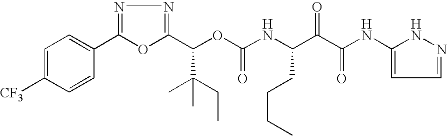

### Example 65a

### Preparation of (1R)-2,2-dimethyl-1-{5-[4-(trifluoromethyl)phenyl]-1,3,4-oxadiazol-2-yl}butyl 4-nitrophenyl carbonate

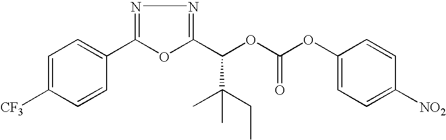

A solution of 432 mg (sample contains 0.22 toluene by ¹H NMR for an effective weight of 0.41g, 1.31 mmol) of (1R)-2,2-dimethyl-1-{5-[4-(trifluoromethyl)phenyl]-1,3,4-oxadiazol-2-yl}butan-1-ol, 527 mg (2.62 mmol) of 4-nitrophenylchloroformate, and 0.21 mL (2.62 mmol) of pyridine in 13 mL of 1,2-dichloroethane was stirred at 95° C. for 16 h. The solution was partitioned between ethyl acetate and saturated aqueous sodium bicarbonate solution. The organic phase was dried over anhydrous magnesium sulfate, filtered, and concentrated. The residue was purified by silica gel column chromatography eluting with a hexanes:ethyl acetate solution (7:3) to give 0.61 g (sample contains 1.0 EtOAc by ¹H NMR for an effective weight of 0.51g, 82%) of the title compound. ¹H NMR (300 MHz, DMSO-d₆) δ 8.34 (d, J=9 Hz, 2H), 8.26 (d, J=8 Hz, 2H), 8.01 (d, J=8 Hz, 2H), 7.62 (d, J=9 Hz, 2H), 5.84 (s, 1H), 1.46 (m, 2H), 1.11 (s, 3H), 1.04 (s, 3H), 0.90 (m, 3H).

### Example 65b

### Preparation of (1R)-2,2-dimethyl-1-{5-[4-(trifluoromethyl)phenyl]-1,3,4-oxadiazol-2-yl}butyl(1S)-1-[(1S)-1-hydroxy-2-oxo-2-(1H-pyrazol-5-ylamino)ethyl]pentylcarbamate and (1R)-2,2-dimethyl-1-{5-[4-(trifluoromethyl)phenyl]-1,3,4oxadiazol-2-yl}butyl(1S)-1-[(1R)-1-hydroxy-2-oxo-2-(1H-pyrazol-5-ylamino)ethyl]pentylcarbamate

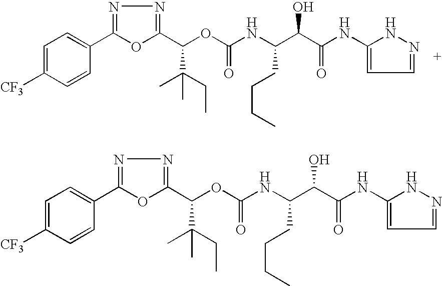

To a solution of 155 mg (0.47 mmol) of tert-butyl (1S)-1-[(1R)-1-hydroxy-2-oxo-2-(1H-pyrazol-5-ylamino)ethyl]pentylcarbamate Et tert-butyl (1S)-1-[(1S)-1-hydroxy-2-oxo-2-(1H-pyrazol-5-ylamino)ethyl]pentylcarbamate in 1.25 mL of dioxane at rt was added 5.1 mL (20.4 mmol) of a 4 M solution of hydrogen chloride in dioxane. The mixture was stirred for 1 h, and then concentrated. The residue was dried under vacuum, and dissolved in 4.1 mL of N,N-dimethylformamide. Then, 197 mg (0.41 mmol) of (1S)-2,2-dimethyl-1-{5-[4-(trifluoromethyl)phenyl)-1,3,4oxadiazol-2-yl}butyl 4-nitrophenyl carbonate was added to followed by 0.33 mL (1.64 mmol) of diisopropylethylamine. The resulting yellow solution was stirred for 1 d at 60° C. It was concentrated, and the residue was partitioned between ethyl acetate and saturated aqueous sodium bicarbonate. The organic phase was concentrated, and the residue was purified by silica gel column chromatography eluting with methanol:chloroform solution (1:9) to yield 0.15 g (65%) of the title compound. ES-LCMS m/z 567 (M+H)⁺ retention time=4.1 min.

### Example 65c

### Preparation of (1R)-2,2-dimethyl-1-{5-[4-(trifluoromethyl)phenyl]-1,3,4-oxadiazol-2-yl}butyl(1S)-1-[oxo(1H-pyrazol-5-ylamino)acetyl]pentylcarbamate

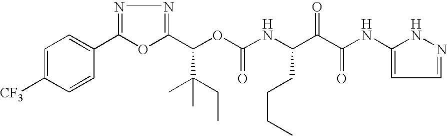

To a solution of 0.144 g (0.25 mmol) of (1R)-2,2-dimethyl-1-{5-[4-(trifluoromethyl)phenyl]-1,3,4-oxadiazol-2-yl}butyl(1S)-1-[(1S)-1-hydroxy-2-oxo-2(1H-pyrazol-5-ylamino)ethyl]pentylcarbamate and (1R)-2,2-dimethyl-1-{5-[4-(trifluoromethyl)phenyl]-1,3,4oxadiazol-2-yl}butyl(1S)-1-[(1R)-1-hydroxy-2-oxo-2-(1H-pyrazol-5-ylamino)ethyl]pentylcarbamate in 2.5 mL of chloroform at rt was added 131 mg (0.31 mmol) of Dess-Martin periodinane. The reaction mixture was stirred for 1.25 h, diluted with ethyl acetate, and then poured into saturated aqueous sodium metabisulfite solution. The resulting mixture was subsequently neutralized with saturated aqueous sodium bicarbonate solution, and the two layers were separated. The organic phase was washed with brine then dried over anhydrous magnesium sulfate, filtered, and concentrated. The residue was purified by silica gel column chromatography eluting with a methanol:chloroform solution (1:9). Fractions containing the product were combined and concentrated yield 60.2 mg (43%) of the title compound. HRMS (C₂₆H₃₁F₃N₆O₅) m/z 565.2386 (M+H)⁺_{Cal}; 565.2395 (M+H)⁺_{Obs}; ES-LCMS m/z 565 (M+H)⁺ retention time=4.2 min.

### Biological Data

The compounds of the present invention elicit important and measurable pharmacological responses. Each of the compounds exemplified in the Examples section bind with high affinity (IC₅₀<10 μM) to the cathepsin K enzyme, as described by the cathepsin K assay recited below.

All assays for cathepsin K were carried out with human and rat recombinant enzyme. Assays for cathepsins S Et V were also carried out with human recombinant enzyme. Assays for human cathepsins B. H. and L were carried out with enzyme, purchased from Athens Research and Technology, Inc., prepared from human liver tissue. Standard assay conditions for the determination of kinetic constants used a fluorogenic peptide substrate, typically (5S,8S)-13-amino-5-benzyl-13-imino-3-methylene-N-(4methyl-2-oxo-2H-chromen-7-yl)-6-oxo-1-phenyl-2-oxa-4,7,12-triazatridecane-8-carboxamide (Cbz-Phe-Arg-AMC), and were determined in 100 mM sodium acetate at pH 5.5 containing 10 mM dithiothreitol and 120 mM sodium chloride. A stock substrate solution of Cbz-Phe-Arg-AMC was prepared at a concentration of 50 mM in dimethyl sulfoxide. This substrate was diluted into the assay for a final substrate concentration of 10 μM in the rat cathepsin K, human cathepsin K, and human cathepsin B assays; a final substrate concentration of 5 μM in the human cathepsin L assay; and a final substrate concentration of 2 μM in the human cathepsin V assay.

A stock substrate solution of benzyl (1S)-1-{[((1S)-1-{[((1S)-4-{[amino(imino)methyl]amino}-1-{[(4-methyl-2-oxo-2H-chromen-7-yl)amino]carbonyl}butyl)amino]carbonyl}-2-methylpropyl)amino]carbonyl}-2-methylpropylcarbamate (Cbz-Val-Val-Arg-AMC) was prepared at a concentration of 10 mM in dimethyl sulfoxide. This substrate was diluted into the assay for a final substrate concentration of 10 μM in the human cathepsin S assay.

A stock substrate solution of (2S)-2-amino-5-{[amino(imino)methyl]amino}-N-(2-naphthyl)pentanamide hydrochloride (L-Arg-β-naphthalamide.HCl) was prepared at a concentration of 10 mM in dimethyl sulfoxide. This substrate was diluted into the assay for a final substrate concentration of 50 ELM in the cathepsin H assay.

All assays contained 10% dimethyl sulfoxide. Independent experiments found that this level of dimethyl sulfoxide had no effect on kinetic enzymatic constants. All assays were conducted at 30° C. Product fluorescence (excitation at 360 nm; emission at 440 nm, (except cathepsin H which used excitation at 340 nm; emission at 420 nm)) was monitored with a PerSeptive Biosystems Cytofluor II fluorescence plate reader. Product progress curves were generated over 2.3 h monitoring the formation of 7-amino-4-methylcoumarin product (or β-naphthalamide for cathepsin H).

Human and Rat Cathepsin K:

Scale-Up and Fermentation: The method of O'Reilly et al. (1994) was used for baculovirus expression with the following details. Two liters of Spodoptera frugiperda (Sf-9) cells (ATCC) were grown in Grace's Supplemented medium (Life Technologies) supplemented with 2 g/L glucose, 10% a fetal bovine serum (HyClone) and 0.10% pluronic F-68 (Life Technologies). Cells were grown in a 6 L shake flask at 150 RPM at 28° C. for 24 h to a density of 106 cells/mL, and then infected at a multiplicity of infection (MOI) of 0.1. The cells continued to grow for 72 h post-infection, before the virus was harvested by centrifugation at 1400× g for 30 min. Virus was titered as described (Summers and Smith, 1987).

One and one-half liters of Trichoplusia ni (T. ni) High Five (TM) cells [JRH Biosciences, Woodland, Calif. (adapted to suspension and serum-free medium)] grown in Excell 405 (TM) medium (JRH Biosciences) with 50 ug/mL gentamicin (Life Technologies) were added to a 15 L stirred tank reactor (Quark Enterprises, Inc) at a density of ˜0.5×106 cells/mL The cells were grown for 24 h at 28° C., 50 RPM, and 50% dissolved oxygen. Cells were then infected at a density of ˜106 cells/mL with an MOI of 1 and grown for 48 h post-infection. Media were separated from cells at a rate of 1 L/min using the Centritech 100 (TM) continuous-flow centrifuge (DuPont) operating at 200× g.

Protein Purification: Media (human and rat) were filtered through a Whatman 3 filter, and then loaded onto a 25 mL Poros HS II (26 mm×47 mm) cation exchange column equilibrated in 25 mM sodium acetate at pH 5.5 (equilibration buffer). The column was washed until the absorbance reached the baseline value, and then the protein was eluted with a linear gradient from 0-2 M sodium chloride in the equilibration buffer. Column fractions were analyzed by SDS-PAGE, N-terminal sequencing, and mass spectrometry. Fractions containing the proform of cathepsin K were pooled and frozen at −80° C. The proform was concentrated in an Amicon Centriprep 10 and fractionated with a Superdex 75 column (26 mm×600 mm, Pharmacia) equilibrated in 400 mM sodium chloride, 25 mM sodium acetate at pH 5.5.

Cathepsin K Activation: The proform of cathepsin K was converted to mature cathepsin K by brief exposure to pH 4 in the presence of 5 mM L-cysteine. Typically, 5 mM L-cysteine was added to 10 mL of approximately 1 mg/mL procathepsin K. One mL of this solution was diluted ten-fold into 450 mM sodium acetate at pH 4.0 containing 5 mM L-cysteine. This solution was reacted at 23° C. for 2 min before neutralization with 2 mL 1.8 M sodium acetate at pH 6.0. The neutralized sample was added to the remaining 9 mL of procathepsin K. The mixture was incubated at 4° C. for 2-3 days. The activated cathepsin K was chromatographed on a Poros HS II column as described above.

Inhibition Studies

Potential inhibitors were evaluated using the progress curve method. Assays were carried out in the presence of variable concentrations of test compound. Reactions were initiated by addition of buffered solutions of inhibitor and substrate to enzyme. Data analysis was conducted according to one of two procedures depending on the appearance of the progress curves in the presence of inhibitors. For those compounds whose progress curves were linear, the enzymatic activity (RATE) was plotted against the concentration of test compound, including inhibitor concentration of zero ([I]=0), and the IC₅₀ determined from a fit of equation 1 to the data,
RATE=Vₘₐₓ/(1+([I]/IC50)) (1)
where Vₘₐₓ is the best fit estimate of the maximal enzymatic activity. Kᵢ values were calculated from IC₅₀ values using equation 2 assuming a competitive model.

$${K}_{i} = {IC}_{50} * [ 1 - \frac{S}{( S + {K}_{m} )} ] \qquad ( 2 )$$

For those compounds whose progress curves showed downward curvature characteristic of time-dependent inhibition, the data from individual sets was analyzed using the computer program DynaFit (Kuzmic, P. Anal. Biochem. 1996, 237, 260-273) to give Ki values according to the following kinetic mechanism:
E+S [symbol] ES
ES→E+P
E→EX
E+I [symbol] EI

TABLE 1
Inhibition of Cathepsin K (Ki in nM)
Cathepsin Inhibitory Activity

| Example | hCat K Ki |
|---|---|
| 1 | +++ |
| 2 | ++ |
| 3 | +++ |
| 4 | ++ |
| 5 | ++ |
| 6 | +++ |
| 7 | +++ |
| 8 | ++ |
| 9 | + |
| 10 | +++ |
| 11 | + |
| 12 | +++ |
| 13 | + |
| 14 | +++ |
| 15 | +++ |
| 16 | + |
| 17 | +++ |
| 18 | + |
| 19 | +++ |
| 20 | + |
| 21 | + |
| 22 | ++ |
| 23 | +++ |
| 24 | ++ |
| 25 | +++ |
| 26 | +++ |
| 27 | +++ |
| 28 | +++ |
| 29 | +++ |
| 30 | +++ |
| 31 | +++ |
| 32 | + |
| 33 | +++ |
| 34 | +++ |
| 35 | ++ |
| 36 | ++ |
| 37 | +++ |
| 38 | +++ |
| 39 | +++ |
| 40 | + |
| 41 | + |
| 42 | +++ |
| 43 | + |
| 44 | +++ |
| 45 | +++ |
| 46 | +++ |
| 47 | +++ |
| 48 | +++ |
| 49 | +++ |
| 50 | +++ |
| 51 | ++ |
| 52 | +++ |
| 53 | ++ |
| 54 | +++ |
| 55 | + |

+Potent inhibitors (10,000-100 nM)
++More potent inhibitors (100-1 nM)
+++Most potent inhibitors (1-0.01 nM)

TABLE 2
Inhibition of Cathepsins (Ki in nM)

| | hCat B | hCat | hCat K | rCat K | hCat | hCat S | hCat V |
|---|---|---|---|---|---|---|---|
| Example | Ki | H Ki | Ki | Ki | L Ki | Ki | Ki |
| 1 | ++ | + | +++ | +++ | ++ | +++ | +++ |
| 7 | ++ | + | +++ | +++ | ++ | +++ | +++ |
| 22 | + | −−− | ++ | + | + | + | + |
| 35 | + | + | ++ | ++ | + | ++ | ++ |
| 37 | ++ | + | +++ | +++ | +++ | +++ | +++ |
| 38 | ++ | + | +++ | ++ | ++ | ++ | ++ |
| 39 | ++ | + | +++ | ++ | ++ | ++ | ++ |

+Potent inhibitors (10,000-100 nM)
++More potent inhibitors (100-1 nM)
+++Most potent inhibitors (1-0.01 nM)

1. A compound of Formula (I): 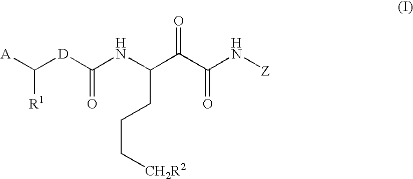
or a salt, solvate, or physiologically functional derivative thereof:
wherein
A is the group defined by (Q³)ₚ-(Q²)ₙ-(Q¹)-(Q)ₘ-, wherein
Q is CH₂ and m is 0, 1, or 2, or
Q is OCH₂ and m is 1, or
Q is N(R′)CH₂ and m is 1, where R′ is hydrogen or C₁-C₆ alkyl;
Q¹ is aryl or heteroaryl;
Q² is CH₂ and n is 0, or 1, or
Q² is CH₂O and n is 1, or
Q² is N(R′) and n is 1, where R′ is hydrogen or C₁-C₆ alkyl;
Q³ is aryl or heteroaryl and p is 0 or 1;
R¹ is C₁-C₆ alkyl, C₃-C₆ cycloalkyl or C₃-C₆ cycloalkyl substituted with C₁-C₆ alkyl;
D is O or S;
R² is hydrogen or —NR³R⁴;
R³, R⁶, and R⁷ are independently selected from hydrogen or C₁-C₆ alkyl;
R⁴ is hydrogen, C₁-C₆ alkyl, —C(O)R⁵, —C(O)OR⁵, —S(O)₂R⁵;
R⁵ is hydrogen, C₁-C₆ alkyl, or —NR⁶R⁷;
Z is the group defined by —(X)ₘ—(X¹), wherein
X is C(R″)(R′″), wherein R″ is hydrogen or C₁-C₆ alkyl, R′″ is hydrogen or C₁-C₆ alkyl, and m is 0, 1, or 2; and
X¹ is aryl, heteroaryl, or heterocyclyl.

2. A compound of Formula (II): 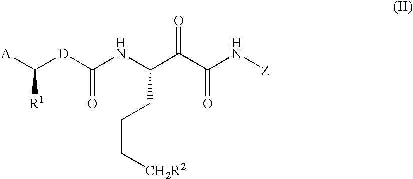
or a salt, solvate, or physiologically functional derivative thereof:
wherein
A is the group defined by (Q³)ₚ-(Q²)ₙ-(Q¹)-(Q)ₘ-, wherein
Q is CH₂ and m is 0, 1, or 2, or
Q is OCH₂ and m is 1, or
Q is N(R′)CH₂ and m is 1, where R′ is hydrogen or C₁-C₆ alkyl;
Q¹ is aryl or heteroaryl;
Q² is CH₂ and n is 0, or 1, or
Q² is CH₂O and n is 1, or
Q² is N(R′) and n is 1, where R′ is hydrogen or C₁-C₆ alkyl;
Q³ is aryl or heteroaryl and p is 0 or 1;
R¹ is C₁-C₆ alkyl, C₃-C₆ cycloalkyl or C₃-C₆ cycloalkyl substituted with C₁-C₆ alkyl;
D is O or S;
R² is hydrogen or —NR³R⁴;
R³, R⁶, and R⁷ are independently selected from hydrogen or C₁-C₆ alkyl;
R⁴ is hydrogen, C₁-C₆ alkyl, —C(O)R⁵, —C(O)OR⁵, —S(O)₂R⁵;
R⁵ is hydrogen, C₁-C₆ alkyl, or —NR⁶R⁷;
Z is the group defined by —(X)ₘ—(X¹), wherein
X is C(R″)(R′″), wherein R″ is hydrogen or C₁-C₆ alkyl, R′″ is hydrogen or C₁-C₆ alkyl, and m is 0, 1, or 2, and
X¹ is aryl, heteroaryl, or heterocyclyl.

3-12. (canceled)

13. A compound as claimed in claim 1, wherein Q¹ is selected from the group 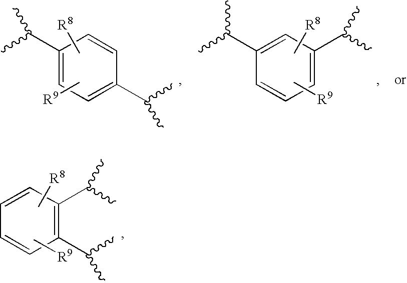
wherein R⁸ and R⁹ are independently selected from hydrogen, halogen, or C₁-C₃ haloalkyl.

14-16. (canceled)

17. A compound as claimed in claim 1, wherein Q¹ is selected from the group

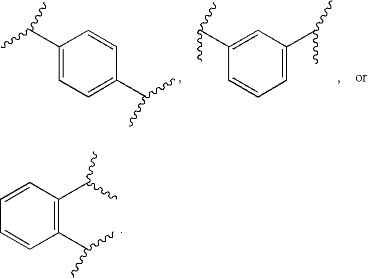

18. A compound as claimed in claim 1, wherein Q¹ is

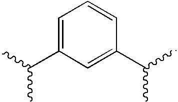

19. (canceled)

20. A compound as claimed in claim 1, wherein Q¹ is selected from

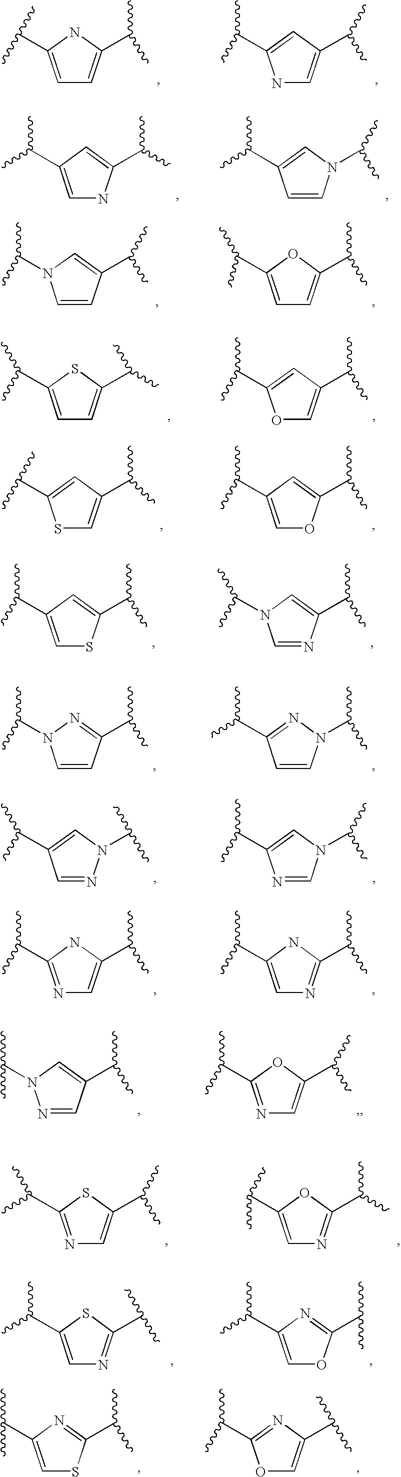

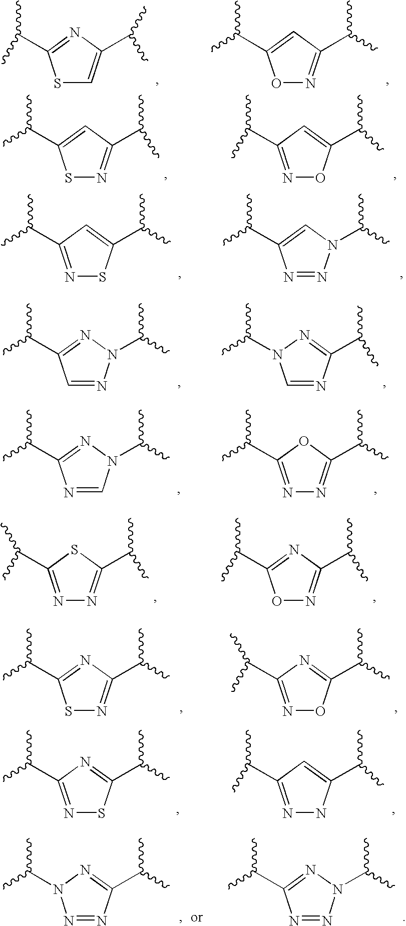

21. A compound as claimed in claim 1, wherein Q¹ is

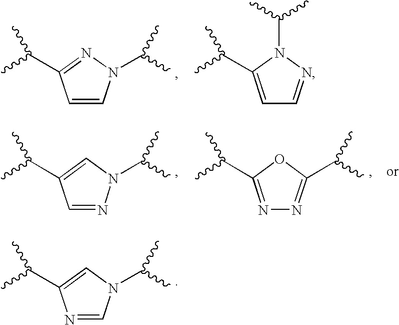

22. A compound as claimed in claim 1, wherein Q¹ is 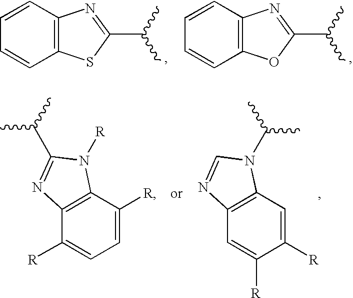
wherein each R is independently hydrogen, halogen, C₁-C₆ alkyl, C₁-C₆ haloalkyl, or C₁-C₆ alkoxy.

23-27. (canceled)

28. A compound as claimed in claim 1, wherein Q³ is selected from the group 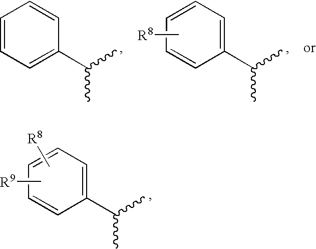
wherein R⁸ and R⁹ are independently selected from halogen or C₁-C₃ haloalkyl.

29. A compound as claimed in claim 1, wherein Q³ is 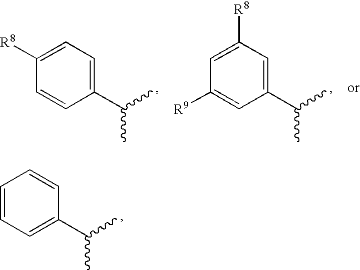
wherein R⁸ and R⁹ are independently selected from halogen or C₁-C₃ haloalkyl.

30-31. (canceled)

32. A compound as claimed in claim 1, wherein Q³ is selected from the group

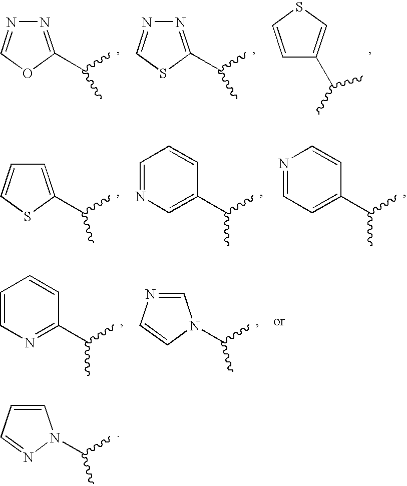

33. A compound as claimed in claim 1, wherein Q³ is selected from the group

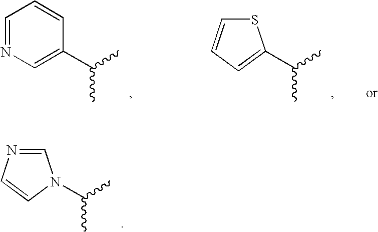

34-39. (canceled)

40. A compound as claimed in claim 1, wherein D is O.

41-46. (canceled)

47. A compound as claimed in claim 1, wherein X¹ is

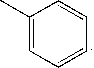

48. (canceled)

49. A compound as claimed in claim 1, wherein X¹ is

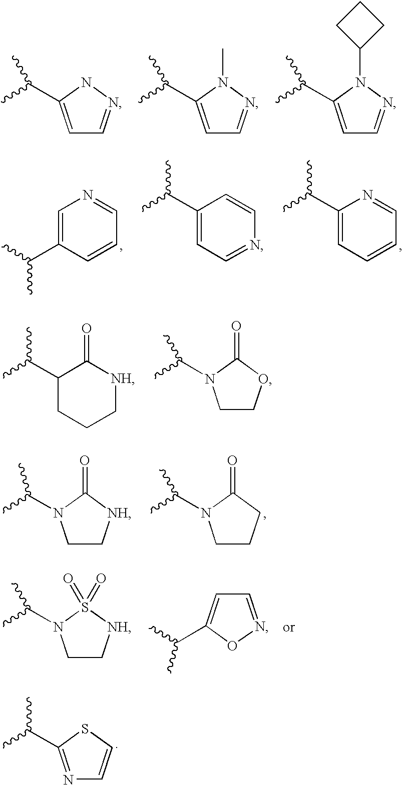

50. A compound as claimed in claim 1, selected from the group consisting of:
(1S)-2,2-dimethyl-1-({3-[4-(trifluoromethyl)phenyl]-1H-pyrazol-1-yl}methyl)propyl(1S)-1-{oxo[(1H-pyrazol-5-ylmethyl)amino]acetyl}pentylcarbamate;
(1R)-2,2-dimethyl-1-({3-[4-(trifluoromethyl)phenyl]-1H-pyrazol-1-yl}methyl)propyl(1S)-1-[oxo(1H-pyrazol-3-ylamino)acetyl]pentylcarbamate;
(1R)-2,2-dimethyl-1-({5-[4-(trifluoromethyl)phenyl]-1,3,4-oxadiazol-2-yl}methyl)propyl(1S)-1-[oxo(1H-pyrazol-3-ylamino)acetyl]pentylcarbamate;
(1R)-1-{[5-(4-fluorophenyl)-1,3,4-oxadiazol-2-yl]methyl}-2,2-dimethylpropyl(1S)-1-{oxo[(3-pyridinylmethyl)amino]acetyl}pentylcarbamate;
(1S)-2,2-dimethyl-1-({3-[4-(trifluoromethyl)phenyl]-1H-pyrazol-1-yl}methyl)propyl(1S)-1-[oxo(2-pyridinylamino)acetyl]pentylcarbamate;
(1S)-1-{[4-(4-fluorophenyl)-1H-imidazol-1-yl]methyl}-2,2-dimethylpropyl(1S)-1-(oxo{[(1R)-1-phenylethyl]amino}acetyl)pentylcarbamate;
(1S)-2,2-dimethyl-1-({4-[4-(trifluoromethyl)phenyl]-1H-imidazol-1-yl}methyl)propyl(1S)-1-[oxo(1H-pyrazol-5-ylamino)acetyl]pentylcarbamate;
(1R)-2,2-dimethyl-1-[(5-phenyl-1,3,4-oxadiazol-2-yl)methyl]butyl(1S)-1-(oxo{[(1R)-1-phenylethyl]amino}acetyl)pentylcarbamate;
(1R)-2,2-dimethyl-1-[(5-phenyl-1,3,4-oxadiazol-2-yl)methyl]butyl(1S)-1-(oxo{[( 1R)-1-phenylethyl]amino}acetyl)pentylcarbamate;
(1R)-2-methyl-1-[(5-phenyl-1,3,4-oxadiazol-2-yl)methyl]propyl(1S)-1-(oxo{[(1R)-1-phenylethyl]amino}acetyl)pentylcarbamate;
(1S)-2-methyl-1-[(5-phenyl-1,3,4-oxadiazol-2-yl)methyl]propyl(1S)-1-(oxo{[(1R)-1-phenylethyl]amino}acetyl)pentylcarbamate;
(1S)-2,2-dimethyl-1-[(4-phenyl-1H-imidazol-1-yl)methyl]propyl(1S)-1-(oxo{[(1R)-1-phenylethyl]amino}acetyl)pentylcarbamate;
(1R)-2,2-dimethyl-1-[(4-phenyl-1H-imidazol-1-yl)methyl]propyl(1S)-1-(oxo{[(1R)-1-phenylethyl]amino}acetyl)pentylcarbamate;
(1S)-2,2-dimethyl-1-({4-[4-(trifluoromethyl)phenyl]-1H-pyrazol-1-yl}methyl)propyl (1S)-1-[oxo(1H-pyrazol-5-ylamino)acetyl]pentylcarbamate;
(1R)-2,2-dimethyl-1-[(5-phenyl-1,3,4-oxadiazol-2-yl)methyl]propyl(1S)-1-(oxo{[(1R)-1-phenylethyl]amino}acetyl)pentylcarbamate;
(1S)-2,2-dimethyl-1-[(5-phenyl-1,3,4-oxadiazol-2-yl)methyl]propyl(1S)-1-(oxo{[(1R)-1-phenylethyl]amino}acetyl)pentylcarbamate;
(1R)-2,2-dimethyl-1-({5-[4-(trifluoromethyl)phenyl]-1,3,4-oxadiazol-2-yl}methyl)propyl(1S)-1-(oxo{[(1R)-1-phenylethyl]amino}acetyl)pentylcarbamate;
(1S)-2,2-dimethyl-1-({5-[4-(trifluoromethyl)phenyl]-1,3,4-oxadiazol-2-yl}methyl)propyl(1S)-1-(oxo{[(1R)-1-phenylethyl]amino}acetyl)pentylcarbamate;
(1R)-1-{[5-(4-fluorophenyl)-1,3,4-oxadiazol-2-yl]methyl}-2,2-dimethylpropyl(1S)-1-(oxo{[(1R)-1-phenylethyl]amino}acetyl)pentylcarbamate;
(1S)-1-{[5-(4-fluorophenyl)-1,3,4-oxadiazol-2-yl]methyl}-2,2-dimethylpropyl(1S)-1-(oxo{[(1R)-1-phenylethyl]amino}acetyl)pentylcarbamate;
(1R)-1-{[5-(4-fluorophenyl)-1,3,4-oxadiazol-2-yl]methyl}-2,2-dimethylpropyl(1S)-1-[oxo(2-pyridinylamino)acetyl]pentylcarbamate;
(1R)-1-{[5-(4-fluorophenyl)-1,3,4-oxadiazol-2-yl]methyl}-2,2-dimethylpropyl(1S)-1-[[(1-methyl-1H-pyrazol-5-yl)amino](oxo)acetyl]pentylcarbamate;
(1R)-1-{[5-(4-fluorophenyl)-1,3,4-oxadiazol-2-yl]methyl}-2,2-dimethylpropyl(1S)-1-[oxo(1H-pyrazol-5-ylamino)acetyl]pentylcarbamate;
(1R)-1-{[5-(4-fluorophenyl)-1,3,4-oxadiazol-2-yl]methyl}-2,2-dimethylpropyl(1S)-1-{oxo[(4-pyridinylmethyl)amino]acetyl}pentylcarbamate;
(1R)-1-{[5-(4-fluorophenyl)-1,3,4-oxadiazol-2-yl]methyl}-2,2-dimethylpropyl(1S)-1-(oxo{[(3S)-2-oxopiperidinyl]amino}acetyl)pentylcarbamate;
(1R)-2,2-dimethyl-1-(2-{5-[4-(trifluoromethyl)phenyl]-1,3,4-oxadiazol-2-yl}ethyl)propyl(1S)-1-[oxo(1H-pyrazol-5-ylamino)acetyl]pentylcarbamate;
(1S)-1-(1H-benzimidazol-1-ylmethyl)-2,2-dimethylpropyl(1S)-1-[oxo(1H-pyrazol-5-ylamino)acetyl]pentylcarbamate;
(1R)-2,2-dimethyl-1-({5-[4-(trifluoromethyl)phenyl]-1,3,4-oxadiazol-2-yl}methyl)propyl(1S)-1-{oxo[(2-oxo-1,3-oxazolidin-3-yl)amino]acetyl}pentylcarbamate;
(1S)-2,2-dimethyl-1-{[3-(trifluoromethyl)-1H-pyrazol-1-yl]methyl}propyl(1S)-1-[oxo(1H-pyrazol-5-ylamino)acetyl]pentylcarbamate;
(1S)-2,2-dimethyl-1-({5-[4-(trifluoromethyl)phenyl]-1H-pyrazol-1-yl}methyl)propyl(1S)-1-[oxo(1H-pyrazol-5-ylamino)acetyl]pentylcarbamate;
(1S)-1-(1,3-benzothiazol-2-yl)-2,2-dimethylpropyl(1S)-1-[oxo(1H-pyrazol-3-ylamino)acetyl]pentylcarbamate;
(1R)-1-(1,3-benzothiazol-2-yl)-2,2-dimethylpropyl(1S)-1-[oxo(1H-pyrazol-5-ylamino)acetyl]pentylcarbamate;
(1S)-2,2-dimethyl-1-{[3-(3-pyridinyl)-i H-pyrazol-1-yl]methyl}propyl(1S)-1-[oxo(1,3-thiazol-2-ylamino)acetyl]pentylcarbamate;
(1S)-1-[(4-benzyl-1H-imidazol-1-yl)methyl]-2,2-dimethylpropyl(1R)-1-[oxo(1H-pyrazol-5-ylamino)acetyl]pentylcarbamate;
(1S)-1-[(4-benzyl-1H-imidazol-1-yl)methyl]-2,2-dimethylpropyl(1R)-1-[oxo(1H-pyrazol-5-ylamino)acetyl]pentylcarbamate;
(1S)-2,2-dimethyl-1-({3-[4-(trifluoromethyl)phenyl]-1H-pyrazol-1-yl}methyl)propyl(1S)-1-[[(⁵-isoxazolylmethyl)amino](oxo)acetyl]pentylcarbamate;
(1S)-1-[(5,6-dichloro-1H-benzimidazol-1-yl)methyl]-2,2-dimethylpropyl(1S)-1-[oxo(1H-pyrazol-5-ylamino)acetyl]pentylcarbamate;
(1S)-2,2-dimethyl-1-{5-[4-(trifluoromethyl)phenyl]-1,3,4-oxadiazol-2-yl}propyl(1S)-1-[oxo(1H-pyrazol-5-ylamino)acetyl]pentylcarbamate;
(1S)-2,2-dimethyl-1-{5-[4-(trifluoromethyl)phenyl]-1,3,4-oxadiazol-2-yl}propyl(1S)-1-{oxo[(2-oxo-1,3-oxazolidin-3-yl)amino]acetyl}pentylcarbamate;
(1R)-2,2-dimethyl-1-{5-[4-(trifluoromethyl)phenyl]-1,3,4-oxadiazol-2-yl}propyl(1S)-1-[oxo(1H-pyrazol-5-ylamino)acetyl]pentylcarbamate;
(1R)-1-[1,1′-biphenyl]-3-yl-2,2-dimethylpropyl(1S)-1-[oxo(1H-pyrazol-5-ylamino)acetyl]pentylcarbamate;
(1S)-1-[1,1′-biphenyl]-3-yl-2,2-dimethylpropyl(1S)-1-[oxo(1H-pyrazol-5-ylamino)acetyl]pentylcarbamate;
1-(4,7-diethoxy-1-methyl-1H-benzimidazol-2-yl)-2,2-dimethylpropyl(1S)-1-[(1H-pyrazol-5-ylamino)carbonyl]pentylcarbamate;
(1S)-2,2-dimethyl-1-{[3-(3-pyridinyl)-1H-pyrazol-1-yl]methyl}propyl(1S)-1-[oxo(1H-pyrazol-5-ylamino)acetyl]pentylcarbamate;
(1S)-2,2-dimethyl-1-{[3-(4-pyridinyl)-1H-pyrazol-1-yl]methyl}propyl(1S)-1-[oxo(1H-pyrazol-5-ylamino)acetyl]pentylcarbamate;
(1S)-2,2-dimethyl-1-({3-[4-(trifluoromethyl)phenyl]-1H-pyrazol-1-yl}methyl)propyl(1S)-1-[oxo(1,3-thiazol-2-ylamino)acetyl]pentylcarbamate;
(1S)-1-{[4-(benzyloxy)phenoxy]methyl}-2,2-dimethylpropyl(1S)-1-[oxo(1H-pyrazol-5-ylamino)acetyl]pentylcarbamate;
(1S)-1-{[4-(aminocarbonyl)phenoxy]methyl}-2,2-dimethylpropyl(1S)-1-[oxo(1H-pyrazol-5-ylamino)acetyl]pentylcarbamate;
(1S)-1-{[4-(1H-imimidazol-1-yl)phenoxy]methyl}-2,2-dimethylpropyl(1S)-1-[oxo(1H-pyrazol-5-ylamino)acetyl]pentylcarbamate;
(1S)-1-({4-[3,5-bis(trifluoromethyl)phenyl]-1H-imidazol-1-yl}methyl)-2,2-dimethylpropyl(1S)-1-[oxo(1H-pyrazol-5-ylamino)acetyl]butylcarbamate;
(1S)-2,2-dimethyl-1-({4-[4-(trifluoromethyl)phenyl]-1H-imidazol-1-yl}methyl)propyl(1S)-1-[oxo(1,3-thiazol-2-ylamino)acetyl]pentylcarbamate;
(1S)-2,2-dimethyl-1-[5-(3-pyridinyl)-1,3,4-oxadiazol-2-yl]propyl(1S)-1-[oxo(1H-pyrazol-5-ylamino)acetyl]pentylcarbamate;
(1R)-2,2-dimethyl-1-[5-(3-pyridinyl)-1,3,4-oxadiazol-2-yl]propyl(1S)-1-[oxo(1H-pyrazol-5-ylamino)acetyl]pentylcarbamate;
(1S)-2,2-dimethyl-1-[5-(4-pyridinyl)-1,3,4-oxadiazol-2-yl]propyl(1S)-1-[oxo(1H-pyrazol-5-ylamino)acetyl]pentylcarbamate; and
(1R)-2,2-dimethyl-1-[5-(4-pyridinyl)-1,3,4-oxadiazol-2-yl]propyl(1S)-1-[oxo(1H-pyrazol-5-ylamino)acetyl]pentylcarbamate;
or a salt, solvate, or physiologically functional derivative thereof.

51. A compound as claimed in claim 1, selected from the group consisting of:
(1S)-2,2-dimethyl-1-(3-thien-2-ylphenyl)propyl(1S)-1-[oxo(1H-pyrazol-5-ylamino)acetyl]pentylcarbamate;
(1R)-2,2-dimethyl-1-(3-thien-2-ylphenyl)propyl(1S)-1-[oxo(1H-pyrazol-5-ylamino)acetyl]pentylcarbamate;
(1S)-1-[(5,6-dichloro-1H-benzimidazol-1-yl)methyl]-2,2-dimethylpropyl(1S)-1-[oxo(pyridin-2-ylamino)acetyl]pentylcarbamate;
(1S)-1-[5-(2,6-dichloropyridin-4-yl)-1,3,4-oxadiazol-2-yl]-2,2-dimethylpropyl(1S)-1-[oxo(1H-pyrazol-5-ylamino)acetyl]pentylcarbamate;
(1R)-1-[5-(2,6-dichloropyridin-4-yl)-1,3,4-oxadiazol-2-yl]-2,2-dimethylpropyl(1S)-1-[oxo(1H-pyrazol-5-ylamino)acetyl]pentylcarbamate;
(1S)-1-(4,7-diethoxy-1-methyl-1H-benzimidazol-2-yl)-2,2-dimethylpropyl(1S)-1-[oxo(1H-pyrazol-5-ylamino)acetyl]pentylcarbamate;
(1R)-1-{5-[3,5-bis(trifluoromethyl)phenyl]-1,3,4-oxadiazol-2-yl}-2,2-dimethylpropyl(1S)-1-[oxo(1H-pyrazol-5-ylamino)acetyl]pentylcarbamate;
(1S)-1-{5-[3,5-bis(trifluoromethyl)phenyl]-1,3,4-oxadiazol-2-yl}-2,2-dimethylpropyl(1S)-1-[oxo(1H-pyrazol-5-ylamino)acetyl]pentylcarbamate;
(1S)-2,2-dimethyl-1-{5-[4-(trifluoromethyl)phenyl]-1,3,4-oxadiazol-2-yl}butyl(1S)-1-[oxo(1H-pyrazol-5-ylamino)acetyl]pentylcarbamate; and
(1R)-2,2-dimethyl-1-{5-[4-(trifluoromethyl)phenyl]-1,3,4-oxadiazol-2-yl}butyl(1S)-1-[oxo(1H-pyrazol-5-ylamino)acetyl]pentylcarbamate;
or a salt, solvate, or physiologically functional derivative thereof.

52. A pharmaceutical composition comprising a therapeutically effective amount of a compound as claimed in claim 1, or a salt, solvate, or a physiologically functional derivative thereof and one or more of pharmaceutically acceptable carriers, diluents and excipients.

53. A method of treating a disorder in a mammal, said disorder being characterized by enhanced bone turnover which can ultimately lead to fracture, comprising: administering to said mammal a therapeutically effective amount of a compound as claimed in claim 1 or a salt, solvate or a physiologically functional derivative thereof.

54. A method of treating a disorder in a mammal, said disorder being characterized by bone loss, comprising: administering to said mammal a therapeutically effective amount of a compound as claimed in claim 1 or a salt, solvate or a physiologically functional derivative thereof.

55-56. (canceled)

57. A method of treating osteoporosis, comprising: administering to said mammal a therapeutically effective amount of a compound as claimed in claim 1, or a salt, solvate or physiologically functional derivative thereof.

58. A method of treating osteoporosis, comprising: administering to said mammal therapeutically effective amounts of (i) a compound as claimed in claim 1, or a salt, solvate or physiologically functional derivative thereof and (ii) at least one bone building agent.
